(19) United States
(12) Patent Application Publication
(10) Pub. No.: US 2024/0124422 A1
(43) Pub. Date: Apr. 18, 2024

(54) MUTANT PI3K-ALPHA INHIBITORS AND THEIR USE AS PHARMACEUTICALS

(71) Applicant: Prelude Therapeutics Incorporated, Wilmington, DE (US)

(72) Inventors: Chun Chen, Claymont, DE (US); Ryan Holmes, Wilmington, DE (US); Soham Maity, Wilmington, DE (US); Andrew P. Combs, Kennett Square, PA (US); Andrew W. Buesking, Wilmington, DE (US); Sarah Pawley, Landenburg, PA (US)

(21) Appl. No.: 18/128,117

(22) Filed: Mar. 29, 2023

Related U.S. Application Data
(60) Provisional application No. 63/385,496, filed on Nov. 30, 2022.
(60) Provisional application No. 63/324,745, filed on Mar. 29, 2022.

(51) Int. Cl. C07D 403/04; C07D 239/95; C07D 401/14; C07D 471/04; C07D 471/08; C07D 487/08

(52) U.S. Cl. CPC C07D 403/04; C07D 239/95; C07D 401/14; C07D 471/04; C07D 471/08; C07D 487/08

(57) ABSTRACT

The disclosure is directed to compounds of Formula I

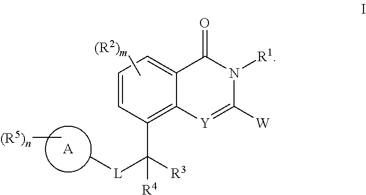

Pharmaceutical compositions comprising compounds of Formula I, as well as methods of their use and preparation, are also described.

### TECHNICAL FIELD

The disclosure is directed to mutant PI3Kα inhibitors and methods of their use.

### BACKGROUND

Class I PI3Ks consist of a p85 regulatory subunit in complex with a p110 catalytic subunit (p110α, β, γ or δ) (1). p110α, coded by the PIK3CA gene shows a broad tissue distribution and the binding of a phosphorylated receptor tyrosine kinase (RTK) activates p110a through the release of a subset of inhibitory contacts with p85. P110α generate phosphatidylinositol3,4,5-trisphosphate (PtdIns(3,4,5)P3; also known as PIP3), which interact with 3-phosphoinositide-binding Pleckstrin homology (PH) domains found in diverse proteins, including protein kinases such as AKT resulting in its phosphorylation at Thr308 and Ser473 triggering a cascade of mitogenic signaling (2). This signaling results in a multitude of cellular effects including proliferation, survival, chemotaxis, cellular trafficking, motility, metabolism, inflammatory and allergic responses, transcription and translation (3).

PIK3CA hotspot mutations are one of the most frequent oncogenic mutations in cancer. Common hotspot mutations in PIK3CA helical (E542K, E545K) and kinase (H1047R) domains function by perturbing local interfaces between p85 and p110a and increasing dynamic events required for catalysis on membranes (1,4). Oncogenic mutations in the PIK3CA gene increase lipid kinase activity and transform cells and are the drivers of the pathology. These mutations are observed in a broad range of cancers including breast, colon, uterine, bladder, cervical, and lung cancer (5,6,7).

Given its key role in cancer PI3Ks have been the focus of extensive drug development. In 2017, the pan-class I PI3K inhibitor copanlisib (Aligopa/BAY 80-6946; Bayer) was approved for follicular lymphoma and in 2019, the PI3Kα inhibitor alpelisib (Piqray/NVP-BYL719; Novartis) was approved for the treatment of advanced breast cancer, in combination with the estrogen receptor (ER) downregulator fulvestrant (1,8). There are a number of PI3Kα selective inhibitors that are in late stage clinical trials (1).

Although these approvals and early clinical data have validated the pathway as a viable drug target, the development of PI3K inhibitors has proved challenging, with progress hampered by poor drug tolerance (9). The lack of Clinical benefit and poor tolerability of pan-class I PI3K and dual PI3Kα/PI3Kδ, or even PI3Kα selective inhibitors have impacted the realization of full clinical utility of these compounds. The toxicity of PI3K inhibitors is dependent on their isoform selectivity profile. Inhibition of PI3Kα is associated with hyperglycemia and rash, whereas inhibition of PI3K6 or RI3Kγ is associated with diarrhea, myelosuppression, and transaminitis (1). A recent study reported that while progression of disease is the largest contributor to Alpelisib discontinuation, adverse events are the leading cause for early drug cessation (10). Shorter Alpelisib exposure is associated with greater cancer progression. Therefore, selective inhibitors of PI3Kα mutants H1047R and/or E545K/E543K while sparing wild type PI3Kα, 3, 7 or 6 could enhance the therapeutic margin and result in therapies that benefit cancer patients that carry these mutations.

Additional small molecule mutant PI3Kα selective inhibitors are needed.
(1) PI3K inhibitors are finally coming of age. Vanhaesebroeck B, Perry M W D, Brown J R, Andre F, Okkenhaug K. Nat Rev Drug Discov. 2021 October; 20(10):741-769. doi: 10.1038/s41573-021-00209-1. Epub 2021 Jun. 14. PMID: 34127844.
(2) AKT/PKB Signaling: Navigating the Network. Manning B D, Toker A. Cell. 2017 Apr. 20; 169(3):381-405. doi: 10.1016/j.cell.2017.04.001. PMID: 28431241.
(3) Fruman, D. A. et al. The PI3K pathway in human disease. Cell 170, 605-635 (2017). PMID: 28802037 PMCID: PMC5726441 DOI: 10.1016/j.cell.2017.07.029.
(4) PIK3C A mutations in advanced cancers: characteristics and outcomes. Janku F, Wheler J J, Naing A, Stepanek V M, Falchook G S, Fu S, Garrido-Laguna I, Tsimberidou A M, Piha-Paul S A, Moulder S L, Lee J J, Luthra R, Hong D S, Kurzrock R. Oncotarget. 2012 December; 3(12):1566-75. doi: 10.18632/oncotarget.716. PMID: 23248156.
(5) Frequency and spectrum of PIK3C A somatic mutations in breast cancer. Martinez-Sáez O, Chic N, Pascual T, Adamo B, Vidal M, González-Farre B, Sanfeliu E, Schettini F, Conte B, Brasó-Maristany F, Rodriguez A, Martinez D, Galván P, Rodriguez A B, Martinez A, Muñoz M, Prat A. Breast Cancer Res. 2020 May 13; 22(1):45. doi: 10.1186/s13058-020-01284-9. PMID: 32404150.
(6) Cancer-specific mutations in PIK3CA are oncogenic in vivo. Bader A G, Kang S, Vogt P K. Proc Natl Acad Sci USA. 2006 Jan. 31; 103(5):1475-9. doi: 10.1073/pnas.0510857103. Epub 2006 Jan. 23. PMID: 16432179.
(7) Oncogenic Signaling Pathways in The Cancer Genome Atlas. Sanchez-Vega F, Mina M, Armenia J, Chatila W K, Luna A, La K C, Dimitriadoy S, Liu D L, Kantheti H S, Saghafinia S, Chakravarty D, Daian F, Gao Q, Bailey M H, Liang W W, Foltz S M, Shmulevich I, Ding L, Heins Z, Ochoa A, Gross B, Gao J, Zhang H, Kundra R, Kandoth C, Bahceci I, Dervishi L, Dogrusoz U, Zhou W, Shen H, Laird P W, Way G P, Greene C S, Liang H, Xiao Y, Wang C, Iavarone A, Berger A H, Bivona T G, Lazar A J, Hammer G D, Giordano T, Kwong L N, McArthur G, Huang C, Tward A D, Frederick M J, McCormick F, Meyerson M; Cancer Genome Atlas Research Network, Van Allen E M, Cherniack A D, Ciriello G, Sander C, Schultz N. Cell. 2018 Apr. 5; 173(2):321-337.e10. doi: 10.1016/j.cell.2018.03.035. PMID: 29625050.
(8) Alpelisib for PIK3C A-Mutated, Hormone Receptor-Positive Advanced Breast Cancer. André F, Ciruelos E, Rubovszky G, Campone M, Loibl S, Rugo H S, Iwata H, Conte P, Mayer I A, Kaufman B, Yamashita T, Lu Y S, Inoue K, Takahashi M, Pipai Z, Longin A S, Mills D, Wilke C, Hirawat S, Juric D; SOLAR-1 Study Group. N Engl J Med. 2019 May 16; 380(20):1929-1940. doi: 10.1056/NEJMoa1813904. PMID: 31091374.
(9) A multidisciplinary approach to optimizing care of patients treated with alpelisib. Rugo H S, Lacouture M E, Goncalves M D, Masharani U, Aapro M S, O'Shaughnessy J A. Breast. 2022 February; 61:156-167. doi: 10.1016/j.breast.2021.12.016. Epub 2021 Dec. 27. PMID: 35016012.
(10) Factors leading to alpelisib discontinuation in patients with hormone receptor positive, human epidermal growth factor receptor-2 negative breast cancer. Cheung Y M, Cromwell G E, Tolaney S M, Min L, McDonnell M E. Breast Cancer Res Treat. 2022 April; 192(2):303-311. doi: 10.1007/s10549-021-06476-1. Epub 2022 Jan. 9. PMID: 35000092.

### SUMMARY OF THE INVENTION

The disclosure is directed to compounds of Formula I:

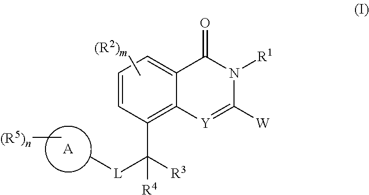

or a pharmaceutically acceptable salt thereof, wherein
   ring A is aryl, a 3-8 membered cycloalkyl ring, a 5-7 membered heteroaryl ring comprising 1-4 heteroatoms selected from N, O, and S, or a 5-7 membered heterocyclic group comprising 1-4 heteroatoms selected from N, O, and S;
   W is a 5-10 membered heteroaryl ring comprising 1-4 heteroatoms selected from N, O, and S, or a 5-12 membered heterocyclic group comprising 1-4 heteroatoms selected from N, O, and S, C₁-C₈ alkyl, haloalkyl, —C₂-C₆ alkenyl, —C₂-C₆ alkynyl, aryl, heteroaryl, cycloalkyl, cycloalkenyl, heterocycloalkenyl, NR^{c}R^{d}, OR^{b}, or SR^{b}; wherein each group is optionally substituted by 1-6 R^{f} groups;
   Y is CR² or N;
   n is 1, 2, 3, 4 or 5;
   m is 1, 2 or 3;
   L is a bond, O, S, NR^{a} or C₁-C₆ alkylene;
   R¹ is H, D, OR^{a}, C₁-C₈ alkoxide, C₁-C₈ alkyl, haloalkyl, —C₃-C₈ cycloalkyl, —C₃-C₁₀ cycloalkenyl, aryl, or heteroaryl; wherein said C₁-C₈ alkoxide, C₁-C₈ alkyl, haloalkyl, C₃-C₈ cycloalkyl, —C₃-C₁₀ cycloalkenyl, aryl, and heteroaryl are optionally substituted by 1-6 R^{f} groups;
   each R² and R⁵ is independently H, D, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR)R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
   each R³ and R⁴ is independently H, D, halogen, C₁-C₈ alkoxide, C₁-C₈ alkyl, haloalkyl, or CN; wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
   or R³ and R⁴ together with the atom to which they are attached are combined to form a C₃-C₇ cycloalkyl or C₄-C₈ heterocycloalkyl;
   each R^{a} is independently H, D, —C(O)R^{b}, —C(O)OR^{b}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{b}R^{b}, —P(O)ORORb, —S(O)R^{b}, —S(O)NR^{c}R^{d}, —S(O)₂R^{b}, —S(O)₂NR^{c}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
   or R^{c} and R^{d}, together with the atom to which they are both attached, form a monocyclic or multicyclic heterocycloalkyl, or a monocyclic or multicyclic heterocyclo-alkenyl group.
   R^{e} is C₃-C₅ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
   each R^{f} is independently H, D, oxo, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups of the are optionally substituted.

Unless otherwise specified, in those embodiments wherein the alkyl group is substituted, the alkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the alkyl group is optionally substituted by 1-6 R groups selected from H, D, halogen, —OH, —CN, —OR^{a}, —SR^{a}, —NR^{a}R^{d}, or NR^{c}R^{d}; or the alkyl group is optionally substituted by 1-6 R^{f} groups.

The term “halo” or halogen refers to chloro, fluoro, bromo, or iodo.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms (e.g., fused or bridged). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure are optionally substituted. Unless otherwise specified, in those embodiments wherein the cycloalkyl group is substituted, the cycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkyl group is optionally substituted by 1-6 R groups selected from H, D, halogen, —OH, —CN, —OR^{a}, —SR^{a}, —NR^{a}R^{d}, or NR^{c}R^{d}; or the cycloalkyl group is optionally substituted by 1-6 R^{f} groups.

The term “cycloalkenyl” when used alone or as part of a substituent group refers to monocyclic or multicyclic, partially saturated ring structure having from 3 to 10 carbon atoms (“C₃-C₁₀”), preferably from 3 to 6 carbon atoms (“C₃-C₆”). Cycloalkenyl groups of the disclosure include monocyclic groups, as well as multicyclic groups such as bicyclic and tricyclic groups. In those embodiments having at least one multicyclic cycloalkenyl group, the cyclic groups can share one common atom (i.e., spirocyclic). In other embodiments having at least one multicyclic cycloalkenyl group, the cyclic groups share two common atoms (e.g., fused or bridged). The term —C₃-C₆ cycloalkenyl refers to a cycloalkenyl group having between three and six carbon atoms. The cycloalkenyl group may be attached at any carbon atom of the partially saturated ring such that the result is a stable structure. Cycloalkenyl groups include groups in which the partially saturated ring is fused to an aryl group. Examples of cycloalkenyl groups include, for example, cyclopropenyl (C₃), cyclobutenyl (C₄), cyclopropenylmethyl (C₄), cyclopentenyl (C₅), cyclohexenyl (C₆), 1-methylcyclopropenyl (C₄), 2-methylcyclopentenyl (C₄), adamantenyl (C₁₀), spiro[3.3]heptenyl, bicyclo[3.3.0]octenyl, indanyl, and the like. Cycloalkenyl groups of the disclosure are optionally substituted. Unless otherwise specified, in those embodiments wherein the cycloalkenyl group is substituted, the cycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkenyl group is optionally substituted by 1-6 R groups selected from H, D, halogen, —OH, —CN, —OR^{a}, —SR^{a}, —NR^{a}R^{d}, or NR^{c}R^{d}; or the cycloalkenyl group is optionally substituted by 1-6 R^{f} groups.

The term “heterocycloalkyl” when used alone or as part of a substituent group refers to any three to twelve membered monocyclic or multicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms (e.g., fused or bridged). The term —C₃-C₆ heterocycloalkyl refers to a heterocycloalkyl group having between three and six carbon ring atoms. The heterocycloalkyl group may be attached at any heteroatom or carbon atom of the group such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, 3-azabicyclo[3.1.0]hexanyl,

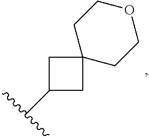 [SMILES: *C1CC2(CCOCC2)C1]

and the like. Heteroycloalkyl groups of the disclosure are optionally substituted. Unless otherwise specified, in those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkyl group is optionally substituted by 1-6 R groups selected from H, D, halogen, —OH, —CN, —OR^{a}, —SR^{a}, —NR^{a}R^{d}, or NR^{c}R^{d}; or the heterocycloalkyl group is optionally substituted by 1-6 R groups.

The term “heterocycloalkenyl” when used alone or as part of a substituent group refers to any three to twelve membered monocyclic or multicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms (e.g., fused or bridged). The term —C₃-C₆ heterocycloalkenyl refers to a heterocycloalkenyl group having between three and six carbon atoms. The heterocycloalkenyl group may be attached at any heteroatom or carbon atom of the partially saturated ring such that the result is a stable structure. Heterocycloalkenyl groups include groups in which the partially saturated ring is fused to an aryl group, such as, for example isoindoline,

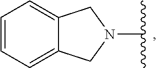 [SMILES: *N1Cc2ccccc2C1]

or in which the partially saturated ring is fused to a heteroaryl group, such as, for example, 6,7-dihydro-5H-pyrrolo[3,4-b]pyridine,

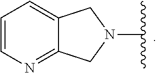 [SMILES: *N1Cc2cccnc2C1]

Heteroycloalkenyl groups of the disclosure are optionally substituted. Unless otherwise specified, in those embodiments wherein the heterocycloalkenyl group is substituted, the heterocycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkenyl group is optionally substituted by 1-6 R groups selected from H, D, halogen, —OH, —CN, —OR^{a}, —SR^{a}, —NR^{a}R^{d}, or NR^{c}R^{d}; or the heterocycloalkenyl group is optionally substituted by 1-6 R^{f} groups.

The term “heterocyclic group,” when used alone or as part of a substituent group, refers to a heterocycloalkyl group or a heterocycloalkenyl group.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure are optionally substituted. Unless otherwise specified, in those embodiments wherein the heteroaryl group is substituted, the heteroaryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heteroaryl group is optionally substituted by 1-6 R groups selected from H, D, halogen, —OH, —CN, —OR^{a}, —SR^{a}, —NR^{a}R^{d}, or NR^{c}R^{d}; or the heteroaryl group is optionally substituted by 1-6 R^{f} groups.

The term “aryl” when used alone or as part of a substituent group refers to a mono- or bicyclic-aromatic carbon ring structure. Aryl rings can include a total of 5, 6, 7, 8, 9, or 10 ring atoms. Examples of aryl groups include but are not limited to, phenyl, napthyl, and the like. Aryl groups of the disclosure are optionally substituted. Unless otherwise specified, in those embodiments wherein the aryl group is substituted, the aryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the aryl group is optionally substituted by 1-6 R groups selected from H, D, halogen, —OH, —CN, —OR^{a}, —SR^{a}, —NR^{a}R^{d}, or NR^{c}R^{d}; or the aryl group is optionally substituted by 1-6 R^{f} groups.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Unless otherwise specified, in those embodiments wherein the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are substituted, they can be optionally substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are optionally substituted by 1-6 R groups selected from H, D, halogen, —OH, —CN, —OR^{a}, —SR^{a}, —NR^{a}R^{d}, or NR^{c}R^{d}; or the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are optionally substituted by 1-6 R groups.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 1 (1977) p. 1-19, each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., entantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof.

Unless indicated otherwise, the description or naming of a particular compound in the specification and claims is intended to include all stereoisomers and mixtures, racemic or otherwise, thereof.

Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

The disclosure is directed to compounds of Formula I.

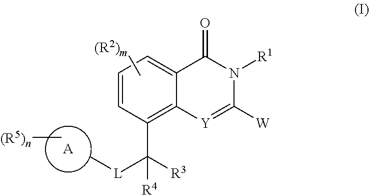

or a pharmaceutically acceptable salt thereof, wherein
   ring A is aryl, a 3-8 membered cycloalkyl ring, a 5-7 membered heteroaryl ring comprising 1-4 heteroatoms selected from N, O, and S, or a 5-7 membered heterocyclic group comprising 1-4 heteroatoms selected from N, O, and S;
   W is a 5-10 membered heteroaryl ring comprising 1-4 heteroatoms selected from N, O, and S, a 5-12 membered heterocyclic group comprising 1-4 heteroatoms selected from N, O, and S, C₁-C₈ alkyl, haloalkyl, —C₂-C₆ alkenyl, —C₂-C₆ alkynyl, aryl, heteroaryl, cycloalkyl, cycloalkenyl, heterocycloalkenyl, NR^{c}R^{d}, OR^{b}, or SR^{b}; wherein each group is optionally substituted by 1-6 R^{f} groups;
   Y is CR² or N;
   n is 1, 2, 3, 4 or 5;
   m is 1, 2 or 3;
   L is a bond, O, S, NR^{a} or C₁-C₆ alkylene;
   R¹ is H, D, OR^{a}, C₁-C₈ alkoxide, C₁-C₈ alkyl, haloalkyl, —C₃-C₈ cycloalkyl, —C₃-C₁₀ cycloalkenyl, aryl, or heteroaryl; wherein said C₁-C₈ alkoxide, C₁-C₈ alkyl, haloalkyl, C₃-C₈ cycloalkyl, —C₃-C₁₀ cycloalkenyl, aryl, and heteroaryl are optionally substituted by 1-6 R^{f} groups;
   each R² and R⁵ is independently H, D, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR)R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
   each R³ and R⁴ is independently H, D, halogen, C₁-C₈ alkoxide, C₁-C₈ alkyl, haloalkyl, or CN; wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
   or R³ and R⁴ together with the atom to which they are attached are combined to form a C₃-C₇ cycloalkyl or C₄-C₈ heterocycloalkyl;
   each R^{a} is independently H, D, —C(O)R^{b}, —C(O)OR^{c}, —C(O)NR^{c}R^{d}, —C(═NR^{b})NR^{b}R^{c}, —C(═NOR^{b})R^{b}R^{c}, —C(═NCN)NR^{b}R^{c}, —P(OR^{c})₂, —P(O)R^{b}R^{b}, —P(O)OR^{c}OR^{b}, —S(O)R^{b}, —S(O)NR^{c}R^{d}, —S(O)₂R^{b}, —S(O)₂NR^{c}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
   or R^{c} and R^{d}, together with the atom to which they are both attached, form a monocyclic or multicyclic heterocycloalkyl, or a monocyclic or multicyclic heterocyclo-alkenyl group.
   R^{e} is C₃-C₅ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
   each R^{f} is independently H, D, oxo, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, ring A in Formula (I) is a 5-7-membered heteroaryl. In some embodiments, ring A is a 5-membered heteroaryl. In other embodiments, ring A is a 6-membered heteroaryl. In yet other embodiments, ring A is a 7-membered heteroaryl.

In some embodiments, ring A in Formula (I) is aryl, a 3-8 membered cycloalkyl ring, a 5-7 membered heteroaryl ring comprising 1-4 heteroatoms selected from N, O, and S, or a 5-7 membered heterocyclic group comprising 1-4 heteroatoms selected from N, O, and S.

In some embodiments, ring A in Formula (I) is aryl. In some embodiments, ring A in Formula (I) is a phenyl ring. In other embodiments, ring A in Formula (I) is a 3-8 membered cycloalkyl ring. In other embodiments, ring A in Formula (I) is a 5-7 membered heteroaryl ring comprising 1-4 heteroatoms selected from N, O, and S. In other embodiments, ring A in Formula (I) is a 5-7 membered heterocyclic group comprising 1-4 heteroatoms selected from N, O, and S.

In some embodiments, W in Formula (I) is a 5-10 membered heteroaryl ring comprising 1-4 heteroatoms selected from N, O, and S, a 5-12 membered heterocyclic group comprising 1-4 heteroatoms selected from N, O, and S, C₁-C₈ alkyl, haloalkyl, —C₂-C₆ alkenyl, —C₂-C₆ alkynyl, aryl, heteroaryl, cycloalkyl, cycloalkenyl, heterocycloalkenyl, NR^{c}R^{d}, OR^{b}, or SR^{b}, wherein each group is optionally substituted by 1-6 R^{f} groups.

In some embodiments, W in Formula (I) is a 5-12 membered heterocyclic group comprising 1-4 heteroatoms selected from N, O, and S. In some embodiments, the 5-12 membered heterocyclic group is an isoindoline group or a piperidine group. In some embodiments, the 5-12 membered heterocyclic group is an isoindoline group. In some embodiments, the 5-12 membered heterocyclic group is a piperidine group. In some embodiments, W in Formula (I) is NR^{c}R^{d}. In some embodiments, W in Formula (I) is substituted by 1-6 R groups.

In other embodiments, W in Formula (I) is a 5-10 membered heteroaryl ring comprising 1-4 heteroatoms selected from N, O, and S. In other embodiments, W in Formula (I) is C₁-C₈ alkyl. In other embodiments, W in Formula (I) is haloalkyl. In other embodiments, W in Formula (I) is —C₂-C₆ alkenyl. In other embodiments, W in Formula (I) is —C₂-C₆ alkynyl. In other embodiments, W in Formula (I) is aryl. In other embodiments, W in Formula (I) is heteroaryl. In other embodiments, W in Formula (I) is cycloalkyl. In other embodiments, W in Formula (I) is cycloalkenyl. In other embodiments, W in Formula (I) is heterocycloalkenyl. In other embodiments, W in Formula (I) is NR^{c}R^{d}. In other embodiments, W in Formula (I) is NR^{c}R^{d}. In other embodiments, W in Formula (I) is OR^{b}. In other embodiments, W in Formula (I) is SR^{b}. In some embodiments, W in Formula (I) is substituted by 1 or 2 R groups.

In some embodiments, Y in Formula (I) is CR² or N. In some embodiments, Y in Formula (I) is N. In other embodiments, Y in Formula (I) is CR². In other embodiments, Y in Formula (I) is CH.

In some embodiments, n in Formula (I) is 1, 2, 3, 4 or 5. In some embodiments, n in Formula (I) is 1. In other embodiments, n in Formula (I) is 2. In yet other embodiments, n in Formula (I) is 3. In yet other embodiments, n in Formula (I) is 4. In yet other embodiments, n in Formula (I) is 5.

In some embodiments, m in Formula (I) is 1, 2 or 3. In some embodiments, m in Formula (I) is 1. In other embodiments, m in Formula (I) is 2. In yet other embodiments, m in Formula (I) is 3.

In some embodiments, L in Formula I is a bond, O, S, NR^{a} or C₁-C₆ alkylene. In some embodiments, L in Formula (I) is NR^{a}. In some embodiments, L in Formula (I) is NH. In some embodiments, L in Formula (I) is NC₁-C₈ alkyl. In some embodiments, L in Formula (I) is N-methyl. In other embodiments, L in Formula (I) is a bond. In other embodiments, L in Formula (I) is O. In other embodiments, L in Formula (I) is S. In other embodiments, L in Formula (I) is C₁-C₆ alkylene. In other embodiments, L in Formula (I) is methylene.

In some embodiments, R¹ in Formula I is H, D, OR^{a}, C₁-C₈ alkoxide, C₁-C₈ alkyl, haloalkyl, —C₃-C₅ cycloalkyl, —C₃-C₈ cycloalkenyl, aryl, or heteroaryl; wherein said C₁-C₈ alkoxide, C₁-C₈ alkyl, haloalkyl, C₃-C₈ cycloalkyl, —C₃-C₁₀ cycloalkenyl, aryl, and heteroaryl are optionally substituted by 1-6 R groups.

In some embodiments, R¹ in Formula I is H. In some embodiments, R¹ in Formula I is D. In some embodiments, R¹ in Formula I is OR^{a}. In some embodiments, R¹ in Formula I is C₁-C₈ alkoxide. In some embodiments, R¹ in Formula I is C₁-C₈ alkyl. In some embodiments, R¹ in Formula I is haloalkyl. In some embodiments, R¹ in Formula I is C₃-C₈ cycloalkyl. In some embodiments, R¹ in Formula I is C₃-C₈ cycloalkenyl. In some embodiments, R¹ in Formula I is aryl. In some embodiments, R¹ in Formula I is heteroaryl. In some embodiments, the C₁-C₈ alkoxide, C₁-C₈ alkyl, haloalkyl, C₃-C₈ cycloalkyl, —C₃-C₁₀ cycloalkenyl, aryl, or heteroaryl are optionally substituted by 1-6 R groups.

In other embodiments, R¹ in Formula I is H. In other embodiments, R¹ in Formula I is C₁-C₈alkyl. In other embodiments, R¹ in Formula I is methyl.

In some embodiments, each R² in Formula I is independently H, D, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR_{c}R_{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, at least one R² in Formula I is H. In some embodiments, at least one R² in Formula I is D. In some embodiments, at least one R² in Formula I is halogen. In some embodiments, at least one R² in Formula I is C₁-C₈ alkoxide. In some embodiments, at least one R2 in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In other embodiments, at least one R² in Formula I is haloalkyl. In other embodiments, at least one R² in Formula I is —OH. In other embodiments, at least one R² in Formula I is —CN. In other embodiments, at least one R² in Formula I is —NO₂. In other embodiments, at least one R² in Formula I is —C₂-C₆ alkenyl. In other embodiments, at least one R² in Formula I is —C₂-C₆ alkynyl. In other embodiments, at least one R² in Formula I is aryl. In other embodiments, at least one R2 in Formula I is hetereoaryl. In other embodiments, at least one R² in Formula I is cycloalkyl. In other embodiments, at least one R² in Formula I is cycloalkenyl. In other embodiments, at least one R² in Formula I is heterocycloalkyl. In some embodiments, at least one R² in Formula I is heterocycloalkenyl. In other embodiments, at least one R² in Formula I is —OR^{a}. In other embodiments, at least one R² in Formula I is —SR^{a}. In other embodiments, at least one R² in Formula I is —NR^{c}R^{d}. In other embodiments, at least one R² in Formula I is —NR^{a}R^{c}. In other embodiments, at least one R² in Formula I is —C(O)R^{b}. In other embodiments, at least one R² in Formula I is —OC(O)R^{b}. In other embodiments, at least one R² in Formula I is —C(O)OR^{b}. In other embodiments, at least one R² in Formula I is —C(O)NR^{c}R^{d}. In other embodiments, at least one R² in Formula I is —S(O)R^{b}. In other embodiments, at least one R² in Formula I is —S(O)₂NR^{c}R^{d}. In other embodiments, at least one R² in Formula I is —S(O)(═NR^{b})R^{b}. In other embodiments, at least one R² in Formula I is —SF₅. In other embodiments, at least one R² in Formula I is —P(O)R^{b}R^{b}. In other embodiments, at least one R² in Formula I is —P(O)(OR^{b})(OR^{b}). In other embodiments, at least one R² in Formula I is —B(OR^{c})(OR^{d}). In other embodiments, at least one R² in Formula I is —S(O)₂R^{b}. In other embodiments, at least one R² in Formula I is —C(O)NR^{b}OR^{b}. In other embodiments, at least one R2 in Formula I is —S(O)₂OR^{b}. In other embodiments, at least one R² in Formula I is —OS(O)₂OR^{b}. In other embodiments, at least one R² in Formula I is —OPO(OR^{b})(OR^{b}).

In some embodiments, at least one R² in Formula I is H, C₁-C₈ alkyl, CF₃, Br, F, CN or CHF₂. In some embodiments, at least one R² in Formula I is H. In some embodiments, at least one R² in Formula I is C₁-C₈alkyl. In some embodiments, at least one R² in Formula I is methyl. In some embodiments, at least one R² in Formula I is CF₃. In some embodiments, at least one R² in Formula I is Br. In some embodiments, at least one R² in Formula I is F. In some embodiments, at least one R² in Formula I is CN. In some embodiments, at least one R² in Formula I is CHF₂.

In some embodiments, R³ in Formula I is H, D, halogen, C₁-C₈ alkoxide C₁-C₈ alkyl, haloalkyl, or CN; wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, R³ in Formula I is H. In some embodiments, R³ in Formula I is D. In some embodiments, R³ in Formula I is halogen. In some embodiments, R³ in Formula I is C₁-C₈ alkoxide. In some embodiments, R³ in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}. In some embodiments, R³ in Formula I is haloalkyl. In some embodiments, R³ in Formula I is CN.

In other embodiments, R³ in Formula I is H or C₁-C₈ alkyl. In other embodiments, R³ in Formula I is H. In other embodiments, R³ in Formula I is C₁-C₈alkyl. In other embodiments, R³ in Formula I is methyl.

In some embodiments, R⁴ in Formula I is H, D, halogen, C₁-C₈ alkoxide C₁-C₈ alkyl, haloalkyl, or CN; wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, R⁴ in Formula I is H. In some embodiments, R⁴ in Formula I is D. In some embodiments, R⁴ in Formula I is halogen. In some embodiments, R⁴ in Formula I is C₁-C₈ alkoxide. In some embodiments, R⁴ in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}. In some embodiments, R⁴ in Formula I is haloalkyl. In some embodiments, R⁴ in Formula I is CN.

In other embodiments, R⁴ in Formula I is H or C₁-C₈ alkyl. In other embodiments, R⁴ in Formula I is H. In other embodiments, R⁴ in Formula I is C₁-C₈alkyl. In other embodiments, R4 in Formula I is methyl.

In some embodiments, R³ and R⁴ in Formula I together with the atom to which they are attached are combined to form a C₃-C₇ cycloalkyl or C₄-C₈ heterocycloalkyl. In some embodiments, R³ and R⁴ in Formula I together with the atom to which they are attached are combined to form a C₃-C₇ cycloalkyl. In some embodiments, R³ and R⁴ in Formula I together with the atom to which they are attached are combined to form a C₄-C₈ heterocycloalkyl.

In some embodiments, each R⁵ in Formula I is independently H, D, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, at least one R⁵ in Formula I is H. In some embodiments, at least one R⁵ in Formula I is D. In some embodiments, at least one R⁵ in Formula I is halogen. In some embodiments, at least one R⁵ in Formula I is C₁-C₈ alkoxide. In some embodiments, at least one R⁵ in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In other embodiments, at least one R⁵ in Formula I is haloalkyl. In other embodiments, at least one R⁵ in Formula I is —OH. In other embodiments, at least one R⁵ in Formula I is —CN. In other embodiments, at least one R⁵ in Formula I is —NO₂. In other embodiments, at least one R⁵ in Formula I is —C₂-C₆ alkenyl. In other embodiments, at least one R⁵ in Formula I is —C₂-C₆ alkynyl. In other embodiments, at least one R⁵ in Formula I is aryl. In other embodiments, at least one R⁵ in Formula I is hetereoaryl. In other embodiments, at least one R⁵ in Formula I is cycloalkyl. In other embodiments, at least one R⁵ in Formula I is cycloalkenyl. In other embodiments, at least one R⁵ in Formula I is heterocycloalkyl. In some embodiments, at least one R⁵ in Formula I is heterocycloalkenyl. In other embodiments, at least one R⁵ in Formula I is —OR^{a}. In other embodiments, at least one R⁵ in Formula I is —SR^{a}. In other embodiments, at least one R⁵ in Formula I is —NR^{c}R^{d}. In other embodiments, at least one R⁵ in Formula I is —NR^{a}R^{c}. In other embodiments, at least one R⁵ in Formula I is —C(O)R^{b}. In other embodiments, at least one R⁵ in Formula I is —OC(O)R^{b}. In other embodiments, at least one R⁵ in Formula I is —C(O)OR^{b}. In other embodiments, at least one R⁵ in Formula I is —C(O)NR^{c}R^{d}. In other embodiments, at least one R⁵ in Formula I is —S(O)R^{b}. In other embodiments, at least one R⁵ in Formula I is —S(O)₂NR^{c}R^{d}. In other embodiments, at least one R⁵ in Formula I is —S(O)(═NR^{b})R^{b}. In other embodiments, at least one R⁵ in Formula I is —SF₅. In other embodiments, at least one R⁵ in Formula I is —P(O)R^{b}R^{b}. In other embodiments, at least one R⁵ in Formula I is —P(O)(OR^{b})(OR^{b}). In other embodiments, at least one R⁵ in Formula I is —B(OR^{c})(OR^{d}). In other embodiments, at least one R⁵ in Formula I is —S(O)₂R^{b}. In other embodiments, at least one R⁵ in Formula I is —C(O)NR^{b}OR. In other embodiments, at least one R⁵ in Formula I is —S(O)₂OR^{b}. In other embodiments, at least one R⁵ in Formula I is —OS(O)₂OR^{b}. In other embodiments, at least one R⁵ in Formula I is —OPO(OR^{b})(OR^{b}).

In some embodiments, at least one R⁵ in Formula I is a carboxylic acid group or isostere thereof. In some embodiments, at least one R⁵ in Formula I is a carboxylic acid group. In some embodiments, at least one R⁵ in Formula I is —CO₂H. In other embodiments, at least one R⁵ in Formula I is an isostere of a carboxylic acid group.

In some embodiments, each R^{a} in Formula I is independently H, D, —C(O)R^{b}, —C(O)OR^{c}, —C(O)NR^{c}R^{d}, —C(═NR^{b})NR^{b}R^{c}, —C(═NOR)NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl.

In some embodiments, R^{a} in Formula I is H. In some embodiments, R^{a} in Formula I is D. In some embodiments, R^{a} in Formula I is —C(O)R^{b}. In some embodiments, R^{a} in Formula I is —C(O)OR^{c}. In some embodiments, R^{a} in Formula I is —C(O)NR^{c}R^{d}. In some embodiments, R^{a} in Formula I is —C(═NR^{b})NR^{b}R^{c}. In some embodiments, R^{a} in Formula I is C(═NO)NR^{b}R^{c}. In some embodiments, R^{a} in Formula I is —C(═NCN)NR^{b}R^{c}.

In other embodiments, R^{a} in Formula I is —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR₃, and the like. In yet other embodiments, R^{a} in Formula I is —C₁-C₁₀alkyl, —C₂-C₁₀ alkenyl, —C₂-C₁₀ alkynyl, aryl, cycloalkyl, cycloalkenyl, heteroaryl, heterocycloalkyl, heterocycloalkenyl, and the like.

In some embodiments, each R^{b} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{b} in Formula I is H. In some embodiments, R^{b} in Formula I is D. In some embodiments, R^{b} in Formula I is —C₁-C₆ alkyl. In some embodiments, R^{b} in Formula I is —C₂-C₆ alkenyl. In some embodiments, R^{b} in Formula I is —C₂-C₆ alkynyl. In other embodiments, R^{b} in Formula I is aryl. In other embodiments, R^{b} in Formula I is cycloalkyl. In other embodiments, R^{b} in Formula I is cycloalkenyl. In other embodiments, R^{b} in Formula I is heteroaryl. In other embodiments, R^{b} in Formula I is heterocycloalkyl. In other embodiments, R^{b} in Formula I is heterocycloalkenyl.

In some embodiments, each R^{c} or R^{d} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{c} or R^{d} in Formula I is H. In some embodiments, R^{c} or R^{d} in Formula I is D. In some embodiments, R^{c} or R^{d} in Formula I is —C₁-C₁₀ alkyl. In some embodiments, R^{c} or R^{d} in Formula I is —C₂-C₆ alkenyl. In some embodiments, R^{c} or R^{d} in Formula I is —C₂-C₆ alkynyl. In other embodiments, R^{c} or R^{d} in Formula I is —OC₁-C₆alkyl. In other embodiments, R^{c} or R^{d} in Formula I is —O-cycloalkyl. In other embodiments, R^{c} or R^{d} in Formula I is aryl. In other embodiments, R^{c} or R^{d} in Formula I is cycloalkyl. In other embodiments, R^{c} or R^{d} in Formula I is cycloalkenyl. In other embodiments, R^{c} or R^{d} in Formula I is heteroaryl. In other embodiments, R^{c} or R^{d} in Formula I is heterocycloalkyl. In other embodiments, R^{c} or R^{d} in Formula I is heterocycloalkenyl.

In yet other embodiments, R^{c} and R^{d} in Formula I, together with the atom to which they are both attached, form a monocyclic or multicyclic heterocycloalkyl, or a monocyclic or multicyclic heterocyclo-alkenyl group. In yet other embodiments, R^{c} and R^{d} in Formula I form a monocyclic heterocycloalkyl. In yet other embodiments, R^{c} and R^{d} in Formula I form a multicyclic heterocycloalkyl. In yet other embodiments, R^{c} and R^{d} in Formula I form a monocyclic heterocyclo-alkenyl group. In yet other embodiments, R^{c} and R^{d} in Formula I form a multicyclic heterocyclo-alkenyl group.

In some embodiments, R^{e} in Formula I is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups.

In some embodiments, R^{e} in Formula I is C₃-C₈ cycloalkyl optionally substituted by 1-6 R^{f} groups. In some embodiments, R^{e} in Formula I is heterocycloalkyl optionally substituted by 1-6 R^{f} groups. In some embodiments, the heterocycloalkyl is attached to (C₁-C₆-alkyl) through a carbon atom of the heterocycloalkyl group. In some embodiments, the heterocycloalkyl is attached to (C₁-C₆-alkyl) through a sulfur atom of the heterocycloalkyl group. In other embodiments, R^{e} in Formula I is cycloalkenyl optionally substituted by 1-6 R^{f} groups. In other embodiments, R^{e} in Formula I is heterocycloalkenyl optionally substituted by 1-6 R^{f} groups. In other embodiments, the heterocycloalkenyl is attached to (C₁-C₆-alkyl) through a carbon atom of the heterocycloalkenyl group. In other embodiments, the heterocycloalkenyl is attached to (C₁-C₆-alkyl) through a sulfur atom of the heterocycloalkenyl group. In yet other embodiments, R^{e} in Formula I is aryl optionally substituted by 1-6 R^{f} groups. In yet other embodiments, R^{e} in Formula I is heteroaryl optionally substituted by 1-6 R^{f} groups.

In some embodiments, R^{e} in Formula I is azetidine or piperidine optionally substituted by 1-6 R^{f} groups, pyrazole optionally substituted by 1-6 R^{f} groups, phenyl optionally substituted by 1-6 R^{f} groups or cycloalkyl optionally substituted by 1-6 R^{f} groups. In some embodiments, R^{e} in Formula I is azetidine optionally substituted by 1-6 R^{f} groups. In some embodiments, R^{e} in Formula I is piperidine optionally substituted by 1-6 R^{f} groups. In some embodiments, R^{e} in Formula I is phenyl optionally substituted by 1-6 R^{f} groups. In some embodiments, R^{e} in Formula I is cycloalkyl optionally substituted by 1-6 R^{f} groups.

In some embodiments, each R^{f} in Formula I is independently H, D, oxo, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, R^{f} in Formula I is H. In some embodiments, R^{f} in Formula I is D. In some embodiments, R^{f} in Formula I is oxo. In some embodiments, R^{f} in Formula I is halogen. In some embodiments, R^{f} in Formula I is C₁-C₈ alkoxide. In some embodiments, R^{f} in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}. In some embodiments, R^{f} in Formula I is haloalkyl. In some embodiments, R^{f} in Formula I is —OH. In some embodiments, R^{f} in Formula I is —CN. In some embodiments, R^{f} in Formula I is —NO₂. In some embodiments, R^{f} in Formula I is —C₂-C₆ alkenyl. In some embodiments, R^{f} in Formula I is —C₂-C₆ alkynyl. In some embodiments, R^{f} in Formula I is aryl. In some embodiments, R^{f} in Formula I is heteroaryl. In some embodiments, R^{f} in Formula I is cycloalkyl. In other embodiments, R^{f} in Formula I is cycloalkenyl. In other embodiments, R^{f} in Formula I is heterocycloalkyl. In other embodiments, R^{f} in Formula I is heterocycloalkenyl. In other embodiments, R^{f} in Formula I is —OR^{a}. In other embodiments, R^{f} in Formula I is —SR^{a}. In other embodiments, R^{f} in Formula I is —NR^{c}R^{d}. In other embodiments, R^{f} in Formula I is —NR^{a}R^{c}. In other embodiments, R^{f} in Formula I is —C(O)R^{b}. In other embodiments, R^{f} in Formula I is —OC(O)R^{b}. In other embodiments, R^{f} in Formula I is —C(O)OR^{b}. In other embodiments, R^{f} in Formula I is —C(O)NR^{c}R^{d}. In yet other embodiments, R^{f} in Formula I is —S(O)R^{b}. In yet other embodiments, R^{f} in Formula I is —S(O)₂NR^{c}R^{d}. In yet other embodiments, R^{f} in Formula I is —S(O)(═NR^{b})R^{b}. In yet other embodiments, R^{f} in Formula I is —SF₅. In yet other embodiments, R^{f} in Formula I is —P(O)R^{b}R^{b}. In yet other embodiments, R^{f} in Formula I is —P(O)(OR^{b})(OR^{b}). In yet other embodiments, R^{f} in Formula I is —B(OR^{c})(OR^{d}). In yet other embodiments, R^{f} in Formula I is —S(O)₂R^{b}. In yet other embodiments, R^{f} in Formula I is —C(O)NR^{b}OR^{b}. In yet other embodiments, R^{f} in Formula I is —S(O)₂OR^{b}. In yet other embodiments, R^{f} in Formula I is —OS(O)₂OR^{b}. In yet other embodiments, R^{f} in Formula I is —OPO(OR^{b})(OR^{b}).

In some embodiments, the compounds of Formula (I) are the pharmaceutically acceptable salts. In some embodiments, the compounds of Formula (I) are solvates. In some embodiments, the compounds of Formula (I) are N-oxides. In some embodiments, the compounds of Formula (I) are stereoisomers.

In some embodiments, the compounds of Formula (I) are represented by compounds of Formula II

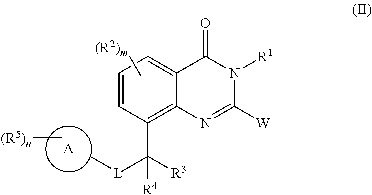

or a pharmaceutically acceptable salt or stereoisomer thereof; wherein each R¹, R², R³, R⁴, R⁵, m, n, W, L, and ring A are defined with respect to Formula (I).

In some embodiments, the compounds of Formula (I) are represented by compounds of Formula III

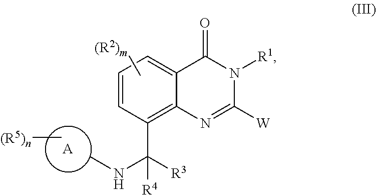

or a pharmaceutically acceptable salt or stereoisomer thereof; wherein each R¹, R², R³, R⁴, R⁵, m, n, W and ring A are defined with respect to Formula (I).

In some embodiments, the compounds of Formula (I) are represented by compounds of Formula IV

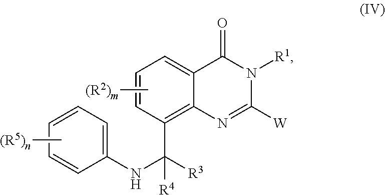

or a pharmaceutically acceptable salt or stereoisomer thereof; wherein each R¹, R², R³, R⁴, R⁵, m, n, and W are defined with respect to Formula (I).

In some embodiments, the compounds of Formula (I) are represented by compounds of Formula V

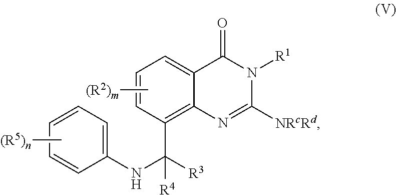

or a pharmaceutically acceptable salt or stereoisomer thereof; wherein each R¹, R², R³, R⁴, R⁵, R^{c}, R^{d}, m, and n are defined with respect to Formula (I).

In some embodiments, the compounds of Formula (I) are represented by compounds of Formula VI

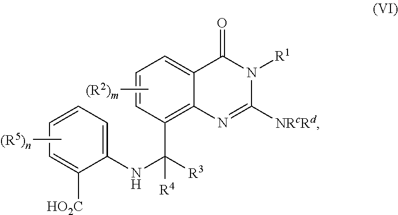

or a pharmaceutically acceptable salt or stereoisomer thereof; wherein each R¹, R², R³, R⁴, R⁵, R^{c}, R^{d}, m, and n are defined with respect to Formula (I).

In yet further embodiments, the compounds of Formula (I) are:
2-((1-(2-(isoindolin-2-yl)-3,6-dimethyl-4-oxo-3,4-dihydroquinazolin-8-yl)ethyl)amino) benzoic acid;
2-((1-(2-(5-fluoroisoindolin-2-yl)-3,6-dimethyl-4-oxo-3,4-dihydroquinazolin-8-yl)ethyl) amino)benzoic acid;
2-((1-(2-(4,4-dimethylpiperidin-1-yl)-3,6-dimethyl-4-oxo-3,4-dihydroquinazolin-8-yl)ethyl) amino)benzoic acid;
   or a pharmaceutically acceptable salt thereof.

In yet further embodiments, the compounds of Formula (I) are:
2-((1-(2-(isoindolin-2-yl)-3,6-dimethyl-4-oxo-3,4-dihydroquinazolin-8-yl)ethyl)amino) benzamide;
2-((1-(2-(isoindolin-2-yl)-3,6-dimethyl-4-oxo-3,4-dihydroquinazolin-8-yl)ethyl)amino)-N-methoxybenzamide;
2-((1-(2-(isoindolin-2-yl)-3,6-dimethyl-4-oxo-3,4-dihydroquinazolin-8-yl)ethyl)amino) benzenesulfonamide;
2-((1-(2-(isoindolin-2-yl)-3,6-dimethyl-4-oxo-3,4-dihydroquinazolin-8-yl)ethyl)amino)-N-methylbenzenesulfonamide;
2-(isoindolin-2-yl)-3,6-dimethyl-8-(1-((2-(methylsulfonyl)phenyl)amino)ethyl) quinazolin-4(3H)-one;
8-(1-((2-(1H-tetrazol-5-yl)phenyl)amino)ethyl)-2-(isoindolin-2-yl)-3,6-dimethylquinazolin-4(3H)-one;
N-hydroxy-2-((1-(2-(isoindolin-2-yl)-3,6-dimethyl-4-oxo-3,4-dihydroquinazolin-8-yl)ethyl) amino)benzamide;
8-(1-((2,4-difluoro-3-hydroxyphenyl)amino)ethyl)-2-(isoindolin-2-yl)-3,6-dimethyl-quinazolin-4(3H)-one;
2-(isoindolin-2-yl)-3,6-dimethyl-8-(1-((2-(2,2,2-trifluoro-1-hydroxyethyl)phenyl)amino)
ethyl)quinazolin-4(3H)-one;
2-((1-(2-(isoindolin-2-yl)-3,6-dimethyl-4-oxo-3,4-dihydroquinazolin-8-yl)ethyl)thio) benzoic acid;
2-((1-(2-(3-azabicyclo[3.1.0]hexan-3-yl)-5-cyano-3,6-dimethyl-4-oxo-3,4-dihydro-quinazolin-8-yl)ethyl)amino)benzoic acid;
2-((1-(2-(isoindolin-2-yl)-3-methyl-4-oxo-6-(trifluoromethyl)-3,4-dihydroquinazolin-8-yl)ethyl)amino)benzoic acid;
2-((1-(2-(3-azabicyclo[3.1.0]hexan-3-yl)-3-methyl-4-oxo-6-(trifluoromethyl)-3,4-dihydro-quinazolin-8-yl)ethyl)amino)benzoic acid;
2-((1-(2-(4,4-dimethylpiperidin-1-yl)-3,6-dimethyl-4-oxo-3,4-dihydroquinazolin-8-yl)
ethyl)amino)benzoic acid;
2-((1-(2-(isoindolin-2-yl)-6-methyl-4-oxo-3-(pyridin-3-yl)-3,4-dihydroquinazolin-8-yl) ethyl)amino)benzoic acid;
2-((1-(2-(isoindolin-2-yl)-3-(2-methoxyethyl)-6-methyl-4-oxo-3,4-dihydroquinazolin-8-yl) ethyl)amino)benzoic acid;
2,3-difluoro-6-((1-(3-(3-fluoropropyl)-2-(isoindolin-2-yl)-6-methyl-4-oxo-3,4-dihydro-quinazolin-8-yl)ethyl)amino)benzoic acid;
2-((1-(2-(isoindolin-2-yl)-3-isopropyl-6-methyl-4-oxo-3,4-dihydroquinazolin-8-yl)ethyl) amino)benzoic acid;
2-((1-(2-(isoindolin-2-yl)-6-methyl-4-oxo-3,4-dihydroquinazolin-8-yl)ethyl)amino)benzoic acid;
2-((1-(2-(3-azabicyclo[3.1.0]hexan-3-yl)-3,6-dimethyl-4-oxo-3,4-dihydroquinazolin-8-yl) ethyl)amino)-4-cyanobenzoic acid;
2-((1-(2-(3-azabicyclo[3.1.0]hexan-3-yl)-3,6-dimethyl-4-oxo-3,4-dihydroquinazolin-8-yl) ethyl)amino)benzoic acid;
2-((1-(3,6-dimethyl-4-oxo-2-(1H-pyrazol-5-yl)-3,4-dihydroquinazolin-8-yl)ethyl)amino) benzoic acid;
2-((1-(2-amino-3,6-dimethyl-4-oxo-3,4-dihydroquinazolin-8-yl)ethyl)amino)benzoic acid;
2-((1-(2-(isoindolin-2-yl)-3-isopropyl-6-methyl-4-oxo-3,4-dihydroquinazolin-8-yl)ethyl) amino)benzoic acid;
3-((1-(2-(isoindolin-2-yl)-3,6-dimethyl-4-oxo-3,4-dihydroquinazolin-8-yl)ethyl)amino) picolinic acid;
2-((1-(2-(isoindolin-2-yl)-3,6-dimethyl-4-oxo-3,4-dihydroquinazolin-8-yl)ethyl)amino)-6-methoxybenzoic acid;
2-((1-(2-(isoindolin-2-yl)-3,6-dimethyl-4-oxo-3,4-dihydroquinazolin-8-yl)ethyl)amino)-5-(trifluoromethyl)benzoic acid;
5-cyano-2-((1-(2-(isoindolin-2-yl)-3,6-dimethyl-4-oxo-3,4-dihydroquinazolin-8-yl)ethyl) amino)benzoic acid;
4-fluoro-2-((1-(2-(isoindolin-2-yl)-3,6-dimethyl-4-oxo-3,4-dihydroquinazolin-8-yl)ethyl) amino)benzoic acid;
5-chloro-2-((1-(2-(isoindolin-2-yl)-3,6-dimethyl-4-oxo-3,4-dihydroquinazolin-8-yl)ethyl) amino)benzoic acid;
4-((1-(2-(isoindolin-2-yl)-3,6-dimethyl-4-oxo-3,4-dihydroquinazolin-8-yl)ethyl)amino)-1-methyl-1H-pyrazole-3-carboxylic acid;
2,3-difluoro-6-((1-(2-(isoindolin-2-yl)-3,6-dimethyl-4-oxo-3,4-dihydroquinazolin-8-yl) ethyl)amino)benzoic acid;
2-((1-(2-(isoindolin-2-yl)-3,6-dimethyl-4-oxo-3,4-dihydroquinazolin-8-yl)ethyl)amino) nicotinic acid;
2-((1-(2-(4,4-difluoropiperidin-1-yl)-3,6-dimethyl-4-oxo-3,4-dihydroquinazolin-8-yl) ethyl)amino)benzoic acid;
2-((1-(5-acrylamido-2-(3-azabicyclo[3.1.0]hexan-3-yl)-3,6-dimethyl-4-oxo-3,4-dihydro-quinazolin-8-yl)ethyl)amino)benzoic acid;
2-[1-[2-(5-azaspiro[2.4]heptan-5-yl)-3,6-dimethyl-4-oxoquinazolin-8-yl]ethylamino] benzoic acid;
2-[1-(2-methoxy-3,6-dimethyl-4-oxoquinazolin-8-yl)ethylamino]benzoic acid;
2-((1-(2-hydroxy-3,6-dimethyl-4-oxo-3,4-dihydroquinazolin-8-yl)ethyl)amino)benzoic acid;
2-[1-(3,6-dimethyl-2-methylsulfanyl-4-oxoquinazolin-8-yl)ethylamino]benzoic acid;
2-((1-(2-(4-cyanopiperidin-1-yl)-3,6-dimethyl-4-oxo-3,4-dihydroquinazolin-8-yl)ethyl) amino)benzoic acid;
3-fluoro-2-((1-(2-(isoindolin-2-yl)-3,6-dimethyl-4-oxo-3,4-dihydroquinazolin-8-yl)ethyl) amino)benzoic acid;
2-((1-(2-(4-acetylpiperazin-1-yl)-3,6-dimethyl-4-oxo-3,4-dihydroquinazolin-8-yl)ethyl) amino)benzoic acid;
2-[[1-[2-(3-azabicyclo[3.1.0]hexan-3-yl)-3,6-dimethyl-4-oxoquinazolin-8-yl]-2,2,2-trifluoroethyl]amino]benzoic acid;
((1-(3-((1r,3r)-3-Hydroxycyclobutyl)-2-(isoindolin-2-yl)-6-methyl-4-oxo-3,4-dihydro-quinazolin-8-yl)ethyl)amino)benzoic acid;
2-((1-(2-(3-azabicyclo[3.1.0]hexan-3-yl)-6-fluoro-3-methyl-4-oxo-3,4-dihydro-quinazolin-8-yl)ethyl)amino)benzoic acid;
2-((1-(6-cyano-2-(isoindolin-2-yl)-3-methyl-4-oxo-3,4-dihydroquinazolin-8-yl)ethyl) amino)benzoic acid;
2-[1-[2-(3,4-dihydro-1H-isoquinolin-2-yl)-3,6-dimethyl-4-oxoquinazolin-8-yl]ethyl-amino]benzoic acid
2-[1-[3,6-dimethyl-4-oxo-2-(3-phenylpyrrolidin-1-yl)quinazolin-8-yl]ethylamino] benzoic acid;
2-[1-[3,6-dimethyl-4-oxo-2-(3-phenylpyrrolidin-1-yl)quinazolin-8-yl]ethylamino] benzoic acid;
2-[1-[2-(dimethylamino)-3,6-dimethyl-4-oxoquinazolin-8-yl]ethylamino]benzoic acid;
2-((1-(2-(azepan-1-yl)-3,6-dimethyl-4-oxo-3,4-dihydroquinazolin-8-yl)ethyl)amino) benzoic acid;
2-((1-(2-(1,1-dioxidothiomorpholino)-3,6-dimethyl-4-oxo-3,4-dihydroquinazolin-8-yl) ethyl)amino)benzoic acid;
2-((1-(2-(4-hydroxypiperidin-1-yl)-3,6-dimethyl-4-oxo-3,4-dihydroquinazolin-8-yl) ethyl)amino)benzoic acid;
   or a pharmaceutically acceptable salt thereof.

In yet further embodiments, the compounds of Formula (I) are:
2-((1-(2-((1R,5S,6s)-6-((methoxycarbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6s)-6-(((benzyloxy)carbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-4-oxo-2-((1R,5S,6s)-6-(((2,2,2-trifluoroethoxy)carbonyl)amino)-3-azabicyclo[3.1.0]hexan-3-yl)-3,4-dihydroquinazolin-8-yl)ethyl)amino)benzoic acid;
2-((1-(3,6-dimethyl-2-((1R,5S,6s)-6-(((2-morpholinoethoxy)carbonyl)amino)-3-azabicyclo[3.1.0]hexan-3-yl)-4-oxo-3,4-dihydroquinazolin-8-yl)ethyl)amino)benzoic acid;
2-((1-(2-((1R,5S,6s)-6-(((4-methoxyphenoxy)carbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6s)-6-((cyclopropoxycarbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-4-oxo-2-((1R,5S,6s)-6-((((tetrahydrofuran-2-yl)methoxy)carbonyl) amino)-3-azabicyclo[3.1.0]hexan-3-yl)-3,4-dihydroquinazolin-8-yl)ethyl)amino)benzoic acid;
2-((1-(2-(5-(tert-butoxycarbonyl)-2,5-diazabicyclo[2.2.1]heptan-2-yl)-3,6-dimethyl-4-oxo-3,4-dihydroquinazolin-8-yl)ethyl)amino)benzoic acid;
2-((1-(2-((3aR,6aR)-5-acetylhexahydropyrrolo[3,4-c]pyrrol-2(1H)-yl)-3,6-dimethyl-4-oxo-3,4-dihydroquinazolin-8-yl)ethyl)amino)benzoic acid;
2-((1-(2-(5,7-dihydro-6H-pyrrolo[3,4-b]pyridin-6-yl)-3,6-dimethyl-4-oxo-3,4-dihydro-quinazolin-8-yl)ethyl)amino)benzoic acid;
methyl 2-[1-[2-(3-azabicyclo[3.1.0]hexan-3-yl)-3-methyl-4-oxo-6-(trideuteriomethyl) quinazolin-8-yl]ethylamino]benzoate;
2-((1-(2-(isoindolin-2-yl)-3,6-dimethyl-4-oxo-3,4-dihydroquinazolin-8-yl)ethyl)amino)-4-methylbenzoic;
2-((1-(6-bromo-2-(isoindolin-2-yl)-3-methyl-4-oxo-3,4-dihydroquinazolin-8-yl)ethyl) amino)benzoic acid;
2-[1-[3,6-dimethyl-4-oxo-2-(1-prop-2-enoylpyrrolidin-2-yl)quinazolin-8-yl]ethylamino]benzoic acid;
2-((1-(2-(isoindolin-2-yl)-3-methyl-4-oxo-3,4-dihydroquinazolin-8-yl)ethyl)amino) benzoic acid;
2-[1-[2-(1-fluorosulfonylpyrrolidin-2-yl)-3,6-dimethyl-4-oxoquinazolin-8-yl]ethylamino]benzoic acid;
2-((1-(6-bromo-2-(4,4-difluoropiperidin-1-yl)-3-methyl-4-oxo-3,4-dihydroquinazolin-8-yl)ethyl)amino)benzoic acid;
2-((1-(3-((1r,3r)-3-cyanocyclobutyl)-2-(isoindolin-2-yl)-6-methyl-4-oxo-3,4-dihydro-quinazolin-8-yl)ethyl)amino)benzoic acid;
2-((1-(2-(3-azabicyclo[3.1.0]hexan-3-yl)-6-cyclopropyl-3-methyl-4-oxo-3,4-dihydro-quinazolin-8-yl)ethyl)amino)benzoic acid;
2-((1-(2-(3-azabicyclo[3.1.0]hexan-3-yl)-6-methoxy-3-methyl-4-oxo-3,4-dihydroquinazolin-8-yl)ethyl)amino)benzoic acid;
2-((1-(2-(3-azabicyclo[3.1.0]hexan-3-yl)-6-ethyl-3-methyl-4-oxo-3,4-dihydroquinazolin-8-yl)ethyl)amino)benzoic acid;
2-((1-(2-(3-azabicyclo[3.1.0]hexan-3-yl)-6-(difluoromethyl)-3-methyl-4-oxo-3,4-dihydro-quinazolin-8-yl)ethyl)amino)benzoic acid;
3-((1-(2-(3-azabicyclo[3.1.0]hexan-3-yl)-3,6-dimethyl-4-oxo-3,4-dihydroquinazolin-8-yl)ethyl)amino)-6-(trifluoromethyl)picolinic acid;
2-((1-(2-((1R,5S,6R)-6-(isopropyl(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-(cyclobutyl(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-(methoxy(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-((2,2-difluoroethyl)(methyl)carbamoyl)-3-azabicyclo[3.1.0] hexan-3-yl)-3,6-dimethyl-4-oxo-3,4-dihydroquinazolin-8-yl)ethyl)amino)benzoic acid;
2-((1-(3,6-dimethyl-2-((1R,5S,6R)-6-((S)-2-methylpyrrolidine-1-carbonyl)-3-azabicyclo [3.1.0]hexan-3-yl)-4-oxo-3,4-dihydroquinazolin-8-yl)ethyl)amino)benzoic acid;
2-((1-(3,6-dimethyl-2-((1R,5S,6R)-6-((S)-3-methylmorpholine-4-carbonyl)-3-azabicyclo [3.1.0]hexan-3-yl)-4-oxo-3,4-dihydroquinazolin-8-yl)ethyl)amino)benzoic acid;
2-((1-(2-((1R,5S,6R)-6-((2-methoxyethyl)(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-2-((1R,5S,6R)-6-(methyl(pyridin-3-yl)carbamoyl)-3-azabicyclo[3.1.0]hexan-3-yl)-4-oxo-3,4-dihydroquinazolin-8-yl)ethyl)amino)benzoic acid;
2-((1-(3,6-dimethyl-2-((1R,5S,6R)-6-(methylcarbamoyl)-3-azabicyclo[3.1.0]hexan-3-yl)-4-oxo-3,4-dihydroquinazolin-8-yl)ethyl)amino)benzoic acid;
2-((1-(2-((1R,5S,6R)-6-(dimethylcarbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-(4-(cyclopentanecarbonyl)piperazin-1-yl)-3,6-dimethyl-4-oxo-3,4-dihydro-quinazolin-8-yl)ethyl)amino)benzoic acid;
2-((1-(2-(4-(3-fluorobicyclo[1.1.1]pentane-1-carbonyl)piperazin-1-yl)-3,6-dimethyl-4-oxo-3,4-dihydroquinazolin-8-yl)ethyl)amino)benzoic acid;
2-((1-(2-(4-(2-methoxyacetyl)piperazin-1-yl)-3,6-dimethyl-4-oxo-3,4-dihydroquinazolin-8-yl)ethyl)amino)benzoic acid;
2-((1-(3,6-dimethyl-2-(4-(6-methylnicotinoyl)piperazin-1-yl)-4-oxo-3,4-dihydroquinazolin-8-yl)ethyl)amino)benzoic acid;
2-((1-(2-(3-azabicyclo[3.1.0]hexan-3-yl)-5-fluoro-3,6-dimethyl-4-oxo-3,4-dihydro-quinazolin-8-yl)ethyl)amino)benzoic acid;
2-((1-(2-(3-azabicyclo[3.1.0]hexan-3-yl)-3,6-dimethyl-4-oxo-3,4-dihydroquinazolin-8-yl)-2,2-difluoroethyl)amino)benzoic acid;
2-((1-(2-(3-azabicyclo[3.1.0]hexan-3-yl)-3,6-dimethyl-4-oxo-3,4-dihydroquinazolin-8-yl)propyl)amino)benzoic acid;
2-((1-(3-(3,3-bis(hydroxymethyl)cyclobutyl)-2-(isoindolin-2-yl)-6-methyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(3-azabicyclo[3.1.0]hexan-3-yl)-3-(difluoromethyl)-6-methyl-4-oxo-3,4-dihydroquinazolin-8-yl)ethyl)amino)benzoic acid;
2-((1-(2-(3-azabicyclo[3.1.0]hexan-3-yl)-6-methyl-4-oxo-3-(2,2,2-trifluoroethyl)-3,4-dihydroquinazolin-8-yl)ethyl)amino)benzoic acid;
2-((1-(2-(3-azabicyclo[3.1.0]hexan-3-yl)-6-methyl-3-(methyl-d3)-4-oxo-3,4-dihydro-quinazolin-8-yl)ethyl)amino)benzoic acid;
2-((1-(2-(3-azabicyclo[3.1.0]hexan-3-yl)-6-chloro-3-methyl-4-oxo-3,4-dihydroquinazolin-8-yl)ethyl)amino)benzoic acid;
2-[1-[2-(7,7-difluoro-2-azaspiro[3.3]heptan-2-yl)-3,6-dimethyl-4-oxoquinazolin-8-yl]ethyl-amino]benzoic acid;
2-[1-[3,6-dimethyl-2-[2-[(2-methylpropan-2-yl)oxycarbonylamino]-7-azaspiro[3.5] nonan-7-yl]-4-oxoquinazolin-8-yl]ethylamino]benzoic acid;
2-[1-[3,6-dimethyl-2-[2-[(2-methylpropan-2-yl)oxycarbonyl]-2,7-diazaspiro[3.5]nonan-7-yl]-4-oxoquinazolin-8-yl]ethylamino]benzoic acid;
2-[1-[2-(6,6-difluoro-2-azaspiro[3.3]heptan-2-yl)-3,6-dimethyl-4-oxoquinazolin-8-yl]ethyl-amino]benzoic acid;
2-[1-[3,6-dimethyl-2-[3-[(2-methylpropan-2-yl)oxycarbonylamino]pyrrolidin-1-yl]-4-oxoquinazolin-8-yl]ethylamino]benzoic acid;
2-[1-[2-(7-azabicyclo[2.2.1]heptan-7-yl)-3,6-dimethyl-4-oxoquinazolin-8-yl]ethyl-amino]benzoic acid;
2-[1-[2-(3-azabicyclo[3.2.0]heptan-3-yl)-3,6-dimethyl-4-oxoquinazolin-8-yl]ethyl-amino]benzoic acid;
2-[1-[3,6-dimethyl-4-oxo-2-[rac-(3aR,6aS)-3,3a,4,5,6,6a-hexahydro-1H-cyclopenta[c]pyrrol-2-yl]quinazolin-8-yl]ethylamino]benzoic acid;
2-((1-(3,6-dimethyl-4-oxo-2-(4-azaspiro[2.4]heptan-4-yl)-3,4-dihydroquinazolin-8-yl)ethyl) amino)benzoic acid;
2-((1-(2-(2,2-dimethylpyrrolidin-1-yl)-3,6-dimethyl-4-oxo-3,4-dihydroquinazolin-8-yl)ethyl) amino)benzoic acid;
2-((1-(2-(3-azabicyclo[3.1.1]heptan-3-yl)-3,6-dimethyl-4-oxo-3,4-dihydroquinazolin-8-yl)ethyl)amino)benzoic acid;
2-((1-(2-(2-azabicyclo[2.1.1]hexan-2-yl)-3,6-dimethyl-4-oxo-3,4-dihydroquinazolin-8-yl)ethyl)amino)benzoic acid;
5-((1-(2-(3-azabicyclo[3.1.0]hexan-3-yl)-3,6-dimethyl-4-oxo-3,4-dihydroquinazolin-8-yl)ethyl)amino)-2-(methylthio)pyrimidine-4-carboxylic acid;
3-[1-[2-(3-azabicyclo[3.1.0]hexan-3-yl)-3,6-dimethyl-4-oxoquinazolin-8-yl]ethyl-amino]-6-methoxypyridine-2-carboxylic acid;
3-[1-[2-(3-azabicyclo[3.1.0]hexan-3-yl)-3,6-dimethyl-4-oxoquinazolin-8-yl]ethyl-amino]-6-methylpyridine-2-carboxylic acid;
2-[1-[2-(3-azabicyclo[3.1.0]hexan-3-yl)-3,6-dimethyl-4-oxoquinazolin-8-yl]ethyl-amino]-6-fluorobenzoic acid;
2-[1-[2-(3-azabicyclo[3.1.0]hexan-3-yl)-3,6-dimethyl-4-oxoquinazolin-8-yl]ethyl-amino]-5-methoxybenzoic acid;
2-[1-[2-(3-azabicyclo[3.1.0]hexan-3-yl)-3,6-dimethyl-4-oxoquinazolin-8-yl]ethyl-amino]-5-fluorobenzoic acid;
3-[1-[2-(3-azabicyclo[3.1.0]hexan-3-yl)-3,6-dimethyl-4-oxoquinazolin-8-yl]ethyl-amino]-6-methylsulfanylpyridine-2-carboxylic acid;
4-((1-(2-(3-azabicyclo[3.1.0]hexan-3-yl)-3,6-dimethyl-4-oxo-3,4-dihydroquinazolin-8-yl)ethyl)amino)-6-fluoronicotinic acid;
4-((1-(2-(3-azabicyclo[3.1.0]hexan-3-yl)-3,6-dimethyl-4-oxo-3,4-dihydroquinazolin-8-yl)ethyl)amino)-6-chloropyridazine-3-carboxylic acid;
   or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

In some embodiments, the disclosure is directed to pharmaceutical compositions comprising compounds of Formula I, or a pharmaceutically acceptable salt or solvate thereof.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxy-propyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)-aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor or microorganism) by half. In other words, it is the half maximal (50%) inhibitory concentration (IC) of a substance (50% IC, or IC₅₀). EC₅₀ refers to the plasma concentration required for obtaining 50% of a maximum effect in vivo.

In some aspects, the present disclosure provides a method of modulating PI3K (e.g., PI3Kα) activity (e.g., in vitro or in vivo), comprising contacting a cell with a therapeutically effective amount of a compound as described herein or a pharmaceutically acceptable salt thereof.

In some aspects, the present disclosure provides a method of treating or preventing a disease or disorder disclosed herein in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound as described herein or a pharmaceutically acceptable salt thereof, or a pharmaceutical composition of the present disclosure.

In some embodiments, the disease or disorder is associated with an implicated PI3K activity. In some embodiments, the disease or disorder is a disease or disorder in which PI3K activity is implicated.

In some embodiments, the disease or disorder is a cancer.

In some embodiments, the cancer is selected from acute lymphoblastic leukemia (ALL), acute myeloid leukemia (AMIL), adrenocortical carcinoma, aids-related cancers, aids-related lymphoma, anal cancer, astrocytoma, basal cell carcinoma, bile duct cancer, bladder cancer, bone cancer, osteosarcoma, malignant fibrous histiocytoma, brain tumors, breast cancer, bronchial tumors, Burkitt lymphoma, carcinoid tumor, cancer of unknown primary, cardiac (heart) tumors, atypical teratoid/rhabdoid tumor, primary CNS lymphoma, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, mycosis fungoides, Sezary syndrome, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, fallopian tube cancer, gallbladder cancer, gastric cancer, gastrointestinal carcinoid tumor, malignant gastrointestinal stromal tumors (GIST), germ cell tumors, gestational trophoblastic disease, hairy cell leukemia, head and neck cancer, hepatocellular cancer, Langerhans cell histiocytosis, Hodgkin lymphoma, islet cell tumors, pancreatic neuroendocrine tumors, Kaposi sarcoma, kidney cancer, laryngeal cancer, leukemia, liver cancer, lung cancer, lymphoma, male breast cancer, intraocular melanoma, Merkel cell carcinoma, malignant mesothelioma, metastatic cancer, metastatic squamous neck cancer, midline tract carcinoma with nut gene changes, mouth cancer, multiple endocrine neoplasia syndromes, multiple myeloma/plasma cell neoplasms, myelodysplastic syndromes, myelodysplastic neoplasms, myeloproliferative neoplasms, chronic myeloproliferative neoplasm, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, malignant fibrous histiocytoma of bone, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, multiple myeloma, pleuropulmonary blastoma, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood vascular tumors, skin cancer, small cell lung cancer, small intestine cancer, soft tissue sarcoma, squamous cell carcinoma of the skin, testicular cancer, oropharyngeal cancer, hypopharyngeal cancer, thymoma, thymic carcinoma, thyroid cancer, tracheobronchial tumors, transitional cell cancer of the renal pelvis and ureter, urethral cancer, uterine sarcoma, vaginal cancer, vascular tumors, vulvar cancer, and Wilms tumor.

In some embodiments, the cancer is Endometrial cancer, Breast cancer, Oesophageal squamous-cell cancer, Cervical squamous-cell carcinoma, Cervical adenocarcinoma, Colorectal adenocarcinoma, Bladder Urothelial Carcinoma, Glioblastoma, Ovarian cancer, Non-small-cell Lung cancer, Esophagogastric cancer, Nerve-sheath tumor, Head and neck squamous-cell carcinoma, Melanoma, Esophagogastric adenocarcinoma, Soft-tissue sarcoma, Prostate cancer, Fibrolamellar carcinoma, Hepatocellular carcinoma, Diffuse glioma, Colorectal cancer, Pancreatic cancer, Cholangiocarcinoma, B-cell lymphoma, Mesothelioma, Adrenocortical carcinoma, Renal non-clear-cell carcinoma, Renal clear-cell carcinoma, Germ-cell carcinoma, Thymic tumor, Pheochromocytoma, Miscellaneous neuroepithelial tumor, thyroid cancer, leukemia, or encapsulated glioma.

In some embodiments, the cancer is endometrial cancer, gastric cancer, leukemia, lymphoma, sarcoma, colorectal cancer, lung cancer, ovarian cancer, skin cancer, head and neck cancer, breast cancer, brain cancer, cervical cancer, bladder cancer, esophageal cancer, pancreatic cancer, bone cancer, hepatobiliary cancer, medulloblastoma, kidney cancer or prostate cancer.

In some embodiments, the cancer is a breast cancer, a prostate cancer, or a brain cancer. In some embodiments, the cancer is a breast cancer. In some embodiments, the cancer is a prostate cancer. In some embodiments, the cancer is a brain cancer.

In some embodiments, the breast cancer is metastatic breast cancer. In some embodiments, the breast cancer is ductal carcinoma in situ (DCIS). In some embodiments, the breast cancer is invasive ductal carcinoma. In some embodiments, the breast cancer is triple negative breast cancer. In some embodiments, the breast cancer is medullary carcinoma. In some embodiments, the breast cancer is tubular carcinoma. In some embodiments, the breast cancer is mucinous carcinoma. In some embodiments, the breast cancer is Paget disease of the breast or nipple. In some embodiments, the breast cancer is inflammatory breast cancer (IBC).

In some embodiments, the prostate cancer is an adenocarcinoma. In some embodiments, the prostate cancer is a small cell carcinoma. In some embodiments, the prostate cancer is a neuroendocrine tumor. In some embodiments, the prostate cancer is a transitional cell carcinoma. In some embodiments, the prostate cancer is a sarcoma.

In some embodiments, the brain cancer is an acoustic neuroma. In some embodiments, the brain cancer is an astrocytoma. In some embodiments, the brain cancer is a brain metastasis. In some embodiments, the brain cancer is choroid plexus carcinoma. In some embodiments, the brain cancer is craniopharyngioma. In some embodiments, the brain cancer is an embryonal tumor. In some embodiments, the brain cancer is an ependymoma. In some embodiments, the brain cancer is a glioblastoma. In some embodiments, the brain cancer is a glioma. In some embodiments, the brain cancer is a medulloblastoma. In some embodiments, the brain cancer is a meningioma. In some embodiments, the brain cancer is an oligodendroglioma. In some embodiments, the brain cancer is a pediatric brain tumor. In some embodiments, the brain cancer is a pineoblastoma. In some embodiments, the brain cancer is a pituitary tumor.

In some embodiments, the disease or disorder associated with PI3K includes, but is not limited to, CLOVES syndrome (congenial lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the diseases or disorder associated with PI3K is CLOVES syndrome (congenital lipomatous overgrowth, vascular malformations, epidermal naevi, scoliosis/skeletal and spinal syndrome).

In some embodiments, the disease or disorder associated with PI3K is PIK3CA-related overgrowth syndrome (PROS).

In some embodiments, the disease or disorder associated with PI3K is breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the disease or disorder associated with PI3K is breast cancer, brain cancer, prostate cancer, endometrial cancer, gastric cancer, colorectal cancer, lung cancer, ovarian cancer, skin cancer, or head and neck cancer.

In some embodiments, the disease or disorder associated with PI3K is leukemia, lymphoma, or sarcoma.

In some embodiments, the cancer is endometrial cancer, head and neck cancer, or a sarcoma.

In some embodiments, the cancer is endometrial cancer. In some embodiments the cancer is head and neck cancer. In some embodiments, the cancer is a sarcoma.

In some embodiments, the sarcoma is soft tissue sarcoma, osteosarcoma, chondrosarcoma, Ewing sarcoma, hemangioendothelioma, angiosarcoma, fibrosarcoma, myofibrosarcoma, chordoma, adamantinoma, liposarcoma, leiomyosarcoma, malignant peripheral nerve sheath tumor, rhabdomyosarcoma, synovial sarcoma, or malignant solitary fibrous tumor.

In some embodiments, the sarcoma is soft tissue sarcoma. In some embodiments the soft tissue sarcoma is liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant gastrointestinal stromal tumor (GIST), malignant peripheral nerve sheath tumor, malignant Triton tumor, malignant granular cell tumor, malignant ossifying fibromyxoid tumor, stromal sarcoma, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, or undifferentiated sarcoma, not otherwise specified.

In some aspects, the present disclosure provides a method of treating or preventing a cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating or preventing a breast cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a breast cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating or preventing a prostate cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a prostate cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating or preventing a brain cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a brain cancer in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a compound as described herein or a pharmaceutically acceptable salt thereof for use in modulating PI3K (e.g., PI3Kα) activity (e.g., in vitro or in vivo).

In some aspects, the present disclosure provides a compound as described herein or a pharmaceutically acceptable salt thereof for use in treating or preventing a disease or disorder disclosed herein.

In some aspects, the present disclosure provides a compound as described herein or a pharmaceutically acceptable salt thereof for use in treating a disease or disorder disclosed herein.

In some aspects, the present disclosure provides a compound as described herein or a pharmaceutically acceptable salt thereof for use in treating or preventing a cancer in a subject in need thereof.

In some aspects, the present disclosure provides a compound as described herein or a pharmaceutically acceptable salt thereof for use in treating a cancer in a subject in need thereof.

In some aspects, the present disclosure provides a compound as described herein or a pharmaceutically acceptable salt thereof for use in treating or preventing a breast cancer in a subject in need thereof.

In some aspects, the present disclosure provides a compound as described herein or a pharmaceutically acceptable salt thereof for use in treating a breast cancer in a subject in need thereof.

In some aspects, the present disclosure provides a compound as described herein or a pharmaceutically acceptable salt thereof for use in treating or preventing a prostate cancer in a subject in need thereof.

In some aspects, the present disclosure provides a compound as described herein or a pharmaceutically acceptable salt thereof for use in treating a prostate cancer in a subject in need thereof.

In some aspects, the present disclosure provides a compound as described herein or a pharmaceutically acceptable salt thereof for use in treating or preventing a brain cancer in a subject in need thereof.

In some aspects, the present disclosure provides a compound as described herein or a pharmaceutically acceptable salt thereof for use in treating a brain cancer in a subject in need thereof.

In some aspects, the present disclosure provides use of a compound as described herein or a pharmaceutically acceptable salt thereof in the manufacture of a medicament for modulating PI3K (e.g., PI3Kα) activity (e.g., in vitro or in vivo).

In some aspects, the present disclosure provides use of a compound as described herein or a pharmaceutically acceptable salt thereof in the manufacture of a medicament for treating or preventing a disease or disorder disclosed herein.

In some aspects, the present disclosure provides use of a compound as described herein or a pharmaceutically acceptable salt thereof in the manufacture of a medicament for treating a disease or disorder disclosed herein.

In some aspects, the present disclosure provides use of a compound as described herein or a pharmaceutically acceptable salt thereof in the manufacture of a medicament for treating or preventing a cancer in a subject in need thereof.

In some aspects, the present disclosure provides use of a compound as described herein or a pharmaceutically acceptable salt thereof in the manufacture of a medicament for treating a cancer in a subject in need thereof.

In some aspects, the present disclosure provides use of a compound as described herein or a pharmaceutically acceptable salt thereof in the manufacture of a medicament for treating or preventing a breast cancer in a subject in need thereof.

In some aspects, the present disclosure provides use of a compound as described herein or a pharmaceutically acceptable salt thereof in the manufacture of a medicament for treating a breast cancer in a subject in need thereof.

In some aspects, the present disclosure provides use of a compound as described herein or a pharmaceutically acceptable salt thereof in the manufacture of a medicament for treating or preventing a prostate cancer in a subject in need thereof.

In some aspects, the present disclosure provides use of a compound as described herein or a pharmaceutically acceptable salt thereof in the manufacture of a medicament for treating a prostate cancer in a subject in need thereof.

In some aspects, the present disclosure provides use of a compound as described herein or a pharmaceutically acceptable salt thereof in the manufacture of a medicament for treating or preventing a brain cancer in a subject in need thereof.

In some aspects, the present disclosure provides use of a compound as described herein or a pharmaceutically acceptable salt thereof in the manufacture of a medicament for treating a brain cancer in a subject in need thereof.

The present disclosure provides compounds that function as modulators of PI3K activity. The present disclosure therefore provides a method of modulating PI3K activity in vitro or in vivo, said method comprising contacting a cell with a therapeutically effective amount of a compound, or a pharmaceutically acceptable salt thereof, as defined herein.

In some embodiments, PI3K is modulation is inhibition of PI3K.

In some embodiments, the PI3K inhibitor is a compound as described herein or a pharmaceutically acceptable salt thereof. In some embodiments, the PI3K inhibitor is a PI3Kα inhibitor. In some embodiments, the PI3K inhibitor is a PI3Kα H1047R mutant inhibitor. In some embodiments, the PI3K inhibitor is alpha/beta non-selective. In some embodiments, the PI3K inhibitor is alpha selective. In some embodiments, the PI3K inhibitor is beta selective.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PI3K activity is implicated in a patient in need of such treatment, said method comprising administering to said patient a therapeutically effective amount of a compound, or a pharmaceutically acceptable salt thereof, or a pharmaceutical composition as defined herein.

The disclosure provides a method of modulating the activity of the PI3Kα allosteric active site, wherein the modulation is induced through peripheral site targeting. In some embodiments, the peripheral site is targeted with an agent selected from a small molecule, a peptide, a peptidomimetic, a protein, a protein mimetic, a nucleic acid, an antibody, an antibody-drug conjugate, a nucleoprotein complex, an immunotherapy, or a combination thereof.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., 1H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g., a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

Compounds of Formula (I) can be prepared from optionally protected 1-1 as shown in Scheme I. Compounds 1-1 where Lg is halogen (e.g., Cl, Br, or I) or pseudohalogen (e.g., OTf, OTs, or OMs) can be transformed into compounds 1-2 under standard Stille conditions (e.g., palladium(0) catalyst, such as bis(triphenylphosphine)palladium(II) dichloride and an appropriate stannane, such as tri-n-butyl(1-ethoxyvinyl)tin), or Heck conditions (e.g., palladium catalyst, such as palladium(II) acetate, a ligand, such as 1,3-bis(diphenylphosphino)propane, and an appropriate olefin, such as butyl vinyl ether), or carbonylation coupling conditions (e.g., palladium catalyst, such as tetrakis(triphenylphosphine)palladium(0), a carbonyl source, such as carbon monoxide, and suitable boronic acid, such as phenylboronic acid). Compounds 1-2 can be converted to compounds 1-4 under either reductive conditions (e.g., in the presence of a suitable reducing agent such as sodium borohydride) or nucleophilic addition conditions with nucleophiles 1-3 where M¹ is a metal (e.g., Li, MgCl, MgBr, ZnCl, or ZnBr). Alcohols 1-4 can be transformed to compounds 1-5 where Lg¹ is halogen (e.g., Cl, Br, or I) under standard deoxygenative halogenation conditions (e.g., thionyl chloride, phosphorous tribromide, or triphenylphosphine and iodine) or a pseudohalogen (e.g., OTf, OTs, or OMs) under standard sulfonylation conditions (e.g., in the presence of a sulfonylating agent, such as methanesulfonyl chloride, p-toluenesulfonyl chloride, or trifluoromethanesulfonic anhydride, and a base, such as triethylamine). Alkylation of compounds 1-5 with nucleophiles 1-6 optionally in the presence of a base (e.g., triethylamine) can provide compound of Formula (I). Alternatively, reaction of compounds 1-5 with compounds 1-7 where the ring A contains an NH, or SH, or OH group) under standard alkylation conditions optionally in the presence of a base (e.g., sodium hydride) can provide compound 1-8.

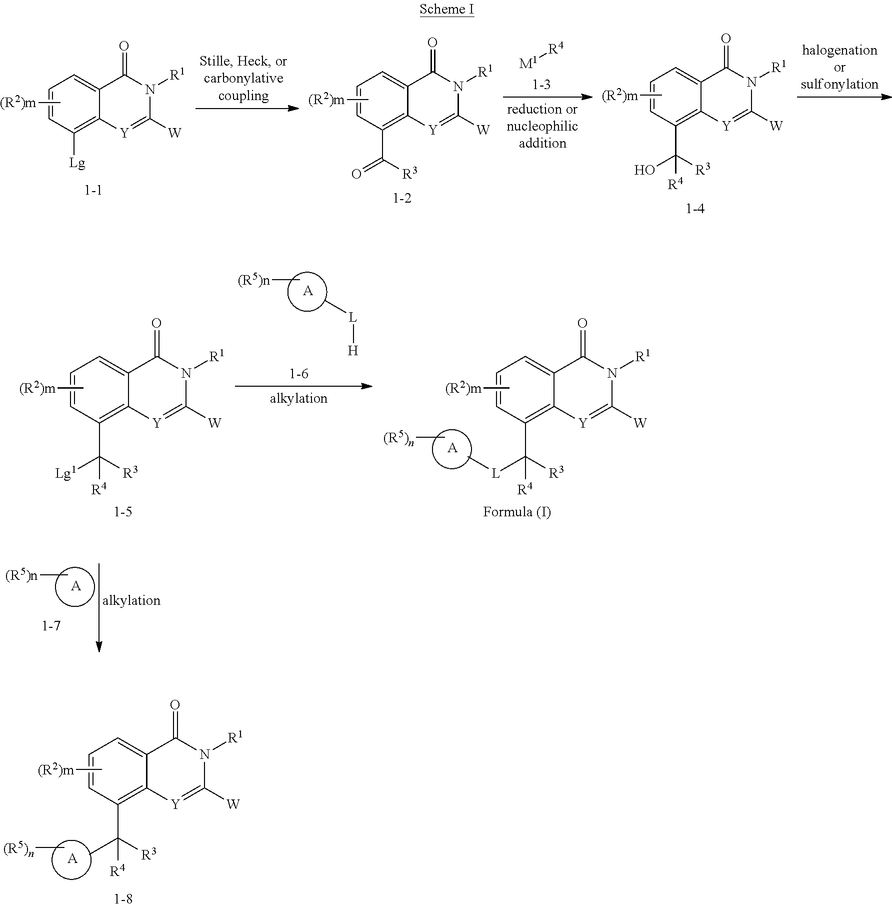

Compounds of Formula (I) can be prepared from optionally protected compounds 1-2 as shown in Scheme II. Reaction of compounds 1-2 with compounds 2-1 under standard reductive amination conditions (e.g., reducing agent such as, sodium triacetoxyborohydride and optionally an acid, such as acetic acid) can provide compounds 2-2. Alternatively, compounds 2-2 can be converted to compounds 2-5 under reductive condition (e.g., in the presence of a suitable reducing agent, such as sodium borohydride). Alcohols 2-5 can be transformed to compounds 2-6 where Lg² is halogen (e.g., Cl, Br, or I) under standard deoxygenative halogenation conditions (e.g., thionyl chloride, phosphorous tribromide, or triphenylphosphine and iodine) or a pseudohalogen (e.g., OTf, OTs, or OMs) under sulfonylation standard conditions (e.g., in the presence of a sulfonylating agent, such as methanesulfonyl chloride, p-toluenesulfonyl chloride, or trifluoromethanesulfonic anhydride, and a base, such as triethylamine). Alkylation of compounds 2-6 with nucleophiles 2-1 optionally in the presence of a base (e.g., triethylamine) can provide compounds 2-2. Alternatively, compounds 1-2 can be reacted with compounds 2-3 under standard reductive amination conditions (e.g., reducing agent such as, sodium triacetoxyborohydride and optionally an acid, such as acetic acid) to provide compounds 2-4.

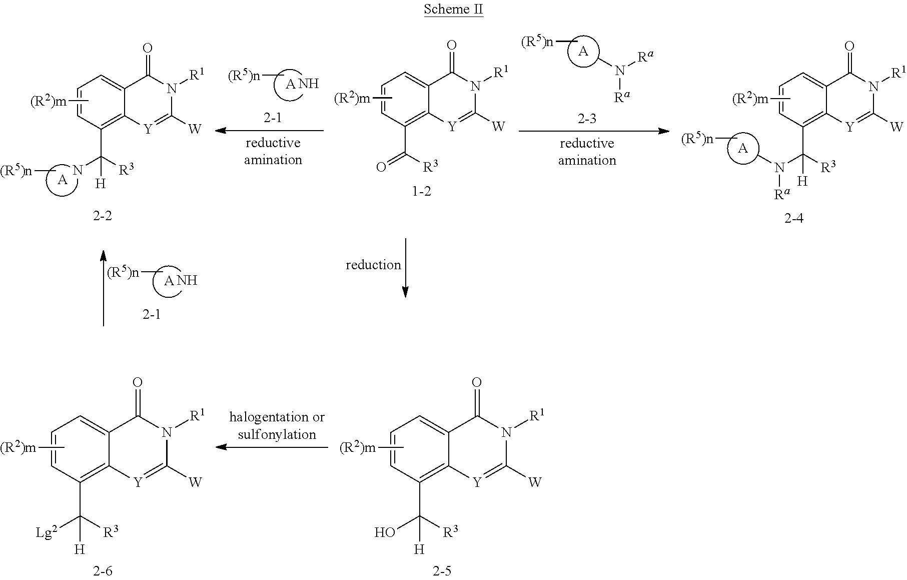

Intermediates for the synthesis of compounds of Formula (I) can be prepared as shown in Scheme III. Compounds 3-1 where Lg³ is halogen (e.g., Cl, Br, or I) or pseudohalogen (e.g., OTf or OMs) can be coupled with an amine 3-2 under standard amide bond formation conditions such as in the presence of an amide coupling reagent (e.g., N,N′-cabonyldiimidazole or HATU and a base (e.g., diisopropylethylamine) to provide compounds 3-3. Anilines 3-3 can be converted to heterocycles 3-4 under standard acylation conditions (e.g., in the presence of N,N′-cabonyldiimidazole or triphosgene and a suitable base, such as 1,8-diazabicyclo[5.4.0]undec-7-ene). Diones 3-4 can be halogenated with suitable reagents, such as phosphoryl chloride or phosphoryl bromide, to provide compounds 3-5 where X² is halogen (e.g., Cl or Br). Reaction of halides 3-5 with a compounds 3-6 under standard nucleophilic aromatic substitution conditions, such as in the presence of a suitable base (e.g., N,N-diisoproylethylamine), or under standard Buchwald-Hartwig coupling conditions (e.g., palladium catalyst such as, XPhos Pd G3, and a base, such as Cs₂CO₃ or K₃PO₄) can afford compounds 3-7.

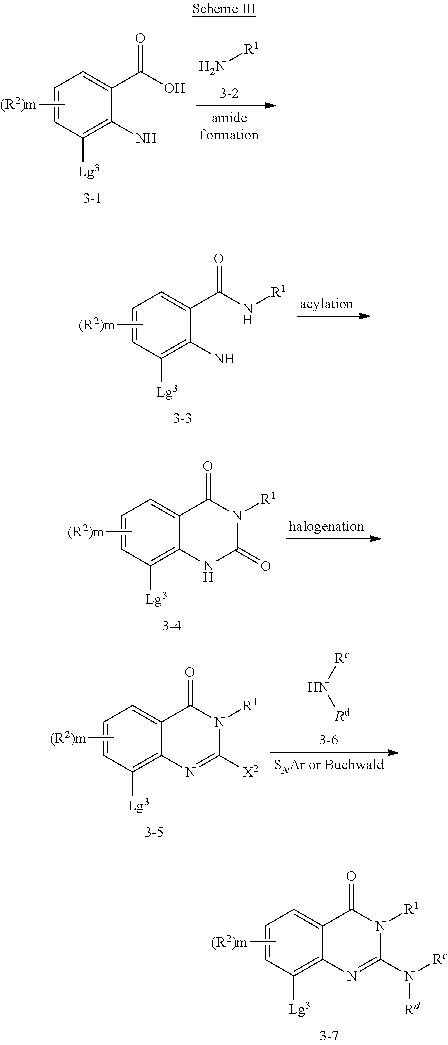

Intermediates for the synthesis of compounds of Formula (I) can be prepared as shown in Scheme IV. Reaction of compounds 4-1 where Lg⁴ is halogen (e.g., Cl, Br, or I) or pseudohalogen (e.g., OTf or OMs) with nitrites 4-2 where R^{e} is C₃-C₈ alkyl in the presence of an acid (e.g., HCl) can provide oximes 4-3. Acylation of oximes 4-3 with an acyl chloride 4-4 where R^{f} is C₁-C₈ alkyl, such as acetyl chloride, optionally in the presence of a base, such as triethylamine can afford compounds 4-5. Compounds 4-5 can be converted to an ester 4-6 where R⁹ is C₁-C₈ alkyl by Beckmann fragmentation in the presence of a base (e.g., diethylamine) in a suitable solvent (e.g., methanol). Treatment of compounds 4-6 with an amine 3-2 can afford compounds 4-7. Alkylation of compounds 4-7 with an electrophile 4-8 where Lg⁵ is halogen (e.g., Cl, Br, or I) or pseudohalogen (e.g., OTf or OMs) in the presence of a base (e.g. triethylamine or K₂CO₃) can afford compounds 4-9 where R^{h} is H, D, —C₁-C₁₀ alkyl, —C₂-C₆ alkenyl, —C₂-C₆ alkynyl, cycloalkyl, cycloalkenyl, heterocycloalkyl, or heterocycloalkenyl or two R^{h}, together with the atom to which they are both attached, form a monocyclic or multicyclic heterocycloalkyl, or a monocyclic or multicyclic heterocyclo-alkenyl group.

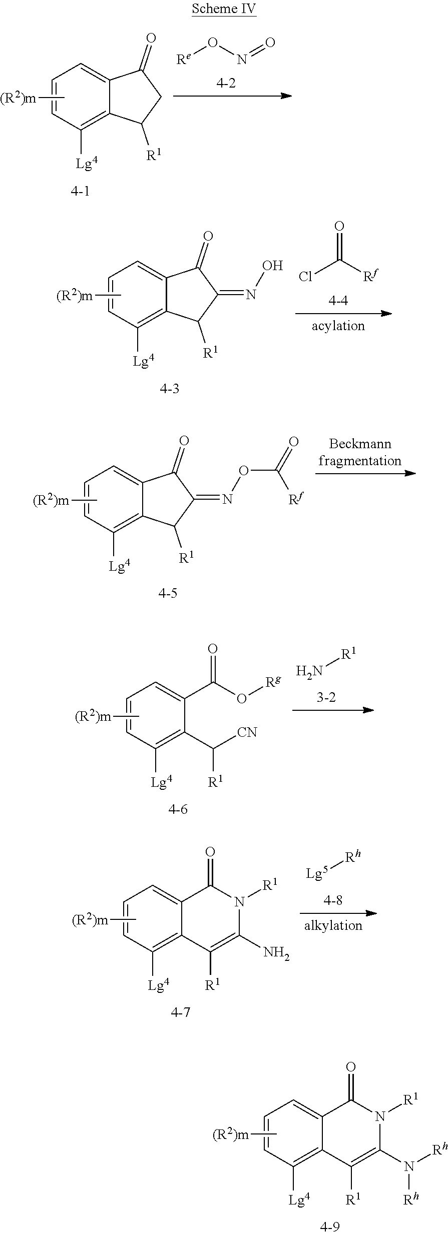

Intermediates for the synthesis of compounds of Formula (I) can be prepared as shown in Scheme V. Beckmann rearrangement of compounds 4-5 in the presence of an activating reagent (e.g., phosphorous pentachloride) can provide compounds 5-1. Reaction of compounds 5-1 with electrophiles 5-2 where Lg⁶ is halogen (e.g., Cl, Br, or I) or pseudohalogen (e.g., OTf or OMs) under standard conditions (e.g., base, such as triethylamine or K₂CO₃) can afford compounds 5-3. Reaction of compounds 5-3 with a compound 5-4 under standard nucleophilic aromatic substitution conditions in the presence of a suitable base (e.g., N,N-diisopropylethylamine, K₂CO₃, KHMDS, or NaH) or Buchwald-Hartwig coupling under standard conditions (e.g., palladium catalyst such as, XPhos Pd G3, and a base, such as Cs₂CO₃ or K₃PO₄) can afford compounds 5-5.

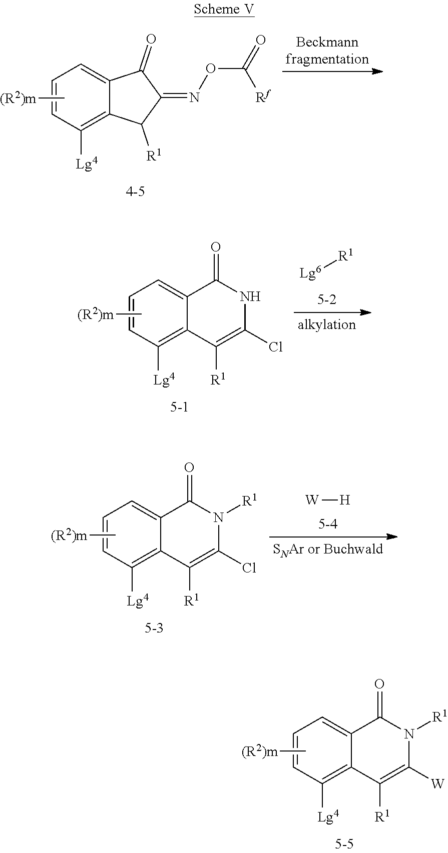

Compounds of Formula (I) can be prepared from optionally protected 2-6 as shown in Scheme VI. Alkylation of compounds 2-6 with an anthranilic acid derivative 6-1 where j is 1, 2, 3, or 4 optionally in the presence of a base (e.g., triethylamine) can provide compounds 6-2.

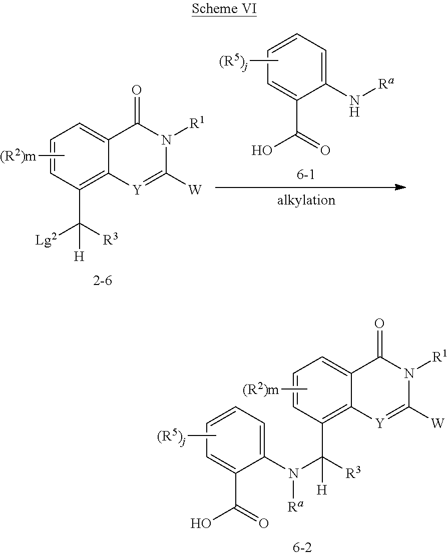

Intermediates for the synthesis of compounds of Formula (I) can be prepared as shown in Scheme VII. Reaction of heterocycles 3-4 with amines 3-6 in the presence of an amide coupling reagent (e.g., (benzotriazol-1-yl-oxy)tri-pyrrolidinophosphonium hexafluorophosphate or bromotripyrrolidinophosphonium hexafluorophosphate) and a base (e.g., 1,8-diazabicyclo[5.4.0]undec-7-ene) can provide compound 3-7.

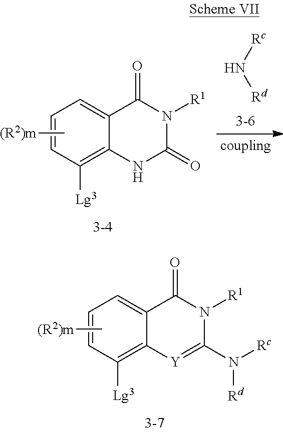

Intermediates for the synthesis of compounds of Formula (I) can be prepared as shown in Scheme VIII. Anilines 3-3 can be converted to heterocycles 8-1 under standard conditions (e.g., in the presence of 1,1-thiocarbonyldiimidazole or carbon disulfide and a base (e.g., potassium tert-butoxide or KOH)). Reaction of heterocycles 8-1 with electrophiles 8-2 where Lg⁷ is halogen (e.g., Cl, Br, or I) or pseudohalogen (e.g., OTf or OMs) and R¹ is C₁-C₈ alkyl under standard conditions (e.g., base, such as triethylamine or K₂CO₃) can afford compounds 8-3. Oxidation of compounds 8-3 using a standard oxidant (e.g., m-chloroperoxybenzoic acid) can provide sulfones 8-4. Reaction of sulfones 8-4 with nucleophiles 5-4 under standard nucleophilic aromatic substitution conditions in the presence of a suitable base (e.g., N,N-diisopropylethylamine, K₂CO₃, KHMDS, or NaH) can afford compounds 1-1.

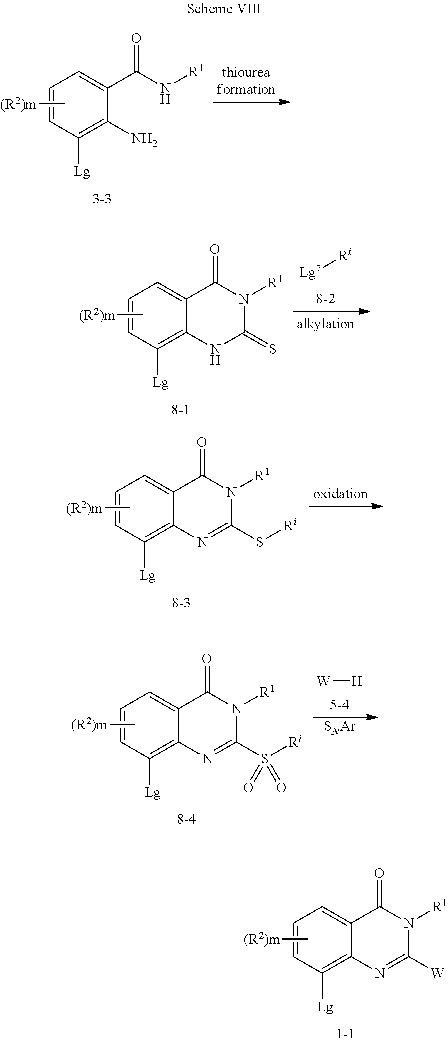

Intermediates for the synthesis of compounds of Formula (I) can be prepared as shown in Scheme IX. Preparation of intermediates 9-8 from benzoic acids 9-1 can be achieved by methods analogous to those described in Y. Tamura, et al. J. Org. Chem. 1985, 50, 2273-2277, the disclosure of which is incorporated herein by reference to its entirety. Phenols 9-1 can be protected under standard conditions to provide compounds 9-2 where PG¹ is a suitable phenol protecting group including but not limited to those described in Wuts and Greene, p. 246-287. Compounds 9-2 can be transformed to heterocycles 9-3 by acid chloride formation under standard conditions (e.g., thionyl chloride), subsequent amide formation with 2-amino-2-methyl-1-propanol, and then condensation under standard conditions, such as in the presence of a dehydrating agent (e.g., thionyl chloride). Alkylation of compounds 9-3 in the presence of a base (e.g., butyllithium) with electrophiles 9-4 where Lg⁸ is halogen (e.g., Cl, Br, or I) or pseudohalogen (e.g., OTf or OMs) can provide compounds 9-5. Reaction of compounds 9-5 in the presence of a base (e.g., butyllithium) with carbonate 9-6 where R¹ is C₁-C₈ alkyl can provide esters 9-7. Hydrolysis of compounds 9-7 under standard conditions (e.g., in the presence of aq. HCl or a aq. NaOH) can provide carboxylic acids 9-8.

Intermediates 9-8 can be transformed into heterocycles 9-10 by urea formation with amines 9-9 under standard conditions such as in the presence of a dehydrating agent (e.g., acetic anhydride). Diones 9-10 can be deoxygenatively halogenated with suitable reagents, such as phosphoryl chloride or phosphoryl bromide, to provide compounds 9-11 where X³ is halogen (e.g., Cl or Br). Reaction of halides 9-11 with a compound 5-4 under standard nucleophilic aromatic substitution conditions, such as in the presence of a suitable base (e.g., N,N-diisoproylethylamine or K₂CO₃), or under standard Buchwald-Hartwig coupling conditions (e.g., in the presence of a palladium catalyst such as, xPhos Pd G3 or Pd₂(dba)₃, and a base, such as KOtBu, Cs₂CO₃, or K₃PO₄, and optionally a ligand, such as rac-BINAP) can afford compounds 9-12. Deprotections of compounds 9-12 under standard conditions including but not limited to those described in Wuts and Greene, p. 246-287 can provide phenols 9-13. Sulfonylation of compounds 9-13 using a standard sulfonylating agent (e.g., methanesulfonyl chloride, p-toluenesulfonyl chloride, or trifluoromethanesulfonic anhydride) and a base (e.g., triethylamine or K₂CO₃) can provide intermediates 9-14 where Lg⁹ is pseudohalogen (e.g., OMs, OTs, or OTf).

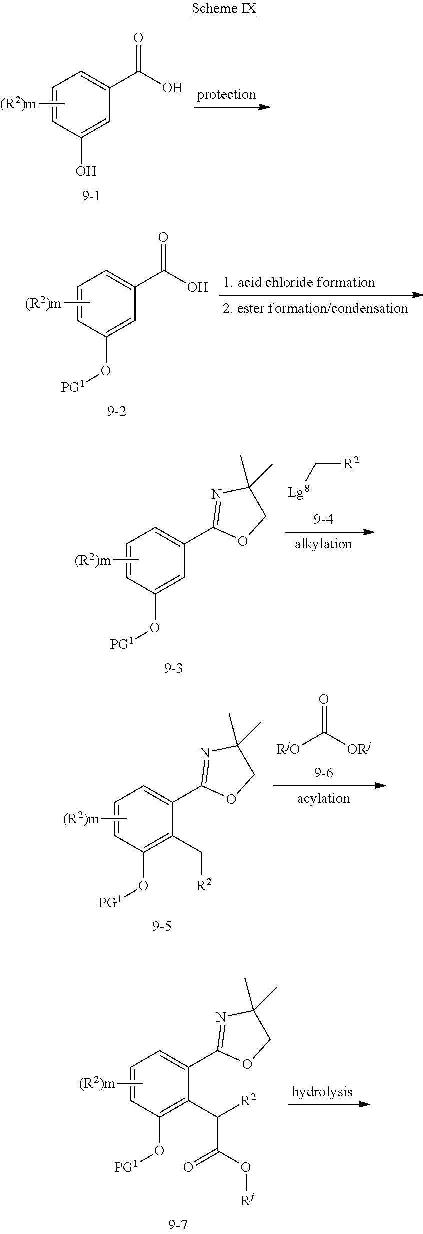

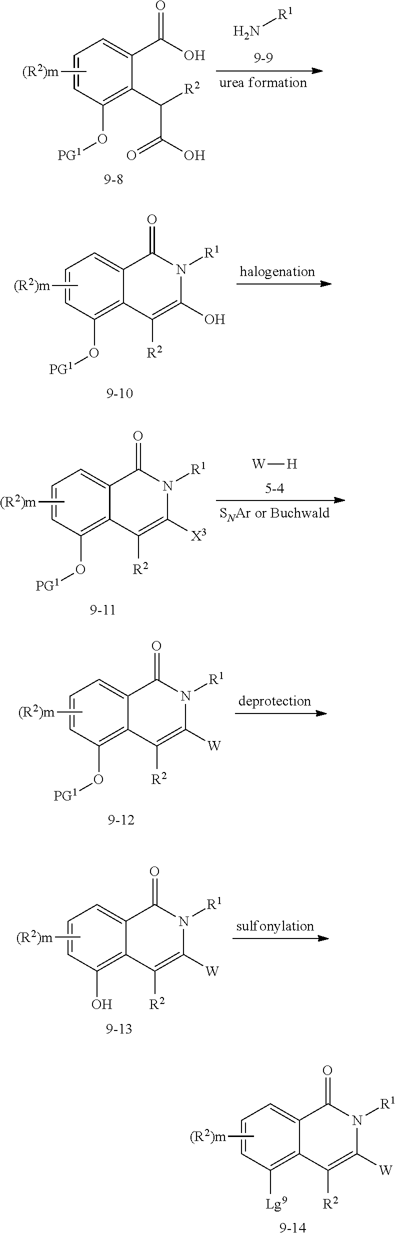

Compounds of Formula (I) can be prepared from optionally protected 2-6 as shown in Scheme X. Alkylation of optionally protected compounds 10-1 with compounds 2-6 optionally in the presence of a base (e.g., triethylamine) and subsequent optional deprotection can provide compounds 10-2.

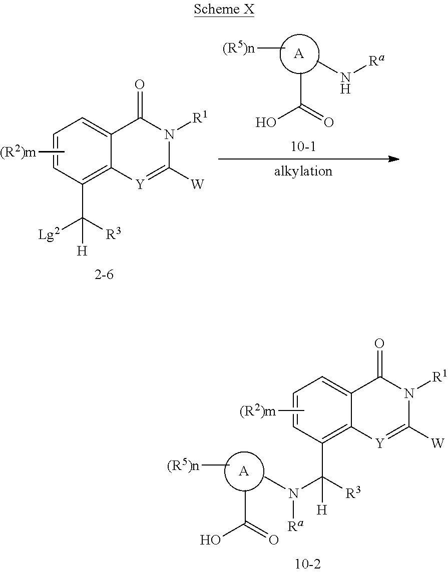

Compounds of Formula (I) can be prepared from as shown in Scheme XI. Annulation of amino acids 11-1 where ring B is an aryl or a 5-7 membered heteroaryl ring comprising 1-4 heteroatoms selected from N, O, and S with triphosgene affords bicycles 11-2. Reaction of compounds 11-2 with alcohols 2-6 under standard Mitsunobu conditions, such as in the presence of an azodicarboxylate (e.g., diisopropyl azodicarboxylate) and a phosphine (e.g., triphenylphosphine), can afford compounds 11-3. Hydrolysis under standard conditions, such as in the presence of a base (e.g., K₂CO₃ or NaOH) or an acid (e.g., HCl) or upon workup, can afford compounds 11-4.

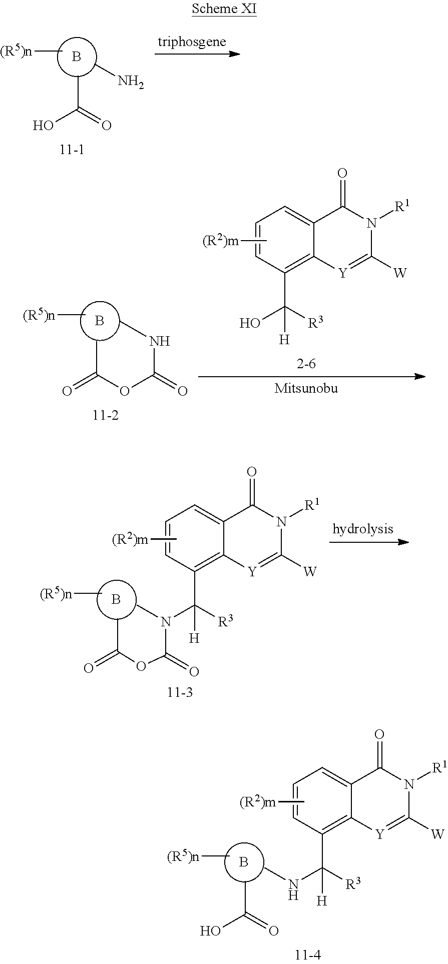

Intermediates for the synthesis of compounds of Formula (I) can be prepared as from benzoic acids 12-1 as shown in Scheme XII. Benzoic acids 12-1 can be transformed into hydroxamic acids 12-2 by acid chloride formation under standard conditions, such as in the presence of thionyl chloride, and subsequent acylation of hydroxyl amine in the presence of suitable base (e.g., triethylamine). Acylation of hydroxamic acids 12-2 with acid chlorides 12-3 where R^{z} is a C₁-C₆ alkyl group in the presence of suitable base (e.g., triethylamine) can afford compounds 12-4. Compounds 12-4 can react with an alkyne 12-5 where R^{y} is C₁-C₈ alkyl, haloalkyl, —C₂-C₆ alkenyl, —C₂-C₆ alkynyl, aryl, heteroaryl, cycloalkyl, cycloalkenyl, or heterocycloalkenyl wherein each group is optionally substituted by 1-6 R^{f} groups via metal-catalyzed C—H activation in the presence of a metal catalyst (e.g., pentmathylcyclopentadienylrhodium(III) chloride dimer) and a base (e.g., cesium acetate) to provide bicyclic heterocycles 12-6. Selective alkylation of compounds 12-6 with electrophiles 5-2 in the presence of base (e.g., cesium carbonate) can provide compounds 12-7.

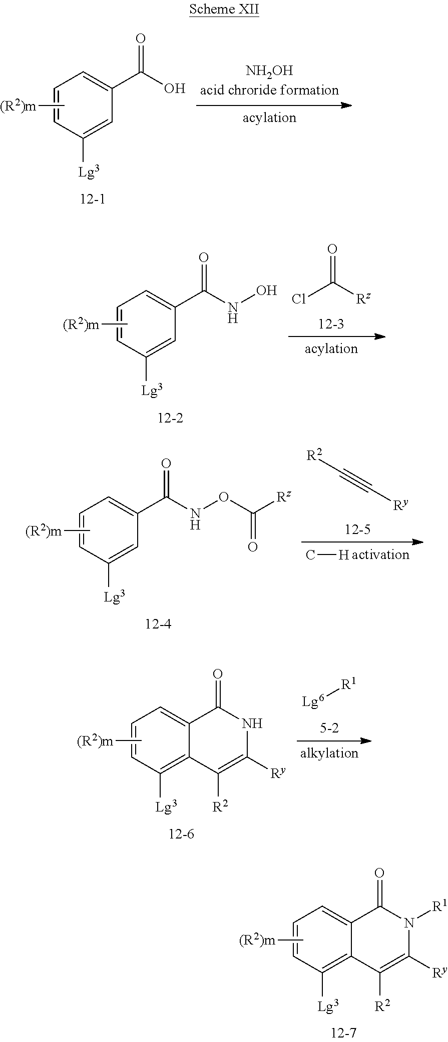

Intermediates for the synthesis of compounds of Formula (I) can be prepared as shown in Scheme XIII. Reaction of compounds 3-3 with aldehydes 13-1, where W¹ is W and wherein this group is connected to the aldehyde functional group through a carbon atom, under standard conditions, such as in the presence of a Lewis acid and an oxidant (e.g., FeCl₃ or TFA and air), can afford quinazolin-4(3H)-ones 13-2. Alternatively, amide coupling of anilines 3-3 with carboxylic acids 13-3, where W^{t} is W and wherein this group is connected to the carboxylic acid functional group through a carbon atom, under standard conditions, such as in the presence of a peptide coupling reagent (e.g., EDC or HATU) and a base (e.g., 4-dimethylaminopyridine or triethylamine) can provide amides 13-4. Cyclization of compounds 13-4 under various conditions, such as in the presence of an acid (e.g., p-toluenesulfonic acid or sulfuric acid), in the presence of a base (e.g., cesium carbonate or sodium hydroxide), in the presence of a dehydrating agent (e.g., acetic anhydride), or in the presence of 12 and hexamethyldisilazane, can provide quinazolin-4(3H)-ones 13-2.

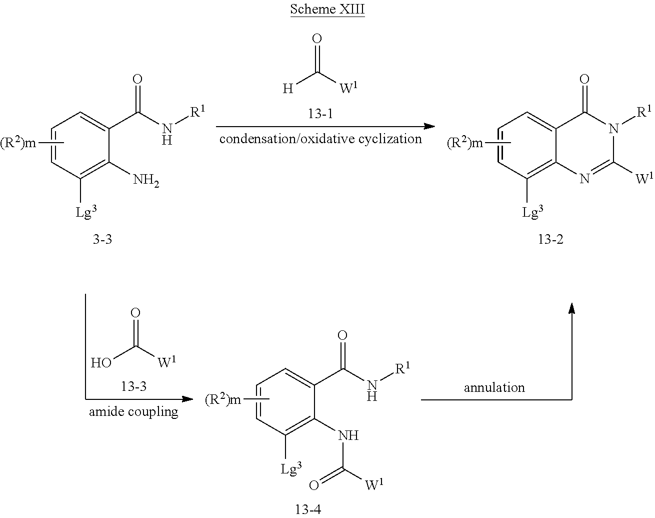

### Example 1: 2-((1-(2-(isoindolin-2-yl)-3,6-dimethyl-4-oxo-3,4-dihydroquinazolin-8-yl)ethyl) amino)benzoic acid

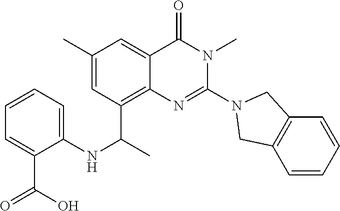 [SMILES: Cc1cc(C(C)Nc2ccccc2C(=O)O)c2nc(N3Cc4ccccc4C3)n(C)c(=O)c2c1]

### Step 1. 2-amino-3-bromo-N,5-dimethylbenzamide

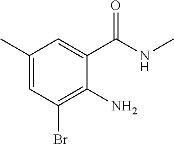

To a solution of 2-amino-3-bromo-5-methylbenzoic acid (2.00 g, 8.69 mmol) in THF (20.0 mL) was added 1,1′-carbonyldiimidazole (1.55 g, 9.56 mmol), and the resulting reaction mixture was stirred at room temperature for 12 h. Then methylamine (6.50 mL, 13 mmol, 2.0 M in THF) was added, and the reaction mixture was stirred for 4 h. The reaction was diluted with ethyl acetate (20.0 mL), and the organic layer was washed with water and brine, dried over Na₂SO₄, and concentrated. The crude material, which contained the title compound, was carried forward without further purification. LC-MS calc. for C₉H₁₂BrN₂O [M+H]⁺: m/z=243.0, 245.0; Found: 242.9, 244.9.

### Step 2. 8-bromo-3,6-dimethylquinazoline-2,4(1H,3H)-dione

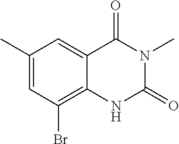

To a solution of 2-amino-3-bromo-N,5-dimethylbenzamide (500 mg, 2.06 mmol) in THF (10.0 mL) was added 1,1′-carbonyldiimidazole (500 mg, 3.09 mmol) and 1,8-diazabicyclo [5.4.0]undec-7-ene (0.614 mL, 4.11 mmol). The mixture was refluxed for 16 h. The reaction mixture was cooled to room temperature and poured into a mixture of ice water (5.0 mL) and sat. NH₄Cl (aq.) (5.0 ml). The suspension was stirred for 10 min, and then the solid was collected by filtration to afford the title compound (495 mg, 1.84 mmol, 89.4% yield) as a white solid. LC-MS calc. for C₁₀H₁₀BrN₂O₂ [M+H]⁺: m/z=269.0, 271.0; Found: 269.0, 271.0.

### Step 3. 8-bromo-2-chloro-3,6-dimethylquinazolin-4(3H)-one

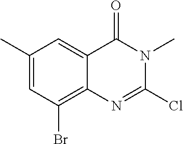

N,N-diisopropylethylamine (0.64 mL, 3.68 mmol) was added dropwise to a mixture of 8-bromo-3,6-dimethylquinazoline-2,4(1H,3H)-dione (495 mg, 1.84 mmol) in phosphorous oxychloride (12.4 mL, 132 mmol) at room temperature. The heterogeneous mixture was heated at 95° C. for 16 h. The reaction was cooled to room temperature and then concentrated. The brown sticky solution was dissolved with toluene (2.0 mL) and concentrated. The brown residue was cooled with an ice bath, and ice was added to the flask. 5 N NaOH (aq.) was added until the mixture was basic (pH >9). The aqueous mixture was extracted with EtOAc (3.0×5 mL). The combined organic layers were washed with brine (2.0×5 mL), dried over Na₂SO₄, filtered, and concentrated. The crude material, which contained the title compound, was carried forward without further purification. LC-MS calc. for C₁₀H₉BrClN₂O [M+H]⁺: m/z=287.0, 289.0; Found: 286.8, 288.8.

### Step 4. 8-bromo-2-(isoindolin-2-yl)-3,6-dimethylquinazolin-4(3H)-one

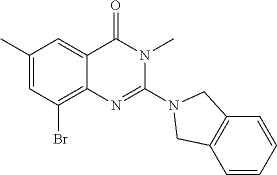 [SMILES: Cc1cc(Br)c2nc(N3Cc4ccccc4C3)n(C)c(=O)c2c1]

To a mixture of isoindoline (0.12 mL, 1.04 mmol) and 8-bromo-2-chloro-3,6-dimethyl-quinazolin-4(3H)-one (200 mg, 0.696 mmol) in THF (5.0 mL) was added N,N-diisopropylethyl-amine (0.24 mL, 1.39 mmol). The mixture was heated at 50° C. for 4 h. The reaction mixture was concentrated. The crude material was suspended in water, and the solid was collected by filtration to afford the title compound (260 mg, 0.702 mmol, quantitative yield). LC-MS calc. for C₁₈H₁₇BrN₃O [M+H]⁺: m/z=370.1, 372.1; Found: 370.0, 372.0.

### Step 5. 8-acetyl-2-(isoindolin-2-yl)-3,6-dimethylquinazolin-4(3H)-one

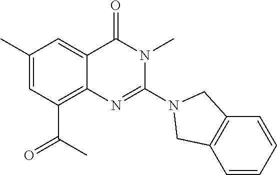 [SMILES: CC(=O)c1cc(C)cc2c(=O)n(C)c(N3Cc4ccccc4C3)nc12]

8-bromo-2-(isoindolin-2-yl)-3,6-dimethylquinazolin-4(3H)-one (260 mg, 0.702 mmol), 1-ethoxyvinyltri-n-butyltin (0.36 mL, 1.05 mmol), and bis(triphenylphosphine)palladium(II) dichloride (98.6 mg, 0.14 mmol) were dissolved in 1,4-dioxane (3.0 mL), and then the reaction mixture was heated at 100° C. for 6 h. The reaction mixture was cooled to room temperature and 6 N HCl (aq.) (0.2 ml) was added. The reaction was heated at 50° C. for 1 h. The crude product was purified by silica gel chromatography (0-100% EtOAc/hexane) to afford the title compound (205 mg, 0.615 mmol, 87.6% yield). LC-MS calc. for C₂₀H₂₀N₃O₂ [M+H]⁺: m/z=334.2; Found: 334.1.

### Step 6. 8-(1-hydroxyethyl)-2-(isoindolin-2-yl)-3,6-dimethylquinazolin-4(3H)-one

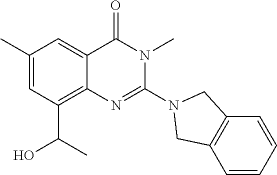 [SMILES: Cc1cc(C(C)O)c2nc(N3Cc4ccccc4C3)n(C)c(=O)c2c1]

To the solution of 8-acetyl-2-(isoindolin-2-yl)-3,6-dimethylquinazolin-4(3H)-one (205 mg, 0.615 mmol) in methanol (5.0 mL) was added sodium borohydride (69.8 mg, 1.84 mmol) at 0° C. The mixture was warmed to room temperature and stirred for 2 h. The reaction was quenched with 0.5 M HCl (aq.) (2.0 mL), and the reaction mixture was extracted with EtOAc (3.0×3 mL). The combined organic layers were washed with brine (10 mL), dried over Na₂SO₄, filtered, and concentrated. The crude material, which contained the title compound, was carried forward without further purification. LC-MS calc. for C₂₀H₂₂N₃O₂ [M+H]⁺: m/z=336.2; Found: 336.1.

### Step 7. 2-((1-(2-(isoindolin-2-yl)-3,6-dimethyl-4-oxo-3,4-dihydroquinazolin-8-yl)ethyl) amino)benzoic acid

To a solution of 8-(1-hydroxyethyl)-2-(isoindolin-2-yl)-3,6-dimethylquinazolin-4(3H)-one (44.0 mg, 0.131 mmol) in DCM (1 mL) was added phosphorus tribromide (0.01 mL, 0.1 mmol) at 0° C. dropwise. The mixture was stirred at room temperature for 1 h. The reaction was diluted with water (1.0 mL), and the two phases were separated. The organic layer was washed with sat. NaHCO₃(aq.) (1.0 mL) and brine (1.0 mL), dried over Na₂SO₄, filtered, and concentrated. To the crude residue was added a solution of anthranilic acid (36.0 mg, 0.262 mmol) in DMF (1.0 mL). The mixture was stirred at 80° C. for 4 h. The reaction mixture was cooled to room temperature, and the crude reaction was purified by prep-HPLC on a CSH Flouro-Phenyl column (43-53% MeCN/0.1% TFA (aq.)) to afford the title compound as a TFA salt (10.8 mg, 0.02 mmol, 10% yield). ¹H NMR (400 MHz, CDCl₃) δ 9.71 (s, 1H, br), 7.97 (d, J=8.3 Hz, 1H), 7.91 (s, 1H), 7.54 (s, 1H), 7.20-7.30 (m, 5H), 6.75 (t, J=7.6 Hz, 1H), 6.63 (d, J=8.5 Hz, 1H), 5.33 (q, J=7.1 Hz, 1H), 5.07 (d, J=13.4 Hz, 2H), 4.95 (d, J=13.2 Hz, 2H), 3.74 (s, 3H), 2.41 (s, 3H), 1.73 (d, J=6.7 Hz, 3H). LC-MS calc. for C₂₇H₂₇N₄O₃ [M+H]⁺: m/z=455.2; Found: 455.1.

### Example 2: 2-((1-(2-(5-fluoroisoindolin-2-yl)-3,6-dimethyl-4-oxo-3,4-dihydroquinazolin-8-yl) ethyl)amino)benzoic acid

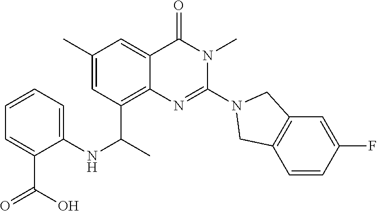 [SMILES: Cc1cc(C(C)Nc2ccccc2C(=O)O)c2nc(N3Cc4ccc(F)cc4C3)n(C)c(=O)c2c1]

The title compound was synthesized by procedures analogous to those outlined in Example 1. ¹H NMR (400 MHz, CDCl₃) δ 8.37 (s, 1H, br), 7.96 (dd, J=8.0, 1.6 Hz, 1H), 7.90 (s, 1H), 7.54 (d, J=2.1 Hz, 1H), 7.26 (m, 2H), 7.00 (dd, J=13.4, 8.0 Hz, 2H), 6.72 (t, J=7.5 Hz, 1H), 6.60 (d, J=8.5 Hz, 1H), 5.39 (q, J=6.7 Hz, 1H), 5.09-4.83 (m, 4H), 3.71 (s, 3H), 2.40 (s, 3H), 1.72 (d, J=6.7 Hz, 3H). LCMS calc. for C₂₇H₂₆FN₄O₃ [M+H]⁺: 473.2; Found: 473.1.

### Example 3: 2-((1-(2-(4,4-dimethylpiperidin-1-yl)-3,6-dimethyl-4-oxo-3,4-dihydroquinazolin-8-yl)ethyl)amino)benzoic acid

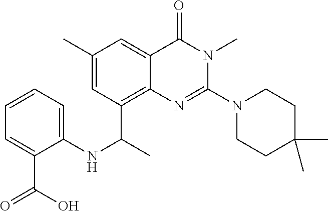 [SMILES: Cc1cc(C(C)Nc2ccccc2C(=O)O)c2nc(N3CCC(C)(C)CC3)n(C)c(=O)c2c1]

The title compound was synthesized by procedures analogous to those outlined in Example 1. LCMS calc. for C₂₆H₃₃N₄O₃ [M+H]⁺: 449.2; Found: 449.2.

### Examples 4-9

Examples 4-9 listed in Tables 1 and 2 were synthesized according to procedures analogous to Example 1. All examples in this table were prepared as the TFA salt.

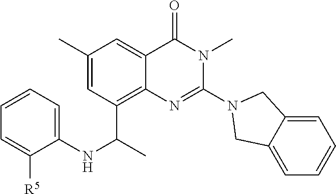

TABLE 1
Examples 4-9

| Example | R⁵ | LCMS [M + H]⁺ |
|---|---|---|
| 4 | 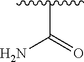 [SMILES: *C(N)=O] | 454.2 |
| 5 | 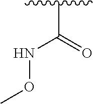 [SMILES: *C(=O)NOC] | 484.1 |
| 6 | 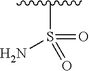 [SMILES: *S(N)(=O)=O] | 490.1 |
| 7 | 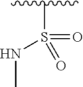 [SMILES: *S(=O)(=O)NC] | 504.1 |
| 8 | 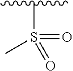 [SMILES: *S(C)(=O)=O] | 489.1 |
| 9 | 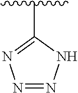 [SMILES: *c1nnn[nH]1] | 479.2 |

TABLE 2
Examples 4-9

| Example | Compound name | NMR |
|---|---|---|
| 4 | 2-((1-(2-(isoindolin-2-yl)- | ¹H NMR (400 MHz, DMSO-d6) δ 7.86 (bs, 1H), 7.69 |
| | 3,6-dimethyl-4-oxo-3,4- | (d, J = 2.1 Hz, 1H), 7.58 (dd, J = 7.9, 1.6 Hz, 1H), |
| | dihydroquinazolin-8- | 7.48-7.37 (m, 3H), 7.35-7.26 (m, 2H), 7.20 (bs, |
| | yl)ethyl)amino)benzamide | 1H), 7.04 (td, J = 7.8, 1.6 Hz, 1H), 6.44 (t, J = 7.4 |
| | | Hz, 1H), 6.37 (d, J = 8.4 Hz, 1H), 5.38 (q, J = 6.6 Hz, |
| | | 1H), 5.26-4.93 (m, 4H), 3.62 (s, 3H), 2.30 (s, 3H), |
| | | 1.52 (d, J = 6.6 Hz, 3H). |
| 5 | 2-((1-(2-(isoindolin-2-yl)- | — |
| | 3,6-dimethyl-4-oxo-3,4- | |
| | dihydroquinazolin-8- | |
| | yl)ethyl)amino)-N- | |
| | methoxybenzamide | |
| 6 | 2-((1-(2-(isoindolin-2-yl)- | ¹H NMR (400 MHz, DMSO-d6) δ 7.71-7.67 (m, |
| | 3,6-dimethyl-4-oxo-3,4- | 1H), 7.61 (dd, J = 7.9, 1.6 Hz, 1H), 7.58 (d, J = 2.1 |
| | dihydroquinazolin-8- | Hz, 1H), 7.50 (s, 2H), 7.42-7.36 (m, 2H), 7.34- |
| | yl)ethyl)amino) | 7.28 (m, 2H), 7.12 (td, J = 7.8, 1.7 Hz, 1H), 6.58 (t, J = |
| | benzenesulfonamide | 7.6 Hz, 1H), 6.43 (d, J = 8.4 Hz, 2H), 5.52-5.40 |
| | | (m, 1H), 5.18-4.95 (m, 4H), 3.62 (s, 3H), 2.30 (s, |
| | | 3H), 1.56 (d, J = 6.6 Hz, 3H). |
| 7 | 2-((1-(2-(isoindolin-2-yl)- | — |
| | 3,6-dimethyl-4-oxo-3,4- | |
| | dihydroquinazolin-8- | |
| | yl)ethyl)amino)-N- | |
| | methylbenzenesulfonamide | |
| 8 | 2-(isoindolin-2-yl)-3,6- | — |
| | dimethyl-8-(1-((2- | |
| | (methylsulfonyl)phenyl)ami- | |
| | no)ethyl)quinazolin- | |
| | 4(3H)-one | |
| 9 | 8-(1-((2-(1H-tetrazol-5- | ¹H NMR (400 MHz, DMSO-d6) δ 7.79 (d, J = 7.8 |
| | yl)phenyl)amino)ethyl)-2- | Hz, 1H), 7.70 (d, J = 2.8 Hz, 1H), 7.46 (d, J = 2.1 Hz, |
| | (isoindolin-2-yl)-3,6- | 1H), 7.43-7.37 (m, 2H), 7.35-7.28 (m, 2H), 7.16 |
| | dimethylquinazolin-4(3H)- | (ddd, J = 8.5, 7.1, 1.5 Hz, 1H), 6.67 (t, J = 7.5 Hz, |
| | one | 1H), 6.59 (d, J = 8.5 Hz, 1H), 5.51 (q, J = 6.6 Hz, |
| | | 1H), 5.35-4.91 (m, 4H), 3.62 (s, 3H), 2.28 (s, 3H), |
| | | 1.66 (d, J = 6.6 Hz, 3H). |

### Example 10: N-Hydroxy-2-((1-(2-(isoindolin-2-yl)-3,6-dimethyl-4-oxo-3,4-dihydroquinazolin-8-yl)ethyl)amino)benzamide

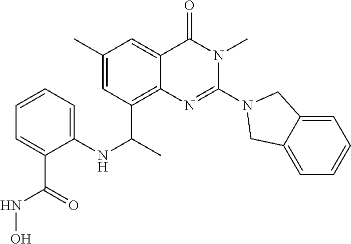 [SMILES: Cc1cc(C(C)Nc2ccccc2C(=O)NO)c2nc(N3Cc4ccccc4C3)n(C)c(=O)c2c1]

Thionyl chloride (3.2 μL, 0.044 mmol) was added to a solution of crude 2-((1-(2-(isoindolin-2-yl)-3,6-dimethyl-4-oxo-3,4-dihydroquinazolin-8-yl)ethyl)amino)benzoic acid (10 mg, Example 1, Step 7) in DCM (1.0 mL), and the resulting mixture was stirred for 15 min. Then hydroxylamine hydrochloride (7.6 mg, 0.11 mmol) and triethylamine (2.0 μL, 0.11 mmol) were added sequentially, and the reaction was stirred for 1 h. The reaction mixture was concentrated, and the crude residue was purified by prep-HPLC on CSH Phenyl-Hexyl column (38-58% MeCN/0.1% TFA (aq.)) to afford the title compound as a TFA salt (2.7 mg). LC-MS calc. for C₂₇H₂₈N₅O₃ [M+H]⁺: m/z=470.2; Found: 470.1.

### Example 11: 8-(1-((2,4-difluoro-3-hydroxyphenyl)amino)ethyl)-2-(isoindolin-2-yl)-3,6-dimethylquinazolin-4(3H)-one

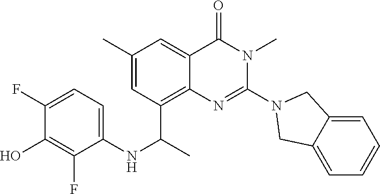 [SMILES: Cc1cc(C(C)Nc2ccc(F)c(O)c2F)c2nc(N3Cc4ccccc4C3)n(C)c(=O)c2c1]

### Step 1. 8-(1-((2,4-difluoro-3-methoxyphenyl)amino)ethyl)-2-(isoindolin-2-yl)-3,6-dimethylquinazolin-4(3H)-one

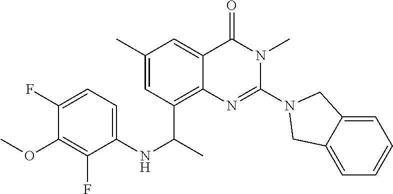 [SMILES: COc1c(F)ccc(NC(C)c2cc(C)cc3c(=O)n(C)c(N4Cc5ccccc5C4)nc23)c1F]

The title compound was synthesized by procedures analogous to those outlined in Example 1, Step 7, substituting 2,4-difluoro-3-methoxyaniline for anthranilic acid, and was isolated as a TFA saltLC-MS calc. for C₂₇H₂₇F₂N₄O₂ [M+H]⁺: m/z=477.2; Found: 477.2.

### Step 2. 8-(1-((2,4-difluoro-3-hydroxyphenyl)amino)ethyl)-2-(isoindolin-2-yl)-3,6-dimethylquinazolin-4(3H)-one

Boron tribromide (0.016 mL, 0.16 mmol) was added to a solution of 8-(1-((2,4-difluoro-3-methoxyphenyl)amino)ethyl)-2-(isoindolin-2-yl)-3,6-dimethylquinazolin-4(3H)-one (19 mg, 0.033 mmol) (from Step 1) in DCM (0.65 mL), and the resulting mixture was stirred for 18 h. The reaction mixture was concentrated under reduced pressure and the crude residue was purified by prep-HPLC on CSH C-18 column (47-67 MeCN/0.1% TFA (aq.)) to afford the title compound as a TFA salt (2.6 mg, 5.5 μmol, 14% yield). ¹H NMR (400 MHz, DMSO-d6) δ 7.67 (d, J=1.9 Hz, 1H), 7.54 (d, J=2.1 Hz, 1H), 7.44-7.37 (m, 2H), 7.36-7.27 (m, 2H), 6.63-6.47 (m, 1H), 5.89 (td, J=9.1, 4.8 Hz, 1H), 5.25 (q, J=6.7 Hz, 1H), 5.12-4.95 (m, 4H), 3.61 (s, 3H), 2.31 (s, 3H), 1.51 (d, J=6.7 Hz, 3H). LC-MS calc. for C₂₆H₂₅F₂N₄O₂ [M+H]⁺: m/z=463.2; Found: 463.1.

### Example 12: 2-(isoindolin-2-yl)-3,6-dimethyl-8-(1-((2-(2,2,2-trifluoro-1-hydroxyethyl)phenyl) amino)ethyl)quinazolin-4(3H)-one

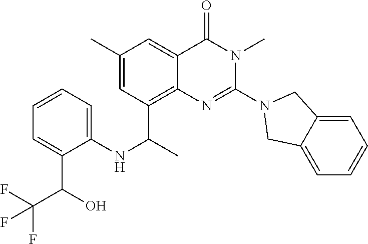 [SMILES: Cc1cc(C(C)Nc2ccccc2C(O)C(F)(F)F)c2nc(N3Cc4ccccc4C3)n(C)c(=O)c2c1]

### Step 1. 1-(2-aminophenyl)-2,2,2-trifluoroethan-1-ol

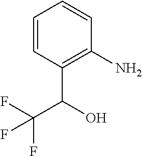 [SMILES: Nc1ccccc1C(O)C(F)(F)F]

Trimethyl(trifluoromethyl)silane (0.29 mL, 2.0 mmol) was added to a solution of 2-nitrobenzaldehyde (200 mg, 1.3 mmol) and sodium acetate (220 mg, 2.7 mmol) in DMF (2.6 mL) at 0° C. The resulting reaction mixture was stirred at 0° C. for 1 h before being diluted with DCM (10 mL) and water (25 mL). The two phases were separated, and the aqueous layer was extracted by DCM (20 mL×2). The combined organic layers were dried over sodium sulfate, filtered, and concentrated. The crude residue was dissolved in methanol (13 mL), and then palladium on carbon (140 mg, 0.13 mmol, 10 wt. %) was added to the solution. The reaction mixture was stirred under an atmosphere of H₂ for 18 h. The mixture was diluted with DCM and filtered through a pad of Celite®. The filtrate was concentrated. The crude material was used in the next step without further purification. LC-MS calc. for C₈H₉F₃NO [M+H]⁺: m/z=192.1; Found: 192.0.

### Step 2. 2-(isoindolin-2-yl)-3,6-dimethyl-8-(1-((2-(2,2,2-trifluoro-1-hydroxyethyl)phenyl) amino)ethyl)quinazolin-4(3H)-one

The title compound was synthesized by procedures analogous to those outlined in Example 1, Step 7. LC-MS calc. for C₂₈H₂₈F3N402 [M+H]⁺: m/z=509.2; Found: 509.2.

### Example 13: 2-((1-(2-(isoindolin-2-yl)-3,6-dimethyl-4-oxo-3,4-dihydroquinazolin-8-yl)ethyl)thio)benzoic acid

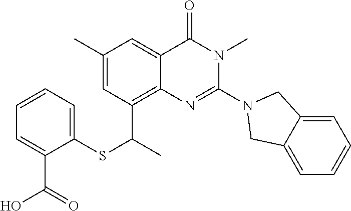 [SMILES: Cc1cc(C(C)Sc2ccccc2C(=O)O)c2nc(N3Cc4ccccc4C3)n(C)c(=O)c2c1]

The title compound was synthesized by procedures analogous to those outlined in Example 1, Step 7, substituting 2-mercaptobenzoic acid for anthranilic acid. ¹H NMR (400 MHz, DMSO-d6) δ 7.79 (dd, J=7.7, 1.6 Hz, 1H), 7.73-7.68 (m, 2H), 7.53 (d, J=8.1 Hz, 1H), 7.37-7.24 (m, 5H), 7.13 (td, J=7.5, 1.0 Hz, 1H), 5.59 (q, J=7.1 Hz, 1H), 5.07-4.89 (m, 4H), 3.60 (s, 3H), 2.35 (s, 3H), 1.65 (d, J=7.0 Hz, 3H). LC-MS calc. for C₂₇H₂₆N303S [M+H]⁺: m/z=472.2; Found: 472.1.

### Example 14: 2-((1-(2-(3-azabicyclo[3.1.0]hexan-3-yl)-5-cyano-3,6-dimethyl-4-oxo-3,4-dihydroquinazolin-8-yl)ethyl)amino)benzoic acid

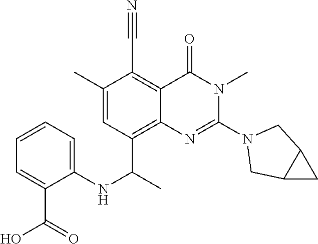 [SMILES: Cc1cc(C(C)Nc2ccccc2C(=O)O)c2nc(N3CC4CC4C3)n(C)c(=O)c2c1C#N]

### Step 1. 6-amino-2-bromo-3-methylbenzoic acid

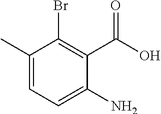

Hydrogen peroxide (4.8 mL, 47 mmol, 35 wt. % in water) was added dropwise to a solution of 4-bromo-5-methyl-1H-indole-2,3-dione (5.0 g, 21 mmol) in NaOH (21 mL, 3N (aq.)), and the reaction mixture was stirred for 18 h. The reaction mixture was adjusted to pH ˜5 using 1N HCl (aq.), and the mixture was concentrated. The resulting residue was suspended in methanol (100 mL) and filtered, and the filtrate was concentrated. The crude solid was used in the next step without further purification. LC-MS calc. for C₈H₉BrNO₂ [M+H]⁺: m/z=230.0, 232.0; Found: 229.9, 231.9.

### Step 2. 6-amino-2-bromo-N,3-dimethylbenzamide

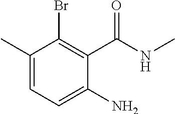

Crude 6-amino-2-bromo-3-methylbenzoic acid, 1-[bis(dimethylamino)methylene]-1H-1,2,3-triazolo[4,5-b]pyridinium 3-oxide hexafluorophosphate (12 g, 31 mmol), triethylamine (15 mL, 100 mmol), and methylamine hydrochloride (2.1 g, 31 mmol) were dissolved in DCM (70 mL), and the solution was stirred at room temperature for 1 h. The mixture was diluted with water (100 mL) and DCM (100 mL). The two phases were separated, and the aqueous layer was extracted by DCM (100 mL×2). The combined organic layers were dried over sodium sulfate, filtered, and concentrated. The crude residue was used without further purification. LC-MS calc. for C₉H₁₂BrN₂O [M+H]⁺: m/z=243.0, 245.0; Found: 242.9, 244.9.

### Step 3. 2-amino-6-bromo-3-iodo-N,5-dimethylbenzamide

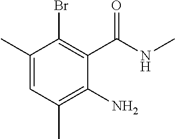

N-Iodosuccinimide (5.2 g, 23 mmol) was added to a mixture of crude 6-amino-2-bromo-N,3-dimethylbenzamide and sodium acetate (2.4 g, 29 mmol) in glacial acetic acid (42 mL), and the reaction mixture was stirred for 1 h. The reaction mixture was diluted with water (200 mL) and DCM (200 mL). The two phases were separated, and the aqueous layer was extracted by DCM (100 mL×2). The combined organic layers were dried over sodium sulfate, filtered, and concentrated. The crude residue was used without further purification. LC-MS calc. for C₉H₁₁BrINO₂ [M+H]⁺: m/z=368.9, 370.9; Found: 368.9, 370.9.

### Step 4. 5-bromo-8-iodo-3,6-dimethylquinazoline-2,4(1H,3H)-dione

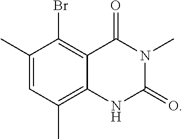 [SMILES: Cc1cc(C)c2[nH]c(=O)n(C)c(=O)c2c1Br]

Triphosgene (6.2 g, 21 mmol) was added to a solution of crude 2-amino-6-bromo-3-iodo-N,5-dimethylbenzamide in THF (70 mL), and the reaction mixture was stirred at room temperature for 18 hours. The reaction was diluted with water (100 mL) and DCM (200 mL). The two phases were separated, and the aqueous layer was extracted by DCM (100 mL×2). The combined organic layers were dried over sodium sulfate, filtered, and concentrated. The crude residue was used without further purification. ¹H NMR (400 MHz, CDCl₃) δ 7.99 (bs, 1H), 7.90 (s, 1H), 3.44 (s, 3H), 2.45 (s, 3H).

### Step 5. 5-bromo-2-chloro-8-iodo-3,6-dimethylquinazolin-4(3H)-one

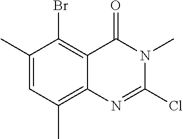 [SMILES: Cc1cc(C)c2nc(Cl)n(C)c(=O)c2c1Br]

The title compound was synthesized by procedures analogous to those outlined in Example 1, Step 3. LC-MS calc. for C₁₀H₈BrClIN₂O [M+H]⁺: m/z=412.9, 414.9; Found: 412.9, 414.8.

### Step 6. 2-(3-azabicyclo[3.1.0]hexan-3-yl)-5-bromo-8-iodo-3,6-dimethylquinazolin-4(3H)-one

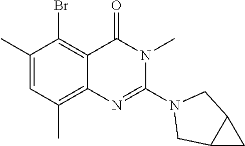 [SMILES: Cc1cc(C)c2nc(N3CC4CC4C3)n(C)c(=O)c2c1Br]

The title compound was synthesized by procedures analogous to those outlined in Example 1, Step 4, substituting 3-azabicyclo[3.1.0]hexane for isoindoline. LC-MS calc. for C₁₅H₁₆BrIN₃O [M+H]⁺: m/z=459.9, 461.9; Found: 459.8, 461.9.

### Step 7. 8-acetyl-2-(3-azabicyclo[3.1.0]hexan-3-yl)-5-bromo-3,6-dimethylquinazolin-4(3H)-one

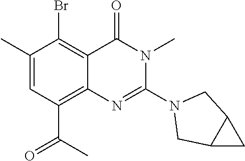 [SMILES: CC(=O)c1cc(C)c(Br)c2c(=O)n(C)c(N3CC4CC4C3)nc12]

2-(3-azabicyclo[3.1.0]hexan-3-yl)-5-bromo-8-iodo-3,6-dimethylquinazolin-4(3H)-one (1.3 g, 2.8 mmol), Tributyl(1-ethoxyvinyl)tin (1.1 g, 3.1 mmol), and bis(triphenylphosphine) palladium(II) dichloride (200 mg, 0.28 mmol) were dissolved in 1,4-dioxane (28 mL). The reaction vessel was sealed, and the reaction mixture degassed with N2 and heated to 80° C. for 18 h. The reaction mixture was cooled to room temperature, and several drops of 6N HCl (aq.) were added. After heating at 50° C. for 1 h, the reaction mixture was cooled to room temperature and diluted with water (50 mL) and DCM (100 mL). The two phases were separated, and the aqueous layer was extracted by DCM (100 mL×2). The combined organic layers were dried over sodium sulfate, filtered, and concentrated. The crude residue was purified by silica gel chromatography (0-100% EtOAc/hexanes) to afford the title compound (400 mg, 1.1 mmol, 38% yield). LC-MS calc. for C₁₇H₁₉BrN₃O₂ [M+H]⁺: m/z=376.1, 378.1; Found: 376.0, 378.0.

### Step 8. 2-(3-azabicyclo[3.1.0]hexan-3-yl)-5-bromo-8-(1-hydroxyethyl)-3,6-dimethylquinazolin-4(3H)-one

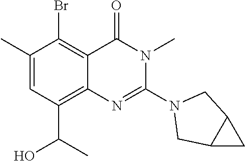 [SMILES: Cc1cc(C(C)O)c2nc(N3CC4CC4C3)n(C)c(=O)c2c1Br]

The title compound was synthesized by procedures analogous to those outlined in Example 1, Step 6. LC-MS calc. for C₁₇H₂₁BrN₃O₂ [M+H]⁺: m/z=378.1, 380.1; Found: 378.1, 380.0.

### Step 9. tert-butyl 2-((1-(2-(3-azabicyclo[3.1.0]hexan-3-yl)-5-bromo-3,6-dimethyl-4-oxo-3,4-dihydroquinazolin-8-yl)ethyl)amino)benzoate

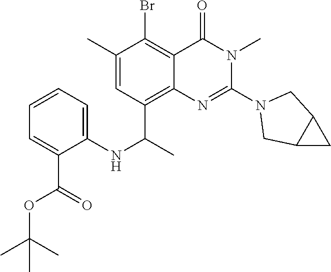 [SMILES: Cc1cc(C(C)Nc2ccccc2C(=O)OC(C)(C)C)c2nc(N3CC4CC4C3)n(C)c(=O)c2c1Br]

The title compound was synthesized by procedures analogous to those outlined in Example 1, Step 7, substituting tert-butyl 2-aminobenzoate for anthranilic acid. ¹H NMR (400 MHz, DMSO-d6) δ 8.19 (bs, 1H), 7.74 (dd, J=8.0, 1.6 Hz, 1H), 7.51 (s, 1H), 7.29-7.10 (m, 1H), 6.50 (t, J=7.5 Hz, 1H), 6.42 (d, J=8.5 Hz, 1H), 5.30 (bs, 1H), 4.00 (d, J=10.6 Hz, 1H), 3.86 (d, J=10.5 Hz, 1H), 3.60-3.44 (m, 2H), 3.38 (s, 3H), 2.33 (s, 3H), 1.65-1.58 (m, 2H), 1.57 (s, 9H), 1.55 (d, J=6.7 Hz, 3H), 0.67-0.53 (m, 1H), 0.35-0.23 (m, 1H). LC-MS calc. for C₂₈H₃₄BrN₄O₃ [M+H]⁺: m/z=553.2, 555.2; Found: 553.1, 555.0.

### Step 10. tert-butyl 2-((J-(2-(3-azabicyclo[3.1.0]hexan-3-yl)-5-cyano-3,6-dimethyl-4-oxo-3,4-dihydroquinazolin-8-yl)ethyl)amino)benzoate

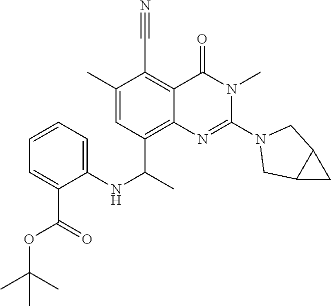 [SMILES: Cc1cc(C(C)Nc2ccccc2C(=O)OC(C)(C)C)c2nc(N3CC4CC4C3)n(C)c(=O)c2c1C#N]

tert-Butyl 2-((1-(2-(3-azabicyclo[3.1.0]hexan-3-yl)-5-bromo-3,6-dimethyl-4-oxo-3,4-dihydroquinazolin-8-yl)ethyl)amino)benzoate (5.0 mg, 9.0 μmol), zinc cyanide (4.1 mg, 45 μmol), and tetrakis(triphenylphosphine)palladium(0) (2.1 mg, 1.8 μmol) were dissolved in DMF (0.30 mL). The reaction vessel was sealed, and the reaction mixture was degassed with N2 and heated at 100° C. for 18 h. The reaction mixture was cooled to room temperature and diluted with water (10 mL) and DCM (10 mL). The two phases were separated, and the aqueous layer was extracted by DCM (10 mL×2). The combined organic layers were dried over sodium sulfate, filtered, and concentrated. The crude residue was purified by prep-HPLC on CSH C-18 column (75-100% MeCN/0.1% TFA (aq.)) to afford the title compound as a TFA salt (3.0 mg, 6.0 μmol, 54% yield). LC-MS calc. for C₂₉H₃₄N₅O₃ [M+H]⁺: m/z=500.3; Found: 500.2.

### Step 11. 2-((1-(2-(3-azabicyclo[3.1.0]hexan-3-yl)-5-cyano-3,6-dimethyl-4-oxo-3,4-dihydroquinazolin-8-yl)ethyl)amino)benzoic acid

TFA (50 μL, 0.65 mmol) was added to a solution of tert-butyl 2-((1-(2-(3-azabicyclo[3.1.0]hexan-3-yl)-5-cyano-3,6-dimethyl-4-oxo-3,4-dihydroquinazolin-8-yl)ethyl) amino)benzoate (3.0 mg, 6.0 μmol) in DCM (0.20 mL) and the reaction mixture was stirred for 18 h. The reaction mixture was concentrated, and the crude residue was purified by prep-HPLC on CSH C-18 column (59-79% MeCN/0.1% TFA (aq.)) to afford the title compound as a TFA salt (1.0 mg, 1.8 μmol, 38% yield). LC-MS calc. for C₂₅H₂₆N₅O₃ [M+H]⁺: m/z=444.2; Found: 444.1.

### Examples 15-21

Examples 15-21 listed in Tables 3 and 4 were synthesized according to procedures analogous to Example 1. All examples in this table were prepared as the TFA salt.

TABLE 3
Examples 15-21

| Example | Structure | LCMS [M + H]⁺ |
|---|---|---|
| 15 | 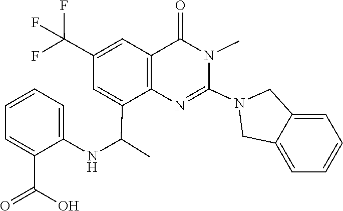 [SMILES: CC(Nc1ccccc1C(=O)O)c1cc(C(F)(F)F)cc2c(=O)n(C)c(N3Cc4ccccc4C3)nc12] | 509.1 |
| 16 | 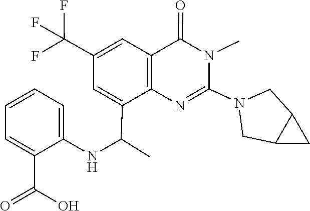 [SMILES: CC(Nc1ccccc1C(=O)O)c1cc(C(F)(F)F)cc2c(=O)n(C)c(N3CC4CC4C3)nc12] | 473.1 |
| 17 | 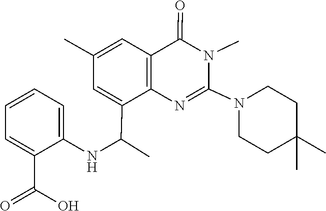 [SMILES: Cc1cc(C(C)Nc2ccccc2C(=O)O)c2nc(N3CCC(C)(C)CC3)n(C)c(=O)c2c1] | 449.3 |
| 18 | 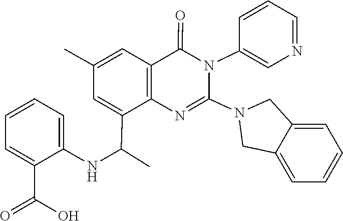 [SMILES: Cc1cc(C(C)Nc2ccccc2C(=O)O)c2nc(N3Cc4ccccc4C3)n(-c3cccnc3)c(=O)c2c1] | 518.2 |
| 19 | 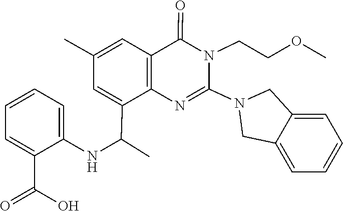 [SMILES: COCCn1c(N2Cc3ccccc3C2)nc2c(C(C)Nc3ccccc3C(=O)O)cc(C)cc2c1=O] | 499.1 |
| 20 | 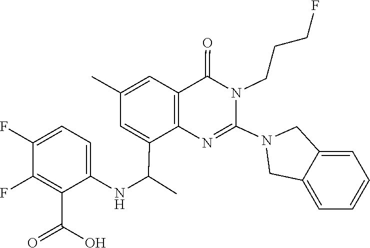 [SMILES: Cc1cc(C(C)Nc2ccc(F)c(F)c2C(=O)O)c2nc(N3Cc4ccccc4C3)n(CCCF)c(=O)c2c1] | 537.1 |
| 21 | 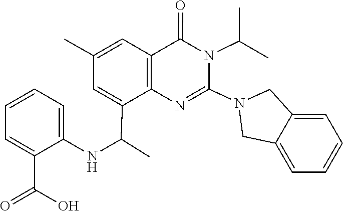 [SMILES: Cc1cc(C(C)Nc2ccccc2C(=O)O)c2nc(N3Cc4ccccc4C3)n(C(C)C)c(=O)c2c1] | 483.1 |

TABLE 4
Examples 15-21

| Example | Compound name | NMR |
|---|---|---|
| 15 | 2-((1-(2-(isoindolin-2-yl)-3- | ¹H NMR (400 MHz, CDCl₃) δ 8.37 (s, 1H), 7.97 (dd, |
| | methyl-4-oxo-6- | J = 8.2, 1.7 Hz, 1H), 7.81 (d, J = 2.2 Hz, 1H), 7.25- |
| | (trifluoromethyl)-3,4- | 7.35 (m, 4H), 7.23-7.13 (m, 1H), 6.60 (t, J = 7.5 Hz, |
| | dihydroquinazolin-8- | 1H), 6.39 (d, J = 8.5 Hz, 1H), 5.47 (t, J = 6.8 Hz, 1H), |
| | yl)ethyl)amino)benzoic acid | 5.15 (d, J = 13.7 Hz, 2H), 5.04 (d, J = 13.3 Hz, 2H), |
| | | 3.75 (s, 3H), 1.68 (d, J = 6.7 Hz, 3H). |
| 16 | 2-((1-(2-(3-azabicyclo[3.1.0] | ¹H NMR (400 MHz, CDCl₃) δ 8.34 (d, J = 1.8 Hz, |
| | hexan-3-yl)-3-methyl-4-oxo- | 1H), 8.02 (d, J = 8.0 Hz, 1H), 7.81 (d, J = 2.3 Hz, |
| | 6-(trifluoromethyl)-3,4- | 1H), 7.26 (t, J = 7.8 Hz, 1H), 6.73 (t, J = 7.8 Hz, 1H), |
| | dihydroquinazolin-8- | 6.49 (d, J = 8.4 Hz, 1H), 5.38 (q, J = 6.7 Hz, 1H), |
| | yl)ethyl)amino)benzoic acid | 4.03 (d, J = 10.5 Hz, 1H), 3.89 (d, J = 10.5 Hz, 1H), |
| | | 3.71-3.58 (m, 2H), 3.57 (s, 3H), 1.68 (d, J = 6.7 Hz, |
| | | 3H), 1.65 (m, 2H), 0.69 (m, 1H), 0.33 (m, 1H). |
| 17 | 2-((1-(2-(4,4-dimethyl- | ¹H NMR (400 MHz, DMSO) δ 12.61 (s, 1H), 8.44 (s, |
| | piperidin-1-yl)-3,6-dimethyl- | 1H), 7.78 (dd, J = 7.8, 1.7 Hz, 1H), 7.71 (s, 1H), 7.46 |
| | 4-oxo-3,4-dihydro- | (d, J = 2.1 Hz, 1H), 7.22-7.14 (m, 1H), 6.49 (t, J = |
| | quinazolin-8-yl) ethyl) | 8.0 Hz, 2H), 5.41 (m, 1H), 3.47 (s, 3H), 3.23 (t, J = |
| | amino)benzoic acid | 5.6 Hz, 4H), 2.32 (s, 3H), 1.59-1.45 (m, 7H), 1.00 |
| | | (s, 6H). |
| 18 | 2-((1-(2-(isoindolin-2-yl)-6- | — |
| | methyl-4-oxo-3-(pyridin-3- | |
| | yl)-3,4-dihydroquinazolin-8- | |
| | yl)ethyl)amino)benzoic acid | |
| 19 | 2-((1-(2-(isoindolin-2-yl)-3- | — |
| | (2-methoxyethyl)-6-methyl- | |
| | 4-oxo-3,4-dihydro- | |
| | quinazolin-8-yl)ethyl)amino) | |
| | benzoic acid | — |
| 20 | 2,3-difluoro-6-((1-(3-(3- | |
| | fluoropropyl)-2-(isoindolin- | |
| | 2-yl)-6-methyl-4-oxo-3,4- | |
| | dihydroquinazolin-8- | |
| | yl)ethyl)amino)benzoic acid | |
| 21 | 2-((1-(2-(isoindolin-2-yl)-3- | ¹H NMR (400 MHz, DMSO) δ 8.42 (s, br 1H), 7.77 |
| | isopropyl-6-methyl-4-oxo- | (dd, J = 8.1, 1.7 Hz, 1H), 7.68 (d, J = 2.1 Hz, 1H), |
| | 3,4-dihydroquinazolin-8- | 7.44-7.35 (m, 3H), 7.30 (dd, J = 5.6, 3.2 Hz, 2H), |
| | yl)ethyl)amino)benzoic acid | 7.14 (td, J = 7.6, 1.7 Hz, 1H), 6.48 (t, J = 7.7 Hz, 2H), |
| | | 5.40 (q, J = 6.7 Hz, 1H), 5.03 (d, J = 13.5 Hz, 2H), |
| | | 4.92 (d, J = 13.5 Hz, 2H), 4.78 (p, J = 6.6 Hz, 1H), |
| | | 2.30 (s, 3H), 1.69 (d, J = 6.6 Hz, 3H), 1.58 (dd, J = |
| | | 15.1, 6.6 Hz, 6H). |

### Example 22: 2-((1-(2-(isoindolin-2-yl)-6-methyl-4-oxo-3,4-dihydroquinazolin-8-yl)ethyl)amino)benzoic acid

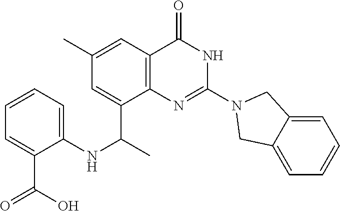 [SMILES: Cc1cc(C(C)Nc2ccccc2C(=O)O)c2nc(N3Cc4ccccc4C3)[nH]c(=O)c2c1]

### Step 1. 2-((1-(2-(isoindolin-2-yl)-3-(4-methoxybenzyl)-6-methyl-4-oxo-3,4-dihydroquinazolin-8-yl)ethyl)amino)benzoic acid

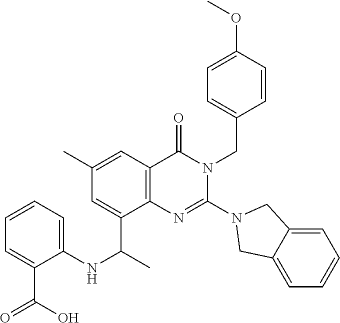 [SMILES: COc1ccc(Cn2c(N3Cc4ccccc4C3)nc3c(C(C)Nc4ccccc4C(=O)O)cc(C)cc3c2=O)cc1]

The title compound was synthesized by procedures analogous to those outlined in Example 1, substituting (4-methoxyphenyl)methanamine for methylamine in Step 1. LC-MS calc. for C₃₄H₃₃N₄O₄ [M+H]⁺: m/z=561.2; Found: 561.2.

### Step 2. 2-((1-(2-(isoindolin-2-yl)-6-methyl-4-oxo-3,4-dihydroquinazolin-8-yl)ethyl) amino)benzoic acid

A solution of 2-((1-(2-(isoindolin-2-yl)-3-(4-methoxybenzyl)-6-methyl-4-oxo-3,4-dihydroquinazolin-8-yl)ethyl)amino)benzoic acid (5.0 mg, 0.010 mmol) in TFA (1 mL) was stirred at 45° C. for 2 h. The reaction mixture was cooled to room temperature and concentrated, and the crude residue was purified by prep-HPLC on CSH C-18 column (40-60% MeCN/0.1% TFA (aq.)) to afford the title compound as a TFA salt (1.1 mg, 2.5 μmol, 28% yield). LC-MS calc. for C₂₆H₂₅N₄O₃ [M+H]⁺: m/z=441.2; Found: 441.2.

### Example 23: 2-((1-(2-(3-azabicyclo[3.1.0]hexan-3-yl)-3,6-dimethyl-4-oxo-3,4-dihydro-quinazolin-8-yl)ethyl)amino)-4-cyanobenzoic acid

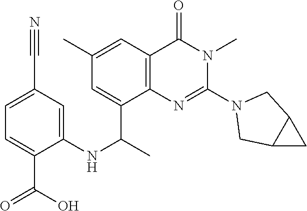 [SMILES: Cc1cc(C(C)Nc2cc(C#N)ccc2C(=O)O)c2nc(N3CC4CC4C3)n(C)c(=O)c2c1]

### Step 1. 2-(3-azabicyclo[3.1.0]hexan-3-yl)-8-(1-hydroxyethyl)-3,6-dimethylquinazolin-4(3H)-one

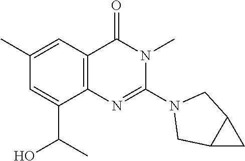 [SMILES: Cc1cc(C(C)O)c2nc(N3CC4CC4C3)n(C)c(=O)c2c1]

The title compound was synthesized by procedures analogous to those outlined in Example 1, Steps 1-6, substituting 3-azabicyclo[3.1.0]hexane for isoindoline in Step 4. LC-MS calc. for C₁₇H₂₂N₃O₂ [M+H]⁺: m/z=300.2; Found: 300.1.

### Step 2. Methyl 2-((1-(2-(3-azabicyclo[3.1.0]hexan-3-yl)-3,6-dimethyl-4-oxo-3,4-dihydroquinazolin-8-yl)ethyl)amino)-4-cyanobenzoate

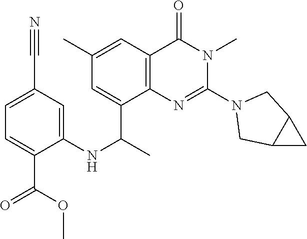 [SMILES: COC(=O)c1ccc(C#N)cc1NC(C)c1cc(C)cc2c(=O)n(C)c(N3CC4CC4C3)nc12]

To a solution of 2-(3-azabicyclo[3.1.0]hexan-3-yl)-8-(1-hydroxyethyl)-3,6-dimethyl-quinazolin-4(3H)-one (20 mg, 0.07 mmol) in DCM (1 mL) was added methanesulfonyl chloride (0.01 mL, 0.08 mmol) and triethylamine dropwise (0.02 mL, 0.1 mmol) at 0° C. The mixture was stirred at 0° C. for 0.5 h and slowly warmed to room temperature over 4 h. Then, methyl 2-amino-4-cyanobenzoate (17.6 mg, 0.100 mmol), and triethylamine (0.02 mL, 0.1 mmol) were added. The mixture was stirred 16 h. The reaction mixture was concentrated and purified by prep-HPLC on CSH Flouro-Phenyl column (53-73% MeCN/0.1% TFA (aq.)) to afford the title compound as a TFA salt (5.0 mg, 11 μmol, 16% yield). LC-MS calc. for C₂₆H₂₈N₅O₃ [M+H]⁺: m/z=458.2; Found: 458.1.

### Step 3. 2-((1-(2-(3-azabicyclo[3.1.0]hexan-3-yl)-3,6-dimethyl-4-oxo-3,4-dihydro-quinazolin-8-yl)ethyl)amino)-4-cyanobenzoic acid

To a solution of methyl 2-[1-[2-(3-azabicyclo[3.1.0]hexan-3-yl)-3,6-dimethyl-4-oxoquinazolin-8-yl]ethylamino]-4-cyanobenzoate (5.0 mg, 0.011 mmol) in 1,4-dioxane (0.5 mL) and water (0.5 mL) was added LiOH (1.3 mg, 0.055 mmol). The mixture was stirred at 40° C. for 14 h. The mixture was adjusted to pH 2 with 1 M HCl and extracted with EtOAc (2.0×2 mL). The combined organic layers were washed with brine (2.0 mL), dried over Na₂SO₄, filtered, and concentrated. The crude residue was purified by prep-HPLC on CSH Flouro-Phenyl column (42-62% MeCN/0.1% TFA (aq.)) to afford the title compound as a TFA salt (2.1 mg, 3.8 μmol, 35% yield). LC-MS calc. for C₂₅H₂₆N₅O₃ [M+H]⁺: m/z=444.2; Found: 444.2.

### Example 24: 2-((1-(2-(3-azabicyclo[3.1.0]hexan-3-yl)-3,6-dimethyl-4-oxo-3,4-dihydro-quinazolin-8-yl)ethyl)amino)benzoic acid (isomer 1)

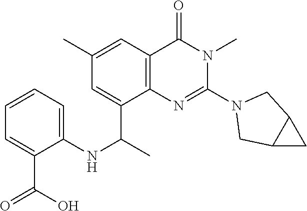 [SMILES: Cc1cc(C(C)Nc2ccccc2C(=O)O)c2nc(N3CC4CC4C3)n(C)c(=O)c2c1]

### Step 1. Methyl 2-(((1-(2-(3-azabicyclo[3.1.0]hexan-3-yl)-3,6-dimethyl-4-oxo-3,4-dihydroquinazolin-8-yl)ethyl)amino)benzoate (isomer 1)

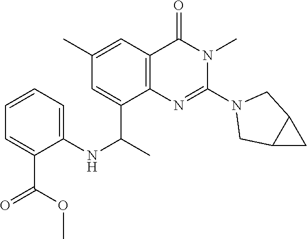 [SMILES: COC(=O)c1ccccc1NC(C)c1cc(C)cc2c(=O)n(C)c(N3CC4CC4C3)nc12]

The title compound was synthesized by procedures analogous to those outlined in Example 1, substituting methyl 2-aminobenzoate for anthranilic acid in Step 7. The isomers were separated using chiral prep-HPLC on a Lux iA3 column (30 mL/min, 88:6:6 hexane/IPA/MeOH) to afford two isomers: isomer 1 (t_{R}=2.83 min) and isomer 2 (t_{R}=3.29 min). Isomer 1: LC-MS calc. for C₂₅H₂₉N₄O₃ [M+H]⁺: m/z=433.2; Found: 433.2.

### Step 2. 2-((-1-(2-(3-azabicyclo[3.1.0]hexan-3-yl)-3,6-dimethyl-4-oxo-3,4-dihydro-quinazolin-8-yl)ethyl)amino)benzoic acid (isomer 1)

To a solution of methyl 2-((-1-(2-(3-azabicyclo[3.1.0]hexan-3-yl)-3,6-dimethyl-4-oxo-3,4-dihydroquinazolin-8-yl)ethyl)amino)benzoate (isomer 1) (50.0 mg, 0.116 mmol) in 1,4-dioxane (1.0 mL) and water (1.0 mL) was added LiOH (13.8 mg, 0.578 mmol). The mixture was stirred at 90° C. for 2 h. The mixture was adjusted to pH 2 with 1N HCl and was extracted with EtOAc (2.0×2 mL). The combined organic layers were washed with brine (2.0 mL), dried over Na₂SO₄, filtered, and concentrated to afford the title compound (23.2 mg, 55.4 μmol, 48.0% yield). ¹H NMR (400 MHz, DMSO-d₆) δ 12.63 (s, 1H), 8.40 (s, 1H), 7.77 (dd, J=7.9, 1.7 Hz, 1H), 7.66 (dd, J=2.1, 1.0 Hz, 1H), 7.41 (d, J=2.1 Hz, 1H), 7.17 (ddd, J=8.7, 7.1, 1.7 Hz, 1H), 6.53-6.42 (m, 2H), 5.36 (m, 1H), 3.99 (d, J=10.5 Hz, 1H), 3.82 (d, J=10.5 Hz, 1H), 3.57-3.44 (m, 2H), 3.43 (s, 3H), 2.29 (s, 3H), 1.65-1.52 (m, 5H), 0.59 (td, J=7.6, 4.5 Hz, 1H), 0.37 (q, J=4.2 Hz, 1H). LC-MS calc. for C₂₄H₂₇N₄O₃ [M+H]⁺: m/z=419.2; Found: 419.1.

### Example 25: 2-((1-(3,6-dimethyl-4-oxo-2-(1H-pyrazol-5-yl)-3,4-dihydroquinazolin-8-yl) ethyl)amino)benzoic acid

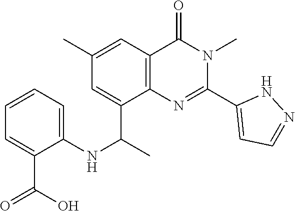 [SMILES: Cc1cc(C(C)Nc2ccccc2C(=O)O)c2nc(-c3ccn[nH]3)n(C)c(=O)c2c1]

### Step 1. 8-bromo-3,6-dimethyl-2-(1H-pyrazol-5-yl)quinazolin-4(3H)-one

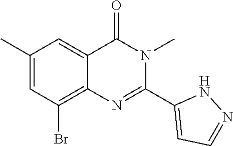 [SMILES: Cc1cc(Br)c2nc(-c3ccn[nH]3)n(C)c(=O)c2c1]

A mixture of 2-amino-3-bromo-N,5-dimethylbenzamide (250 mg, 1.03 mmol), 1H-pyrazole-5-carbaldehyde (98.8 mg, 1.03 mmol), and iron(III) chloride (111 mg, 0.689 mmol) in water (5.0 mL) was heated at 100° C. for 24 h. Then reaction mixture was cooled to room temperature. The resulting solid was collected by filtration to afford the title compound (310 mg, 0.702 mmol, 94.4% yield). LC-MS calc. for C₁₃H₁₂BrN₄O [M+H]⁺: m/z=319.0, 321.0; Found: 319.0, 321.0.

### Step 2. 8-bromo-3,6-dimethyl-2-(1-((2-(trimethylsilyl)ethoxy)methyl)-1H-pyrazol-5-yl)quinazolin-4(3H)-one

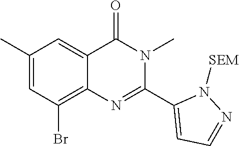 [SMILES: Cc1cc(Br)c2nc(-c3ccnn3COCC[Si](C)(C)C)n(C)c(=O)c2c1]

To a solution of 8-bromo-3,6-dimethyl-2-(1H-pyrazol-5-yl)quinazolin-4(3H)-one (310 mg, 0.971 mmol) in THF (8 mL) was added NaH (58.3 mg, 1.46 mmol) at 0° C. The mixture was stirred at 0° C. for 2 h. Then, =2-(trimethylsilyl)ethoxymethyl chloride (0.19 mL, 1.1 mmol) was added dropwise. The resulting mixture was warmed to room temperature and stirred overnight. The reaction was quenched with water, and the mixture was extracted with EtOAc (2.0×3 mL). The combined organic layers were washed with brine (2.0 mL), dried over Na₂SO₄, filtered, and concentrated. The crude product was purified by silica gel chromatography (0-50% EtOAc/hexane) to afford the title compound (140 mg, 0.312 mmol, 32.1% yield). LC-MS calc. for C₁₉H₂₆BrN₄O₂Si [M+H]⁺: m/z=449.1, 451.1; Found: 449.0, 451.0.

### Step 3. 8-acetyl-3,6-dimethyl-2-(1-((2-(trimethylsilyl)ethoxy)methyl)-1H-pyrazol-5-yl)quinazolin-4(3H)-one

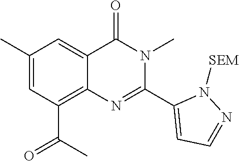 [SMILES: CC(=O)c1cc(C)cc2c(=O)n(C)c(-c3ccnn3COCC[Si](C)(C)C)nc12]

The title compound was synthesized by procedures analogous to those outlined in Example 1, Step 5. LC-MS calc. for C₂₁H₂₉N₄O₃Si [M+H]⁺: m/z=413.2; Found: 413.2.

### Step 4. 8-(1-hydroxyethyl)-3,6-dimethyl-2-(1-((2-(trimethylsilyl)ethoxy)methyl)-1H-pyrazol-5-yl)quinazolin-4(3H)-one

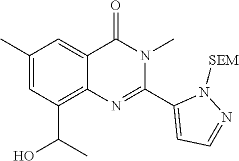 [SMILES: Cc1cc(C(C)O)c2nc(-c3ccnn3COCC[Si](C)(C)C)n(C)c(=O)c2c1]

The title compound was synthesized by procedures analogous to those outlined in Example 1, Step 6. LC-MS calc. for C₂₁H₃₁N₄O₃Si [M+H]⁺: m/z=415.2; Found: 415.2.

### Step 5. 2-((1-(3,6-dimethyl-4-oxo-2-(1H-pyrazol-5-yl)-3,4-dihydroquinazolin-8-yl) ethyl)amino)benzoic acid

The title compound was synthesized by procedures analogous to those outlined in Example 1, Step 7. Deprotection of SEM group occurred spontaneously in this step. LC-MS calc. for C₂₂H₂₂N₅O₃ [M+H]⁺: m/z=404.1; Found: 404.1.

### Example 26: 2-((1-(2-amino-3,6-dimethyl-4-oxo-3,4-dihydroquinazolin-8-yl)ethyl)amino) benzoic acid

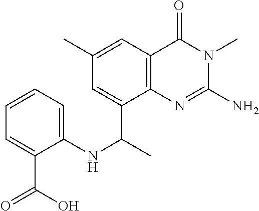 [SMILES: Cc1cc(C(C)Nc2ccccc2C(=O)O)c2nc(N)n(C)c(=O)c2c1]

### Step 1. 2-((1-(2-(bis(4-methoxybenzyl)amino)-3,6-dimethyl-4-oxo-3,4-dihydroquinazolin-8-yl)ethyl)amino)benzoic acid

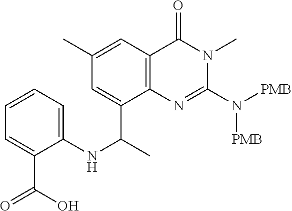

The title compound was synthesized by procedures analogous to those outlined in Example 1, substituting bis(4-methoxybenzyl)amine for isoindoline in Step 4. LC-MS calc. for C₃₅H₃₇N₄O₅ [M+H]⁺: m/z=593.3; Found: 593.3.

### Step 2. 2-((1-(2-amino-3,6-dimethyl-4-oxo-3,4-dihydroquinazolin-8-yl)ethyl)amino) benzoic acid

A solution of 2-[1-[2-[bis[(4-methoxyphenyl)methyl]amino]-3,6-dimethyl-4-oxoquinazolin-8-yl]ethylamino]benzoic acid (5.0 mg, 8.4 μmol) in TFA (1 mL) was stirred at 45° C. for 16 h. The reaction mixture was cooled to room temperature and concentrated, and the crude residue was concentrated purified by prep-HPLC on a CSH Flouro-Phenyl column (18-38% MeCN/0.1% TFA (aq.)) to afford the title compound as a TFA salt (1.1 mg, 2.4 μmol, 28% yield). LC-MS calc. for C₁₉H₂₁N₄O₃ [M+H]⁺: m/z=353.2; Found: 353.1.

### Example 27: 2-((1-(2-(isoindolin-2-yl)-3-isopropyl-6-methyl-4-oxo-3,4-dihydroquinazolin-8-yl)ethyl)amino)benzoic acid (isomer 1)

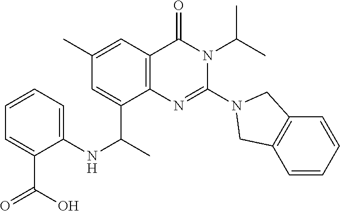 [SMILES: Cc1cc(C(C)Nc2ccccc2C(=O)O)c2nc(N3Cc4ccccc4C3)n(C(C)C)c(=O)c2c1]

### Step 1. Methyl 2-((1-(2-(isoindolin-2-yl)-3-isopropyl-6-methyl-4-oxo-3,4-dihydroquinazolin-8-yl)ethyl)amino)benzoate (isomer 1)

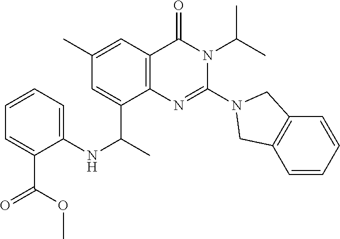 [SMILES: COC(=O)c1ccccc1NC(C)c1cc(C)cc2c(=O)n(C(C)C)c(N3Cc4ccccc4C3)nc12]

The title compound was synthesized by procedures analogous to those outlined in Example 1, substituting propan-2-amine for methylamine in Step 1 and methyl 2-aminobenzoate for anthranilic acid in Step 7. The isomers were separated using chiral prep-HPLC on a Lux iA3 column (30 mL/min, 98:2 hexane/EtOH) to afford two isomers: isomer 1 (t_{R}=2.51 min) and isomer 2 (t_{R} 3.43 min). Isomer 1: LC-MS calc. for C₃₀H₃₃N₄O₃ [M+H]⁺: m/z=497.3; Found: 497.3.

### Step 2. 2-((1-(2-(isoindolin-2-yl)-3-isopropyl-6-methyl-4-oxo-3,4-dihydroquinazolin-8-yl)ethyl)amino)benzoic acid (isomer 1)

To a solution of methyl 2-((1-(2-(isoindolin-2-yl)-3-isopropyl-6-methyl-4-oxo-3,4-dihydroquinazolin-8-yl)ethyl)amino)benzoate (isomer 1) (5.0 mg, 0.010 mmol) in 1,4-dioxane (0.5 mL) and water (0.5 mL) was added LiOH (1.2 mg, 0.050 mmol). The mixture was stirred at 90° C. for 2 h. The mixture was adjusted to pH 2 with 1N HCl and was extracted with EtOAc (lx 2 mL). The combined organic layers were washed with brine (1.0 mL), dried over Na₂SO₄, filtered, and concentrated to afford the title compound (4.3 mg, 8.9 μmol, 89% yield). LC-MS calc. for C₂₄H₂₇N₄O₃ [M+H]⁺: m/z=483.2; Found: 483.2.

### Example 28: 3-((1-(2-(isoindolin-2-yl)-3,6-dimethyl-4-oxo-3,4-dihydroquinazolin-8-yl)ethyl)amino)picolinic acid

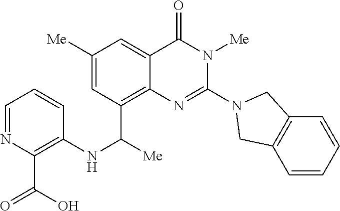 [SMILES: Cc1cc(C(C)Nc2cccnc2C(=O)O)c2nc(N3Cc4ccccc4C3)n(C)c(=O)c2c1]

The title compound was synthesized by procedures analogous to those outlined in Example 1, Step 7 except that Step 7 was carried out at 110° C. in DMF. LC-MS calc. for C₂₆H₂₆N₅O₃ [M+H]⁺: m/z=456.2; Found: 456.1.

### Examples 29-36

Examples 29-36 listed in Tables 5 and 6 were synthesized according to procedures analogous to Example 1 or Example 28. All examples in this table were prepared as the TFA salt.

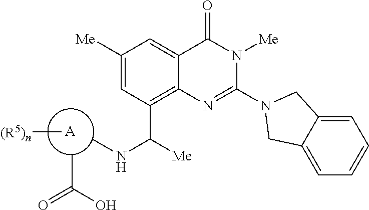

TABLE 5
Examples 29-36

| Example | Amino acid moiety | LCMS [M + H]⁺ |
|---|---|---|
| 29 | 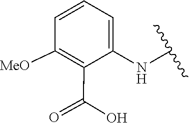 [SMILES: *Nc1cccc(OC)c1C(=O)O] | 485.1 |
| 30 | 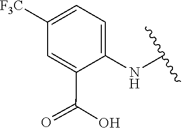 [SMILES: *Nc1ccc(C(F)(F)F)cc1C(=O)O] | 523.1 |
| 31 | 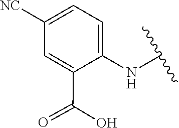 [SMILES: *Nc1ccc(C#N)cc1C(=O)O] | 480.1 |
| 32 | 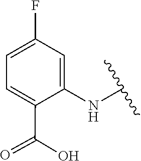 [SMILES: *Nc1cc(F)ccc1C(=O)O] | 473.1 |
| 33 | 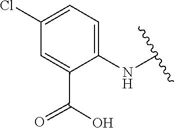 [SMILES: *Nc1ccc(Cl)cc1C(=O)O] | 489.1 |
| 34 | 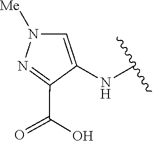 [SMILES: *Nc1cn(C)nc1C(=O)O] | 459.1 |
| 35 | 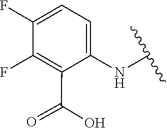 [SMILES: *Nc1ccc(F)c(F)c1C(=O)O] | 491.1 |
| 36 | 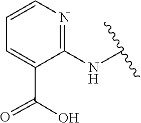 [SMILES: *Nc1ncccc1C(=O)O] | 456.1 |

TABLE 6
Examples 29-36

| Example | Compound name | NMR |
|---|---|---|
| 29 | 2-((1-(2-(isoindolin-2-yl)-3,6- | ¹H NMR (400 MHz, acetone-d₆) δ 7.79 (s, 1H), |
| | dimethyl-4-oxo-3,4- | 7.48 (d, J = 2.1 Hz, 1H), 7.42 (dd, J = 5.5, 3.2 Hz, |
| | dihydroquinazolin-8-yl)ethyl) | 2H), 7.37-7.31 (m, 3H), 7.08 (t, J = 8.4 Hz, 1H), |
| | amino)-6-methoxybenzoic | 6.28 (dd, J = 18.7, 8.4 Hz, 2H), 5.56 (q, J = 6.6 |
| | acid | Hz, 1H), 5.22-5.05 (m, 4H), 4.05 (s, 3H), 3.72 |
| | | (s, 3H), 2.35 (s, 3H), 1.62 (d, J = 6.7 Hz, 3H). |
| 30 | 2-((1-(2-(isoindolin-2-yl)-3,6- | ¹H NMR (400 MHz, CD₃OD) δ 8.11 (s, 1H), 7.81 |
| | dimethyl-4-oxo-3,4- | (s, 1H), 7.49 (d, J = 2.1 Hz, 1H), 7.40-7.28 (m, |
| | dihydroquinazolin-8-yl)ethyl) | 5H), 6.61 (d, J = 8.9 Hz, 1H), 5.56 (q, J = 6.8 Hz, |
| | amino)-5-(trifluoromethyl) | 1H), 5.14-4.99 (m, 4H), 3.72 (s, 3H), 2.36 (s, |
| | benzoic acid | 3H), 1.67 (d, J = 6.7 Hz, 3H). |
| 31 | 5-cyano-2-((1-(2-(isoindolin- | ¹H NMR (400 MHz, CD₃OD) δ 8.17 (d, J = 2.1 |
| | 2-yl)-3,6-dimethyl-4-oxo-3,4- | Hz, 1H), 7.81 (d, J = 2.0 Hz, 1H), 7.47 (s, 1H), |
| | dihydroquinazolin-8- | 7.36 (dt, J = 7.4, 3.6 Hz, 3H), 7.33-7.28 (m, 2H), |
| | yl)ethyl)amino)benzoic acid | 6.62 (d, J = 9.0 Hz, 1H), 5.55 (q, J = 6.7 Hz, 1H), |
| | | 5.14-4.98 (m, 4H), 3.72 (s, 3H), 2.36 (s, 3H), |
| | | 1.68 (d, J = 6.7 Hz, 3H). |
| 32 | 4-fluoro-2-((1-(2-(isoindolin- | ¹H NMR (400 MHz, acetone-d₆) δ 8.71 (s, 1H), |
| | 2-yl)-3,6-dimethyl-4-oxo-3,4- | 7.95 (dd, J = 8.8, 7.0 Hz, 1H), 7.79 (dd, J = 2.1, |
| | dihydroquinazolin-8-yl)ethyl) | 1.0 Hz, 1H), 7.52 (d, J = 2.1 Hz, 1H), 7.39 (dd, J = |
| | amino)benzoic acid | 5.5, 3.3 Hz, 2H), 7.35-7.29 (m, 2H), 6.32- |
| | | 6.22 (m, 2H), 5.51 (q, J = 6.7 Hz, 1H), 5.18-5.07 |
| | | (m, 4H), 3.71 (s, 3H), 2.34 (s, 3H), 1.64 (d, J = |
| | | 6.7 Hz, 3H). |
| 33 | 5-chloro-2-((1-(2-(isoindolin- | ¹H NMR (400 MHz, acetone-d₆) δ 7.83 (d, J = 2.6 |
| | 2-yl)-3,6-dimethyl-4-oxo-3,4- | Hz, 1H), 7.78 (d, J = 2.1 Hz, 1H), 7.49 (d, J = 2.1 |
| | dihydroquinazolin-8-yl)ethyl) | Hz, 1H), 7.40 (dd, J = 5.5, 3.2 Hz, 2H), 7.35- |
| | amino)benzoic acid | 7.28 (m, 2H), 7.12 (dd, J = 9.1, 2.7 Hz, 1H), 6.58 |
| | | (d, J = 9.1 Hz, 1H), 5.54 (q, J = 6.7 Hz, 1H), 5.21- |
| | | 5.02 (m, 4H), 3.71 (s, 3H), 2.33 (s, 3H), 1.63 (d, |
| | | J = 6.7 Hz, 3H). |
| 34 | 4-((1-(2-(isoindolin-2-yl)-3,6- | ¹H NMR (400 MHz, acetone-d₆) δ 7.75 (s, 1H), |
| | dimethyl-4-oxo-3,4- | 7.54 (s, 1H), 7.41 (dd, J = 5.5, 3.2 Hz, 2H), 7.33 |
| | dihydroquinazolin-8-yl)ethyl) | (dd, J = 5.7, 3.1 Hz, 2H), 6.82 (s, 1H), 5.18-4.98 |
| | amino)-1-methyl-1H- | (m, 5H), 3.68 (s, 3H), 3.63 (s, 3H), 2.35 (s, 3H), |
| | pyrazole-3-carboxylic acid | 1.59 (d, J = 6.7 Hz, 3H). |
| 35 | 2,3-difluoro-6-((1-(2- | ¹H NMR (400 MHz, acetone-d₆) δ 7.77 (dd, J = |
| | (isoindolin-2-yl)-3,6-dimethyl- | 2.1, 1.0 Hz, 1H), 7.48 (d, J = 2.2 Hz, 1H), 7.43- |
| | 4-oxo-3,4-dihydroquinazolin- | 7.36 (m, 2H), 7.36-7.29 (m, 2H), 7.09 (td, J = |
| | 8-yl)ethyl)amino)benzoic acid | 9.7, 8.6 Hz, 1H), 6.30 (ddd, J = 9.5, 3.8, 2.0 Hz, |
| | | 1H), 5.50 (q, J = 6.7 Hz, 1H), 5.19-5.04 (m, 4H), |
| | | 3.70 (s, 3H), 2.33 (s, 3H), 1.61 (d, J = 6.7 Hz, |
| | | 3H). |
| 36 | 2-((1-(2-(isoindolin-2-yl)-3,6- | ¹H NMR (400 MHz, acetone-d₆) δ 9.03 (s, 1H), |
| | dimethyl-4-oxo-3,4-dihydro- | 8.26-8.17 (m, 2H), 7.78 (s, 1H), 7.52 (d, J = 2.1 |
| | quinazolin-8-yl)ethyl)amino) | Hz, 1H), 7.41-7.35 (m, 2H), 7.35-7.28 (m, |
| | nicotinic acid | 2H), 6.60 (dd, J = 7.7, 4.9 Hz, 1H), 6.07 (s, 1H), |
| | | 5.15-5.04 (m, 4H), 3.68 (s, 3H), 2.37 (s, 3H), |
| | | 1.71 (d, J = 6.9 Hz, 3H). |

### Example 37: 2-((1-(2-(4,4-difluoropiperidin-1-yl)-3,6-dimethyl-4-oxo-3,4-dihydroquinazolin-8-yl)ethyl)amino)benzoic acid (isomer 1)

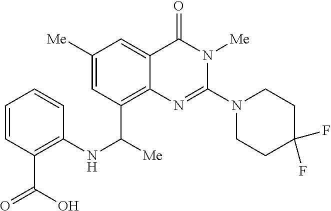 [SMILES: Cc1cc(C(C)Nc2ccccc2C(=O)O)c2nc(N3CCC(F)(F)CC3)n(C)c(=O)c2c1]

### Step 1. Methyl 2-((1-(2-(4,4-difluoropiperidin-1-yl)-3,6-dimethyl-4-oxo-3,4-dihydroquinazolin-8-yl)ethyl)amino)benzoate (isomer 11)

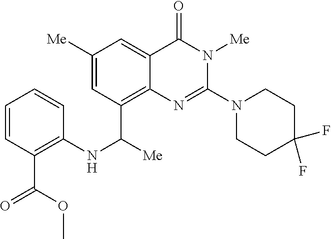 [SMILES: COC(=O)c1ccccc1NC(C)c1cc(C)cc2c(=O)n(C)c(N3CCC(F)(F)CC3)nc12]

The title compound was synthesized by procedures analogous to those outlined in Example 24. The isomers were separated using chiral prep-HPLC on an i-Amylose-3 column (30 mL/min, 80:10:10 Hexanes/IPA/MeOH) to afford two isomers: isomer 1 (t_{R}=1.90 min) and isomer 2 (t_{R}=2.74 min). Isomer 1: LC-MS calc. for C₂₅H₂₉F₂N₄O₃ [M+H]⁺: m/z=471.2; Found: 471.1.

### Step 2. 2-((1-(2-(4,4-difluoropiperidin-1-yl)-3,6-dimethyl-4-oxo-3,4-dihydroquinazolin-8-yl)ethyl)amino)benzoic acid (isomer 1)

To a solution of methyl 2-((1-(2-(4,4-difluoropiperidin-1-yl)-3,6-dimethyl-4-oxo-3,4-dihydroquinazolin-8-yl)ethyl)amino)benzoate (45.0 mg, 0.0956 mmol) in dioxane (2.0 mL) and water (2.0 mL) was added lithium hydroxide (32.1 mg, 0.765 mmol). The mixture was stirred at 90° C. for 2 h. The mixture was acidified with acetic acid and purified by prep HPLC on a CSH Phenyl-Hexyl column (52-66% MeCN/0.1% TFA (aq.)) to afford the title compound as a TFA salt (26.0 mg, 0.0456 mmol, 48% yield). ¹H NMR (400 MHz, DMSO-d₆) δ 8.40 (s, 1H), 7.77 (dd, J=7.9, 1.7 Hz, 1H), 7.74-7.70 (m, 1H), 7.48 (d, J=2.1 Hz, 1H), 7.26-6.95 (m, 2H), 6.52-6.44 (m, 2H), 5.39 (q, J=6.6 Hz, 1H), 3.51 (s, 3H), 3.39 (t, J=5.7 Hz, 4H), 2.32 (s, 3H), 2.19 (d, J=18.8 Hz, 4H), 1.55 (d, J=6.7 Hz, 3H). ¹⁹F NMR (376 MHz, DMSO-d₆) δ −95.17, −74.61. LC-MS calc. for C₂₄H₂₇F₂N₄O₃ [M+H]⁺: m/z=457.2; Found: 457.1.

### Example 38: 2-((1-(5-acrylamido-2-(3-azabicyclo[3.1.0]hexan-3-yl)-3,6-dimethyl-4-oxo-3,4-dihydroquinazolin-8-yl)ethyl)amino)benzoic acid

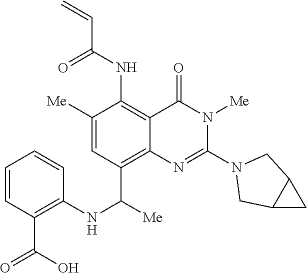 [SMILES: C=CC(=O)Nc1c(C)cc(C(C)Nc2ccccc2C(=O)O)c2nc(N3CC4CC4C3)n(C)c(=O)c12]

### Step 1. tert-butyl 2-((1-(5-acrylamido-2-(3-azabicyclo[3.1.0]hexan-3-yl)-3,6-dimethyl-4-oxo-3,4-dihydroquinazolin-8-yl)ethyl)amino)benzoate

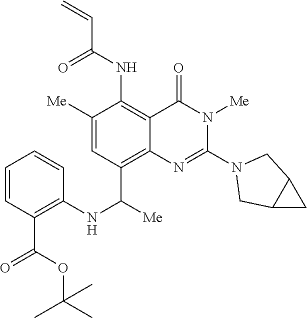 [SMILES: C=CC(=O)Nc1c(C)cc(C(C)Nc2ccccc2C(=O)OC(C)(C)C)c2nc(N3CC4CC4C3)n(C)c(=O)c12]

tert-Butyl-2-[1-[2-(3-azabicyclo[3.1.0]hexan-3-yl)-5-bromo-3,6-dimethyl-4-oxoquinazolin-8-yl]ethylamino]benzoate (35.0 mg, 0.0632 mmol) (Example 14, Step 9), prop-2-enamide (27.0 mg, 0.379 mmol), K₃PO₄ (40.3 mg, 0.190 mmol), copper(I) iodide (6.0 mg, 0.032 mmol) and N1,N2-dimethylethane-1,2-diamine (6.81 uL, 0.0632 mmol) were added to a 4 mL scintillation vial. 1,4-Dioxane (1.0 mL) was added, the vial was capped, and the reaction mixture was flushed with N2. After stirring at 100° C. overnight, the reaction mixture was cooled to room temperature, concentrated, diluted with water, and extracted with ethyl acetate (10 mL×2). The combined organic layers were washed with brine, concentrated, and purified by prep HPLC on a CSH C18 column (75-100% MeCN/0.1% TFA (aq.)) to afford the title compound as a TFA salt (15.0 mg, 0.0228 mmol). LC-MS calc. for C₃₁H₃₇N₅NaO₄ [M+Na]⁺: m/z=566.3; Found: 566.1.

### Step 2. 2-((1-(5-acrylamido-2-(3-azabicyclo[3.1.0]hexan-3-yl)-3,6-dimethyl-4-oxo-3,4-dihydroquinazolin-8-yl)ethyl)amino)benzoic acid

A solution of tert-butyl 2-((1-(5-acrylamido-2-(3-azabicyclo[3.1.0]hexan-3-yl)-3,6-dimethyl-4-oxo-3,4-dihydroquinazolin-8-yl)ethyl)amino)benzoate (8.0 mg, 0.015 mmol) in DCM (0.5 mL) and TFA (0.5 mL) was stirred overnight. The reaction mixture was concentrated and purified by prep IPLC on a CSH C18 column (75-100% MeCN/0.1% TFA (aq.)) to afford the title compound as a TFA salt (1.0 mg, 2.0 μmol, 14% yield). LC-MS calc. for C₂₇H₂₉N5NaO₄ [M+Na]⁺: m/z=510.2; Found: 510.1.

### Example 39: 2-[1-[2-(5-azaspiro[2.4]heptan-5-yl)-3,6-dimethyl-4-oxoquinazolin-8-yl]ethyl-amino]benzoic acid

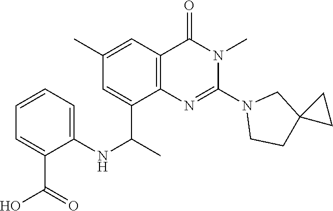 [SMILES: Cc1cc(C(C)Nc2ccccc2C(=O)O)c2nc(N3CCC4(CC4)C3)n(C)c(=O)c2c1]

### Step 1: 2-(5-azaspiro[2.4]heptan-5-yl)-8-bromo-3,6-dimethylquinazolin-4-one

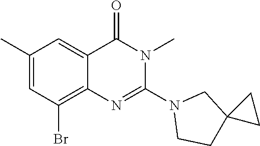 [SMILES: Cc1cc(Br)c2nc(N3CCC4(CC4)C3)n(C)c(=O)c2c1]

To a solution of 8-bromo-2-chloro-3,6-dimethylquinazolin-4-one (80.0 mg, 0.278 mmol) in THF (2 mL) was added diisopropylethylamine (0.145 mL, 0.835 mmol) and 5-azaspiro[2.4]heptane (0.0614 mL, 0.556 mmol). The resulting mixture was stirred overnight. The mixture was concentrated, and the crude product was directly purified by silica gel chromatography (0-30% EtOAc/heptane) to afford the tile compound (93.1 mg, 0.267 mmol, 96.1% yield) as a white solid. ¹H NMR (300 MHz, CDCl₃) δ 7.89 (dd, J=2.1, 1.0 Hz, 1H), 7.73 (d, J=2.0 Hz, 1H), 3.77 (t, J=6.8 Hz, 2H), 3.54 (s, 3H), 3.50 (s, 2H), 2.39 (s, 3H), 1.90 (t, J=6.8 Hz, 2H), 0.65 (d, J=2.4 Hz, 4H). LC-MS calc. for C₁₆H₁₉BrN₃O [M+H]⁺: m/z=348.1/350.1; Found 347.8/349.8.

### Step 2: 8-acetyl-2-(5-azaspiro[2.4]heptan-5-yl)-3,6-dimethylquinazolin-4-one

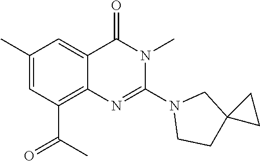 [SMILES: CC(=O)c1cc(C)cc2c(=O)n(C)c(N3CCC4(CC4)C3)nc12]

To a solution of 2-(5-azaspiro[2.4]heptan-5-yl)-8-bromo-3,6-dimethylquinazolin-4-one (93.0 mg, 0.267 mmol) in 1,4-dioxane (2 mL) was added bis(triphenylphosphine)palladium(II) dichloride (37.5 mg, 0.0534 mmol). The resulting mixture was purged with N2 (3×). Tributyl(1-ethoxyvinyl)tin (0.226 mL, 0.668 mmol) was added, and the mixture was stirred at 100° C. for 5 h. The mixture was cooled to room temperature and quenched with sat. KF (aq.) (5 mL), and the mixture was stirred for 2 h. The reaction mixture was diluted with water (5 mL) and EtOAc (10 mL). The layers were separated, and the aqueous layer was extracted with EtOAc (2×10 mL). The combined organic layers were washed with brine, dried over Na₂SO₄, filtered, and concentrated. The crude material was diluted with 1N HCl (3 mL) and THE (3 mL). The reaction was stirred for 2 h. The aqueous layer was adjusted to pH ˜8-9 with sat. NaHCO₃ and further diluted with EtOAc (10 mL). The layers were separated, and the aqueous layer was extracted with EtOAc (2×10 mL). The combined organic layers were washed with brine, dried over Na₂SO₄, filtered, and concentrated. The crude product was purified by silica gel chromatography (10-40% EtOAc/heptane) to afford the title compound (59.6 mg, 0.191 mmol, 71.7% yield) as a white solid. ¹H NMR (300 MHz, CDCl₃) δ 8.12 (dd, J=2.3, 1.1 Hz, 1H), 7.84 (d, J=2.3 Hz, 1H), 3.74 (t, J=6.8 Hz, 2H), 3.56 (s, 3H), 3.44 (s, 2H), 2.85 (s, 3H), 2.42 (s, 3H), 1.90 (t, J=6.8 Hz, 2H), 0.66 (s, 4H). LC-MS calc. for C₁₈H₂₂N₃O₂ [M+H]⁺: m/z=312.2; Found 312.2.

### Step 3: 2-(5-azaspiro[2.4]heptan-5-yl)-8-(1-hydroxyethyl)-3,6-dimethylquinazolin-4-one

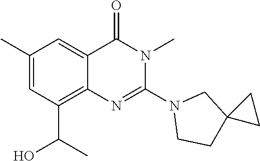 [SMILES: Cc1cc(C(C)O)c2nc(N3CCC4(CC4)C3)n(C)c(=O)c2c1]

To a solution of 8-acetyl-2-(5-azaspiro[2.4]heptan-5-yl)-3,6-dimethylquinazolin-4-one (52.0 mg, 0.167 mmol) in methanol (2 mL) was added sodium borohydride (12.6 mg, 0.334 mmol). The resulting mixture was stirred for 2 h. The reaction was quenched with water (5 mL), and the reaction mixture was diluted with EtOAc (5 mL). The layers were separated, and the aqueous layer was extracted with EtOAc (2×5 mL). The combined organic layers were washed with brine, dried over Na₂SO₄, filtered, and concentrated. The crude material was purified by silica gel chromatography (10-60% EtOAc/heptane) to afford the title compound (50.0 mg, 0.160 mmol, 95.5% yield) as a white solid. ¹H NMR (300 MHz, CDCl₃) δ 7.85 (dd, J=2.1, 1.0 Hz, 1H), 7.28 (d, J=2.1 Hz, 1H), 5.84 (d, J=6.7 Hz, 1H), 5.10 (t, J=6.5 Hz, 1H), 3.79-3.64 (m, 2H), 3.55 (s, 3H), 3.47 (d, J=9.7 Hz, 1H), 3.37 (d, J=9.7 Hz, 1H), 2.40 (s, 3H), 1.91 (ddt, J=14.3, 12.1, 6.3 Hz, 2H), 1.62 (d, J=6.6 Hz, 3H), 0.66 (s, 4H). LC-MS calc. for C₁₈H₂₄N₃O₂ [M+H]⁺: m/z=314.2; Found 314.2.

### Step 4: 2-(5-azaspiro[2.4]heptan-5-yl)-8-(1-bromoethyl)-3,6-dimethylquinazolin-4-one

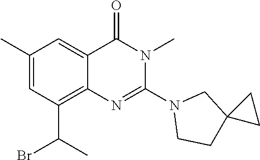 [SMILES: Cc1cc(C(C)Br)c2nc(N3CCC4(CC4)C3)n(C)c(=O)c2c1]

To a solution of 2-(5-azaspiro[2.4]heptan-5-yl)-8-(1-hydroxyethyl)-3,6-dimethyl-quinazolin-4-one (25.0 mg, 0.0798 mmol) in DCM (1 mL) was added phosphorus tribromide (0.0227 mL, 0.239 mmol). The resulting mixture was stirred at 35° C. for 2 h. The reaction was quenched with sat. NaHCO₃ solution (10 mL), and the reaction mixture was diluted with EtOAc (10 mL). The layers were separated, and the aqueous layer was extracted with EtOAc (2×10 mL). The combined organic layers were washed with brine, dried over Na₂SO₄, filtered, and concentrated to afford the title compound (30.0 mg, 0.0797 mmol, >99%) as a colorless oil. The crude product was used in the next step directly without further purification.

### Step 5: 2-[1-[2-(5-azaspiro[2.4]heptan-5-yl)-3,6-dimethyl-4-oxoquinazolin-8-yl]ethyl-amino]benzoic acid

To a solution of 2-(5-azaspiro[2.4]heptan-5-yl)-8-(1-bromoethyl)-3,6-dimethylquinazolin-4-one (30.0 mg, 0.0797 mmol) in DCM (1 mL) was added anthranilic acid (54.7 mg, 0.399 mmol) and diisopropylethylamine (0.0694 mL, 0.399 mmol). The mixture was stirred overnight. The reaction mixture was directly purified by prep-HPLC on C18 column (20-100% MeCN/0.1% TFA (aq.)) to afford the title compound as a TFA salt (19.0 mg, 0.0438 mmol, 44.2% yield), a white solid. ¹H NMR (300 MHz, DMSO-d₆) δ 12.53 (s, 1H), 8.35 (s, 1H), 7.77 (dd, J=7.9, 1.7 Hz, 1H), 7.66 (dd, J=2.1, 1.0 Hz, 1H), 7.40 (d, J=2.1 Hz, 1H), 7.18 (ddd, J=8.6, 7.2, 1.8 Hz, 1H), 6.53-6.41 (m, 2H), 5.35 (q, J=6.6 Hz, 1H), 3.76 (td, J=6.8, 1.8 Hz, 2H), 3.54 (d, J=10.2 Hz, 1H), 3.52-3.46 (m, 4H), 2.29 (s, 3H), 1.97-1.76 (m, 2H), 1.55 (d, J=6.6 Hz, 3H), 0.69-0.55 (m, 4H). LC-MS calc. for C₂₅H₂₉N₄O₃ [M+H]⁺: m/z=433.2; Found 433.1.

### Example 41: 2-[1-(2-methoxy-3,6-dimethyl-4-oxoquinazolin-8-yl)ethylamino]benzoic acid

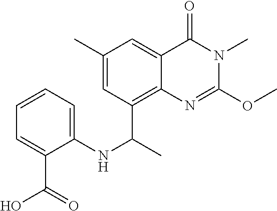 [SMILES: COc1nc2c(C(C)Nc3ccccc3C(=O)O)cc(C)cc2c(=O)n1C]

### Step 1: 8-bromo-2-methoxy-3,6-dimethylquinazolin-4-one

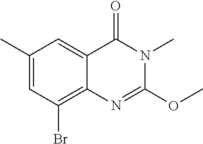

To a solution of 8-bromo-2-chloro-3,6-dimethylquinazolin-4-one (200 mg, 0.696 mmol) in methanol (4 mL) was added sodium methoxide (113 mg, 2.09 mmol). The resulting mixture was stirred overnight. Additional sodium methoxide (0.042 mL, 0.209 mmol, 5 N in MeOH) was added, and the mixture was stirred overnight. The reaction was quenched with sat. NH₄Cl solution (20 mL), and the reaction mixture was diluted with EtOAc (20 mL). The layers were separated, and the aqueous layer was extracted with EtOAc (2×20 mL). The combined organic layers were washed with brine, dried over Na₂SO₄, filtered, and concentrated. The crude material was purified by silica gel chromatography (0-30% EtOAc/heptane) to afford the title compound (147 mg, 0.518 mmol, 74.4% yield) as a white solid. t_{R}=6.6 min (C18 column; 5-95% MeCN/0.1% TFA (aq.) for 1 min, 5-95% MeCN/0.1% TFA (aq.) over 4 min, 95% MeCN/0.1% TFA (aq.) for 5 min; 1 mL/min).

### Step 2: 8-acetyl-2-methoxy-3,6-dimethylquinazolin-4-one

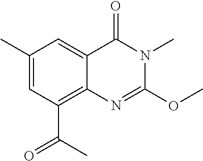

The title compound was synthesized by procedures analogous to Example 39, Step 2. LC-MS calc. for C₁₃H₁₅N₂O₃ [M+H]⁺: m/z=247.1; Found 247.1.

### Step 3: 8-(1-hydroxyethyl)-2-methoxy-3,6-dimethylquinazolin-4-one

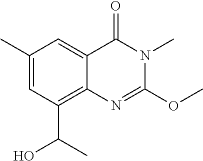

The title compound was synthesized by procedures analogous to Example 39, Step 3. LC-MS calc. for C₁₃H₁₆N₂NaO₃ [M+Na]⁺: m/z=271.1; Found 271.1.

### Step 4: 2-[1-(2-methoxy-3,6-dimethyl-4-oxoquinazolin-8-yl)ethylamino]benzoic acid

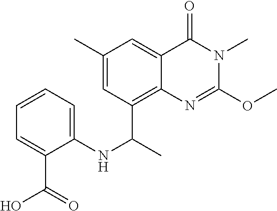 [SMILES: COc1nc2c(C(C)Nc3ccccc3C(=O)O)cc(C)cc2c(=O)n1C]

To a solution of 8-(1-hydroxyethyl)-2-methoxy-3,6-dimethylquinazolin-4-one (31.2 mg, 0.126 mmol) in DCM (3 mL) was added methanesulfonyl chloride (0.0290 mL, 0.375 mmol) and triethylamine (0.0710 mL, 0.509 mmol). The mixture was stirred at 35° C. for 0.5 h. The reaction mixture was cooled to room temperature and diluted with water (3 mL). The layers were separated, and the aqueous phase was extracted with DCM (3×3 mL). The combined organic layers were washed with brine, dried over Na₂SO₄, filtered, concentrated, and redissolved in DCM (6 mL). To the mixture was added anthranilic acid (86.0 mg, 0.627 mmol) and triethylamine (0.125 mL, 0.897 mmol). After stirring at 35° C. for 2 h and then 30° C. for 2 d, the mixture was concentrated and purified by prep-HPLC on C18 column (10-90% MeCN/0.1% TFA (aq.)) to afford the title compound as a TFA salt (1.7 mg, 0.0043 mmol, 3.4% yield), a white solid. ¹H NMR (300 MHz, CD₃OD) δ 7.88 (dd, J=8.0, 1.7 Hz, 1H), 7.80 (d, J=2.0 Hz, 1H), 7.52 (d, J=2.1 Hz, 1H), 7.13 (ddd, J=8.7, 7.1, 1.7 Hz, 1H), 6.58-6.36 (m, 2H), 5.48 (q, J=6.7 Hz, 1H), 4.18 (s, 3H), 3.53 (s, 3H), 2.35 (s, 3H), 1.63 (d, J=6.7 Hz, 3H). LC-MS calc. for C₂₀H₂₂N₃O₄ [M+H]: m/z=368.2; Found 368.0.

### Example 42: 2-((1-(2-hydroxy-3,6-dimethyl-4-oxo-3,4-dihydroquinazolin-8-yl)ethyl)amino) benzoic acid

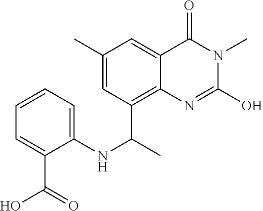 [SMILES: Cc1cc(C(C)Nc2ccccc2C(=O)O)c2nc(O)n(C)c(=O)c2c1]

To a solution of 8-(1-hydroxyethyl)-2-methoxy-3,6-dimethylquinazolin-4-one (31.2 mg, 0.126 mmol) in DCM (2 mL) was added phosphorus tribromide (0.0360 mL, 0.379 mmol). The mixture was stirred at 40° C. for 1 h. The reaction mixture was diluted with sat. NaHCO₃(aq.) (2 mL) and extracted with DCM (3×3 mL). The combined organic layers were washed with brine, dried over Na₂SO₄, filtered, and concentrated. The crude material was redissolved in DCM (3 mL). Anthranilic acid (81.4 mg, 0.594 mmol) and triethylamine (0.0900 mL, 0.646 mmol) were added. Upon stirring at 35° C. for 2 h, the mixture was concentrated and purified by prep-HPLC on a C18 column (20-70% MeCN/0.1 TFA (aq.)) to afford the title compound as a TFA salt (14 mg, 0.039 mmol, 31% yield), a white solid. ¹H NMR (300 MHz, DMSO-d₆) δ 12.79 (s, 1H), 10.97 (s, 1H), 8.33 (d, J=5.6 Hz, 1H), 7.81 (dd, J=8.0, 1.7 Hz, 1H), 7.68 (d, J=2.0 Hz, 1H), 7.39 (d, J=2.1 Hz, 1H), 7.22 (ddd, J=8.7, 7.1, 1.7 Hz, 1H), 6.55 (t, J=7.5 Hz, 1H), 6.31 (d, J=8.4 Hz, 1H), 5.24-5.20 (m, 1H), 3.29 (s, 3H), 2.25 (s, 3H), 1.42 (d, J=6.3 Hz, 3H). LC-MS calc. for C₁₉H₂₀N₃O₄ [M+H]⁺: m/z=354.1; Found 354.1.

### Example 43: 2-[1-(3,6-dimethyl-2-methylsulfanyl-4-oxoquinazolin-8-yl)ethylamino]benzoic acid

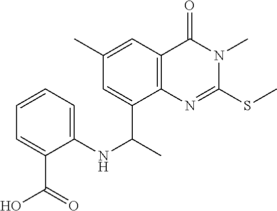 [SMILES: CSc1nc2c(C(C)Nc3ccccc3C(=O)O)cc(C)cc2c(=O)n1C]

### Step 1: 8-bromo-3,6-dimethyl-2-sulfanylidene-1H-quinazolin-4-one

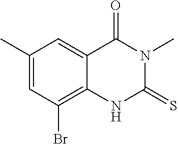

To a solution of 2-amino-3-bromo-N,5-dimethylbenzamide (10.0 g, 41.1 mmol) in THF (100 mL) was added 1,1′-thiocarbonyldiimidazole (11.0 g, 61.7 mmol) and 1,8-diazabicyclo[5.4.0]undec-7-ene (12.5 g, 82.3 mmol. The resulting mixture was stirred at 70° C. for 27 h. The reaction mixture was cooled to 0° C. and diluted with sat. NH₄Cl solution. The resulting solid was collected by filtration, triturated with water (3×), and dried by co-evaporating with toluene to afford the title compound (10.8 g, 37.9 mmol, 92.1% yield) as a brown solid. LC-MS calc. for C₁₀H₁₀BrN₂OS [M+H]⁺: m/z=285.0; Found 285.4.

### Step 2: 8-bromo-3,6-dimethyl-2-methylsulfanylquinazolin-4-one

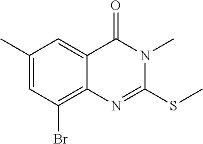

To a solution of 8-bromo-3,6-dimethyl-2-sulfanylidene-1H-quinazolin-4-one (10.8 g, 37.9 mmol) in THF (108 mL) was added iodomethane (8.06 g, 56.8 mmol) and triethylamine (7.66 g, 75.8 mmol) at 0° C. The resulting mixture was stirred for 18 h at room temperature. The mixture was poured into water. The resulting solid was collected by filtration, washed with water (3×), and co-evaporated with toluene (3×) to afford the title compound (10.0 g, 33.4 mmol, 88.3% yield) as a brown solid. LC-MS calc. for C₁₁H₁₂BrN₂OS [M+H]⁺: m/z=299.0; Found 299.5.

### Step 3: 8-acetyl-3,6-dimethyl-2-methylsulfanylquinazolin-4-one

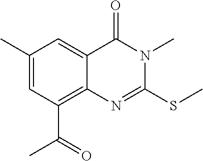

To a solution of 8-bromo-3,6-dimethyl-2-methylsulfanylquinazolin-4-one (10.0 g, 33.4 mmol) in 1,4-dioxane (120 mL) was added tributyl(1-ethoxyvinyl)tin (18.1 mL, 53.5 mmol) and bis-(triphenylphosphine)palladium(II) dichloride (4.69 g, 6.68 mmol). The reaction was stirred at 100° C. for 18 h. The reaction mixture was cooled to 0° C., and 1 N HCl was added to acidify to pH 2-3. The resulting mixture was stirred at room temperature for 1 h. The reaction mixture was filtered. The layers were separated, and the aqueous layer was extracted with EtOAc (3×50 mL). The combined organic layers were dried over Na₂SO₄, filtered, concentrated, and purified by silica gel chromatography (20% EtOAc/hexanes) to afford the title compound (4.93 g, 18.8 mmol, 56.2% yield) as a yellow solid. LC-MS calc. for C₁₃H₁₅N₂O₂S [M+H]⁺: m/z=263.1; Found 263.5.

### Step 4: 8-(1-hydroxyethyl)-3,6-dimethyl-2-methylsulfanylquinazolin-4-one

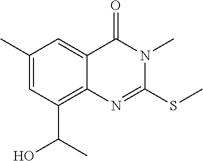

The title compound was synthesized by procedures analogous to Example 39, Step 3. LC-MS calc. for C₁₃H₁₇N₂O₂S [M+H]⁺: m/z=265.1; Found 265.5.

### Step 5: Methyl 2-[1-(3,6-dimethyl-2-methylsulfanyl-4-oxoquinazolin-8-yl)ethylamino]benzoate

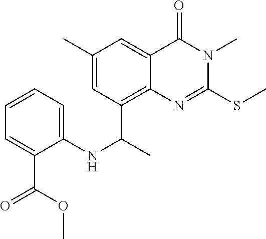 [SMILES: COC(=O)c1ccccc1NC(C)c1cc(C)cc2c(=O)n(C)c(SC)nc12]

The title compound was synthesized by procedures analogous to Example 41, Step 4. LC-MS calc. for C₂₁H₂₄N₃O₃S [M+H]⁺: m/z=398.2; Found 398.1.

### Step 6: 2-[1-(3,6-dimethyl-2-methylsulfanyl-4-oxoquinazolin-8-yl)ethylamino]benzoic acid

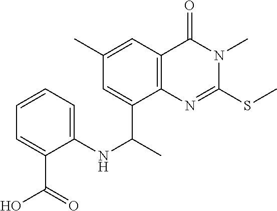 [SMILES: CSc1nc2c(C(C)Nc3ccccc3C(=O)O)cc(C)cc2c(=O)n1C]

To a solution of methyl 2-[1-(3,6-dimethyl-2-methylsulfanyl-4-oxoquinazolin-8-yl)ethyl-amino]benzoate (20.0 mg, 0.0503 mmol) in methanol (1 mL), THE (1 mL), and water (0.5 mL) was added NaOH (20.1 mg, 0.503 mmol). After stirring at 50° C. for 7 h, the reaction mixture was cooled to 0° C., and 1N HCl was added to adjust to pH 2-3. The mixture was extracted with EtOAc (3×5 mL). The combined organic layers were washed with brine, dried over Na₂SO₄, filtered, concentrated, and purified by prep-HPLC on a C18 column (50-100% MeCN/0.1% TFA (aq.)) to afford the title compound as a TFA salt (16 mg, 0.042 mmol, 83% yield), a white solid. ¹H NMR (400 MHz, DMSO-d₆) δ 12.68 (s, 1H), 8.43 (s, 1H), 7.77 (dd, J=8.0, 1.6 Hz, 2H), 7.52 (d, J=2.0 Hz, 1H), 7.18 (ddd, J=8.7, 7.1, 1.8 Hz, 1H), 6.49 (t, J=7.5 Hz, 1H), 6.43 (d, J=8.5 Hz, 1H), 5.45 (q, J=7.0 Hz, 1H), 3.52 (s, 3H), 2.68 (s, 3H), 2.33 (s, 3H), 1.58 (d, J=6.7 Hz, 3H). LC-MS calc. for C₂₀H₂₀N₃O₃S [M−H]⁻: m/z=384.1; Found 384.1.

### Example 44: 2-((1-(2-(4-cyanopiperidin-1-yl)-3,6-dimethyl-4-oxo-3,4-dihydroquinazolin-8-yl)ethyl)amino)benzoic acid

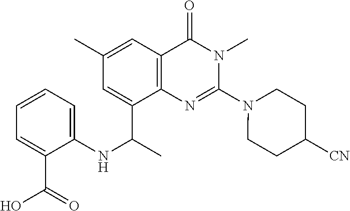 [SMILES: Cc1cc(C(C)Nc2ccccc2C(=O)O)c2nc(N3CCC(C#N)CC3)n(C)c(=O)c2c1]

### Step 1: Methyl 2-((1-(3,6-dimethyl-2-(methylsulfonyl)-4-oxo-3,4-dihydroquinazolin-8-yl)ethyl)amino)benzoate

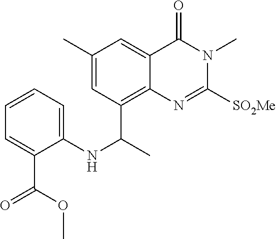 [SMILES: COC(=O)c1ccccc1NC(C)c1cc(C)cc2c(=O)n(C)c(S(C)(=O)=O)nc12]

To a solution of methyl 2-[1-(3,6-dimethyl-2-methylsulfanyl-4-oxoquinazolin-8-yl)ethyl-amino]benzoate (110 mg, 0.277 mmol, Example 43, Step 5) in DCM (5 mL) was added 3-chloro-perbenzoic acid (95.5 mg, 0.553 mmol) at 0° C. Upon stirring at 40° C. for 4 h, the mixture was diluted with water (20 mL) and extracted with EtOAc (2×20 mL). The combined organic layers were washed with sat. NaHCO₃(aq.) and brine (2×30 mL), dried over Na₂SO₄, filtered, and concentrated to afford the title compound (150 mg), which was used to the next step directly without further purification. LC-MS calc. for C₂₁H₂₄N₃O₅S [M+H]⁺: m/z=430.1; Found 430.7.

### Step 2: Methyl 2-((1-(2-(4-cyanopiperidin-1-yl)-3,6-dimethyl-4-oxo-3,4-dihydro-quinazolin-8-yl)ethyl)amino)benzoate

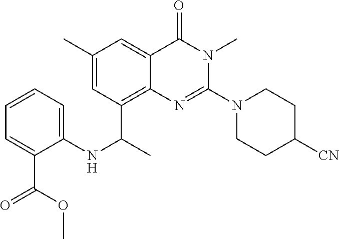 [SMILES: COC(=O)c1ccccc1NC(C)c1cc(C)cc2c(=O)n(C)c(N3CCC(C#N)CC3)nc12]

To a solution of methyl 2-[1-(3,6-dimethyl-2-methylsulfonyl-4-oxoquinazolin-8-yl)ethyl-amino]benzoate (15.0 mg, 0.0349 mmol) in THF (2 mL) was added N,N-diisopropylethylamine (13.5 mg, 0.105 mmol) and piperidine-4-carbonitrile (3.85 mg, 0.0349 mmol) at 35° C. under a nitrogen atmosphere. The reaction was stirred at 70° C. for 14 h. The crude material was purified by prep-HPLC on a C18 column (20-80% MeCN/0.1% TFA (aq.)) to afford the title compound as a TFA salt (10 mg, 0.022 mmol, 62% yield). LC-MS calc. for C₂₆H₂₉N₅NaO₃ [M+Na]⁺: m/z=462.2; Found 462.5.

### Step 3: 2-((1-(2-(4-cyanopiperidin-1-yl)-3,6-dimethyl-4-oxo-3,4-dihydroquinazolin-8-yl)ethyl)amino)benzoic acid

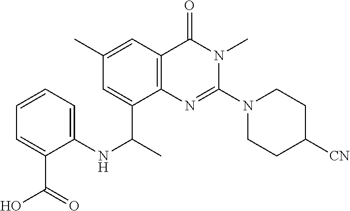 [SMILES: Cc1cc(C(C)Nc2ccccc2C(=O)O)c2nc(N3CCC(C#N)CC3)n(C)c(=O)c2c1]

To a solution of methyl 2-[1-[2-(4-cyanopiperidin-1-yl)-3,6-dimethyl-4-oxoquinazolin-8-yl]ethylamino]benzoate (10.0 mg, 0.0218 mmol) in methanol (1 mL), THE (1 mL), and water (0.3 mL) was added NaOH (2.61 mg, 0.0653 mmol). The mixture was stirred at 40° C. for 4 h. The mixture was acidified to pH 5-6 with 1N HCl, concentrated, and purified by prep-HPLC on a C18 column (10-90% MeCN/0.1% TFA (aq.)) to afford the title compound as a TFA salt (4.4 mg, 0.0098 mmol, 45% yield). LC-MS calc. for C₂₅H₂₈N₅O₃ [M+H]⁺: m/z=446.2; Found 446.0.

### Example 45: 3-fluoro-2-((1-(2-(isoindolin-2-yl)-3,6-dimethyl-4-oxo-3,4-dihydroquinazolin-8-yl)ethyl)amino)benzoic acid

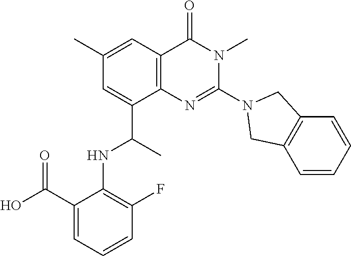 [SMILES: Cc1cc(C(C)Nc2c(F)cccc2C(=O)O)c2nc(N3Cc4ccccc4C3)n(C)c(=O)c2c1]

### Step 1: 8-fluoro-1-(1-(2-(isoindolin-2-yl)-3,6-dimethyl-4-oxo-3,4-dihydroquinazolin-8-yl)ethyl)-2H-benzo[d][1,3]oxazine-2,4(1H)-dione

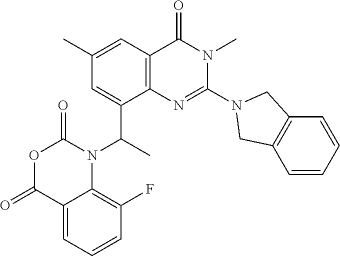 [SMILES: Cc1cc(C(C)n2c(=O)oc(=O)c3cccc(F)c32)c2nc(N3Cc4ccccc4C3)n(C)c(=O)c2c1]

To a solution of 8-fluoro-1H-3,1-benzoxazine-2,4-dione (19.4 mg, 0.107 mmol) and triphenylphosphine (28.2 mg, 0.107 mmol) in THF (1 mL) was added diisopropyl azodicarboxylate (21.1 μL, 0.107 mmol) at 0° C. The mixture was stirred for 10 min. 2-(1,3-Dihydroisoindol-2-yl)-8-(1-hydroxyethyl)-3,6-dimethylquinazolin-4-one (20.0 mg, 0.0596 mmol) was added. After stirring for 4 h, the mixture was concentrated and purified by prep-HPLC on a C18 column (10-80% MeCN/0.1% TFA (aq.)) to afford the title compound as the TFA salt (17 mg, 0.034 mmol, 57% yield) as a colorless solid. LC-MS calc. for C₂₈H₂₄FN₄O₄ [M+H]⁺: m/z=499.2; Found 499.0.

### Step 2: 3-fluoro-2-((1-(2-(isoindolin-2-yl)-3,6-dimethyl-4-oxo-3,4-dihydroquinazolin-8-yl)ethyl)amino)benzoic acid

To a suspension of 1-[1-[2-(1,3-dihydroisoindol-2-yl)-3,6-dimethyl-4-oxoquinazolin-8-yl]ethyl]-8-fluoro-3,1-benzoxazine-2,4-dione (10.0 mg, 0.0201 mmol, from Step 1) in MeCN (0.5 mL) was added 2 N NaOH (5 drops). The mixture was stirred for 30 min. To the mixture was added 2 N HCl (0.5 mL). The mixture was diluted with MeCN and purified by prep-HPLC on a C18 column (10-70% MeCN/0.1% TFA (aq.)) to afford the title compound as a TFA salt (2.3 mg, 0.0049 mmol, 24% yield), a colorless solid. LC-MS calc. for C₂₇H₂₆FN₄O₃ [M+H]⁺: m/z=473.2; Found 473.0.

### Example 46: 2-((1-(2-(4-acetylpiperazin-1-yl)-3,6-dimethyl-4-oxo-3,4-dihydroquinazolin-8-yl)ethyl)amino)benzoic acid

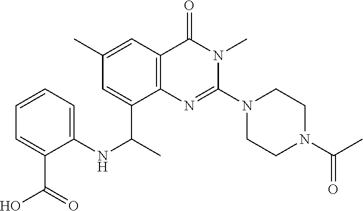 [SMILES: CC(=O)N1CCN(c2nc3c(C(C)Nc4ccccc4C(=O)O)cc(C)cc3c(=O)n2C)CC1]

### Step 1: tert-butyl 4-(8-(1-hydroxyethyl)-3,6-dimethyl-4-oxo-3,4-dihydroquinazolin-2-yl)piperazine-1-carboxylate

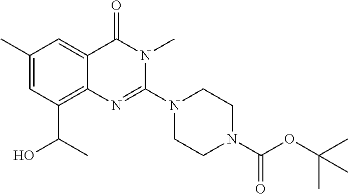 [SMILES: Cc1cc(C(C)O)c2nc(N3CCN(C(=O)OC(C)(C)C)CC3)n(C)c(=O)c2c1]

The title compound was synthesized by procedures analogous to Example 39, Steps 1-3. LC-MS calc. for C₂₁H₃₁N₄O₄ [M+H]⁺: m/z=403.2; Found 403.2.

### Step 2: tert-butyl 4-(8-(1-((2-(methoxycarbonyl)phenyl)amino)ethyl)-3,6-dimethyl-4-oxo-3,4-dihydroquinazolin-2-yl)piperazine-1-carboxylate

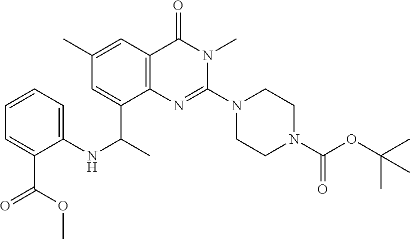 [SMILES: COC(=O)c1ccccc1NC(C)c1cc(C)cc2c(=O)n(C)c(N3CCN(C(=O)OC(C)(C)C)CC3)nc12]

The title compound was synthesized by procedures analogous to Example 41, Step 4. ¹H NMR (400 MHz, DMSO-d₆) δ 7.76 (dd, J=8.0, 4.0 Hz, 1H), 7.71-7.69 (m, 1H), 7.64 (d, J=8.0, 1H), 7.49 (d, J=4.0, 1H), 7.32 (d, J=4.0, 1H), 6.56 (d, J=8.0, 1H), 6.49 (t, J=8.0, 1H), 5.37-5.32 (m, 1H), 3.80 (s, 3H), 3.53-3.50 (m, 4H), 3.48 (s, 3H), 3.20-3.15 (m, 4H), 2.31 (s, 3H), 1.57 (d, J=8.0, 3H), 1.40 (s, 9H). LC-MS calc. for C₂₉H₃₈N₅O₅ [M+H]⁺: m/z=536.3; Found 536.1.

### Step 3: methyl 2-((1-(3,6-dimethyl-4-oxo-2-(piperazin-1-yl)-3,4-dihydroquinazolin-8-yl) ethyl)amino)benzoate

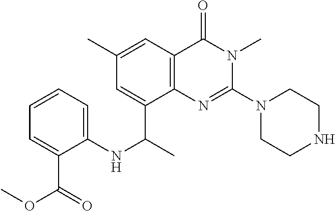 [SMILES: COC(=O)c1ccccc1NC(C)c1cc(C)cc2c(=O)n(C)c(N3CCNCC3)nc12]

To a solution of tert-butyl 4-[8-[1-(2-methoxycarbonylanilino)ethyl]-3,6-dimethyl-4-oxoquinazolin-2-yl]piperazine-1-carboxylate (160 mg, 0.299 mmol) in DCM (2 mL) was added TFA (0.4 mL, 5.23 mmol). The reaction mixture was stirred at 35° C. for 2 h. The mixture was acidified to pH 5-6 with 1 N HCl and extracted with DCM (3×20 mL). The combined organic layers were washed with brine (60 mL), dried over Na₂SO₄, filtered, and concentrated. The crude material was purified by prep-HPLC on a C18 column (10-90% MeCN/0.1% TFA (aq.)) to afford the title compound as a TFA salt (120 mg, 0.276 mmol, 92.2% yield), a yellow solid. LC-MS calc. for C₂₄H₃₀N₅O₃ [M+H]⁺: m/z=436.2; Found 436.2.

### Step 4: methyl 2-((1-(2-(4-acetylpiperazin-1-yl)-3,6-dimethyl-4-oxo-3,4-dihydro-quinazolin-8-yl)ethyl)amino)benzoate

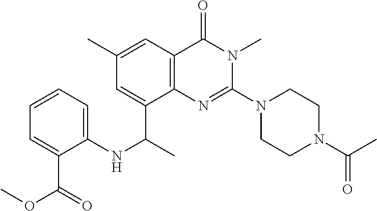 [SMILES: COC(=O)c1ccccc1NC(C)c1cc(C)cc2c(=O)n(C)c(N3CCN(C(C)=O)CC3)nc12]

To a solution of methyl 2-[1-(3,6-dimethyl-4-oxo-2-piperazin-1-ylquinazolin-8-yl)ethyl-amino] benzoate (30.0 mg, 0.0689 mmol, from Step 3) in DCM (10 mL) was added triethylamine (0.0200 mL, 0.207 mmol) and acetic anhydride (7.74 mg, 0.0758 mmol). The mixture was stirred at 40° C. for 14 h. The mixture was diluted with water (10 mL) and extracted with EtOAc (3×10 mL). The combined organic layers were washed with brine (30 mL), dried over Na₂SO₄, filtered, and concentrated. The residue was purified by prep-HPLC on a C18 column (10-90% MeCN/0.1% TFA (aq.)) to afford the title compound as a TFA salt (20 mg, 0.042 mmol, 61% yield), a yellow solid. LC-MS calc. for C₂₆H₃₂N₅O₄ [M+H]⁺: m/z=478.3; Found 478.9.

### Step 5: 2-((1-(2-(4-acetylpiperazin-1-yl)-3,6-dimethyl-4-oxo-3,4-dihydroquinazolin-8-yl)ethyl)amino)benzoic acid

To a solution of methyl 2-[1-[2-(4-acetylpiperazin-1-yl)-3,6-dimethyl-4-oxoquinazolin-8-yl]ethylamino]benzoate (20.0 mg, 0.0419 mmol, from Step 4) in methanol (1 mL), THE (0.5 mL), and water (0.5 mL) was added lithium hydroxide (7.03 mg, 0.168 mmol). The mixture was stirred at 40° C. for 14 h. The reaction mixture was acidified to pH 5-6 with 1N HCl, concentrated, and purified by prep-HPLC on a C18 column (10-90% MeCN/0.1% TFA (aq.)) to afford the title compound as a TFA salt (4.0 mg, 0.0079 mmol, 19% yield), a white solid. LC-MS calc. for C₂₅H₃₀N₅O₄ [M+H]⁺: m/z=464.2; Found 464.2.

### Example 47: 2-[[1-[2-(3-azabicyclo[3.1.0]hexan-3-yl)-3,6-dimethyl-4-oxoquinazolin-8-yl]-2,2,2-trifluoroethyl]amino]benzoic acid

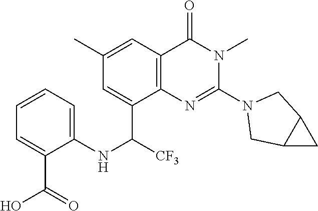 [SMILES: Cc1cc(C(Nc2ccccc2C(=O)O)C(F)(F)F)c2nc(N3CC4CC4C3)n(C)c(=O)c2c1]

### Step 1: 2-(3-azabicyclo[3.1.0]hexan-3-yl)-8-bromo-3,6-dimethylquinazolin-4-one

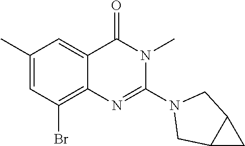 [SMILES: Cc1cc(Br)c2nc(N3CC4CC4C3)n(C)c(=O)c2c1]

The title compound was synthesized by procedures analogous to Example 39, Step 1. ¹H NMR (300 MHz, CDCl₃) δ 7.89 (dd, J=2.0, 1.0 Hz, 1H), 7.73 (d, J=2.0 Hz, 1H), 3.89-3.86 (m, 2H), 3.58-3.54 (m, 2H), 3.49 (s, 3H), 2.39 (s, 3H), 0.93-0.81 (m, 2H), 0.67-0.60 (m, 1H), 0.46-0.42 (m, 1H). LC-MS calc. for C₁₅H₁₆BrN₃O [M+H]⁺: m/z=334.1; Found 334.6.

### Step 2: 2-(3-azabicyclo[3.1.0]hexan-3-yl)-8-ethenyl-3,6-dimethylquinazolin-4-one

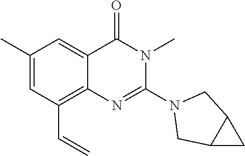 [SMILES: C=Cc1cc(C)cc2c(=O)n(C)c(N3CC4CC4C3)nc12]

To a solution of 2-(3-azabicyclo[3.1.0]hexan-3-yl)-8-bromo-3,6-dimethylquinazolin-4-one (200 mg, 0.598 mmol) in 1,4-dioxane (0.9 mL) and water (0.1 mL) was added 4,4,5,5-tetra-methyl-2-vinyl-1,3,2-dioxaborolane (0.205 mL, 1.20 mmol), potassium carbonate (248 mg, 1.80 mmol), and tetrakis(triphenylphosphine)palladium(0) (138 mg, 0.120 mmol). The resulting mixture was sparged with N2 (3×) and stirred at 100° C. for 2 h. The reaction mixture was cooled to room temperature and diluted with water (20 mL) and EtOAc (20 mL). The layers were separated, and the aqueous layer was extracted with EtOAc (2×20 mL). The combined organic layers were washed with brine, dried over sodium sulfate, filtered, and concentrated. The crude material was purified by silica gel chromatography (0-40% EtOAc/heptane) to afford the title compound (164 mg, 0.581 mmol, 97.1% yield) as a yellow liquid. ¹H NMR (300 MHz, CDCl₃) δ 7.93-7.85 (m, 1H), 7.66 (d, J=2.1 Hz, 1H), 7.55 (dd, J=17.9, 11.2 Hz, 1H), 5.89 (dd, J=17.9, 1.6 Hz, 1H), 5.36 (dd, J=11.2, 1.5 Hz, 1H), 3.81-3.78 (m 2H), 3.53-3.49 (m, 2H), 3.50 (s, 3H), 2.42 (s, 3H), 0.93-0.81 (m, 2H), 0.65-0.58 (m, 1H), 0.51-0.47 (m, 1H). LC-MS calc. for C₁₇H₂₀N₃O [M+H]⁺: m/z=282.2; Found 282.2.

### Step 3: 2-(3-azabicyclo[3.1.0]hexan-3-yl)-3,6-dimethyl-4-oxoquinazoline-8-carbaldehyde

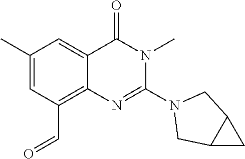 [SMILES: Cc1cc(C=O)c2nc(N3CC4CC4C3)n(C)c(=O)c2c1]

To a solution of 2-(3-azabicyclo[3.1.0]hexan-3-yl)-8-ethenyl-3,6-dimethylquinazolin-4-one (172 mg, 0.611 mmol) in 1,4-dioxane (3 mL) and water (1 mL) was added osmium teteraoxide (0.620 mL, 0.0611 mmol, 2.5 wt. % in tert-butanol). The resulting mixture was stirred for 10 min. 4-Methylmorpholine N-oxide (71.6 mg, 0.611 mmol) was added, and the reaction mixture was stirred at 35° C. for 1 h. Sodium periodate (131 mg, 0.611 mmol) was added, and the reaction was stirred at room temperature for 1 h. The reaction mixture was diluted with sat. Na₂SO₃ (aq.) (10 mL) and EtOAc (10 mL). The layers were separated, and the aqueous layer was extracted with EtOAc (2×10 mL). The combined organic layers were washed with brine, dried over Na₂SO₄, filtered, and concentrated. The crude material was purified by silica gel chromatography (0-40% EtOAc/heptane) to afford the title compound (144 mg, 0.508 mmol, 83.1% yield) as a light brown solid. ¹H NMR (300 MHz, CDCl₃) δ 10.98 (s, 1H), 8.19 (dd, J=2.2, 0.9 Hz, 1H), 8.07-7.96 (m, 1H), 3.92-3.88 (m, 2H), 3.58-3.53 (m, 2H), 3.52 (s, 3H), 2.44 (s, 3H), 0.93-0.81 (m, 2H), 0.69-0.62 (m, 1H), 0.44-0.39 (m, 1H). LC-MS calc. for C₁₆H₁₈N₃O₂ [M+H]: m/z=284.1; Found 284.2.

### Step 4: 2-(3-azabicyclo[3.1.0]hexan-3-yl)-3,6-dimethyl-8-(2,2,2-trifluoro-1-hydroxyethyl)quinazolin-4-on

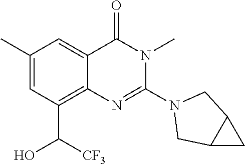 [SMILES: Cc1cc(C(O)C(F)(F)F)c2nc(N3CC4CC4C3)n(C)c(=O)c2c1]

To a solution of 2-(3-azabicyclo[3.1.0]hexan-3-yl)-3,6-dimethyl-4-oxoquinazoline-8-carbaldehyde (72.0 mg, 0.254 mmol) in THF (1.5 mL) was added cesium fluoride (57.9 mg, 0.381 mmol) and trimethyl(trifluoromethyl)silane (58.5 μL, 0.381 mmol). The resulting mixture was stirred for 3 min. The reaction was quenched with 1 N HCl (0.5 mL), and the mixture was stirred for 0.5 h. The reaction mixture was diluted with sat. NaHCO₃ (5 mL), water (5 mL), and EtOAc (5 mL). The layers were separated, and the aqueous layer was extracted with EtOAc (3×10 mL). The combined organic layers were washed with brine, dried over Na₂SO₄, filtered, and concentrated. The crude material was purified by silica gel chromatography (10-60% EtOAc/heptane) to afford the title compound (95.5 mg, 0.270 mmol, quantitative) as a white solid. ¹H NMR (300 MHz, CDCl₃) δ 7.98 (s, 1H), 7.34 (s, 1H), 5.35 (s, 1H), 5.11 (q, J=7.7 Hz, 1H), 3.88-3.85 (m, 1H), 3.68-3.65 (m, 1H), 3.56-3.41 (m, 2H) and 3.51 (s, 3H), 2.43 (s, 3H), 0.91-0.81 (m, 2H), 0.70-0.63 (m, 1H), 0.47-0.43 (m, 1H). LC-MS calc. for C₁₇H₁₉F3N302 [M+H]: m/z=354.1; Found 354.1.

### Step 5: 2-[[1-[2-(3-azabicyclo[3.1.0]hexan-3-yl)-3,6-dimethyl-4-oxoquinazolin-8-yl]-2,2,2-trifluoroethyl]amino]benzoic acid

The title compound was synthesized by procedures analogous to Example 41, Step 4. ¹H NMR (300 MHz, DMSO-d₆) δ 13.04 (s, 1H), 9.03 (d, J=9.0 Hz, 1H), 7.85-7.81 (m, 2H), 7.58 (s, 1H), 7.38-7.32 (m, 1H), 6.80 (d, J=8.5 Hz, 1H), 6.71-6.66 (m, 1H), 6.40-6.35 (m, 1H), 4.08-4.06 (m, 1H), 3.80-3.77 (m, 1H), 3.59-3.47 (m, 2H), 3.42 (s, 3H), 2.36 (s, 3H), 2.00-1.97 (m, 1H), 1.53-1.37 (m, 1H), 0.63-0.57 (m, 1H), 0.33-0.29 (m, 1H). LC-MS calc. for C₂₄H₂₄F3N403 [M+H]⁺: m/z=473.2; Found 473.1.

### Example 48: ((1-(3-((1r,3r)-3-hydroxycyclobutyl)-2-(isoindolin-2-yl)-6-methyl-4-oxo-3,4-dihydroquinazolin-8-yl)ethyl)amino)benzoic acid

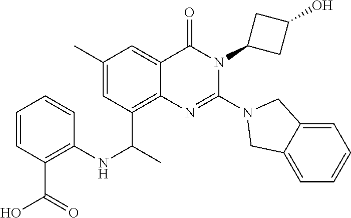 [SMILES: Cc1cc(C(C)Nc2ccccc2C(=O)O)c2nc(N3Cc4ccccc4C3)n([C@H]3C[C@H](O)C3)c(=O)c2c1]

### Step 1: 2-Amino-3-brom o-N-(3-hydroxycyclobutyl)-5-methylbenzamide

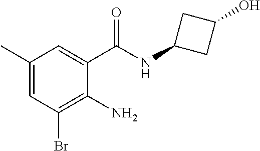 [SMILES: Cc1cc(Br)c(N)c(C(=O)N[C@H]2C[C@H](O)C2)c1]

To a solution of 2-amino-3-bromo-5-methylbenzoic acid (500 mg, 2.17 mmol) in THF (5 mL) was added 1,1′-carbonyldiimidazole (388 mg, 2.39 mmol). The reaction mixture was stirred for 17 h. 3-Aminocyclobutan-1-ol hydrochloride (403 mg, 3.26 mmol) and triethylamine (330 mg, 3.26 mmol) were added. The mixture was stirred for 3 h. The reaction mixture was diluted with water and extracted with EtOAc (5 mL×3). The combined organic layers were washed with brine, dried over Na₂SO₄, filtered, concentrated, and purified by silica gel chromatography (30% petroleum ether/EtOAc) to afford the title compound (380 mg, 1.27 mmol, 58.4% yield), a white solid. ¹H NMR (400 MHz, DMSO-d₆) δ 8.47 (d, J=6.7 Hz, 1H), 7.35 (m, 2H), 6.12 (m, 2H), 4.99 (s, 1H), 4.39-4.25 (m, 2H), 2.27-2.20 (m, 2H), 2.17 (s, 3H), 2.15-2.08 (m, 2H). LC-MS calc. for C₁₂H₁₆BrN₂O₂ [M+H]⁺: m/z=299.0; Found 299.5.

### Step 2: 2-amino-3-bromo-N-[3-[tert-butyl(dimethyl)silyl]oxycyclobutyl]-5-methyl-benzamide

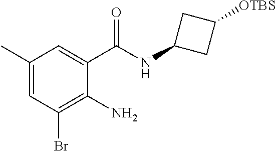 [SMILES: Cc1cc(Br)c(N)c(C(=O)N[C@H]2C[C@H](O[Si](C)(C)C(C)(C)C)C2)c1]

To a solution of 2-amino-3-bromo-N-(3-hydroxycyclobutyl)-5-methylbenzamide (200 mg, 0.67 mmol) in DCM (4 mL) was added imidazole (91 mg, 1.3 mmol). The mixture was cooled to 0° C., and tert-butyldimethylsilyl chloride (120 mg, 0.82 mmol) was added in portions. The resulting mixture was stirred at 45° C. for 5 h. The reaction mixture was concentrated and purified by silica gel chromatography (6% EtOAc/hexanes) to afford the title compound (190 mg, 0.459 mmol, 68.7% yield) as a white solid. ¹H NMR (400 MHz, DMSO-d₆) δ 8.51 (d, J=6.6 Hz, 1H), 7.38-7.32 (m, 2H), 6.12 (s, 2H), 4.55-4.48 (m, 1H), 4.29 (m, 1H), 2.33-2.25 (m, 2H), 2.23-2.12 (m, 5H), 0.85 (s, 9H), 0.01 (s, 6Hf). LC-MS calc. for C₁₈H₃₀BrN₂O₂Si [M+H]⁺: m/z=413.1; Found 413.7.

### Step 3: 8-bromo-3-[3-[tert-butyl(dimethyl)silyl]oxycyclobutyl]-6-methyl-2-sulfanylidene-1H-quinazolin-4-one

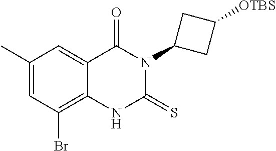 [SMILES: Cc1cc(Br)c2[nH]c(=S)n([C@H]3C[C@H](O[Si](C)(C)C(C)(C)C)C3)c(=O)c2c1]

To a solution of 2-amino-3-bromo-N-[3-[tert-butyl(dimethyl)silyl]oxycyclobutyl]-5-methylbenzamide (190 mg, 0.46 mmol) in THF (4 mL) was added 1,1′-thiocarbonyldiimidazole (160 mg, 0.92 mmol) and 1,8-diazabicyclo[5.4.0]undec-7-ene (210 mg, 1.38 mmol). The reaction was stirred at 70° C. for 16 h. The reaction mixture was cooled to 0° C., and the reaction quenched with sat. NH₄Cl (aq.). The resulting precipitate was collected by filtration and rinsed with water. The crude material was purified by silica gel chromatography (5% EtOAc/hexanes) to give the title compound (160 mg, 0.351 mmol, 76.4% yield) as a white solid. ¹H NMR (400 MHz, DMSO-d₆) δ 10.69-10.62 (m, 1H), 7.86 (s, 1H), 7.77 (s, 1H), 6.36-6.29 (m, 1H), 4.72 (m, 1H), 3.07-3.00 (m, 2H), 2.34 (s, 3H), 2.25 (m, 2H), 0.87 (s, 9H), 0.03 (s, 6H). LC-MS calc. for C₁₉H₂₈BrN₂O₂SSi [M+H]⁺: m/z=455.1; Found 455.6.

### Step 4: 8-bromo-3-[3-[tertbutyl(dimethyl)silyl]oxycyclobutyl]-6-methyl-2-methyl-sulfanylquinazolin-4-one

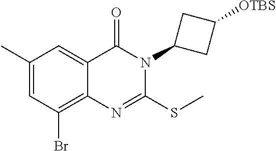 [SMILES: CSc1nc2c(Br)cc(C)cc2c(=O)n1[C@H]1C[C@H](O[Si](C)(C)C(C)(C)C)C1]

To a solution of 8-bromo-3-[3-[tert-butyl(dimethyl)silyl]oxycyclobutyl]-6-methyl-2-sulfanylidene-1H-quinazolin-4-one (40 mg, 0.092 mmol) in THF (2 mL) was added iodomethane (18 mg, 0.13 mmol) and triethylamine (18 mg, 0.18 mmol) at 0° C. The resulting mixture was stirred at 30° C. for 21 h. The reaction mixture was concentrated, and the crude material was purified by prep-TLC (10:1 hexanes/EtOAc) to afford the title compound (25 mg, 0.053 mmol, 61% yield) as a white solid. ¹H NMR (400 MHz, DMSO-d₆) δ 7.92 (m, 1H), 7.83 (m, 1H), 5.10 (m, J=9.5, 6.6, 2.9, 0.9 Hz, 1H), 4.79-4.75 (m, 1H), 3.17-3.10 (m, 2H), 2.62 (s, 3H), 2.38 (s, 3H), 2.33 (m, 2H), 0.87 (s, 9H), 0.04 (s, 6H). LC-MS calc. for C₂₀H₃₀BrN₂O₂SSi [M+H]⁺: m/z=469.1; Found 469.6.

### Step 5: 8-bromo-3-((1r,3r)-3-((tert-butyldimethylsilyl)oxy)cyclobutyl)-6-methyl-2-(methylsulfonyl)quinazolin-4(3H)-one

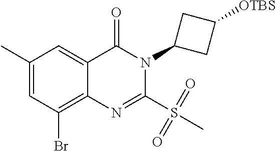 [SMILES: Cc1cc(Br)c2nc(S(C)(=O)=O)n([C@H]3C[C@H](O[Si](C)(C)C(C)(C)C)C3)c(=O)c2c1]

The title compound was synthesized by procedures analogous to Example 44, Step 2. ¹H NMR (400 MHz, DMSO-d₆) δ 8.08 (dd, J=4.0, 1.6 Hz, 1H), 7.97 (dd, J=4.0, 1.6 Hz, 1H), 5.69-5.58 (m, 1H), 4.80-4.75 (m, 1H), 3.64 (s, 3H), 3.26-3.20 (m, 2H), 2.46 (s, 3H), 2.30-2.23 (m, 2H), 0.87 (s, 9H), 0.03 (s, 6H). LC-MS calc. for C₂₀H₃₀BrN₂O₄SSi [M+H]⁺: m/z=501.1; Found 501.7.

### Step 6: 8-bromo-3-((1r,3r)-3-((tert-butyldimethylsilyl)oxy)cyclobutyl)-2-(isoindolin-2-yl)-6-methylquinazolin-4(3H)-one

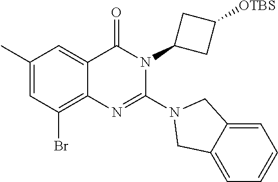 [SMILES: Cc1cc(Br)c2nc(N3Cc4ccccc4C3)n([C@H]3C[C@H](O[Si](C)(C)C(C)(C)C)C3)c(=O)c2c1]

To a solution of 8-bromo-3-[3-[tert-butyl(dimethyl)silyl]oxycyclobutyl]-6-methyl-2-methylsulfonylquinazolin-4-one (65 mg, 0.13 mmol) in THF (5 mL) was added N,N-diisopropyl-ethylamine (50 mg, 0.39 mmol) and isoindoline hydrochloride (30 mg, 0.19 mmol). The mixture was stirred at 50° C. for 14 h. The mixture was cooled to room temperature, diluted with water (20 mL), and extracted with EtOAc (3×20 mL). The combined organic layers were washed with brine (30 mL), dried over Na₂SO₄, filtered, and concentrated. The crude material was purified by prep-TLC (20% EtOAc/petroleum ether) to afford the title compound (65 mg, 0.12 mmol, 93% yield) as a white solid. ¹H NMR (400 MHz, DMSO-d₆) δ 7.81 (dd, J=4.0, 1.6 Hz, 1H), 7.75 (dd, J=4.0, 1.6 Hz, 1H), 7.39-7.37 (m, 2H), 7.29-7.27 (m, 2H), 5.03-4.99 (m, 1H), 4.90 (s, 4H), 4.69-4.65 (m, 1H), 2.86-2.80 (m, 2H), 2.46-2.43 (m, 2H), 2.34 (s, 3H), 0.87 (s, 9H), 0.03 (s, 6H). LC-MS calc. for C₂₇H₃₅BrN₃O₂Si [M+H]⁺: m/z=540.2; Found 540.8.

### Step 7: 8-acetyl-3-((1r,3r)-3-((tert-butyldimethylsilyl)oxy)cyclobutyl)-2-(isoindolin-2-yl)-6-methylquinazolin-4(3H)-one

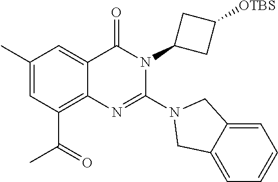 [SMILES: CC(=O)c1cc(C)cc2c(=O)n([C@H]3C[C@H](O[Si](C)(C)C(C)(C)C)C3)c(N3Cc4ccccc4C3)nc12]

The title compound was synthesized by procedures analogous to Example 39, Step 2. R_{f}=0.3 (5:1 petroleum ether/EtOAc).

### Step 8: 3-((1r,3r)-3-((tert-butyldimethylsilyl)oxy)cyclobutyl)-8-(1-hydroxyethyl)-2-(isoindolin-2-yl)-6-methylquinazolin-4(3H)-one

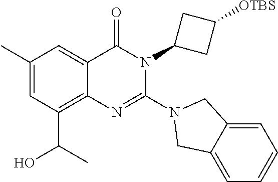 [SMILES: Cc1cc(C(C)O)c2nc(N3Cc4ccccc4C3)n([C@H]3C[C@H](O[Si](C)(C)C(C)(C)C)C3)c(=O)c2c1]

The title compound was synthesized by procedures analogous to Example 39, Step 3. The crude material was purified by prep-TLC (3:1 petroleum ether/EtOAc) to afford the title compound (15 mg, 0.030 mmol, 36% yield) as a yellow solid. ¹H NMR (400 MHz, DMSO-d₆) δ 7.64 (d, J=1.3 Hz, 1H), 7.60 (d, J=2.1 Hz, 1H), 7.39-7.37 (m, 2H), 7.29-7.27 (m, 2H), 5.40-5.38 (m, 1H), 5.05-5.01 (m, 2H), 4.89 (d, J=12.0, 2H), 4.76 (d, J=12.0, 2H), 4.72-4.70 (m, 1H), 2.90-2.85 (m, 2H), 2.46-2.40 (m, 2H), 2.36 (s, 3H), 1.36 (d, J=4.0, 3H), 0.87 (s, 9H), 0.03 (s, 6H). LC-MS calc. for C₂₉H₄₀N₃O₃Si [M+H]⁺: m/z=506.3; Found 506.9.

### Step 9: Methyl 2-((1-(3-((1r,3r)-3-((tert-butyldimethylsilyl)oxy)cyclobutyl)-2-(isoindolin-2-yl)-6-methyl-4-oxo-3,4-dihydroquinazolin-8-yl)ethyl)amino)benzoate

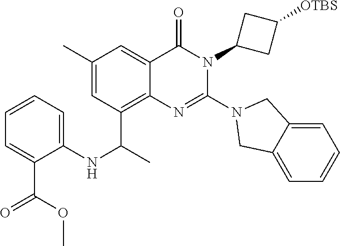 [SMILES: COC(=O)c1ccccc1NC(C)c1cc(C)cc2c(=O)n([C@H]3C[C@H](O[Si](C)(C)C(C)(C)C)C3)c(N3Cc4ccccc4C3)nc12]

The title compound was synthesized by procedures analogous to Example 41, Step 4. LC-MS calc. for C₃₇H₄₇N₄O₄Si [M+H]⁺: m/z=639.3; Found 639.5.

### Step 10: Methyl 2-((1-(3-((1r,3r)-3-hydroxycyclobutyl)-2-(isoindolin-2-yl)-6-methyl-4-oxo-3,4-dihydroquinazolin-8-yl)ethyl)amino)benzoate

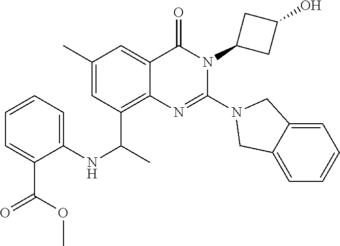 [SMILES: COC(=O)c1ccccc1NC(C)c1cc(C)cc2c(=O)n([C@H]3C[C@H](O)C3)c(N3Cc4ccccc4C3)nc12]

To a solution of methyl 2-[1-[3-[3-[tert-butyl(dimethyl)silyl]oxycyclobutyl]-2-(1,3-dihydroisoindol-2-yl)-6-methyl-4-oxoquinazolin-8-yl]ethylamino]benzoate (19.0 mg, 0.030 mmol) in THF (1 mL) was added tetrabutylammonium fluoride (0.06 mL, 0.06 mmol, 1 N in THF). The reaction mixture was stirred at 40° C. for 14 h. The reaction was quenched with water (20 mL), and the mixture extracted with EtOAc (3×20 mL). The combined organic layers were washed with brine (30 mL), dried over Na₂SO₄, filtered, and concentrated. The residue was purified by prep-TLC (50% petroleum ether/EtOAc) to afford the title compound (15 mg, 0.029 mmol, 96% yield) as a yellow solid. LC-MS calc. for C₃₁H₃₂N₄NaO₄ [M+Na]⁺: m/z=547.2; Found 547.8.

### Step 11: 2-((1-(3-((1r,3r)-3-Hydroxycyclobutyl)-2-(isoindolin-2-yl)-6-methyl-4-oxo-3,4-dihydroquinazolin-8-yl)ethyl)amino)benzoic acid

The title compound was synthesized by procedures analogous to Example 43, Step 6. ¹H NMR (400 MHz, DMSO-d₆) δ 8.38 (s, 1H), 7.74 (dd, J=8.1, 1.7 Hz, 1H), 7.63 (dd, J=2.1, 1.0 Hz, 1H), 7.41-7.35 (m, 3H), 7.29-7.27 (m, 2H), 7.14-7.11 (m, 1H), 6.47-6.44 (m, 2H), 5.37-5.33 (m, 1H), 5.10-5.06 (m, 1H), 4.95 (d, J=12.0 Hz, 2H), 4.83 (d, J=12.0 Hz, 2H), 4.36-4.33 (m, 1H), 2.83-2.78 (m, 2H), 2.41-2.37 (m, 2H), 2.27 (s, 3H), 1.55 (d, J=4.0 Hz, 3H). LC-MS calc. for C₃₀H₃₁N₄O₄ [M+H]⁺: m/z=511.2; Found 511.1.

### Example 49: 2-((1-(2-(3-azabicyclo[3.1.0]hexan-3-yl)-6-fluoro-3-methyl-4-oxo-3,4-dihydro-quinazolin-8-yl)ethyl)amino)benzoic acid

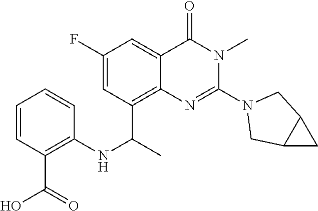 [SMILES: CC(Nc1ccccc1C(=O)O)c1cc(F)cc2c(=O)n(C)c(N3CC4CC4C3)nc12]

The title compound was synthesized by procedures analogous to Example 1, Step 1-4; Example 39, Step 2-3; Example 41, Step 4; and Example 43, Step 6. LC-MS calc. for C₂₃H₂₃FN₄NaO₃ [M+Na]⁺: m/z=445.1; Found 445.7.

### Example 50: 2-((1-(6-cyano-2-(isoindolin-2-yl)-3-methyl-4-oxo-3,4-dihydroquinazolin-8-yl)ethyl)amino)benzoic acid

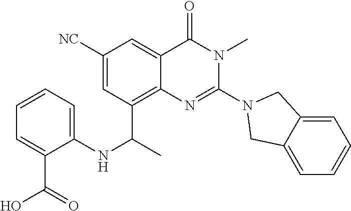 [SMILES: CC(Nc1ccccc1C(=O)O)c1cc(C#N)cc2c(=O)n(C)c(N3Cc4ccccc4C3)nc12]

### Step 1: 2-amino-5-bromo-3-iodobenzoic acid

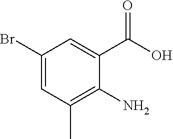

To a suspension of 2-amino-5-bromobenzoic acid (15.0 g, 69.4 mmol) in DCM (200 mL) was added N-iodosuccinimide (34.4 g, 153 mmol). The mixture was stirred overnight, and then heated at 40° C. for 6 h. The mixture was cooled to room temperature and 5% Na₂S₂O₃ solution (aq.) (50 mL) was added. The reaction mixture was stirred for 1 h. The resulting solid was collected by filtration, washed with water, and dried to give the title compound (17.0 g, 49.7 mmol, 71.6% yield) as a dark solid. LC-MS calc. for C₇H₆BrINO₂ [M+H]⁺: m/z=341.9, 343.9; Found 341.8, 343.7.

### Step 2: 6-Bromo-8-iodo-3-methylquinazoline-2,4(1H,3H)-dione

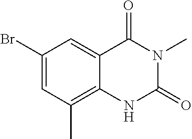

The title compound was synthesized by procedures analogous to Example 14, Steps 2 and 4. LC-MS calc. for C₉H₇BrIN₂O₂ [M+H]⁺: m/z=380.9; Found 380.7.

### Step 3: 6-Bromo-8-iodo-2-(isoindolin-2-yl)-3-methylquinazolin-4(3H)-one

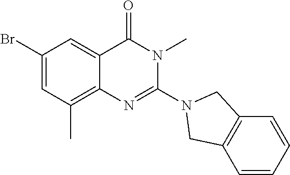 [SMILES: Cc1cc(Br)cc2c(=O)n(C)c(N3Cc4ccccc4C3)nc12]

The title compound was synthesized by procedures analogous to Example 1, Step 3-4. LC-MS calc. for C₁₇H₁₄BrIN₃O [M+H]⁺ m/z=481.9; Found 481.7.

### Step 4: 6-Bromo-8-(1-hydroxyethyl)-2-(isoindolin-2-yl)-3-methylquinazolin-4(3H)-one

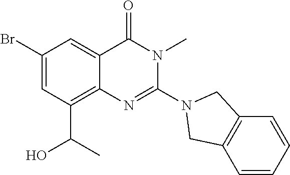 [SMILES: CC(O)c1cc(Br)cc2c(=O)n(C)c(N3Cc4ccccc4C3)nc12]

The title compound was synthesized by procedures analogous to Example 39, Step 2-3. LC-MS calc. for C₁₉H₁₉BrN₃O₂ [M+H]⁺ m/z=401.1, 403.1; Found 401.9, 403.2.

### Step 5: 1-(1-(6-bromo-2-(isoindolin-2-yl)-3-methyl-4-oxo-3,4-dihydroquinazolin-8-yl)ethyl)-2H-benzo[d][1,3]oxazine-2,4(1H)-dione

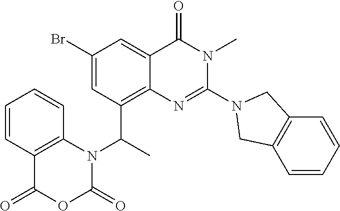 [SMILES: CC(c1cc(Br)cc2c(=O)n(C)c(N3Cc4ccccc4C3)nc12)n1c(=O)oc(=O)c2ccccc21]

The title compound was synthesized by procedures analogous to Example 45, Step 1. LC-MS calc. for C₂₇H₂₂BrN₄O₄ [M+H]⁺ m/z=545.1, 547.1; Found 544.8, 546.8.

### Step 6: 2-((1-(6-cyano-2-(isoindolin-2-yl)-3-methyl-4-oxo-3,4-dihydroquinazolin-8-yl)ethyl)amino)benzoic acid

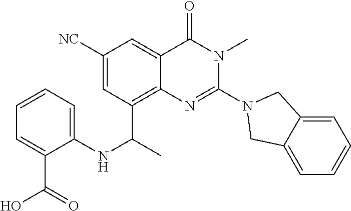 [SMILES: CC(Nc1ccccc1C(=O)O)c1cc(C#N)cc2c(=O)n(C)c(N3Cc4ccccc4C3)nc12]

A mixture of 1-[1-[6-bromo-2-(1,3-dihydroisoindol-2-yl)-3-methyl-4-oxoquinazolin-8-yl]ethyl]-3,1-benzoxazine-2,4-dione (11.0 mg, 0.0202 mmol), tris(dibenzylideneacetone)-dipalladium(0) (1.85 mg, 0.00202 mmol), zinc (1.32 mg, 0.0202 mmol), zinc cyanide (4.74 mg, 0.0403 mmol), and 1,1′-bis(diphenylphosphino)ferrocene (2.24 mg, 0.00403 mmol) in DMF (1 mL) was heated at 100° C. overnight under a N2 atmosphere. The mixture was cooled to room temperature, filtered, and purified by prep-HPLC on a C18 column (10-70% MeCN/0.1% TFA (aq.)) to afford the TFA salt of the title compound (1.3 mg, 0.0028 mmol, 14% yield), a brown solid. ¹H NMR (300 MHz, DMSO-d₆) δ 12.71 (s, 1H), 8.43 (d, J=6.7 Hz, 1H), 8.24 (d, J=2.0 Hz, 1H), 7.84-7.75 (m, 2H), 7.47-7.37 (m, 2H), 7.37-7.30 (m, 2H), 7.18 (ddd, J=8.6, 7.1, 1.7 Hz, 1H), 6.58-6.47 (m, 1H), 6.41 (d, J=8.5 Hz, 1H), 5.46-5.32 (m, 1H), 5.18 (m, 4H), 3.65 (s, 3H), 1.61 (d, J=6.6 Hz, 3H). LC-MS calc. for C₂₇H₂₄N₅O₃ [M+H]⁺ m/z=466.2; Found 466.0.

### Examples 51-57

Examples 51-57 listed in Table 7 were synthesized according to procedures analogous to Example 41, Step 4 (Method A), Example 39, Step 4-5 (Method B), or Example 44 (Method C). The appropriate starting materials for Methods A and B can be prepared according to procedures analogous to Example 1, Steps 1-6. All examples in this table were prepared as the TFA salt.

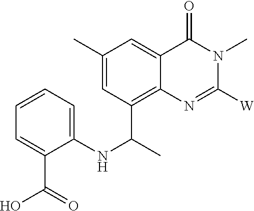

TABLE 7
Examples 51-57

| Example | W | Method | LCMS [M + H]⁺ |
|---|---|---|---|
| 51 | 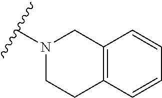 [SMILES: *N1CCc2ccccc2C1] | B | 469.1 |
| 52 | 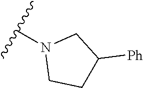 [SMILES: *N1CCC(c2ccccc2)C1] | B | 483.1 |
| 53 | 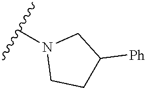 [SMILES: *N1CCC(c2ccccc2)C1] | B | 483.1 |
| 54 | 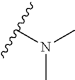 [SMILES: *N(C)C] | B | 381.1 |
| 55 | 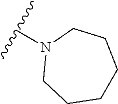 [SMILES: *N1CCCCCC1] | B | 435.0 |
| 56 | 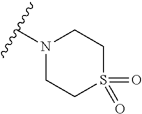 [SMILES: *N1CCS(=O)(=O)CC1] | A | 471.6 |
| 57 | 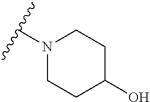 [SMILES: *N1CCC(O)CC1] | C | [M + Na]⁺ 459.9 |

TABLE 8
Examples 51-57

| Example | Compound name | NMR |
|---|---|---|
| 51 | 2-[1-[2-(3,4-dihydro-1H- | ¹H NMR (300 MHz, DMSO-d₆) δ 8.46 (bs, |
| | isoquinolin-2-yl)-3,6- | 1H), 7.93-7.64 (m, 2H), 7.48 (d, J = 2.1 Hz, |
| | dimethyl-4-oxoquinazolin-8- | 1H), 7.40-7.05 (m, 5H), 6.57-6.39 (m, 2H), |
| | yl]ethylamino]benzoic acid | 5.44-5.24 (m, 1H), 4.70-4.40 (m, 2H), 3.65- |
| | | 3.45 (m, 5H), 3.10-2.96 (m, 2H), 2.33 (s, |
| | | 3H), 1.54 (d, J = 6.6 Hz, 3H). |
| 52 | 2-[1-[3,6-dimethyl-4-oxo-2- | ¹H NMR (300 MHz, DMSO-d₆) δ 7.77 (dd, J = |
| | (3-phenylpyrrolidin-1-yl) | 8.1, 1.7 Hz, 1H), 7.71-7.64 (m, 1H), 7.45- |
| | quinazolin-8-yl]ethylamino] | 7.28 (m, 5H), 7.30-7.19 (m, 1H), 7.16 (ddd, J = |
| | benzoic acid (isomer 1) | 8.7, 7.1, 1.7 Hz, 1H), 6.52-6.42 (m, 2H), |
| | | 5.36 (q, J = 6.5 Hz, 1H), 3.97-3.80 (m, 2H), |
| | | 3.77-3.60 (m, 2H), 3.50 (s, 3H), 3.48-3.37 |
| | | (m 1H), 2.39-2.30 (m, 1H), 2.29 (s, 3H), 2.16- |
| | | 1.98 (m, 1H), 1.56 (d, J = 6.6 Hz, 3H). |
| 53 | 2-[1-[3,6-dimethyl-4-oxo-2- | ¹H NMR (300 MHz, DMSO-d₆) δ 12.64 (bs, |
| | (3-phenylpyrrolidin-1-yl) | 1H), 8.42 (bs, 1H), 7.77 (dd, J = 8.2, 1.7 Hz, |
| | quinazolin-8-yl]ethylamino] | 1H), 7.67 (dd, J = 2.1, 0.9 Hz, 1H), 7.44-7.29 |
| | benzoic acid (isomer 2) | (m, 5H), 7.29-7.14 (m, 2H), 6.55-6.41 (m, |
| | | 2H), 5.41-5.21 (m, 1H), 4.02-3.81 (m, 2H), |
| | | 3.76-3.60 (m, 2H), 3.49 (s, 3H), 3.47-3.40 |
| | | (m, 1H), 2.41-2.31 (m, 1H), 2.29 (s, 3H), |
| | | 2.16-1.94 (m, 1H), 1.55 (d, J = 6.6 Hz, 3H). |
| 54 | 2-[1-[2-(dimethylamino)- | ¹H NMR (300 MHz, CD₃OD) δ 7.91 (dd, J = |
| | 3,6-dimethyl-4- | 7.9, 1.7 Hz, 1H), 7.84-7.76 (m, 1H), 7.55 (d, |
| | oxoquinazolin-8-yl] | J = 2.0 Hz, 1H), 7.17 (ddd, J = 8.7, 7.1, 1.7 Hz, |
| | ethylamino]benzoic acid | 1H), 6.59 (ddd, J = 8.1, 7.1, 1.1 Hz, 1H), 6.52 |
| | | (d, J = 8.5 Hz, 1H), 5.41 (q, J = 6.7 Hz, 1H), |
| | | 3.58 (s, 3H), 3.00 (s, 6H), 2.37 (s, 3H), 1.64 (d, |
| | | J = 6.7 Hz, 3H). |
| 55 | 2-((1-(2-(azepan-1-yl)-3,6- | ¹H NMR (300 MHz, DMSO-d₆) δ 7.77 (dd, J = |
| | dimethyl-4-oxo-3,4- | 8.0, 1.7 Hz, 1H), 7.71-7.64 (m, 1H), 7.42 (d, |
| | dihydroquinazolin-8- | J = 2.1 Hz, 1H), 7.17 (ddd, J = 8.6, 7.1, 1.7 Hz, |
| | yl)ethyl)amino)benzoic acid | 1H), 6.54-6.33 (m, 2H), 5.38 (q, J = 6.6 Hz, |
| | | 1H), 3.50 (t, J = 5.8 Hz, 4H), 3.46 (s, 3H), 2.30 |
| | | (s, 3H), 1.94-1.73 (m, 4H), 1.70-1.59 (m, |
| | | 4H), 1.54 (d, J = 6.6 Hz, 3H). |
| 56 | 2-((1-(2-(1,1- | — |
| | dioxidothiomorpholino)-3,6- | |
| | dimethyl-4-oxo-3,4- | |
| | dihydroquinazolin-8- | |
| | yl)ethyl)amino)benzoic acid | |
| 57 | 2-((1-(2-(4-hydroxypiperidin- | — |
| | 1-yl)-3,6-dimethyl-4-oxo- | |
| | 3,4-dihydroquinazolin-8- | |
| | yl)ethyl)amino)benzoic acid | |

### Example 58: 2-((1-(2-((1R,5S,6s)-6-((methoxycarbonyl)amino)-3-azabicyclo[3.1.0]hexan-3-yl)-3,6-dimethyl-4-oxo-3,4-dihydroquinazolin-8-yl)ethyl)amino)benzoic acid

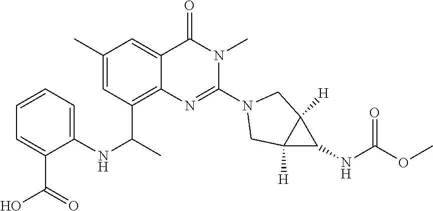

### Step 1. tert-Butyl ((1R,5S,6s)-3-(8-bromo-3,6-dimethyl-4-oxo-3,4-dihydroquinazolin-2-yl)-3-azabicyclo[3.1.0]hexan-6-yl)carbamate

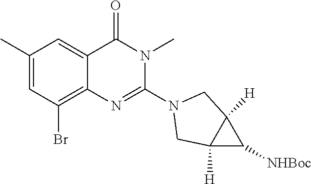

The title compound was synthesized by procedures analogous to those outlined in Example 74, Step 1. LC-MS calc. for C₂₀H₂₆BrN₄O₃ [M+H]⁺ m/z=451.1; Found 451.0.

### Step 2. tert-butyl ((1R,5S,6s)-3-(8-(1-hydroxyethyl)-3,6-dimethyl-4-oxo-3,4-dihydroquinazolin-2-yl)-3-azabicyclo[3.1.0]hexan-6-yl)carbamate

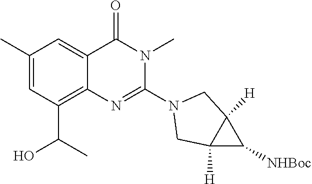 [SMILES: Cc1cc(C(C)O)c2nc(N3C[C@@H]4[C@H](C3)[C@H]4NC(=O)OC(C)(C)C)n(C)c(=O)c2c1]

The title compound was synthesized by procedures analogous to those outlined in Example 1, Steps 5-6. LC-MS calc. for C₂₂H₃₁N₄O₄ [M+H]⁺ m/z=415.2; Found 415.1.

### Step 3. tert-Butyl 2-((1-(2-((1R,5S,6s)-6-((tert-butoxycarbonyl)amino)-3-azabicyclo [3.1.0]hexan-3-yl)-3,6-dimethyl-4-oxo-3,4-dihydroquinazolin-8-yl)ethyl)amino)benzoate

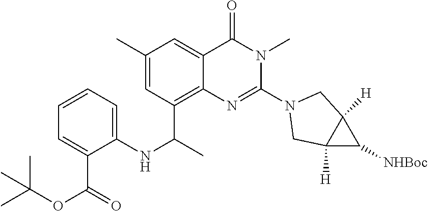

The title compound was synthesized by procedures analogous to those outlined in Example 23, Step 2. LC-MS calc. for C₃₃H₄₄N₅O₅ [M+H]⁺ m/z=590.3; Found 590.3.

### Step 4. tert-Butyl 2-((1-(2-((1R,5S,6s)-6-amino-3-azabicyclo[3.1.0]hexan-3-yl)-3,6-dimethyl-4-oxo-3,4-dihydroquinazolin-8-yl)ethyl)amino)benzoate

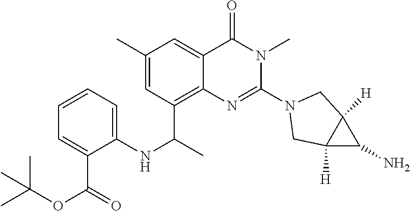

To a solution of tert-butyl 2-((1-(2-((1R,5S,6s)-6-((tert-butoxycarbonyl)amino)-3-azabicyclo[3.1.0]hexan-3-yl)-3,6-dimethyl-4-oxo-3,4-dihydroquinazolin-8-yl)ethyl)amino)benzoate in DCM (2 mL) was added TFA (2 mL). The mixture was stirred at room temperature for 1.5 h. The mixture was concentrated and purified by prep-HPLC (15-60% MeCN/0.1% TFA (aq)) to afford the title compound (76.0 mg, 0.155 mmol, 41.3% yield), a colorless solid. LC-MS calc. for C₂₈H₃₆N₅O₃ [M+H]⁺ m/z=490.3; Found 490.1.

### Step 5. tert-Butyl 2-((1-(2-((1R,5S,6s)-6-((methoxycarbonyl)amino)-3-azabicyclo[3.1.0]hexan-3-yl)-3,6-dimethyl-4-oxo-3,4-dihydroquinazolin-8-yl)ethyl)amino)benzoate

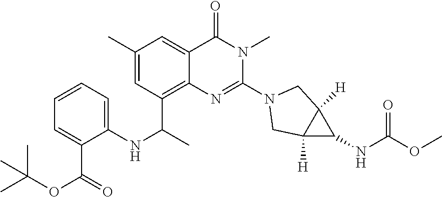

To a solution of tert-butyl 2-((1-(2-((1R,5S,6s)-6-amino-3-azabicyclo[3.1.0]hexan-3-yl)-3,6-dimethyl-4-oxo-3,4-dihydroquinazolin-8-yl)ethyl)amino)benzoate (38 mg, 0.070 mmol) in DCM (2 mL) was added methoxycarbonyl methyl carbonate (9.1 mg, 0.070 mmol) and triethylamine (0.020 mL, 0.16 mmol). The mixture was stirred at room temperature for 2 h. The mixture was quenched with water (5 mL). The organic phase was separated, and the aqueous phase was extracted with DCM (3×10 mL). The combined organic phases were washed with brine, dried over Na₂SO₄, and concentrated. The crude material was purified by silica gel chromatography (20-80% EtOAc/heptane) to afford the title compound (42.5 mg, 0.0776 mmol, quantitative yield), a white solid. LC-MS calc. for C₃₀H₃₈N₅O₅ [M+H]⁺ m/z=548.3; Found 548.2.

### Step 6. 2-((1-(2-((1R,5S,6s)-6-((Methoxycarbonyl)amino)-3-azabicyclo[3.1.0]hexan-3-yl)-3,6-dimethyl-4-oxo-3,4-dihydroquinazolin-8-yl)ethyl)amino)benzoic acid

The title compound was synthesized by procedures analogous to those outlined in Example 38, Step 2. ¹H NMR (300 MHz, DMSO-d₆) δ 8.42 (s, 1H), 7.77 (d, J=8.2 Hz, 1H), 7.67 (s, 1H), 7.42 (s, 2H), 7.16 (s, 1H), 6.45 (t, J=7.6 Hz, 2H), 5.35 (d, J=12.8 Hz, 1H), 4.02 (d, J=10.6 Hz, 2H), 3.84 (d, J=10.7 Hz, 2H), 3.51 (s, 3H), 3.43 (s, 3H), 2.29 (s, 3H), 1.99 (s, 1H), 1.74 (s, 2H), 1.55 (d, J=6.5 Hz, 3H). LC-MS calc. for C₂₆H₃₀N₅O₅ [M+H]⁺ m/z=492.2; Found 492.1.

### Examples 59-64

Examples 59-64 listed in Tables 9 and 10 were synthesized according to procedures analogous to Example 58. All examples in this table were prepared as the TFA salt.

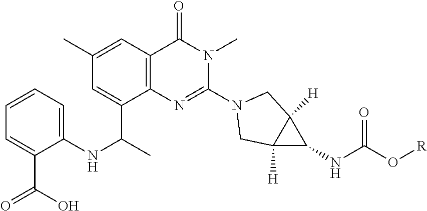

TABLE 9
Examples 59-64

| Example | R | LCMS [M + H]⁺ |
|---|---|---|
| 59 | 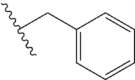 [SMILES: *Cc1ccccc1] | 568.1 |
| 60 | 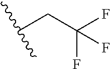 [SMILES: *CC(F)(F)F] | 560.0 |
| 61 | 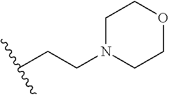 [SMILES: *CCN1CCOCC1] | 591.1 |
| 62 | 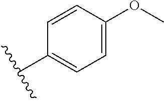 [SMILES: *c1ccc(OC)cc1] | 584.1 |
| 63 | 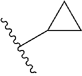 [SMILES: *C1CC1] | 518.0 |
| 64 | 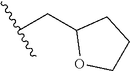 [SMILES: *CC1CCCO1] | 562.1 |

TABLE 10
Examples 59-64

| Exam- | |
|---|---|
| ple | Compound Name |
| 59 | 2-((1-(2-((1R,5S,6s)-6-(((benzyloxy)carbonyl)amino)-3- |
| | azabicyclo[3.1.0]hexan-3-yl)-3,6-dimethyl-4-oxo-3,4-dihydro- |
| | quinazolin-8-yl)ethyl)amino)benzoic acid |
| 60 | 2-((1-(3,6-dimethyl-4-oxo-2-((1R,5S,6s)-6-(((2,2,2- |
| | trifluoroethoxy)carbonyl)amino)-3-azabicyclo[3.1.0]hexan-3- |
| | yl)-3,4-dihydro-quinazolin-8-yl)ethyl)amino)benzoic acid |
| 61 | 2-((1-(3,6-dimethyl-2-((1R,5S,6s)-6-(((2-morpholinoethoxy) |
| | carbonyl)amino)-3-azabicyclo[3.1.0]hexan-3-yl)-4-oxo-3,4- |
| | dihydroquinazolin-8-yl)ethyl)amino)benzoic acid |
| 62 | 2-((1-(2-((1R,5S,6s)-6-(((4-methoxyphenoxy)carbonyl)amino)- |
| | 3-azabicyclo[3.1.0]hexan-3-yl)-3,6-dimethyl-4-oxo-3,4- |
| | dihydroquinazolin-8-yl)ethyl)amino)benzoic acid |
| 63 | 2-((1-(2-((1R,5S,6s)-6-((cyclopropoxycarbonyl)amino)-3- |
| | azabicyclo[3.1.0]hexan-3-yl)-3,6-dimethyl-4-oxo-3,4- |
| | dihydroquinazolin-8-yl)ethyl)amino)benzoic acid |
| 64 | 2-((1-(3,6-dimethyl-4-oxo-2-((1R,5S,6s)-6-((((tetrahydrofuran- |
| | 2-yl)methoxy)carbonyl)amino)-3-azabicyclo[3.1.0]hexan-3- |
| | yl)-3,4-dihydroquinazolin-8-yl)ethyl)amino)benzoic acid |

### Example 65: 2-((1-(2-(5-(tert-Butoxycarbonyl)-2,5-diazabicyclo[2.2.1]heptan-2-yl)-3,6-dimethyl-4-oxo-3,4-dihydroquinazolin-8-yl)ethyl)amino)benzoic acid

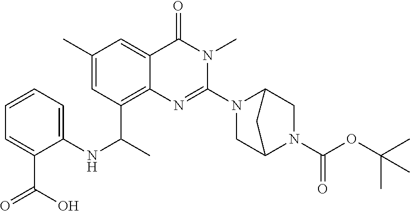 [SMILES: Cc1cc(C(C)Nc2ccccc2C(=O)O)c2nc(N3CC4CC3CN4C(=O)OC(C)(C)C)n(C)c(=O)c2c1]

### Step 1. 8-Acetyl-3,6-dimethylquinazoline-2,4(1H,3H)-dione

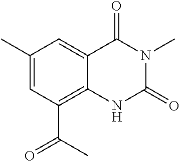 [SMILES: CC(=O)c1cc(C)cc2c(=O)n(C)c(=O)[nH]c12]

To a nitrogen sparged solution of 8-bromo-3,6-dimethyl-1H-quinazoline-2,4-dione (1.00 g, 3.72 mmol) in 1,4-dioxane (35 mL) was added tetrakis(triphenylphosphine)palladium(0) (859 mg, 0.743 mmol) and tributyl(1-ethoxyvinyl)tin (1.88 mL, 5.57 mmol). The reaction was stirred at 100° C. overnight. The reaction mixture was cooled to 50° C., 6 N HCl (2 mL) was added, and was stirred for 1 h. The reaction mixture was cooled to room temperature and purified directly by silica gel chromatography (0-100% EtOAc/hexanes) to afford the title compound (961 mg, 4.14 mmol, 98.0% yield) as a white solid. LC-MS calc. for C₁₂H₁₃N₂O₃ [M+H]⁺: m/z=233.1; Found: 232.9.

### Step 2. tert-Butyl 5-(8-acetyl-3,6-dimethyl-4-oxo-3,4-dihydroquinazolin-2-yl)-2,5-diazabicyclo[2.2.1]heptane-2-carboxylate

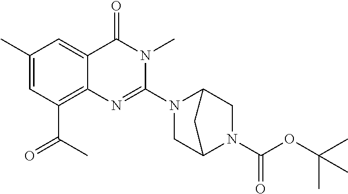 [SMILES: CC(=O)c1cc(C)cc2c(=O)n(C)c(N3CC4CC3CN4C(=O)OC(C)(C)C)nc12]

To a solution of 8-acetyl-3,6-dimethyl-1H-quinazoline-2,4-dione (150 mg, 0.646 mmol) in MeCN (3.25 mL) was added 1,8-diazabicyclo[5.4.0]undec-7-ene (0.290 mL, 1.94 mmol) and bromo-tris-pyrrolidino-phosphonium hexafluorophosphate (361 mg, 0.775 mmol). The reaction was stirred for 1 h. tert-Butyl 2,5-diazabicyclo[2.2.1]heptane-2-carboxylate (192 mg, 0.969 mmol) was added, and the reaction mixture was stirred at 60° C. overnight. The reaction mixture was cooled to room temperature and diluted with water and EtOAc. The layers were separated, and the aqueous layer was extracted with EtOAc (2×10 mL). The combined organic layers were washed with brine, dried over MgSO₄, and concentrated. The crude material was purified by silica gel chromatography (0-100% EtOAc/hexanes) to afford tert-butyl 5-(8-acetyl-3,6-dimethyl-4-oxoquinazolin-2-yl)-2,5-diazabicyclo[2.2.1]heptane-2-carboxylate (61 mg, 0.15 mmol, 23% yield) as a yellow solid. LC-MS calc. for C₂₂H₂₉N₄O₄ [M+H]⁺: m/z=413.2; Found: 413.0.

### Step 3. tert-Butyl 5-(8-(1-hydroxyethyl)-3,6-dimethyl-4-oxo-3,4-dihydroquinazolin-2-yl)-2,5-diazabicyclo[2.2.1]heptane-2-carboxylate

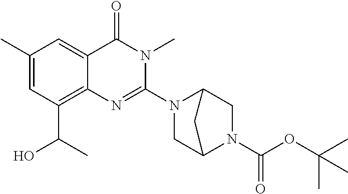 [SMILES: Cc1cc(C(C)O)c2nc(N3CC4CC3CN4C(=O)OC(C)(C)C)n(C)c(=O)c2c1]

The title compound was synthesized by procedures analogous to those outlined in Example 1, Step 6. LC-MS calc. for C₂₂H₃₁N₄O₄ [M+H]⁺: m/z=415.2; Found: 415.1.

### Step 4. 2-[1-[3,6-Dimethyl-2-[5-[(2-methylpropan-2-yl)oxycarbonyl]-2,5-diazabicyclo [2.2.1]heptan-2-yl]-4-oxoquinazolin-8-yl]ethylamino]benzoic acid

The title compound was synthesized by procedures analogous to those outlined in Example 41, Step 4. LC-MS calc. for C₂₉H₃₆N₅O₅ [M+H]⁺: m/z=534.3; Found: 534.0.

### Example 66: 2-((1-(2-((3aR,6aR)-5-Acetylhexahydropyrrolo[3,4-c]pyrrol-2(1H)-yl)-3,6-dimethyl-4-oxo-3,4-dihydroquinazolin-8-yl)ethyl)amino)benzoic acid

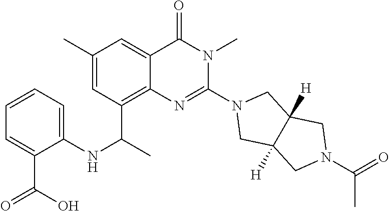 [SMILES: CC(=O)N1C[C@H]2CN(c3nc4c(C(C)Nc5ccccc5C(=O)O)cc(C)cc4c(=O)n3C)C[C@@H]2C1]

The title compound was synthesized by procedures analogous to Example 46, Steps 1-5, substituting tert-butyl (3as,6as)-rel-octahydropyrrolo[3,4-c]pyrrole-2-carboxylate hemioxalate (Astatech, cat #P15553) for 1-Boc-piperazine. LC-MS calc. for C₂₇H₃₂N₅O₄ [M+H]⁺: m/z=490.2; Found: 490.1.

### Example 67: 2-((1-(2-(5,7-Dihydro-6H-pyrrolo[3,4-b]pyridin-6-yl)-3,6-dimethyl-4-oxo-3,4-dihydroquinazolin-8-yl)ethyl)amino)benzoic acid (isomer 1)

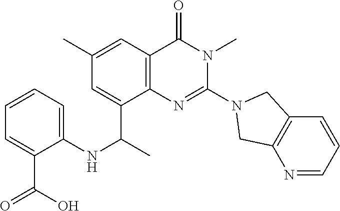 [SMILES: Cc1cc(C(C)Nc2ccccc2C(=O)O)c2nc(N3Cc4cccnc4C3)n(C)c(=O)c2c1]

### Step 1: 8-Bromo-2-(5,7-dihydro-6H-pyrrolo[3,4-b]pyridin-6-yl)-3,6-dimethylquinazolin-4(3H)-one

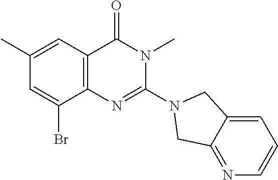 [SMILES: Cc1cc(Br)c2nc(N3Cc4cccnc4C3)n(C)c(=O)c2c1]

The title compound was synthesized by procedures analogous to those outlined in Example 65, Step 2. LC-MS calc. for C₁₇H₁₆BrN₄O [M+H]⁺: m/z=371.0; Found 371.4.

### Step 2: 2-(5,7-Dihydro-6H-pyrrolo[3,4-b]pyridin-6-yl)-8-(1-hydroxyethyl)-3,6-dimethylquinazolin-4(3H)-one

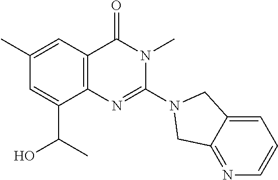 [SMILES: Cc1cc(C(C)O)c2nc(N3Cc4cccnc4C3)n(C)c(=O)c2c1]

The title compound was synthesized by procedures analogous to those outlined in Example 1, Step 5-6. ¹H NMR (400 MHz, DMSO-d₆) δ 8.47 (dd, J=4.0, 1.6 Hz, 1H), 7.81 (dd, J=4.0, 1.2 Hz, 1H), 7.68-7.66 (m, 1H), 7.63-7.62 (m, 1H), 7.31 (dd, J=8.0, 4.0 Hz, 1H), 5.46-5.40 (m, 1H), 5.12-5.08 (m, 2H), 5.04-4.97 (m, 2H), 4.94-4.84 (m, 2H), 3.57 (s, 3H), 2.37 (s, 3H), 1.38 (d, J=8.0 Hz, 3H). LC-MS calc. for C₁₉H₂₁N₄O₂ [M+H]⁺: m/z=337.1; Found 337.5.

### Step 3: Methyl 2-((1-(2-(5,7-dihydro-6H-pyrrolo[3,4-b]pyridin-6-yl)-3,6-dimethyl-4-oxo-3,4-dihydroquinazolin-8-yl)ethyl)amino)benzoate (isomer 1)

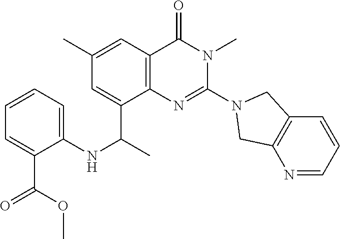 [SMILES: COC(=O)c1ccccc1NC(C)c1cc(C)cc2c(=O)n(C)c(N3Cc4cccnc4C3)nc12]

The title compound was synthesized by procedures analogous to those outlined in Example 23, Step 2. The isomers were separated by chiral prep-HPLC on a Lux i-Amylose-3 column (60:20:20 hexane/IPA/MeOH) to afford two isomers: isomer 1 (t_{R}=5.89 min) and isomer 2 (t_{R}=6.88 min). Isomer 1: LC-MS calc. for C₂₇H₂₈N₅O₃ [M+H]⁺: m/z=470.2; Found 470.6.

### Step 4: Methyl (S)-2-((1-(2-(5,7-dihydro-6H-pyrrolo[3,4-b]pyridin-6-yl)-3,6-dimethyl-4-oxo-3,4-dihydroquinazolin-8-yl)ethyl)amino)benzoate (isomer 1)

The title compound was synthesized by procedures analogous to those outlined in Example 24, Step 2. LC-MS calc. for C₂₆H₂₆N₅O₃ [M+H]⁺ m/z=456.2; Found 456.1.

### Example 68: Methyl 2-[1-[2-(3-azabicyclo[3.1.0]hexan-3-yl)-3-methyl-4-oxo-6-(trideuterio-methyl)quinazolin-8-yl]ethylamino]benzoate (isomer 1)

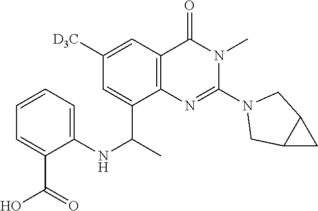 [SMILES: [2H]C([2H])([2H])c1cc(C(C)Nc2ccccc2C(=O)O)c2nc(N3CC4CC4C3)n(C)c(=O)c2c1]

### Step 1: 8-Acetyl-2-(3-azabicyclo[3.1.0]hexan-3-yl)-3-methyl-6-(4,4,5,5-tetramethyl-1,3,2-dioxaborolan-2-yl)quinazolin-4-one

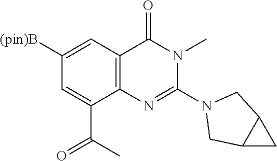

To a solution of 8-acetyl-2-(3-azabicyclo[3.1.0]hexan-3-yl)-6-bromo-3-methylquinazolin-4-one (1.50 g, 4.14 mmol) in 1,4-dioxane (4 mL) was added bis(pinacolato)diboron (1.58 g, 6.21 mmol), [1,1′-bis(diphenylphosphino)ferrocene]dichloropalladium(II), complex with dichloromethane (0.34 g, 0.41 mmol) and potassium acetate (1.22 g, 12.4 mmol). The resulting mixture was subjected to vacuum-nitrogen cycle 3 times. The resulting mixture was stirred at 90° C. overnight. The reaction mixture was cooled to room temperature and diluted with water (100 mL) and EtOAc (100 mL). The layers were separated, and the aqueous layer was extracted with EtOAc (100 mL×3). The combined organic layers were washed with brine, dried over sodium sulfate, filtered, and concentrated. The crude product was further purified by silica gel chromatography (5-30% EtOAc/heptane) to afford the title compound as a beige solid (1.81 g, 4.40 mmol, >99%). ¹H NMR (300 MHz, CDCl₃) δ 8.74 (d, J=1.6 Hz, 1H), 8.34 (d, J=1.6 Hz, 1H), 3.89 (d, J=10.3 Hz, 2H), 3.60-3.48 (m, 5H), 2.81 (s, 3H), 1.62 (dd, J=4.0, 1.9 Hz, 2H), 1.34 (s, 12H), 0.68-0.62 (m, 1H), 0.42-0.37 (m, 1H). LC-MS calc. for C₂₂H₂₉BN₃O₄ [M+H]⁺ m/z=410.2; Found 409.9.

### Step 2: 8-Acetyl-2-(3-azabicyclo[3.1.0]hexan-3-yl)-3-methyl-6-(trideuteriomethyl) quinazolin-4-one

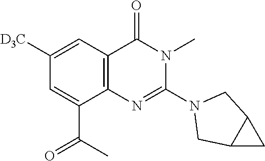 [SMILES: [2H]C([2H])([2H])c1cc(C(C)=O)c2nc(N3CC4CC4C3)n(C)c(=O)c2c1]

To a solution of 8-acetyl-2-(3-azabicyclo[3.1.0]hexan-3-yl)-3-methyl-6-(4,4,5,5-tetramethyl-1,3,2-dioxaborolan-2-yl)quinazolin-4-one (1.6 g, 3.91 mmol) in THF (30 mL) was added [1,1′-bis(diphenylphosphino)ferrocene]dichloropalladium(II), complex with dichloromethane (0.641 g, 0.782 mmol), potassium carbonate (1.62 g, 11.7 mmol) and water (0.210 mL, 11.7 mmol). The resulting mixture was subjected to vacuum-nitrogen cycle 3 times. CD₃I (0.729 mL, 11.7 mmol) was added to the mixture. The resulting solution was stirred at room temperature for 1 h. To the mixture was added water (150 mL) and EtOAc (150 mL). The layers were separated, and the aqueous layer was extracted with EtOAc (150 mL×2). The combined organic layers were washed with brine, dried over sodium sulfate, filtered, and concentrated. The crude product was purified by silica gel chromatography (0-60% EtOAc/heptane) to afford the title compound as a white solid (1.0 g, 3.4 mmol, 85% yield). ¹H NMR (300 MHz, CDCl₃) δ 8.11 (d, J=2.3 Hz, 1H), 7.84 (d, J=2.3 Hz, 1H), 3.82 (d, J=10.2 Hz, 2H), 3.55-3.49 (m, 5H), 2.85 (s, 3H), 1.66-1.60 (m, 2H), 0.69-0.58 (m, 1H), 0.48-0.41 (m, 1H). LC-MS calc. for C₁₇H₁₇D₃N₃O₂ [M+H]⁺ m/z=301.2; Found 301.1.

### Step 3: 2-(3-Azabicyclo[3.1.0]hexan-3-yl)-8-(1-hydroxyethyl)-3-methyl-6-(trideuteriomethyl)quinazolin-4-one

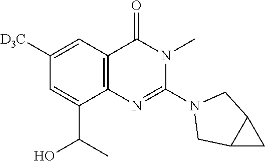 [SMILES: [2H]C([2H])([2H])c1cc(C(C)O)c2nc(N3CC4CC4C3)n(C)c(=O)c2c1]

The title compound was synthesized by procedures analogous to those outlined in Example 1, Step 6. ¹H NMR (300 MHz, CDCl₃) δ 7.85 (d, J=2.1 Hz, 1H), 7.33-7.28 (m, 1H), 5.69 (d, J=6.8 Hz, 1H), 5.16-5.05 (m, 1H), 3.77 (dd, J=13.7, 10.1 Hz, 2H), 3.60-3.42 (m, 5H), 1.66-1.57 (m, 5H), 0.70-0.59 (m, 1H), 0.49-0.42 (m, 1H). LC-MS calc. for C₁₇H₁₉D3N302 [M+H]⁺ m/z=303.2; Found 303.4.

### Step 4: Methyl 2-[1-[2-(3-azabicyclo[3.1.0]hexan-3-yl)-3-methyl-4-oxo-6-(trideuteriomethyl)quinazolin-8-yl]ethylamino]benzoate (isomer 1)

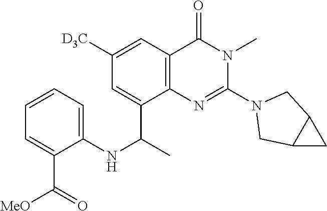 [SMILES: [2H]C([2H])([2H])c1cc(C(C)Nc2ccccc2C(=O)OC)c2nc(N3CC4CC4C3)n(C)c(=O)c2c1]

The title compound was synthesized by procedures analogous to those outlined in Example 1, Step 7, substituting methyl 2-aminobenzoate for anthranilic acid. The isomers were separated using chiral prep-HPLC on a Lux i-Amylose-3 column (85:7.5:7.5 hexane/IPA/MeOH) to afford two isomers: isomer 1 (t_{R}=2.47 min) and isomer 2 (t_{R}=2.85 min). Isomer 1: LC-MS calc. for C₂₅H₂₆D₃N₄O₃ [M+H]⁺ m/z=436.2; Found 436.8.

### Step 5. 2-[[Rac-(1R)-1-[2-(3-azabicyclo[3.1.0]hexan-3-yl)-3-methyl-4-oxo-6-(trideuteriomethyl)quinazolin-8-yl]ethyl]amino]benzoic acid (isomer 1)

The title compound was synthesized by procedures analogous to those outlined in Example 24, Step 2. LC-MS calc. for C₂₄H₂₄D3N403 [M+H]⁺ m/z=422.2; Found 422.1.

### Example 69: 2-((1-(2-(Isoindolin-2-yl)-3,6-dimethyl-4-oxo-3,4-dihydroquinazolin-8-yl)ethyl)amino)-4-methylbenzoic

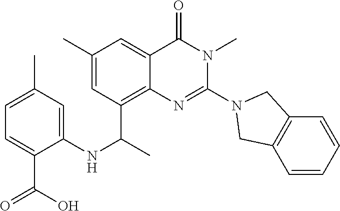 [SMILES: Cc1ccc(C(=O)O)c(NC(C)c2cc(C)cc3c(=O)n(C)c(N4Cc5ccccc5C4)nc23)c1]

To a solution of 8-(1-hydroxyethyl)-2-(isoindolin-2-yl)-3,6-dimethylquinazolin-4(3H)-one (80 mg, 0.24 mmol) in DCM (3.0 mL) was added phosphorus tribromide (196 mg, 0.72 mmol) and stirred at 40° C. for 2 h. To the mixture was added triethylamine (170 mg, 1.68 mmol) and 2-amino-4-methylbenzoic acid (254 mg, 1.68 mmol). The mixture was stirred at 40° C. for 12 h. The mixture was quenched with water (10 mL) and extracted with DCM (10 mL×2), The combined organic phases were concentrated in reduce pressure and purified by prep-HPLC on a C18 column (10-100% MeCN/0.1% TFA (aq)) to afford the title compound (9.1 mg, 7.8% yield), a white solid. LC-MS calc. for C₂₈H₂₇N₄O₃ [M−H]⁻ m/z=467.2; Found 467.9.

### Example 70: 2-((1-(6-Bromo-2-(isoindolin-2-yl)-3-methyl-4-oxo-3,4-dihydroquinazolin-8-yl)ethyl)amino)benzoic acid

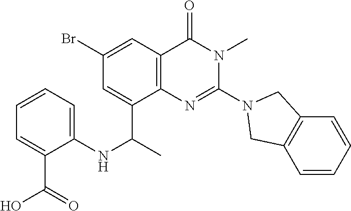 [SMILES: CC(Nc1ccccc1C(=O)O)c1cc(Br)cc2c(=O)n(C)c(N3Cc4ccccc4C3)nc12]

### Step 1: 1-(1-(6-Bromo-2-(isoindolin-2-yl)-3-methyl-4-oxo-3,4-dihydroquinazolin-8-yl)ethyl)-2H-benzo[d][1,3]oxazine-2,4(1H)-dione

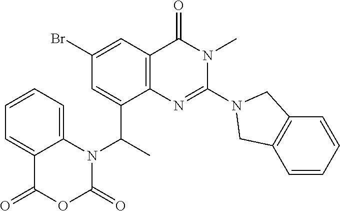 [SMILES: CC(c1cc(Br)cc2c(=O)n(C)c(N3Cc4ccccc4C3)nc12)n1c(=O)oc(=O)c2ccccc21]

To a solution of isatoic anhydride (48.2 mg, 0.295 mmol) and triphenylphosphine (77.4 mg, 0.295 mmol) in THF (2 mL) was added diisopropyl azodicarboxylate (58.1 mL, 0.295 mmol) at 0° C. The mixture was stirred for 10 min. 6-bromo-2-(1,3-dihydroisoindol-2-yl)-8-(1-hydroxyethyl)-3-methylquinazolin-4-one (66 mg, 0.16 mmol) was added. The mixture was stirred for 4 h. To the mixture was added saturated aqueous NaHCO₃ (2 mL). The organic phase was separated, and the aqueous phase was extracted with EtOAc (3×3 mL). The combined organic phases were dried over Na₂SO₄, concentrated, and purified by silica gel chromatography (0-30% EtOAc/heptane) to afford the title compound (55 mg, 0.10 mmol, 62% yield), a colorless solid. LC-MS calc. for C₂₇H₂₂BrN₄O₄ [M+H]⁺ m/z=545.0; Found 544.9.

### Step 2: 2-((1-(6-Bromo-2-(isoindolin-2-yl)-3-methyl-4-oxo-3,4-dihydroquinazolin-8-yl)ethyl)amino)benzoic acid

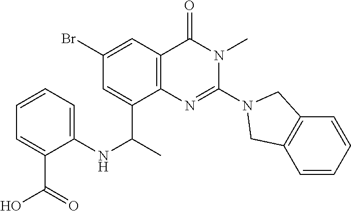 [SMILES: CC(Nc1ccccc1C(=O)O)c1cc(Br)cc2c(=O)n(C)c(N3Cc4ccccc4C3)nc12]

To a solution of 1-[1-[6-bromo-2-(1,3-dihydroisoindol-2-yl)-3-methyl-4-oxoquinazolin-8-yl]ethyl]-3,1-benzoxazine-2,4-dione (12 mg, 0.022 mmol) in DMSO (1 mL) was added 2 N NaOH (5 drops). The mixture was stirred for 0.5 h. The mixture was acidified with 1 N HCl to pH-5, diluted with MeCN, and purified by prep-HPLC on a C18 column (10-70% MeCN/0.1% TFA (aq)) to afford the title compound (2.4 mg, 0.0046 mmol, 21% yield), a colorless solid. ¹H NMR (300 MHz, DMSO-d₆) δ 12.74 (s, 1H), 8.41 (s, 1H), 7.96 (d, J=2.4 Hz, 1H), 7.80 (dd, J=7.9, 1.7 Hz, 1H), 7.63 (d, J=2.4 Hz, 1H), 7.45-7.36 (m, 2H), 7.36-7.29 (m, 2H), 7.18 (ddd, J=8.7, 7.1, 1.7 Hz, 1H), 6.52 (t, J=7.5 Hz, 1H), 6.41 (d, J=8.5 Hz, 1H), 5.51-5.29 (m, 1H), 5.11 (q, J=14.0 Hz, 4H), 3.63 (s, 3H), 1.60 (d, J=6.6 Hz, 3H). LC-MS calc. for C₂₆H₂₄BrN₄O₃ [M+H]⁺ m/z=519.1; Found 519.0.

### Example 71: 2-[1-[3,6-Dimethyl-4-oxo-2-(1-prop-2-enoylpyrrolidin-2-yl)quinazolin-8-yl]ethylamino]benzoic acid

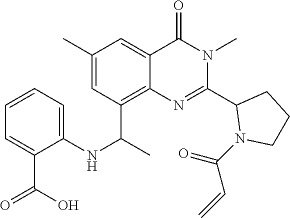 [SMILES: C=CC(=O)N1CCCC1c1nc2c(C(C)Nc3ccccc3C(=O)O)cc(C)cc2c(=O)n1C]

To a solution of 2-[1-(3,6-dimethyl-4-oxo-2-pyrrolidin-2-ylquinazolin-8-yl)ethylamino]benzoic acid (8.4 mg, 0.013 mmol) in DCM (1 mL) was added triethylamine (11.1 μL, 0.0794 mmol) and acrylic anhydride (1.11 μL, 0.00927 mmol). The resulting mixture was stirred for 0.5 h. Additional portions of triethylamine (11.1 μL, 0.0794 mmol) and acrylic anhydride (1.11 μL, 0.00927 mmol) were added and the mixture was stirred for 1 h. The mixture was concentrated under reduced pressure and purified by prep-HPLC on C18 column (20-100% MeCN/0.1% TFA (aq)) to afford the title compound (2.3 mg, 0.0046 mmol, 35% yield), a white solid. ¹H NMR (300 MHz, DMSO-d₆) δ 12.68 (s, 1H), 8.39-8.37 (m, 1H), 7.86-7.69 (m, 2H), 7.49-7.45 (m, 1H), 7.11-6.91 (m, 1H), 6.70-6.65 (m, 1H), 6.53-6.39 (m, 1H), 6.24-6.18 (m, 1H), 6.14-5.98 (m, 1H), 5.67-5.51 (m, 1H), 5.49-5.21 (m, 2H), 3.97-3.75 (m, 2H), 3.66-3.62 (m, 3H), 2.35-2.32 (m, 3H), 2.24-1.89 (m, 4H), 1.47-1.42 (m, 3H). LC-MS calc. for C₂₆H₂₉N₄O₄ [M+H]⁺ m/z=461.2; Found 461.1.

### Example 72: 2-((1-(2-(Isoindolin-2-yl)-3-methyl-4-oxo-3,4-dihydroquinazolin-8-yl)ethyl)amino)benzoic acid

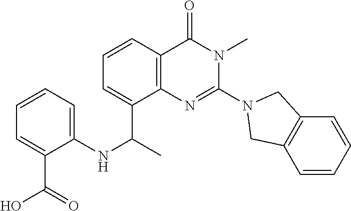 [SMILES: CC(Nc1ccccc1C(=O)O)c1cccc2c(=O)n(C)c(N3Cc4ccccc4C3)nc12]

To a suspension of 2-[1-[6-bromo-2-(1,3-dihydroisoindol-2-yl)-3-methyl-4-oxoquinazolin-8-yl]ethylamino]benzoic acid (10 mg, 0.019 mmol) and sodium bicarbonate (3 mg, 0.04 mmol) in DCM (1 ml) was added 10% Pd/C (2.0 mg, 0.098 mmol). Methanol (3 mL) was added. The mixture was stirred under a hydrogen atmosphere for 1 h. The mixture was filtered, concentrated, and purified by prep-HPLC on a C18 column (10-70% MeCN/0.1% TFA (aq)) to afford the title compound (5 mg, 0.01 mmol, 60% yield), a colorless solid. ¹H NMR (300 MHz, DMSO-d₆) δ 12.67 (s, 1H), 8.43 (s, 1H), 7.88 (dd, J=7.9, 1.5 Hz, 1H), 7.77 (dd, J=8.0, 1.7 Hz, 1H), 7.56 (dd, J=7.4, 1.6 Hz, 1H), 7.47-7.36 (m, 2H), 7.36-7.28 (m, 2H), 7.22-7.08 (m, 2H), 6.53-6.38 (m, 2H), 5.46 (q, J=6.6 Hz, 1H), 5.09 (q, J=13.7 Hz, 4H), 3.62 (s, 3H), 1.59 (d, J=6.6 Hz, 3H). LC-MS calc. for C₂₆H₂₅N₄O₃ [M+H]⁺ m/z=441.2; Found 441.0.

### Example 73: 2-[1-[2-(1-Fluorosulfonylpyrrolidin-2-yl)-3,6-dimethyl-4-oxoquinazolin-8-yl]ethylamino]benzoic acid

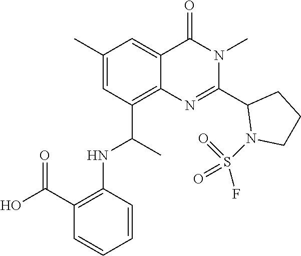 [SMILES: Cc1cc(C(C)Nc2ccccc2C(=O)O)c2nc(C3CCCN3S(=O)(=O)F)n(C)c(=O)c2c1]

### Step 1: tert-Butyl 2-[[2-bromo-4-methyl-6-(methylcarbamoyl)phenyl]carbamoyl]pyrrolidine-1-carboxylate

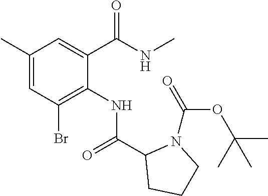 [SMILES: CNC(=O)c1cc(C)cc(Br)c1NC(=O)C1CCCN1C(=O)OC(C)(C)C]

To a solution of 2-amino-3-bromo-N,5-dimethylbenzamide (900 mg, 3.70 mmol) in DCM (18 mL) was added 1-[(2-methylpropan-2-yl)oxycarbonyl]pyrrolidine-2-carboxylic acid (1.59 g, 7.40 mmol) followed by the addition of 1-ethyl-3-(3-dimethylaminopropyl)carbodiimide hydrochloride (1.42 g, 7.40 mmol) and 4-(dimethylamino)pyridine (1.81 g, 14.8 mmol). The resulting mixture was stirred for 48 h. The mixture was quenched with 1 N HCl (50 mL) and diluted with EtOAc (50 mL). The layers were separated, and the aqueous layer was extracted with EtOAc (50 mL×2). The combined organic layers were washed with brine, dried over Na₂SO₄, filtered, and concentrated under reduced pressure. The crude material was purified by silica gel chromatography (30-100% EtOAc/heptane) to afford the title compound (390 mg, 0.89 mmol, 24% yield), a white solid. ¹H NMR (300 MHz, CDCl₃) δ 8.52 (s, 1H), 7.54-7.43 (m, 1H), 7.33 (s, 1H), 6.61 (s, 1H), 4.44 (s, 1H), 3.61-3.38 (m, 2H), 2.90 (d, J=4.9 Hz, 3H), 2.32 (s, 4H), 2.03-1.87 (m, 2H), 1.71 (d, J=9.1 Hz, 1H), 1.49 (s, 9H). LC-MS calc. for C₁₉H₂₇BrN₃O₄ [M+H]⁺ m/z=440.1/442.1; Found 439.9/441.9.

### Step 2: tert-Butyl 2-(8-bromo-3,6-dimethyl-4-oxo-3,4-dihydroquinazolin-2-yl)pyrrolidine-1-carboxylate

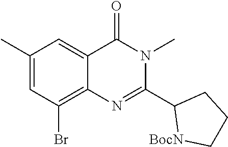 [SMILES: Cc1cc(Br)c2nc(C3CCCN3C(=O)OC(C)(C)C)n(C)c(=O)c2c1]

To a solution of tert-butyl 2-[[2-bromo-4-methyl-6-(methylcarbamoyl)phenyl]carbamoyl]pyrrolidine-1-carboxylate (370 mg, 0.840 mmol) in DCM (7 mL) was added iodine (320 mg, 1.26 mmol). The reaction was stirred until all iodine was dissolved. The reaction mixture was cooled to 0° C. and [dimethyl-(trimethylsilylamino)silyl]methane (0.264 mL, 1.26 mmol) was added dropwise. The reaction mixture was warmed to room temperature and stirred for 7 h. The reaction mixture was diluted with DCM (30 mL) 10% aq. Na₂S₂O₃ (30 mL). The layers were separated, and the aqueous layer was extracted with DCM (30 mL). The combined organic layers were dried over Na₂SO₄, filtered, and concentrated. The crude material was purified by silica gel chromatography (0-100% EtOAc/heptane) to afford the title compound (211 mg, 0.499 mmol, 59.0% yield), a beige solid. LC-MS calc. for C₁₉H₂₅BrN₃O₃ [M+H]⁺ m/z=422.1/424.1; Found 422.0/424.0.

### Step 3: tert-Butyl 2-[8-(1-hydroxyethyl)-3,6-dimethyl-4-oxoquinazolin-2-yl]pyrrolidine-1-carboxylate

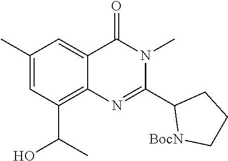 [SMILES: Cc1cc(C(C)O)c2nc(C3CCCN3C(=O)OC(C)(C)C)n(C)c(=O)c2c1]

The title compound was synthesized by procedures analogous to those outlined in Example 39, Step 2-3. LC-MS calc. for C₂₁H₃₀N₃O₄ [M+H]⁺ m/z=388.2; Found 388.2.

### Step 4: 2-[1-(3,6-Dimethyl-4-oxo-2-pyrrolidin-2-ylquinazolin-8-yl)ethylamino]benzoic acid

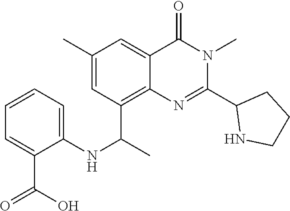 [SMILES: Cc1cc(C(C)Nc2ccccc2C(=O)O)c2nc(C3CCCN3)n(C)c(=O)c2c1]

The title compound was synthesized by procedures analogous to those outlined in Example 41, Step 4. LC-MS calc. for C₂₃H₂₇N₄O₃ [M+H]⁺ m/z=407.2; Found 407.0.

### Step 5: 2-[1-[2-(1-Fluorosulfonylpyrrolidin-2-yl)-3,6-dimethyl-4-oxoquinazolin-8-yl]ethylamino]benzoic acid

To a solution of 2-[1-(3,6-dimethyl-4-oxo-2-pyrrolidin-2-ylquinazolin-8-yl)ethylamino]benzoic acid (18.4 mg, 0.0290 mmol) in MeCN (1 mL) was added triethylamine (0.0121 mL, 0.0870 mmol) followed by 1-(fluorosulfonyl)-2,3-dimethyl-1H-imidazol-3-ium trifluoromethanesulfonate (9.52 mg, 0.0290 mmol). The resulting mixture was stirred for 1 h. Additional portions of triethylamine (0.012 mL, 0.087 mmol) and 1-(fluorosulfonyl)-2,3-dimethyl-1H-imidazol-3-ium trifluoromethanesulfonate (9.5 mg, 0.029 mmol) were added and the mixture was stirred for 1 h. The mixture was concentrated and purified by prep-HPLC on a C18 column (20-80% MeCN/0.1% TFA (aq)) to afford the title compound (3.3 mg, 0.0068 mmol, 22% yield), a white solid. LC-MS calc. for C₂₃H₂₆FN₄O₅S [M+H]⁺ m/z=489.2; Found 489.1.

### Example 74: 2-((1-(6-Bromo-2-(4,4-difluoropiperidin-1-yl)-3-methyl-4-oxo-3,4-dihydroquinazolin-8-yl)ethyl)amino)benzoic acid

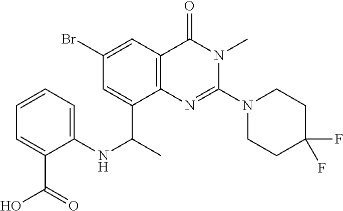 [SMILES: CC(Nc1ccccc1C(=O)O)c1cc(Br)cc2c(=O)n(C)c(N3CCC(F)(F)CC3)nc12]

### Step 1: 6-Bromo-2-(4,4-difluoropiperidin-1-yl)-8-iodo-3-methylquinazolin-4(3H)-one

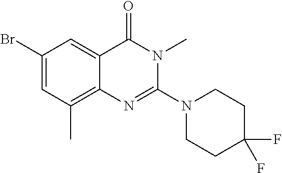 [SMILES: Cc1cc(Br)cc2c(=O)n(C)c(N3CCC(F)(F)CC3)nc12]

To a suspension of 6-bromo-2-chloro-8-iodo-3-methylquinazolin-4-one (795 mg, 1.99 mmol) and 4,4-difluoropiperidine hydrochloride (565 mg, 3.58 mmol) in THF (20 mL) was added N,N-diisopropylethylamine (1.39 mL, 7.96 mmol). The mixture was stirred at 60° C. for 4 h. The mixture was cooled to room temperature and diluted with water (20 mL) and EtOAc (20 mL). The layers were separated, and the aqueous layer was extracted with EtOAc (3×20 mL). The combined organic layers were dried over Na₂SO₄, concentrated, and purified by silica gel chromatography (0-50% EtOAc/heptane) to afford the title compound (870 mg, 1.8 mmol, 91% yield), a colorless solid. LC-MS calc. for C₁₄H₁₄BrF₂IN₃O [M+H]⁺ m/z=483.9; Found 483.7.

### Step 2: 6-Bromo-2-(4,4-difluoropiperidin-1-yl)-8-(1-hydroxyethyl)-3-methylquinazolin-4(3H)-one

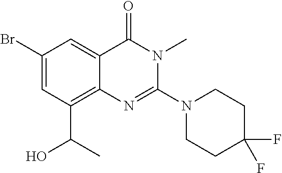 [SMILES: CC(O)c1cc(Br)cc2c(=O)n(C)c(N3CCC(F)(F)CC3)nc12]

The title compound was synthesized by procedures analogous to those outlined in Example 39, Steps 2-3. LC-MS calc. for C₁₆H₁₉BrF₂N₃O₂ [M+H]⁺ m/z=402.1; Found 401.9.

### Step 3: 2-((1-(6-Bromo-2-(4,4-difluoropiperidin-1-yl)-3-methyl-4-oxo-3,4-dihydro-quinazolin-8-yl)ethyl)amino)benzoic acid

### Example 75: 2-((1-(3-((1r,3r)-3-Cyanocyclobutyl)-2-(isoindolin-2-yl)-6-methyl-4-oxo-3,4-dihydroquinazolin-8-yl)ethyl)amino)benzoic acid

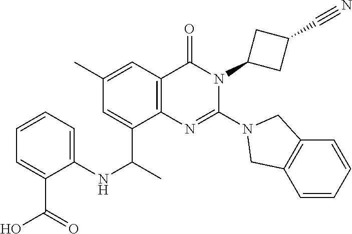 [SMILES: Cc1cc(C(C)Nc2ccccc2C(=O)O)c2nc(N3Cc4ccccc4C3)n([C@H]3C[C@H](C#N)C3)c(=O)c2c1]

### Step 1: Methyl 2-((1-(2-(isoindolin-2-yl)-6-methyl-3-((1s,3s)-3-((4-nitrobenzoyl)oxy) cyclobutyl)-4-oxo-3,4-dihydroquinazolin-8-yl)ethyl)amino)benzoate

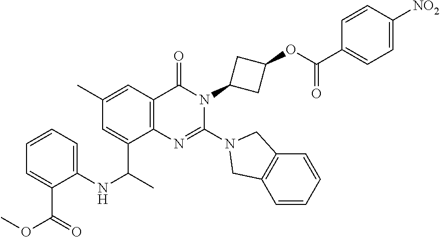 [SMILES: COC(=O)c1ccccc1NC(C)c1cc(C)cc2c(=O)n([C@H]3C[C@@H](OC(=O)c4ccc([N+](=O)[O-])cc4)C3)c(N3Cc4ccccc4C3)nc12]

To a solution of 4-nitrobenzoic acid (71 mg, 0.42 mmol), methyl 2-[1-[2-(1,3-dihydroisoindol-2-yl)-3-(3-hydroxycyclobutyl)-6-methyl-4-oxoquinazolin-8-yl]ethylamino]benzoate (150 mg, 0.286 mmol) and tri-n-butylphosphine (87 mg, 0.43 mmol) in THF (3 mL) was added a solution of di-tert-butyl azodicarboxylate (99 mg, 0.43 mmol) in THF (1 mL) dropwise. The reaction was refluxed under a nitrogen atmosphere for 48 h. The mixture was concentrated and purified by silica gel column chromatography (2% EtOAc/hexanes) to afford the title compound (60 mg, 0.089 mmol, 31% yield) as a light-yellow solid. ¹H NMR (400 MHz, DMSO-d₆) δ 8.36 (d, J=8.0 Hz, 2H), 8.30-8.28 (m, 1H), 8.26 (d, J=8.0 Hz, 2H), 7.75 (dd, J=8.0, 4.0 Hz, 1H), 7.72-7.71 (m, 1H), 7.44 (d, J=2.0 Hz, 1H), 7.38-7.35 (m, 2H), 7.30-7.26 (m, 2H), 7.19-7.15 (m, 1H), 6.53 (d, J=8.0 Hz, 1H), 6.51-6.47 (m, 1H), 5.63-5.57 (m, 1H), 5.41-5.34 (m, 1H), 5.29-5.21 (m, 1H), 4.94 (q, J=12.0 Hz, 4H), 3.76 (s, 3H), 3.23-3.14 (m, 2H), 2.94-2.82 (m, 2H), 2.30 (s, 3H), 1.57 (d, J=4.0 Hz, 3H). LC-MS calc. for C₃₈H₃₆N₅O₇ [M+H]+: m/z=674.2; Found 673.5.

### Step 2: Methyl 2-((1-(3-((1s,3s)-3-hydroxycyclobutyl)-2-(isoindolin-2-yl)-6-methyl-4-oxo-3,4-dihydroquinazolin-8-yl)ethyl)amino)benzoate

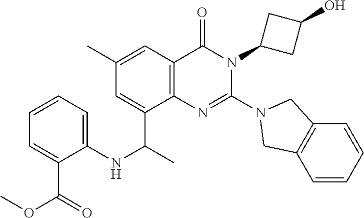 [SMILES: COC(=O)c1ccccc1NC(C)c1cc(C)cc2c(=O)n([C@H]3C[C@@H](O)C3)c(N3Cc4ccccc4C3)nc12]

To a solution of methyl 2-[1-[2-(1,3-dihydroisoindol-2-yl)-6-methyl-3-[3-(4-nitrobenzoyl)oxycyclobutyl]-4-oxoquinazolin-8-yl]ethylamino]benzoate (130 mg, 0.193 mmol) in THE (8 mL) and MeOH (2 mL) was added a solution of Na₂CO₃ (62 mg, 0.59 mmol) in water (2 mL). The reaction was stirred at 25° C. for 2 h. The mixture was diluted with water (20 mL) and extracted with EtOAc (25 mL×2). The combined organic layers were washed with brine (20 mL), dried over by Na₂SO₄, filtered and concentrated. The crude material was purified by prep-TLC (50% EtOAc/hexanes) to give the title compound (82 mg, 0.16 mmol, 81% yield), a white solid. ¹H NMR (400 MHz, DMSO-d₆) δ 8.27 (d, J=4.0 Hz, 1H), 7.74 (dd, J=4.0, 1.2 Hz, 1H), 7.64 (dd, J=1.2, 0.4 Hz, 1H), 7.40 (d, J=4.0 Hz, 1H), 7.39-7.37 (m, 2H), 7.29-7.27 (m, 2H), 7.18-7.15 (m, 1H), 6.52 (d, J=4.0 Hz, 1H), 6.49-6.46 (m, 1H), 5.38-5.34 (m, 1H), 5.11-5.06 (m, 1H), 5.04 (d, J=4.0 Hz, 1H), 4.94 (q, J=12.0 Hz, 4H), 4.37-4.33 (m, 1H), 3.75 (s, 3H), 2.84-2.79 (m 2H), 2.40-2.38 (m, 1H), 2.34-2.30 (m, 1H), 2.27 (s, 3H), 1.55 (d, J=4.0 Hz, 3H). LC-MS calc. for C₃₁H₃₃N₄O₄ [M+H]⁺: m/z=525.2; Found 525.1.

### Step 3: Methyl 2-((1-(2-(isoindolin-2-yl)-6-methyl-3-((1s,3s)-3-((methylsulfonyl)oxy) cyclobutyl)-4-oxo-3,4-dihydroquinazolin-8-yl)ethyl)amino)benzoate

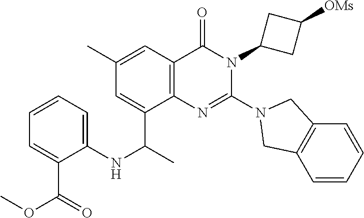 [SMILES: COC(=O)c1ccccc1NC(C)c1cc(C)cc2c(=O)n([C@H]3C[C@@H](OS(C)(=O)=O)C3)c(N3Cc4ccccc4C3)nc12]

To a solution of methyl 2-[1-[2-(1,3-dihydroisoindol-2-yl)-3-(3-hydroxycyclobutyl)-6-methyl-4-oxoquinazolin-8-yl]ethylamino]benzoate (15.8 mg, 0.0247 mmol) in DCM (1 mL) was added triethylamine (20.7 mL, 0.148 mmol) and methanesulfonyl chloride (3.83 mL, 0.0495 mmol). The mixture was stirred for 0.5 h. The mixture was quenched with water (1 mL). The organic phase was separated, and the aqueous phase was extracted with DCM (3×1 mL). The combined organic phases were dried over Na₂SO₄ and concentrated. The crude product was used directly in the next step without any further purification. LC-MS calc. for C₃₂H₃₅N₄O₆S [M+H]⁺: m/z=603.2; Found 603.1.

### Step 4: 2-((1-(3-((1r,3r)-3-Cyanocyclobutyl)-2-(isoindolin-2-yl)-6-methyl-4-oxo-3,4-dihydroquinazolin-8-yl)ethyl)amino)benzoic acid

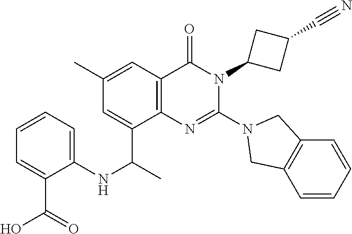 [SMILES: Cc1cc(C(C)Nc2ccccc2C(=O)O)c2nc(N3Cc4ccccc4C3)n([C@H]3C[C@H](C#N)C3)c(=O)c2c1]

To a solution of methyl 2-[1-[2-(1,3-dihydroisoindol-2-yl)-6-methyl-3-(3-methylsulfonyloxycyclobutyl)-4-oxoquinazolin-8-yl]ethylamino]benzoate (15 mg, 0.025 mmol) in DMF (1 mL) was added sodium cyanide (9.68 mg, 0.198 mmol). The mixture was heated to 125° C. for 24 h. The mixture was cooled to room temperature, filtered, and purified by prep-HPLC on a C18 column (10-70% MeCN/0.1% TFA (aq)) to afford the title compound (3.9 mg, 0.0075 mmol, 30% yield), a yellow solid. ¹H NMR (300 MHz, DMSO-d₆) δ 12.67 (br, 1H), 8.41 (s, 1H), 7.77 (dd, J=8.1, 1.6 Hz, 1H), 7.68 (d, J=2.1 Hz, 1H), 7.46-7.38 (m, 3H), 7.34-7.27 (m, 2H), 7.13 (td, J=7.7, 1.7 Hz, 1H), 6.47 (t, J=7.8 Hz, 2H), 5.45-5.30 (m, 1H), 5.30-5.12 (m, 1H), 5.00 (d, J=13.8 Hz, 2H), 4.85 (d, J=13.9 Hz, 2H), 3.61-3.55 (m, 1H), 3.16-2.99 (m, 2H), 2.98-2.70 (m, 2H), 2.30 (s, 3H), 1.56 (d, J=6.6 Hz, 3H). LC-MS calc. for C₃₁H₃₀N₅O₃ [M+H]⁺ m/z=520.2; Found 520.0.

### Example 76: 2-((1-(2-(3-Azabicyclo[3.1.0]hexan-3-yl)-6-cyclopropyl-3-methyl-4-oxo-3,4-dihydroquinazolin-8-yl)ethyl)amino)benzoic acid

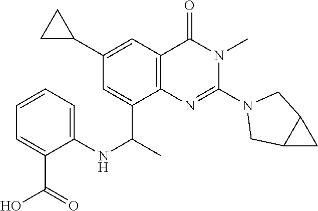 [SMILES: CC(Nc1ccccc1C(=O)O)c1cc(C2CC2)cc2c(=O)n(C)c(N3CC4CC4C3)nc12]

### Step 1: 2-(3-Azabicyclo[3.1.0]hexan-3-yl)-6-bromo-8-iodo-3-methylquinazolin-4(3H)-one

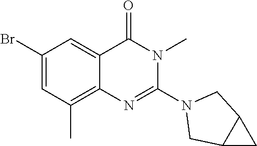 [SMILES: Cc1cc(Br)cc2c(=O)n(C)c(N3CC4CC4C3)nc12]

The title compound was synthesized by procedures analogous to those outlined in Example 74, Step 1. LC-MS calc. for C₁₄H₁₄BrIN₃O [M+H]⁺: m/z=445.9; Found 445.8.

### Step 2: 2-(3-Azabicyclo[3.1.0]hexan-3-yl)-6-bromo-8-(1-hydroxyethyl)-3-methylquinazolin-4(3H)-one

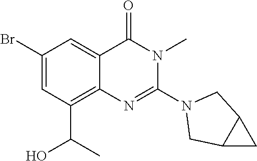 [SMILES: CC(O)c1cc(Br)cc2c(=O)n(C)c(N3CC4CC4C3)nc12]

The title compound was synthesized by procedures analogous to those outlined in Example 39, Steps 2-3. LC-MS calc. for C₁₆H₁₉BrN₃O₂ [M+H]⁺: m/z=364.1; Found 363.9.

### Step 3: 1-(1-(2-(3-Azabicyclo[3.1.0]hexan-3-yl)-6-bromo-3-methyl-4-oxo-3,4-dihydroquinazolin-8-yl)ethyl)-2H-benzo[d][1,3]oxazine-2,4(1H)-dione

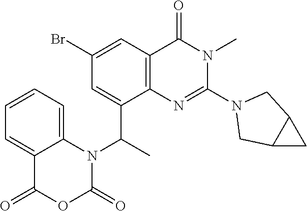 [SMILES: CC(c1cc(Br)cc2c(=O)n(C)c(N3CC4CC4C3)nc12)n1c(=O)oc(=O)c2ccccc21]

The title compound was synthesized by procedures analogous to those outlined in Example 70, Step 1. LC-MS calc. for C₂₄H₂₂BrN₄O₄ [M+H]⁺: m/z=509.0; Found 508.9.

### Step 4: 2-((1-(2-(3-azabicyclo[3.1.0]hexan-3-yl)-6-cyclopropyl-3-methyl-4-oxo-3,4-dihydroquinazolin-8-yl)ethyl)amino)benzoic acid

A solution of 1-[1-[2-(3-azabicyclo[3.1.0]hexan-3-yl)-6-bromo-3-methyl-4-oxoquinazolin-8-yl]ethyl]-3,1-benzoxazine-2,4-dione (13.8 mg, 0.0271 mmol), cyclopropylboronic acid (11.6 mg, 0.135 mmol), potassium carbonate (22.5 mg, 0.163 mmol) and [1,1′-bis(diphenylphosphino)ferrocene]dichloropalladium(II) (1.98 mg, 2.71 mmol) in 1,4-dioxane (0.8 mL) and water (0.2 mL) was heated to 100° C. under a nitrogen atmosphere for 6 h. The mixture was cooled to room temperature, filtered, and purified by prep-HPLC on a C18 column (10-80% MeCN/0.1% TFA (aq)) to afford the title compound (5.5 mg, 0.012 mmol, 46% yield), a colorless solid. ¹H NMR (300 MHz, DMSO-d₆) δ 12.68 (br, 1H), 8.42 (br, 1H), 7.79 (dd, J=7.9, 1.7 Hz, 1H), 7.51 (d, J=2.2 Hz, 1H), 7.35 (d, J=2.2 Hz, 1H), 7.20 (ddd, J=8.6, 7.1, 1.7 Hz, 1H), 6.56-6.44 (m, 2H), 5.36 (q, J=6.7 Hz, 1H), 4.00 (d, J=10.5 Hz, 1H), 3.83 (d, J=10.5 Hz, 1H), 3.51 (dd, J=19.6, 10.3 Hz, 2H), 3.43 (s, 3H), 2.05-1.87 (m, 1H), 1.63 (dd, J=7.2, 3.7 Hz, 2H), 1.57 (d, J=6.6 Hz, 3H), 0.97-0.89 (m, 2H), 0.67-0.48 (m, 3H), 0.38 (q, J=4.2 Hz, 1H). LCMS calc. for C₂₆H₂₉N₄O₃ [M+H]⁺ m/z=445.2; Found 444.9.

### Example 77: 2-((1-(2-(3-azabicyclo[3.1.0]hexan-3-yl)-6-methoxy-3-methyl-4-oxo-3,4-dihydroquinazolin-8-yl)ethyl)amino)benzoic acid

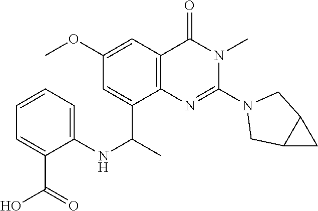 [SMILES: COc1cc(C(C)Nc2ccccc2C(=O)O)c2nc(N3CC4CC4C3)n(C)c(=O)c2c1]

### Step 1: 8-Acetyl-2-(3-azabicyclo[3.1.0]hexan-3-yl)-6-hydroxy-3-methylquinazolin-4(3H)-one

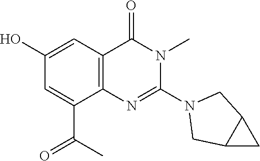 [SMILES: CC(=O)c1cc(O)cc2c(=O)n(C)c(N3CC4CC4C3)nc12]

To a solution of 8-acetyl-2-(3-azabicyclo[3.1.0]hexan-3-yl)-3-methyl-6-(4,4,5,5-tetramethyl-1,3,2-dioxaborolan-2-yl)quinazolin-4-one (100 mg, 0.24 mmol) in acetone (3 mL) was added a solution of Oxone (93 mg, 0.61 mmol) in water (1 mL). The reaction was stirred for 1 h. The mixture was extracted with EtOAc (3×3 mL) and the combined organic layers were dried over MgSO₄, concentrated, and purified by silica gel chromatography (10-90% EtOAc/heptane) to afford the title compound (65 mg, 0.22 mmol, 89% yield), a yellow solid. LC-MS calc. for C₁₆H₁₈N₃O₃ [M+H]⁺ m/z=300.1; Found 300.4.

### Step 2: 8-Acetyl-2-(3-azabicyclo[3.1.0]hexan-3-yl)-6-methoxy-3-methylquinazolin-4(3H)-one

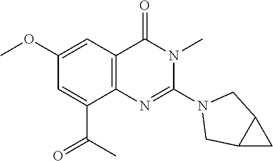 [SMILES: COc1cc(C(C)=O)c2nc(N3CC4CC4C3)n(C)c(=O)c2c1]

To a solution of 8-acetyl-2-(3-azabicyclo[3.1.0]hexan-3-yl)-6-hydroxy-3-methylquinazolin-4-one (63 mg, 0.21 mmol) in DCM (1 mL), acetone (3 mL), and MeOH (2 mL) was added potassium carbonate (291 mg, 2.10 mmol) and iodomethane (197 mL, 3.16 mmol). The mixture was stirred at 35° C. for 2 days. The mixture was filtered, concentrated, and purified by silica gel chromatography (0-60% EtOAc/heptane) to afford the title compound (37 mg, 0.12 mmol, 56% yield), a colorless solid. LC-MS calc. for C₁₇H₂₀N₃O₃ [M+H]⁺ m/z=314.1; Found 314.3.

### Step 3: 2-(3-Azabicyclo[3.1.0]hexan-3-yl)-8-(1-hydroxyethyl)-6-methoxy-3-methylquinazolin-4(3H)-one

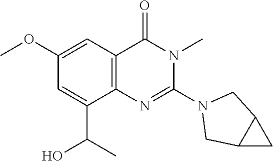 [SMILES: COc1cc(C(C)O)c2nc(N3CC4CC4C3)n(C)c(=O)c2c1]

The title compound was synthesized by procedures analogous to those outlined in Example 1, Steps 6. LC-MS calc. for C₁₇H₂₂N₃O₃ [M+H]⁺ m/z=316.1; Found 316.3.

### Step 4: 2-((1-(2-(3-Azabicyclo[3.1.0]hexan-3-yl)-6-methoxy-3-methyl-4-oxo-3,4-dihydroquinazolin-8-yl)ethyl)amino)benzoic acid

The title compound was synthesized by procedures analogous to those outlined in Example 41, Step 4. ¹H NMR (300 MHz, DMSO-d₆) δ 12.73 (s, 1H), 8.41 (s, 1H), 7.80 (dd, J=8.0, 1.7 Hz, 1H), 7.28 (d, J=3.0 Hz, 1H), 7.23-7.13 (m, 2H), 6.57-6.46 (m, 1H), 6.42 (d, J=8.5 Hz, 1H), 5.46-5.27 (m, 1H), 3.96 (d, J=10.4 Hz, 1H), 3.82 (d, J=10.4 Hz, 1H), 3.75 (s, 3H), 3.56-3.48 (m, 2H), 3.45 (s, 3H), 1.66-1.60 (m, 2H), 1.57 (d, J=6.6 Hz, 3H), 0.60 (td, J=7.6, 4.5 Hz, 1H), 0.42 (q, J=4.1 Hz, 1H). LC-MS calc. for C₂₄H₂₇N₄O₄ [M+H]⁺ m/z=435.2; Found 435.4.

### Example 78: 2-((1-(2-(3-Azabicyclo[3.1.0]hexan-3-yl)-6-ethyl-3-methyl-4-oxo-3,4-dihydroquinazolin-8-yl)ethyl)amino)benzoic acid

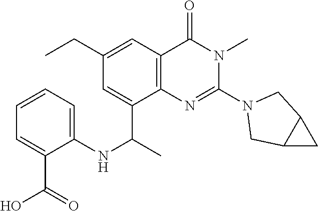 [SMILES: CCc1cc(C(C)Nc2ccccc2C(=O)O)c2nc(N3CC4CC4C3)n(C)c(=O)c2c1]

### Step 1: 2-((1-(2-(3-Azabicyclo[3.1.0]hexan-3-yl)-3-methyl-4-oxo-6-vinyl-3,4-dihydroquinazolin-8-yl)ethyl)amino)benzoic acid

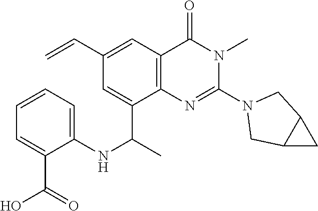 [SMILES: C=Cc1cc(C(C)Nc2ccccc2C(=O)O)c2nc(N3CC4CC4C3)n(C)c(=O)c2c1]

A solution of 1-[1-[2-(3-azabicyclo[3.1.0]hexan-3-yl)-6-bromo-3-methyl-4-oxoquinazolin-8-yl]ethyl]-3,1-benzoxazine-2,4-dione (16.7 mg, 0.0328 mmol), [1,1′-bis(diphenylphosphino)ferrocene]dichloropalladium(II) (2.4 mg, 3.3 mmol), potassium carbonate (13.6 mg, 0.0984 mmol), and vinylboronic acid pinacol ester (11.2 mL, 0.0656 mmol) in 1,4-dioxane (0.75 mL) and water (0.25 mL) was heated to 100° C. under a nitrogen atmosphere for 3 h. The mixture was cooled to room temperature, filtered, concentrated, and purified by prep-HPLC on a C18 column (10-80% MeCN/0.1% TFA (aq)) to afford the title compound (7.5 mg, 0.017 mmol, 53% yield), a colorless solid. LC-MS calc. for C₂₅H₂₇N₄O₃ [M+H]⁺ m/z=431.2; Found 431.3.

### Step 2: 2-((1-(2-(3-Azabicyclo[3.1.0]hexan-3-yl)-6-ethyl-3-methyl-4-oxo-3,4-dihydroquinazolin-8-yl)ethyl)amino)benzoic acid

To a solution of 2-[1-[2-(3-azabicyclo[3.1.0]hexan-3-yl)-6-ethenyl-3-methyl-4-oxoquinazolin-8-yl]ethylamino]benzoic acid (7.5 mg, 0.017 mmol) in DCM (0.5 mL) added 10% Pd/C (10 mg, 0.094 mmol) and methanol (3 mL). The mixture was stirred under a hydrogen atmosphere for 6 h. The mixture was filtered and concentrated to afford the title compound (4.4 mg, 0.010 mmol, 58% yield), a colorless solid. ¹H NMR (300 MHz, DMSO-d₆) δ 12.69 (br, 1H), 8.44 (br, 1H), 7.79 (dd, J=8.1, 1.6 Hz, 1H), 7.69 (d, J=2.1 Hz, 1H), 7.46 (d, J=2.1 Hz, 1H), 7.25-7.13 (m, 1H), 6.55-6.44 (m, 2H), 5.44-5.27 (m, 1H), 4.01 (d, J=10.5 Hz, 1H), 3.84 (d, J=10.5 Hz, 1H), 3.58-3.49 (m, 2H), 3.44 (s, 3H), 2.61 (q, J=7.5 Hz, 2H), 1.67-1.60 (m, 2H), 1.58 (d, J 6.7 Hz, 3H), 1.12 (t, J=7.5 Hz, 3H), 0.61 (td, J=7.7, 4.6 Hz, 1H), 0.38 (q, J=4.2 Hz, 1H). LC-MS calc. for C₂₅H₂₉N₄O₃ [M+H]⁺ m/z=433.2; Found 433.3.

### Example 79: 2-((1-(2-(3-Azabicyclo[3.1.0]hexan-3-yl)-6-(difluoromethyl)-3-methyl-4-oxo-3,4-dihydroquinazolin-8-yl)ethyl)amino)benzoic acid

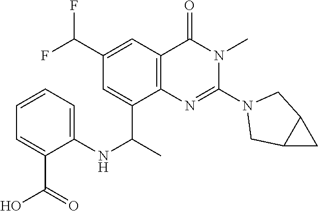 [SMILES: CC(Nc1ccccc1C(=O)O)c1cc(C(F)F)cc2c(=O)n(C)c(N3CC4CC4C3)nc12]

### Step 1: 2-Amino-3-bromo-5-chloro-N-methylbenzamide

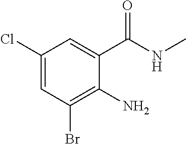

To a solution of 2-amino-3-bromo-5-chlorobenzoic acid (10.0 g, 39.9 mmol) and 1-[bis(dimethylamino)methylene]-1H-1,2,3-triazolo[4,5-b]pyridinium 3-oxid hexafluorophosphate (18.2 g, 47.9 mmol) in DMF (200 mL) was added triethylamine (16.7 mL, 120 mmol). The mixture was stirred for 5 min. To the mixture was added methylamine (2 N in THF, 31.9 mL, 63.9 mmol).

The mixture was stirred for 2 h. The mixture was poured into ice water (300 mL) to give a suspension. The resulting solid was collected by filtration, then redissolved in EtOAc (300 mL), washed with brine, dried over Na₂SO₄, and concentrated to afford the title compound (9.1 g, 34 mmol, 86% yield), a colorless solid. LC-MS calc. for C₈H₉BrClN₂O [M+H]⁺ m/z=262.9; Found 263.2.

### Step 2: 8-Bromo-6-chloro-3-methylquinazoline-2,4(1H,3H)-dione

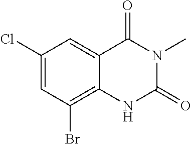

To a solution of 2-amino-3-bromo-5-chloro-N-methylbenzamide (9.1 g, 34 mmol) in THF (100 mL) was added triphosgene (4.1 g, 14 mmol). The mixture was heated to 60° C. for 1 h. The reaction was cooled to room temperature and water (30 mL) was added at 0° C. The mixture was stirred for 30 min. The solid was collected by filtration to give the title compound (9.9 g, 34 mmol, 99% yield), a colorless solid. LC-MS calc. for C₉H₇BrClN₂O₂ [M+H]⁺ m/z=288.9; Found 289.3.

### Step 3: 2-(3-Azabicyclo[3.1.0]hexan-3-yl)-8-bromo-6-chloro-3-methylquinazolin-4(3H)-one

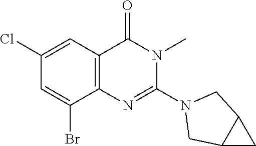 [SMILES: Cn1c(N2CC3CC3C2)nc2c(Br)cc(Cl)cc2c1=O]

The title compound was synthesized by procedures analogous to those outlined in Example 65, Step 2. LC-MS calc. for C₁₄H₁₄BrClN₃O [M+H]⁺ m/z=354.0; Found 354.2.

### Step 4: 8-Acetyl-2-(3-azabicyclo[3.1.0]hexan-3-yl)-6-chloro-3-methylquinazolin-4(3H)-one

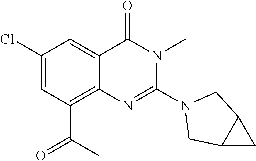 [SMILES: CC(=O)c1cc(Cl)cc2c(=O)n(C)c(N3CC4CC4C3)nc12]

The title compound was synthesized by procedures analogous to those outlined in Example 32, Step 2. LC-MS calc. for C₁₆H₁₇ClN₃O₂ [M+H]⁺ m/z=318.1; Found 318.3.

### Step 5: (8-Acetyl-2-(3-azabicyclo[3.1.0]hexan-3-yl)-3-methyl-4-oxo-3,4-dihydroquinazolin-6-yl)boronic acid

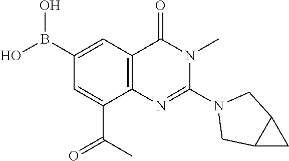 [SMILES: CC(=O)c1cc(B(O)O)cc2c(=O)n(C)c(N3CC4CC4C3)nc12]

A mixture of 8-acetyl-2-(3-azabicyclo[3.1.0]hexan-3-yl)-6-chloro-3-methylquinazolin-4-one (100 mg, 0.31 mmol), bis(pinacolato)diboron (95.9 mg, 0.380 mmol), XPhos (CAS 564483-18-7, 9.0 mg, 0.0189 mmol), palladium(II) acetate (4.24 mg, 0.0189 mmol) and potassium acetate (93 mg, 0.94 mmol) in 1,4-dioxane (3 mL) was heated to 85° C. under a nitrogen atmosphere for 16 h. The mixture was cooled to room temperature, filtered, and concentrated. The crude material was purified by silica gel chromatography (0-70% EtOAc/heptane). The material was dissolved in acetone (8 mL) and a suspension of sodium periodate (200 mg, 0.94 mmol) and ammonium acetate (73 mg, 0.94 mmol) in water (3 mL) was added. The reaction was stirred for 16 h. The mixture was extracted with EtOAc (2×20 mL) and the combined organic layers were dried over MgSO₄, filtered, and concentrated. The crude material was purified by silica gel chromatography (0-10% MeOH/DCM) to afford the title compound (20 mg, 0.061 mmol, 19% yield), a colorless solid. LC-MS calc. for C₁₆H₁₉BN₃O₄ [M+H]⁺ m/z=328.1; Found 328.3.

### Step 6: 8-Acetyl-2-(3-azabicyclo[3.1.0]hexan-3-yl)-6-(difluoromethyl)-3-methyl-quinazolin-4(3H)-one

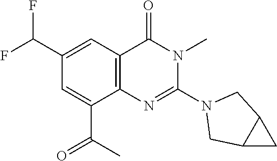 [SMILES: CC(=O)c1cc(C(F)F)cc2c(=O)n(C)c(N3CC4CC4C3)nc12]

To a mixture of bis(triphenylphosphine)palladium(II) dichloride (3.22 mg, 0.00459 mmol), XantPhos (3.98 mg, 0.00688 mmol, CAS 161265-03-8), potassium carbonate (50.7 mg, 0.367 mmol), tris(acetylacetonato)iron(III) (1.13 mg, 0.00321 mmol) and hydroquinone (20.2 mg, 0.183 mmol) under a nitrogen atmosphere was added a mixture of [8-acetyl-2-(3-azabicyclo[3.1.0]hexan-3-yl)-3-methyl-4-oxoquinazolin-6-yl]boronic acid (15 mg, 0.046 mmol), ethyl 2-bromo-2,2-difluoroacetate (24 mL, 0.18 mmol) and styrene (2.11 mL, 0.0183 mmol) in 1,4-dioxane (1.5 mL). The mixture was heated to 80° C. for 16 h. The mixture was cooled to room temperature, filtered, and purified by prep-HPLC on a C18 column (10-70% MeCN/0.1% TFA (aq)) to afford the title compound (7.5 mg, 0.023 mmol, 49% yield), a light purple solid. LC-MS calc. for C₁₇H₁₈F₂N₃O₂ [M+H]⁺ m/z=334.1; Found 334.3.

### Step 7: 2-(3-Azabicyclo[3.1.0]hexan-3-yl)-6-(difluoromethyl)-8-(1-hydroxyethyl)-3-methylquinazolin-4(3H)-one

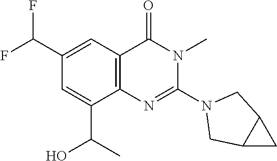 [SMILES: CC(O)c1cc(C(F)F)cc2c(=O)n(C)c(N3CC4CC4C3)nc12]

The title compound was synthesized by procedures analogous to those outlined in Example 1, Step 6. LC-MS calc. for C₁₇H₂₀F₂N₃O₂ [M+H]⁺ m/z=336.1; Found 336.3.

### Step 8: 2-((1-(2-(3-azabicyclo[3.1.0]hexan-3-yl)-6-(difluoromethyl)-3-methyl-4-oxo-3,4-dihydroquinazolin-8-yl)ethyl)amino)benzoic acid

The title compound was synthesized by procedures analogous to those outlined in Example 41, Step 4. LC-MS calc. for C₂₄H₂₅F₂N₄O₃ [M+H]⁺ m/z=455.1; Found 455.2.

### Example 80: 3-((1-(2-(3-azabicyclo[3.1.0]hexan-3-yl)-3,6-dimethyl-4-oxo-3,4-dihydro-quinazolin-8-yl)ethyl)amino)-6-(trifluoromethyl)picolinic acid

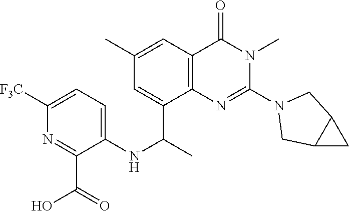 [SMILES: Cc1cc(C(C)Nc2ccc(C(F)(F)F)nc2C(=O)O)c2nc(N3CC4CC4C3)n(C)c(=O)c2c1]

To a solution of 2-(3-azabicyclo[3.1.0]hexan-3-yl)-8-(1-hydroxyethyl)-3,6-dimethylquinazolin-4-one (33 mg, 0.11 mmol) in DCM (7 mL) was added phosphorus tribromide (60 mg, 0.22 mmol). The reaction was stirred for 2.5 h. Sat. aq. NaHCO₃ (5 mL) was added followed by EtOAc (5 mL). The resulting layers were separated, and the aqueous layer was extracted by EtOAc (2×5 mL). The combined organic layers were washed by brine, dried over anhydrous MgSO₄, filtered, and concentrated. The material was dissolved in DCM (3 mL) and methyl 3-amino-6-(trifluoromethyl)pyridine-2-carboxylate (50 mg, 0.23 mmol) was added. The reaction mixture was stirred at 40° C. overnight. The mixture was concentrated, and the crude material was dissolved in THF (1 mL) and methanol (1 mL). Lithium hydroxide monohydrate (47.3 mg, 1.13 mmol) was added. The mixture was heated to 45° C. for 45 min. The mixture was concentrated and dissolved in EtOAc (7 mL), washed with water (2×5 mL), concentrated, and purified by prep-HPLC on a C18 column (20-100% MeCN/0.1% TFA (aq)) to afford the title compound (18 mg, 0.032 mmol, 27% yield), a white solid. ¹H NMR (300 MHz, DMSO-d₆) δ 8.76 (d, J=7.0 Hz, 1H), 7.69 (d, J=9.1 Hz, 2H), 7.51 (d, J=1.9 Hz, 1H), 7.13 (d, J=9.0 Hz, 1H), 5.42-5.33 (m, 1H), 3.97 (d, J=10.5 Hz, 1H), 3.82 (d, J=10.4 Hz, 1H), 3.51 (t, J=10.3 Hz, 2H), 3.43 (s, 3H), 2.32 (s, 3H), 1.63 (t, J=7.2 Hz, 5H), 0.58 (dd, J=12.2, 7.6 Hz, 1H), 0.36 (dd, J=8.2, 4.0 Hz, 1H). LC-MS calc. for C₂₄H₂₅F₃N₅O₃ [M+H]⁺ m/z=488.1; Found 488.3.

### Example 81: 2-((1-(2-((1R,5S,6R)-6-(Isopropyl(methyl)carbamoyl)-3-azabicyclo[3.1.0]hexan-3-yl)-3,6-dimethyl-4-oxo-3,4-dihydroquinazolin-8-yl)ethyl)amino)benzoic acid

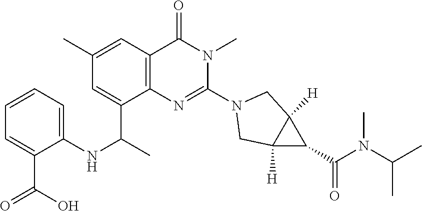 [SMILES: Cc1cc(C(C)Nc2ccccc2C(=O)O)c2nc(N3C[C@@H]4[C@H](C3)[C@H]4C(=O)N(C)C(C)C)n(C)c(=O)c2c1]

### Step 1. methyl (1R,5S,6R)-3-(8-acetyl-3,6-dimethyl-4-oxo-3,4-dihydroquinazolin-2-yl)-3-azabicyclo[3.1.0]hexane-6-carboxylate

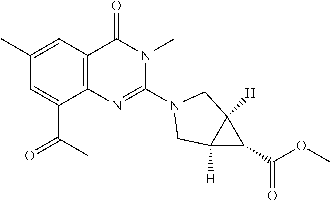

The title compound was synthesized by procedures analogous to Example 65, Step 2. LC-MS calc. for C₁₉H₂₂N₃O₄ [M+H]⁺: m/z=356.1; Found 356.1.

### Step 2. Methyl (1R,5S,6R)-3-(8-(1-((2-(tert-butoxycarbonyl)phenyl)amino)ethyl)-3,6-dimethyl-4-oxo-3,4-dihydroquinazolin-2-yl)-3-azabicyclo[3.1.0]hexane-6-carboxylate

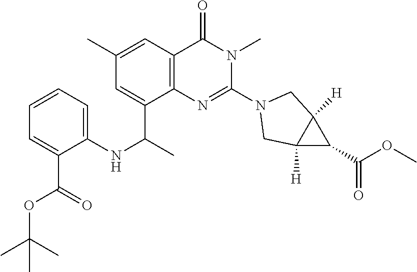 [SMILES: COC(=O)[C@H]1[C@@H]2CN(c3nc4c(C(C)Nc5ccccc5C(=O)OC(C)(C)C)cc(C)cc4c(=O)n3C)C[C@@H]21]

The title compound was synthesized by procedures analogous to Example 14, Steps 8 and 9. LC-MS calc. for C₃₀H₃₇N₄O₅ [M+H]⁺: m/z=533.2; Found 533.1.

### Step 3. (1R,5S,6R)-3-(8-(1-((2-(tert-butoxycarbonyl)phenyl)amino)ethyl)-3,6-dimethyl-4-oxo-3,4-dihydroquinazolin-2-yl)-3-azabicyclo[3.1.0]hexane-6-carboxylic acid

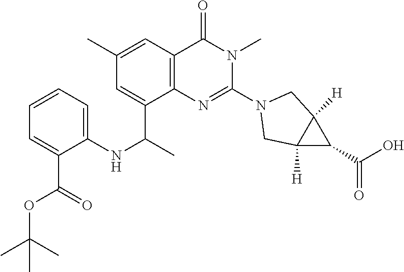 [SMILES: Cc1cc(C(C)Nc2ccccc2C(=O)OC(C)(C)C)c2nc(N3C[C@@H]4[C@H](C3)[C@H]4C(=O)O)n(C)c(=O)c2c1]

The title compound was synthesized by procedures analogous to Example 23, Step 3. LC-MS calc. for C₂₉H₃₅N₄O₅ [M+H]⁺: m/z=519.2; Found 519.1.

### Step 4. 2-((1-(2-((1R,5S,6R)-6-(Isopropyl(methyl)carbamoyl)-3-azabicyclo[3.1.0]hexan-3-yl)-3,6-dimethyl-4-oxo-3,4-dihydroquinazolin-8-yl)ethyl)amino)benzoic acid

To a solution of (1R,5S,6R)-3-(8-(1-((2-(tert-butoxycarbonyl)phenyl)amino)ethyl)-3,6-dimethyl-4-oxo-3,4-dihydroquinazolin-2-yl)-3-azabicyclo[3.1.0]hexane-6-carboxylic acid (15.1 mg, 0.024 mmol, TFA salt) in DCM (1 mL) was added triethylamine (9.91 μL, 0.071 mmol) and 1-[bis(dimethylamino)methylene]-1H-1,2,3-triazolo[4,5-b] pyridinium 3-oxid hexafluorophosphate (9.92 mg, 0.026 mmol). The resulting mixture was stirred for 15 min. N-Isopropylmethylamine (5.21 mg, 0.071 mmol) was added, and the reaction was stirred for 30 min. Progress of the reaction was monitored by LCMS. Upon completion of the reaction, the reaction mixture was concentrated and to the concentrated crude reaction mixture was added trifluoroacetic acid (1 mL). The reaction mixture was stirred for 3 h. The crude material was diluted with MeCN (5 mL) and water (5 mL) and was purified by prep-HPLC on a C18 column (10-90% MeCN/0.1% TFA (aq.)) to afford the title compound as a TFA salt (6.10 mg, 0.012 mmol, 48.9% yield), a white solid. LC-MS calc. for C₂₉H₃₆N₅O₄ [M+H]⁺: m/z=518.2; Found 518.0.

### Examples 82-90

Examples 82-90 listed in Tables 11 and 12 were synthesized according to procedures analogous to Example 81. All examples in this table were prepared as the TFA salt.

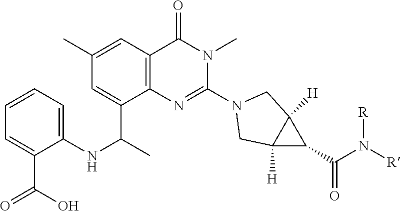

TABLE 11
Examples 82-90

| Example | NRR′ | LCMS [M + H]⁺ |
|---|---|---|
| 82 | 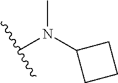 [SMILES: *N(C)C1CCC1] | 530.1 |
| 83 | 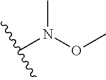 [SMILES: *N(C)OC] | 506.1 |
| 84 | 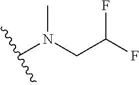 [SMILES: *N(C)CC(F)F] | 540.1 |
| 85 | 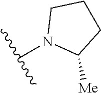 [SMILES: *N1CCC[C@@H]1C] | 530.1 |
| 86 | 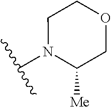 [SMILES: *N1CCOC[C@@H]1C] | 546.1 |
| 87 | 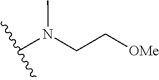 [SMILES: *N(C)CCOC] | 534.1 |
| 88 | 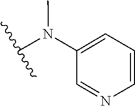 [SMILES: *N(C)c1cccnc1] | 553.1 |
| 89 | 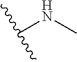 [SMILES: *NC] | 476.1 |
| 90 | 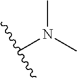 [SMILES: *N(C)C] | 490.1 |

TABLE 12
Examples 82-90

| Ex- | |
|---|---|
| am- | |
| ple | Compound Name |
| 82 | 2-((1-(2-((1R,5S,6R)-6-(Cyclobutyl(methyl)carbamoyl)-3- |
| | azabicyclo[3.1.0]hexan-3-yl)-3,6-dimethyl-4-oxo-3,4- |
| | dihydroquinazolin-8-yl)ethyl)amino)benzoic acid |
| 83 | 2-((1-(2-((1R,5S,6R)-6-(Methoxy(methyl)carbamoyl)-3- |
| | azabicyclo[3.1.0]hexan-3-yl)-3,6-dimethyl-4-oxo-3,4- |
| | dihydroquinazolin-8-yl)ethyl)amino)benzoic acid |
| 84 | 2-((1-(2-((1R,5S,6R)-6-((2,2-Difluoroethyl)(methyl)carbamoyl)-3- |
| | azabicyclo[3.1.0]hexan-3-yl)-3,6-dimethyl-4-oxo-3,4- |
| | dihydroquinazolin-8-yl)ethyl)amino)benzoic acid |
| 85 | 2-((1-(3,6-Dimethyl-2-((1R,5S,6R)-6-((S)-2-methylpyrrolidine-1- |
| | carbonyl)-3-azabicyclo[3.1.0]hexan-3-yl)-4-oxo-3,4- |
| | dihydroquinazolin-8-yl)ethyl)amino)benzoic acid |
| 86 | 2-((1-(3,6-Dimethyl-2-((1R,5S,6R)-6-((S)-3-methylmorpholine-4- |
| | carbonyl)-3-azabicyclo[3.1.0]hexan-3-yl)-4-oxo-3,4- |
| | dihydroquinazolin-8-yl)ethyl)amino)benzoic acid |
| 87 | 2-((1-(2-((1R,5S,6R)-6-((2-Methoxyethyl)(methyl)carbamoyl)-3- |
| | azabicyclo[3.1.0]hexan-3-yl)-3,6-dimethyl-4-oxo-3,4- |
| | dihydroquinazolin-8-yl)ethyl)amino)benzoic acid |
| 88 | 2-((1-(3,6-Dimethyl-2-((1R,5S,6R)-6-(methyl(pyridin-3- |
| | yl)carbamoyl)-3-azabicyclo[3.1.0]hexan-3-yl)-4-oxo-3,4- |
| | dihydroquinazolin-8-yl)ethyl)amino)benzoic acid |
| 89 | 2-((1-(3,6-Dimethyl-2-((1R,5S,6R)-6-(methylcarbamoyl)-3- |
| | azabicyclo[3.1.0]hexan-3-yl)-4-oxo-3,4-dihydroquinazolin-8- |
| | yl)ethyl)amino)benzoic acid |
| 90 | 2-((1-(2-((1R,5S,6R)-6-(Dimethylcarbamoyl)-3- |
| | azabicyclo[3.1.0]hexan-3-yl)-3,6-dimethyl-4-oxo-3,4- |
| | dihydroquinazolin-8-yl)ethyl)amino)benzoic acid |

### Examples 91-92

Examples 91-92 listed in Tables 13 and 14 were synthesized according to procedures analogous to Example 46. All examples in this table were prepared as the TFA salt.

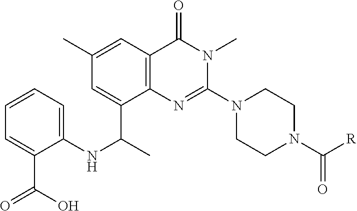

TABLE 13
Examples 91-92

| Example | R | LCMS [M + H]⁺ |
|---|---|---|
| 91 | 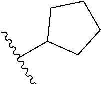 [SMILES: *C1CCCC1] | 518.1 |
| 92 | 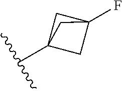 [SMILES: *C12CC(F)(C1)C2] | 534.1 |

TABLE 14
Examples 91-92

| Exam- | |
|---|---|
| ple | Compound Name |
| 91 | 2-((1-(2-(4-(Cyclopentanecarbonyl)piperazin-1-yl)-3,6-dimethyl- |
| | 4-oxo-3,4-dihydroquinazolin-8-yl)ethyl)amino)benzoic acid |
| 92 | 2-((1-(2-(4-(3-Fluorobicyclo[1.1.1]pentane-1-carbonyl)piperazin- |
| | 1-yl)-3,6-dimethyl-4-oxo-3,4-dihydroquinazolin-8-yl)ethyl) |
| | amino)benzoic acid |

### Example 93: 2-((1-(2-(4-(2-Methoxyacetyl)piperazin-1-yl)-3,6-dimethyl-4-oxo-3,4-dihydro-quinazolin-8-yl)ethyl)amino)benzoic acid

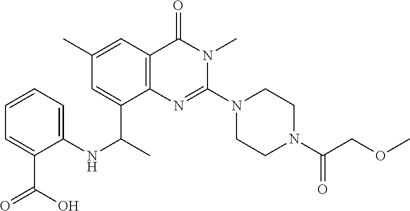 [SMILES: COCC(=O)N1CCN(c2nc3c(C(C)Nc4ccccc4C(=O)O)cc(C)cc3c(=O)n2C)CC1]

### Step 1. tert-Butyl 4-(8-(1-hydroxyethyl)-3,6-dimethyl-4-oxo-3,4-dihydroquinazolin-2-yl)piperazine-1-carboxylate

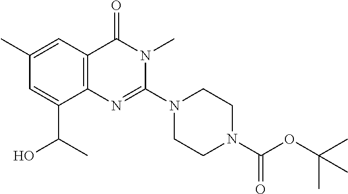 [SMILES: Cc1cc(C(C)O)c2nc(N3CCN(C(=O)OC(C)(C)C)CC3)n(C)c(=O)c2c1]

The title compound was synthesized by procedures analogous to Example 39, Steps 1-3. LC-MS calc. for C₂₁H₃₁N₄O₄ [M+H]⁺: m/z=403.2; Found 403.2.

### Step 2. tert-Butyl 4-(8-(1-((2-(tert-butoxycarbonyl)phenyl)amino)ethyl)-3,6-dimethyl-4-oxo-3,4-dihydroquinazolin-2-yl)piperazine-1-carboxylate

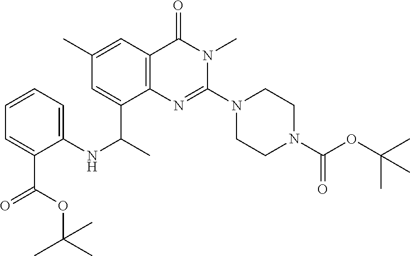 [SMILES: Cc1cc(C(C)Nc2ccccc2C(=O)OC(C)(C)C)c2nc(N3CCN(C(=O)OC(C)(C)C)CC3)n(C)c(=O)c2c1]

To a solution of tert-butyl 4-[8-(1-hydroxyethyl)-3,6-dimethyl-4-oxoquinazolin-2-yl]piperazine-1-carboxylate (230 mg, 0.571 mmol) in DCM (6 mL) was added triethylamine (0.398 mL, 2.86 mmol). The reaction mixture was cooled to 0° C., and methane sulfonyl chloride (0.053 mL, 0.69 mmol) was added dropwise. The reaction mixture was allowed to warm to room temperature and stirred for 1 h. A solution of tert-butyl-2-aminobenzoate (133 mg, 0.686 mmol) in DCM (1 mL) was added. The reaction mixture was stirred at 40° C. overnight and then cooled to room temperature. The reaction mixture was diluted with water (10 mL), and the layers were separated. The aqueous layer was extracted with DCM (2×10 mL). The combined organic layers were washed with brine (20 mL), dried over Na₂SO₄, filtered, and concentrated. The crude material was purified by silica gel chromatography (0-80% EtOAc/hexane) to afford the title compound (130 mg, 0.225 mmol, 39.4% yield) as an off-white solid. LC-MS calc. for C₃₂H₄₄N₅O₅ [M+H]⁺: m/z=578.3; Found 578.1.

### Step 3. 2-((1-(3,6-Dimethyl-4-oxo-2-(piperazin-1-yl)-3,4-dihydroquinazolin-8-yl)ethyl)amino)benzoic acid

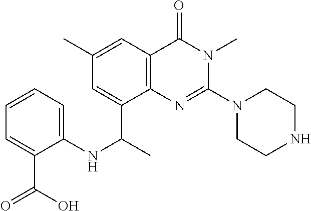 [SMILES: Cc1cc(C(C)Nc2ccccc2C(=O)O)c2nc(N3CCNCC3)n(C)c(=O)c2c1]

To a solution of tert-butyl 4-(8-(1-((2-(tert-butoxycarbonyl)phenyl)amino)ethyl)-3,6-dimethyl-4-oxo-3,4-dihydroquinazolin-2-yl)piperazine-1-carboxylate (130 mg, 0.225 mmol) in DCM (2 mL) was added trifluoroacetic acid (1 mL). The reaction mixture was stirred for 4 h. The reaction mixture was diluted with water (10 mL), and the layers were separated. The aqueous layer was extracted with DCM (2×10 mL). The combined organic layers were washed with brine (20 mL), dried over Na₂SO₄, filtered, and concentrated. The crude material was purified by prep-HPLC on a C18 column (10-90% MeCN/0.1% TFA (aq.)) to afford the title compound as a TFA salt (60.0 mg, 0.112 mmol, 49.8% yield), a white solid. LC-MS calc. for C₂₃H₂₈N₅O₃ [M+H]⁺: m/z=422.2; Found 422.2.

### Step 4. 2-((1-(2-(4-(2-Methoxyacetyl)piperazin-1-yl)-3,6-dimethyl-4-oxo-3,4-dihydroquinazolin-8-yl)ethyl)amino)benzoic acid

To a solution of methoxy acetic acid (6.41 mg, 0.071 mmol) in DCM (1 mL) was added triethylamine (29.8 μL, 0.214 mmol) and 1-[bis(dimethylamino)methylene]-1H-1,2,3-triazolo[4,5-b]pyridinium 3-oxid hexafluorophosphate (29.8 mg, 0.078 mmol). The resulting mixture was stirred for 15 min. 2-((1-(3,6-dimethyl-4-oxo-2-(piperazin-1-yl)-3,4-dihydroquinazolin-8-yl)ethyl)amino) benzoic acid (30.0 mg, 0.056 mmol, TFA salt) was added, and the reaction was stirred for 30 min. The reaction mixture was diluted with water (10 mL), and the layers were separated. The aqueous layer was extracted with DCM (2×10 mL). The combined organic layers were washed with brine (20 mL), dried over Na₂SO₄, filtered, and concentrated. The crude material was purified by prep-HPLC on a C18 column (10-90% MeCN/0.1% TFA (aq.)) to afford the title compound as a TFA salt (11.7 mg, 0.019 mmol, 34.4% yield), a white solid. LC-MS calc. for C₂₆H₃₂N₅O₅ [M+H]⁺: m/z=494.2; Found 494.0.

### Example 94: 2-((1-(3,6-Dimethyl-2-(4-(6-methylnicotinoyl)piperazin-1-yl)-4-oxo-3,4-dihydro-quinazolin-8-yl)ethyl)amino)benzoic acid

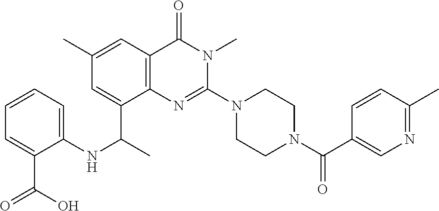 [SMILES: Cc1cc(C(C)Nc2ccccc2C(=O)O)c2nc(N3CCN(C(=O)c4ccc(C)nc4)CC3)n(C)c(=O)c2c1]

The title compound was synthesized according to procedures analogous to those outlined in Example 93. LC-MS calc. for C₃₀H₃₃N₆O₄ [M+H]⁺: m/z=541.3; Found 541.1.

### Example 95: 2-((1-(2-(3-Azabicyclo[3.1.0]hexan-3-yl)-5-fluoro-3,6-dimethyl-4-oxo-3,4-dihydroquinazolin-8-yl)ethyl)amino)benzoic acid (isomer 1)

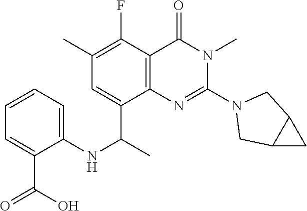 [SMILES: Cc1cc(C(C)Nc2ccccc2C(=O)O)c2nc(N3CC4CC4C3)n(C)c(=O)c2c1F]

### Step 1. 2-(3-Azabicyclo[3.1.0]hexan-3-yl)-5-fluoro-8-(1-hydroxyethyl)-3,6-dimethylquinazolin-4(3H)-one

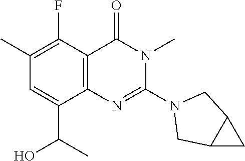 [SMILES: Cc1cc(C(C)O)c2nc(N3CC4CC4C3)n(C)c(=O)c2c1F]

The title compound was synthesized by procedures analogous to those outlined in Example 14, Steps 2-8. LC-MS calc. for C₁₇H₂₁FN₃O₂ [M+H]⁺: m/z=318.2; Found: 318.1.

### Step 2. Methyl 2-((1-(2-(3-azabicyclo[3.1.0]hexan-3-yl)-5-fluoro-3,6-dimethyl-4-oxo-3,4-dihydroquinazolin-8-yl)ethyl)amino)benzoate (isomer 1)

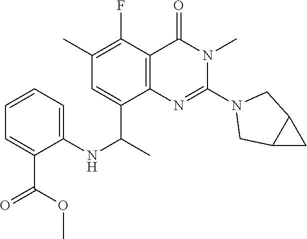 [SMILES: COC(=O)c1ccccc1NC(C)c1cc(C)c(F)c2c(=O)n(C)c(N3CC4CC4C3)nc12]

The title compound was synthesized by procedures analogous to those outlined in Example 24, Step 1. The isomers were separated using chiral prep-HPLC on a Lux i-Amylose-3 column (30 mL/min, 85:7.5:7.5 hexane/IPA/MeOH) to afford two isomers: isomer 1 (t_{R}=2.54 min) and isomer 2 (t_{R}=2.88 min). Isomer 1: LC-MS calc. for C₂₅H₂₈FN₄O₃ [M+H]⁺: m/z=451.2; Found: 451.1.

### Step 4. 2-((1-(2-(3-Azabicyclo[3.1.0]hexan-3-yl)-5-fluoro-3,6-dimethyl-4-oxo-3,4-dihydroquinazolin-8-yl)ethyl)amino)benzoic acid (isomer 1)

The title compound was synthesized by procedures analogous to those outlined in Example 24, Step 2. ¹H NMR (400 MHz, DMSO-d₆) δ 8.33 (broad s, 1H), 7.77 (dd, J=8.0, 1.7 Hz, 1H), 7.43 (d, J=7.6 Hz, 1H), 7.17 (ddd, J=8.7, 7.1, 1.8 Hz, 1H), 6.49 (t, J=7.5 Hz, 1H), 6.42 (d, J=8.5 Hz, 1H), 5.28 (q, J=6.6 Hz, 1H), 4.03-3.97 (m, 2H), 3.58-3.44 (m, 2H), 3.38 (s, 3H), 2.16 (d, J=2.2 Hz, 3H), 1.62-1.59 (m, 2H), 1.52 (d, J=6.7 Hz, 3H), 0.62-0.57 (m, 1H), 0.33-0.30 (m, 1H). LC-MS calc. for C₂₄H₂₆FN₄O₃ [M+H]⁺: m/z=437.2; Found: 437.1.

### Example 96: 2-((1-(2-(3-azabicyclo[3.1.0]hexan-3-yl)-3,6-dimethyl-4-oxo-3,4-dihydro-quinazolin-8-yl)-2,2-difluoroethyl)amino)benzoic acid

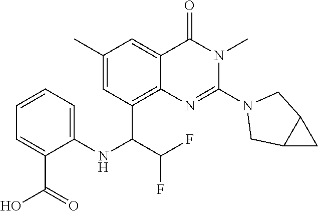 [SMILES: Cc1cc(C(Nc2ccccc2C(=O)O)C(F)F)c2nc(N3CC4CC4C3)n(C)c(=O)c2c1]

### Step 1. 2-(3-Azabicyclo[3.1.0]hexan-3-yl)-8-(2,2-difluoro-1-hydroxyethyl)-3,6-dimethylquinazolin-4(3H)-one

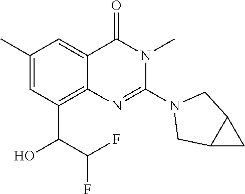 [SMILES: Cc1cc(C(O)C(F)F)c2nc(N3CC4CC4C3)n(C)c(=O)c2c1]

(Difluoromethyl)trimethylsilane (18 μL, 0.13 mmol) was added to a solution of 2-(3-azabicyclo[3.1.0]hexan-3-yl)-3,6-dimethyl-4-oxo-3,4-dihydroquinazoline-8-carbaldehyde (Example 47, Step 3) (25 mg, 0.090 mmol) and cesium fluoride (20 mg, 0.13 mmol) in THF (0.90 mL), and the reaction mixture was stirred for 24 h. The reaction mixture was then heated to 70° C. for 24 h. The reaction mixture was cooled to room temperature and diluted with water and DCM. The two phases were separated, and the aqueous layer was extracted with DCM (10 mL×2). The combined organic layers were dried over sodium sulfate, filtered, and concentrated. The crude residue containing the title compound was used without further purification (20 mg, 0.060 mmol, 68% yield). LC-MS calc. for C₁₇H₂₀F₂N₃O₂ [M+H]⁺: m/z=336.1; Found=336.1.

### Step 2. 2-((1-(2-(3-Azabicyclo[3.1.0]hexan-3-yl)-3,6-dimethyl-4-oxo-3,4-dihydroquinazolin-8-yl)-2,2-difluoroethyl)amino)benzoic acid

Methanesulfonyl chloride (2.8 μL, 0.04 mmol) was added to a solution of 2-(3-azabicyclo[3.1.0]hexan-3-yl)-8-(2,2-difluoro-1-hydroxyethyl)-3,6-dimethylquinazolin-4(3H)-one (6.0 mg, 0.02 mmol) and triethylamine (7.5 μL, 0.05 mmol) in DCM (0.60 mL), and the reaction mixture was stirred for 1 h. Then anthranilic acid (3.7 mg, 0.03 mmol) was added, and the reaction mixture was stirred for 18 h. The reaction mixture was diluted with water and DCM, and the resulting two phases were separated. The aqueous layer was extracted with DCM (5 mL×2), and the combined organic layers were dried over sodium sulfate, filtered, and concentrated. The crude residue was purified by prep-HPLC on a C18 column (56-76% MeCN/0.1% TFA (aq.)) to afford the title compound as a TFA salt (2.2 mg, 4.8 μmol, 22% yield). LC-MS calc. for C₂₄H₂₅F₂N₄O₃ [M+H]⁺: m/z=455.2; Found=455.1.

### Example 97: 2-((1-(2-(3-azabicyclo[3.1.0]hexan-3-yl)-3,6-dimethyl-4-oxo-3,4-dihydro-quinazolin-8-yl)propyl)amino)benzoic acid

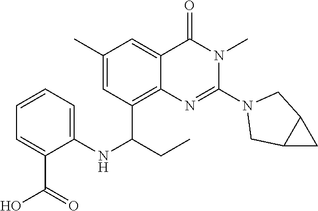 [SMILES: CCC(Nc1ccccc1C(=O)O)c1cc(C)cc2c(=O)n(C)c(N3CC4CC4C3)nc12]

### Step 1. 2-(3-Azabicyclo[3.1.0]hexan-3-yl)-8-(1-hydroxypropyl)-3,6-dimethylquinazolin-4(3H)-one

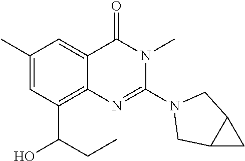 [SMILES: CCC(O)c1cc(C)cc2c(=O)n(C)c(N3CC4CC4C3)nc12]

Ethylmagnesium bromide solution (35 μL, 0.11 mmol, 3.0 M in diethyl ether) was added to a solution of 2-(3-azabicyclo[3.1.0]hexan-3-yl)-3,6-dimethyl-4-oxo-3,4-dihydroquinazoline-8-carbaldehyde (Example 47, Step 3) (25 mg, 0.09 mmol) in THF (0.90 mL) at 0° C., and the reaction mixture was stirred at 0° C. for 15 min. The reaction mixture was diluted with sat. NH₄Cl (aq.) and DCM; then the resulting two phases were separated. The aqueous layer was extracted with DCM (5 mL×2), and the combined organic layers were dried over sodium sulfate, filtered, and concentrated. The crude material containing the title compound was used without further purification. LC-MS calc. for C₁₈H₂₄N₃O₂ [M+H]⁺: m/z=314.2; Found=314.1.

### Step 2. 2-((1-(2-(3-Azabicyclo[3.1.0]hexan-3-yl)-3,6-dimethyl-4-oxo-3,4-dihydro-quinazolin-8-yl)propyl)amino)benzoic acid

The title compound was synthesized by procedures analogous to those outlines in Example 96, Step 2. ¹H NMR (400 MHz, DMSO-d₆) δ 8.47 (bs, 1H), 7.76 (dd, J=7.8, 1.7 Hz, 1H), 7.66 (s, 1H), 7.38 (d, J=2.1 Hz, 1H), 7.24-7.02 (m, 1H), 6.54-6.30 (m, 2H), 5.24-5.13 (m, 1H), 4.01 (d, J=10.5 Hz, 1H), 3.81 (d, J=10.5 Hz, 1H), 3.59-3.51 (m, 1H), 3.50-3.44 (m, 1H), 3.43 (s, 3H), 2.29 (s, 3H), 2.01-1.78 (m, 2H), 1.67-1.57 (m, 2H), 0.92 (t, J=7.3 Hz, 3H), 0.64-0.50 (m, 1H), 0.41-0.32 (m, 1H). LC-MS calc. for C₂₅H₂₉N₄O₃ [M+H]⁺: m/z=433.2; Found=433.1.

### Example 98: 2-((1-(3-(3,3-Bis(hydroxymethyl)cyclobutyl)-2-(isoindolin-2-yl)-6-methyl-4-oxo-3,4-dihydroquinazolin-8-yl)ethyl)amino)benzoic acid

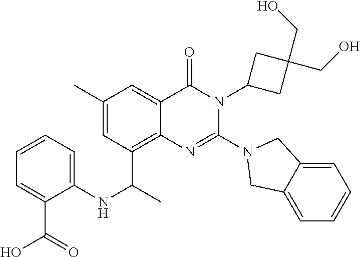 [SMILES: Cc1cc(C(C)Nc2ccccc2C(=O)O)c2nc(N3Cc4ccccc4C3)n(C3CC(CO)(CO)C3)c(=O)c2c1]

### Step 1. 8-Acetyl-2-(isoindolin-2-yl)-6-methyl-3-(2-oxaspiro[3.3]heptan-6-yl)quinazolin-4(3H)-one

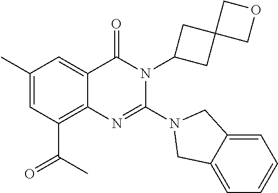 [SMILES: CC(=O)c1cc(C)cc2c(=O)n(C3CC4(COC4)C3)c(N3Cc4ccccc4C3)nc12]

The title compound was synthesized by procedures analogous to those outlines in Example 1, Steps 1-2, substituting 2-oxaspiro[3.3]heptan-6-amine for methylamine in Step 1; Example 65, Step 2, substituting isoindoline hydrochloride for tert-butyl 2,5-diazabicyclo[2.2.1]heptane-2-carboxylate; and Example 1, Step 5, substituting 10% H₂SO₄ (aq.) for 6N HCl (aq.). LC-MS calc. for C₂₅H₂₆N₃O₃ [M+H]⁺: m/z=416.2; Found=416.1.

### Step 2. 8-(1-Hydroxyethyl)-2-(isoindolin-2-yl)-6-methyl-3-(2-oxaspiro[3.3]heptan-6-yl)quinazolin-4(3H)-one

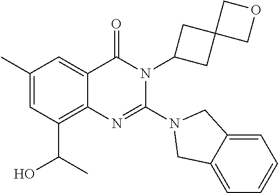 [SMILES: Cc1cc(C(C)O)c2nc(N3Cc4ccccc4C3)n(C3CC4(COC4)C3)c(=O)c2c1]

The title compound was synthesized by procedures analogous to those outlines in Example 1, Step 6. LC-MS calc. for C₂₅H₂₈N₃O₃ [M+H]⁺: m/z=418.2; Found=418.1.

### Step 3. 2-((1-(3-(3,3-Bis(hydroxymethyl)cyclobutyl)-2-(isoindolin-2-yl)-6-methyl-4-oxo-3,4-dihydroquinazolin-8-yl)ethyl)amino)benzoic acid

The title compound was synthesized by procedures analogous to those outlines in Example 41, Step 4 except the displacement was performed at 45° C. for 18 h to afford the product as an ammonium salt. LC-MS calc. for C₃₂H₃₅N₄O₅ [M+H]⁺: m/z=555.3; Found=555.2.

### Example 99: 2-((1-(2-(2-Azabicyclo[2.2.1]heptan-2-yl)-3,6-dimethyl-4-oxo-3,4-dihydro-quinazolin-8-yl)ethyl)amino)benzoic acid (Isomer 2)

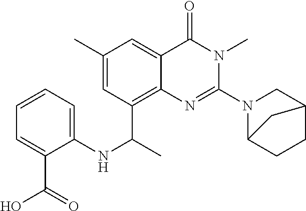 [SMILES: Cc1cc(C(C)Nc2ccccc2C(=O)O)c2nc(N3CC4CCC3C4)n(C)c(=O)c2c1]

### Step 1. 2-(2-Azabicyclo[2.2.1]heptan-2-yl)-8-bromo-3,6-dimethylquinazolin-4(3H)-one

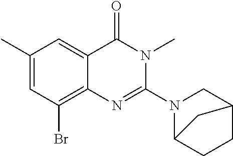 [SMILES: Cc1cc(Br)c2nc(N3CC4CCC3C4)n(C)c(=O)c2c1]

The title compound was synthesized by procedures analogous to those outlined in Example 1, Step 4, substituting 2-azabicyclo[2.2.1]heptane hydrochloride (CAS #: 63838-50-6) for isoindoline. LC-MS calc. for C₁₆H₁₉BrN₃O [M+H]⁺: m/z=348.1, 350.1; Found=348.0, 350.0.

### Step 2. 8-Acetyl-2-(2-azabicyclo[2.2.1]heptan-2-yl)-3,6-dimethylquinazolin-4(3H)-one (Isomer 1 and Isomer 2)

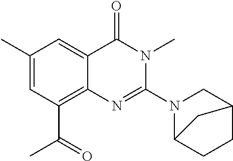 [SMILES: CC(=O)c1cc(C)cc2c(=O)n(C)c(N3CC4CCC3C4)nc12]

The title compound was synthesized from procedures analogous to those outlined in Example 1, Step 5. The crude product was purified by prep-HPLC on a C18 column (12.9-32.9% MeCN/0.1% TFA (aq.)) to afford the title compound as a TFA salt. LC-MS calc. for C₁₈H₂₂N₃O₂ [M+H]⁺: m/z=312.2; Found=312.1. The isomers were separated using chiral prep-HPLC on a Lux Cellulose-4 column (30 mL/min, 80:10:10 hexane/IPA/MeOH) to afford two isomers: isomer 1 (t_{R}=6.15) and isomer 2 (t_{R}=6.87).

### Step 3. 2-(2-Azabicyclo[2.2.1]heptan-2-yl)-8-(1-hydroxyethyl)-3,6-dimethylquinazolin-4(3H)-one (Isomer 1 and Isomer 2)

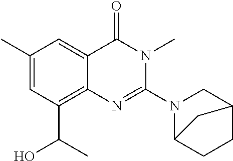 [SMILES: Cc1cc(C(C)O)c2nc(N3CC4CCC3C4)n(C)c(=O)c2c1]

The title compounds were synthesized from 8-acetyl-2-(2-azabicyclo[2.2.1]heptan-2-yl)-3,6-dimethylquinazolin-4(3H)-one (Isomer 1) by procedures analogous to those outlined in Example 1, Step 6 to afford a mixture of isomers 1 and 2. LC-MS calc. for C₁₈H₂₄N₃O₂ [M+H]⁺: m/z=314.2; Found=314.1.

### Step 4. 2-((1-(2-(2-Azabicyclo[2.2.1]heptan-2-yl)-3,6-dimethyl-4-oxo-3,4-dihydroquinazolin-8-yl)ethyl)amino)benzoic acid (Isomer 2)

The title compound was synthesized from a mixture of 2-(2-azabicyclo[2.2.1]heptan-2-yl)-8-(1-hydroxyethyl)-3,6-dimethylquinazolin-4(3H)-one (Isomer land Isomer 2; Example 99, Step 3) by procedures analogous to those outlined in Example 96, Step 2. The isomers were separated on prep-HPLC on a CSH-FP column (31.4-51.4% MeCN/0.1% TFA (aq.)) to afford two isomers: isomer 1 (t_{R}=4.07) and isomer 2 (t_{R}=4.56). Isomer 1: LC-MS calc. for C₂₅H₂₉N₄O₃ [M+H]⁺: m/z=433.2; Found=433.2. Isomer 2: LC-MS calc. for C₂₅H₂₉N₄O₃ [M+H]⁺: m/z=433.2; Found=433.1.

### Example 100. 2-((1-(2-(2-Azabicyclo[2.2.1]heptan-2-yl)-3,6-dimethyl-4-oxo-3,4-dihydro-quinazolin-8-yl)ethyl)amino)benzoic acid (Isomer 3)

### Example 101. 2-((1-(2-(2-Azabicyclo[2.2.1]heptan-2-yl)-3,6-dimethyl-4-oxo-3,4-dihydro-quinazolin-8-yl)ethyl)amino)benzoic acid (Isomer 4)

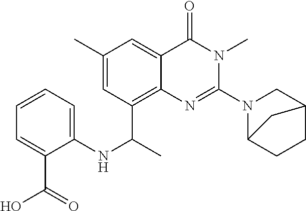 [SMILES: Cc1cc(C(C)Nc2ccccc2C(=O)O)c2nc(N3CC4CCC3C4)n(C)c(=O)c2c1]

### Step 1. 2-(2-Azabicyclo[2.2.1]heptan-2-yl)-8-(1-hydroxyethyl)-3,6-dimethylquinazolin-4(3H)-one (Isomer 3 and Isomer 4)

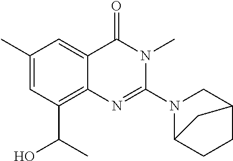 [SMILES: Cc1cc(C(C)O)c2nc(N3CC4CCC3C4)n(C)c(=O)c2c1]

The title compounds were synthesized from 8-acetyl-2-(2-azabicyclo[2.2.1]heptan-2-yl)-3,6-dimethylquinazolin-4(3H)-one (Isomer 2) (Example 99, Step 2) by procedures analogous to those outlined in Example 1, Step 6 to afford a mixture of isomers 3 and 4. LC-MS calc. for C₁₈H₂₄N₃O₂ [M+H]⁺: m/z=314.2; Found=314.1.

### Step 2. 2-((1-(2-(2-Azabicyclo[2.2.1]heptan-2-yl)-3,6-dimethyl-4-oxo-3,4-dihydro-quinazolin-8-yl)ethyl)amino)benzoic acid (Isomer 3) and 2-((1-(2-(2-azabicyclo[2.2.1]heptan-2-yl)-3,6-dimethyl-4-oxo-3,4-dihydroquinazolin-8-yl)ethyl)amino)benzoic acid (Isomer 4)

The title compounds were synthesized from a mixture of 2-(2-azabicyclo[2.2.1]heptan-2-yl)-8-(1-hydroxyethyl)-3,6-dimethylquinazolin-4(3H)-one (Isomer 3 and Isomer 4; Example 100, Step 1) by procedures analogous to those outlined in Example 96, Step 2. The isomers were separated on prep-HPLC on a CSH-FP column (28.9-48.9% MeCN/0.1% TFA (aq.)) to afford two isomers: isomer 3 (t_{R}=4.57) and isomer 4 (t_{R}=5.07). Isomer 3: LC-MS calc. for C₂₅H₂₉N₄O₃ [M+H]⁺: m/z=433.2; Found=433.2. Isomer 4: LC-MS calc. for C₂₅H₂₉N₄O₃ [M+H]⁺: m/z=433.1; Found=433.1.

### Example 102: 2-((1-(2-(3-Azabicyclo[3.1.0]hexan-3-yl)-3-(difluoromethyl)-6-methyl-4-oxo-3,4-dihydroquinazolin-8-yl)ethyl)amino)benzoic acid

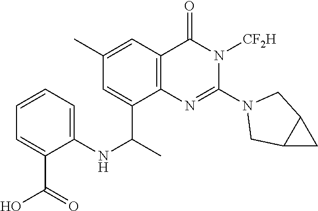 [SMILES: Cc1cc(C(C)Nc2ccccc2C(=O)O)c2nc(N3CC4CC4C3)n(C(F)F)c(=O)c2c1]

### Step 1. 2-(3-Azabicyclo[3.1.0]hexan-3-yl)-8-bromo-3-(4-methoxybenzyl)-6-methylquinazolin-4(3H)-one

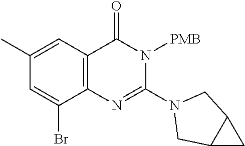

The title compound was synthesized by procedures analogous to those outlined in Example 1, Step 1-4, substituting (4-methoxyphenyl)methanamine for methylamine in Step 1 and substituting 3-azabicyclo[3.1.0]hexane hydrochloride for isoindoline in Step 4. LC-MS calc. for C₂₂H₂₃BrN₃O₂ [M+H]⁺: m/z=440.1, 442.1; Found: 440.1, 442.1.

### Step 2. 2-(3-Azabicyclo[3.1.0]hexan-3-yl)-8-bromo-6-methylquinazolin-4(3H)-one

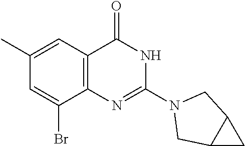 [SMILES: Cc1cc(Br)c2nc(N3CC4CC4C3)[nH]c(=O)c2c1]

A solution of 2-(3-azabicyclo[3.1.0]hexan-3-yl)-8-bromo-3-[(4-methoxyphenyl)methyl]-6-methylquinazolin-4-one (220 mg, 0.50 mmol) in TFA (3 mL) was stirred at 45° C. for 2 h. The reaction mixture was cooled to room temperature and concentrated. The crude material, which contained the title compound, was carried forward without further purification. LC-MS calc. for C₁₄H₁₅BrN₃O [M+H]⁺: m/z=320.0, 322.0; Found: 319.9, 321.9.

### Step 3. 2-(3-Azabicyclo[3.1.0]hexan-3-yl)-8-bromo-3-(difluoromethyl)-6-methylquinazolin-4(3H)-one

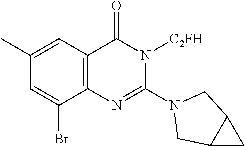

To a solution of 2-(3-azabicyclo[3.1.0]hexan-3-yl)-8-bromo-6-methyl-3H-quinazolin-4-one (120 mg, 0.38 mmol) in diglyme (2.0 mL) was added sodium tert-butoxide (79 mg, 0.83 mmol). The solution was stirred at −15° C. for 10 min. (Bromodifluoromethyl)trimethylsilane (0.07 mL, 0.5 mmol) was added dropwise. The resulting solution was stirred at −15° C. for 1 h. The reaction mixture was warmed to room temperature and diluted with EtOAc (5 mL) and brine (5 mL). The organic phase was collected, and the aqueous phase was extracted again with EtOAc (2×5 mL). The combined organic layer was washed with brine (6×5 mL), dried over Na₂SO₄, and concentrated. The residue was purified by silica gel chromatography (0-100% EtOAc/hexanes) to afford the title compound (55 mg, 0.15 mmol, 40% yield). LC-MS calc. for C₁₅H₁₅BrF₂N₃O [M+H]⁺: m/z=370.0, 372.0; Found: 370.0, 372.0.

### Step 4. 2-((1-(2-(3-Azabicyclo[3.1.0]hexan-3-yl)-3-(difluoromethyl)-6-methyl-4-oxo-3,4-dihydroquinazolin-8-yl)ethyl)amino)benzoic acid

The title compound was synthesized by procedures analogous to those outlined in Example 1, Step 5-7. LC-MS calc. for C₂₄H₂₅F₂N₄O₃ [M+H]⁺: m/z=455.2; Found: 455.1.

### Example 103: 2-((1-(2-(3-Azabicyclo[3.1.0]hexan-3-yl)-6-methyl-4-oxo-3-(2,2,2-trifluoroethyl)-3,4-dihydroquinazolin-8-yl)ethyl)amino)benzoic acid

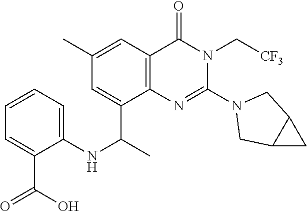 [SMILES: Cc1cc(C(C)Nc2ccccc2C(=O)O)c2nc(N3CC4CC4C3)n(CC(F)(F)F)c(=O)c2c1]

The title compound was synthesized by procedures analogous to those outlined in Example 1, Step 1-7, substituting 2,2,2-trifluoroethylamine hydrochloride for methylamine in Step 1. LCMS calc. for C₂₅H₂₆F₃N₄O₃ [M+H]⁺: 487.2; Found: 487.1.

### Example 104: 2-((1-(2-(3-Azabicyclo[3.1.0]hexan-3-yl)-6-methyl-3-(methyl-d3)-4-oxo-3,4-dihydroquinazolin-8-yl)ethyl)amino)benzoic acid (isomer 2)

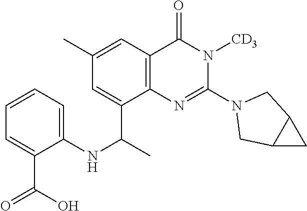 [SMILES: [2H]C([2H])([2H])n1c(N2CC3CC3C2)nc2c(C(C)Nc3ccccc3C(=O)O)cc(C)cc2c1=O]

### Step 1. 2-(3-Azabicyclo[3.1.0]hexan-3-yl)-8-(1-hydroxyethyl)-6-methyl-3-(methyl-d3)quinazolin-4(3H)-one

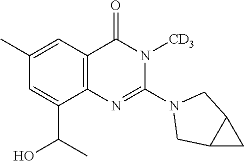 [SMILES: [2H]C([2H])([2H])n1c(N2CC3CC3C2)nc2c(C(C)O)cc(C)cc2c1=O]

The title compound was synthesized by procedures analogous to those outlined in Example 1, Step 1-6, substituting methyl-d3-amine hydrochloride for methylamine in Step 1 and substituting 3-azabicyclo[3.1.0]hexane hydrochloride for isoindoline in Step 4. LC-MS calc. C₁₇H₁₉D₃N₃O₂ [M+H]⁺ m/z=303.2; Found 303.0.

### Step 2. 1-(1-(2-(3-Azabicyclo[3.1.0]hexan-3-yl)-6-methyl-3-(methyl-d3)-4-oxo-3,4-dihydroquinazolin-8-yl)ethyl)-2H-benzo[d][1,3]oxazine-2,4(1H)-dione

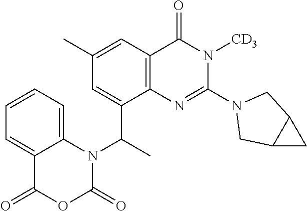 [SMILES: [2H]C([2H])([2H])n1c(N2CC3CC3C2)nc2c(C(C)n3c(=O)oc(=O)c4ccccc43)cc(C)cc2c1=O]

To a solution of isatoic anhydride (572 mg, 3.51 mmol) and triphenylphosphine (919 mg, 3.51 mmol) in THF (5 mL) was added diisopropyl azodicarboxylate (690 uL, 3.51 mmol) at 0° C. 2-(3-Azabicyclo[3.1.0]hexan-3-yl)-8-(1-hydroxyethyl)-6-methyl-3-(trideuteriomethyl)quinazolin-4-one (530 mg, 1.75 mmol) in THF (3 mL) was added. The mixture was stirred at 0° C. for 15 min and warmed up to room temperature over 30 min. The mixture was concentrated and purified by prep-IPLC on C18 column (10-60% MeCN/0.05% TFA (aq))) to afford the title compound (390 mg, 0.87 mmol, 50% yield), a colorless solid. ¹H NMR (300 MHz, DMSO-d₆) δ 8.05 (dd, J=7.8, 1.6 Hz, 1H), 7.87-7.74 (m, 3H), 7.69 (d, J=8.6 Hz, 1H), 7.35 (t, J=7.5 Hz, 1H), 6.32-6.16 (m, 1H), 3.49-3.43 (m, 2H), 3.17-3.04 (m, 2H), 2.43 (s, 3H), 1.89 (d, J=7.0 Hz, 3H), 1.43-1.34 (m, 1H), 1.34-1.27 (m, 1H), 0.38 (td, J=7.7, 4.7 Hz, 1H), 0.00 (q, J=4.2 Hz, 1H). LCMS calc. for C₂₅H₂₂D₃N₄O₄ [M+H]⁺ m/z=448.2; Found 447.9.

### Step 3. 1-(1-(2-(3-Azabicyclo[3.1.0]hexan-3-yl)-6-methyl-3-(methyl-d3)-4-oxo-3,4-dihydroquinazolin-8-yl)ethyl)-2H-benzo[d][1,3]oxazine-2,4(1H)-dione (isomer 2)

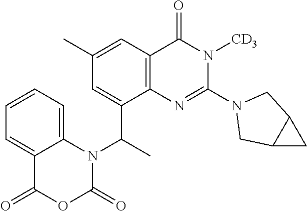 [SMILES: [2H]C([2H])([2H])n1c(N2CC3CC3C2)nc2c(C(C)n3c(=O)oc(=O)c4ccccc43)cc(C)cc2c1=O]

The isomers were separated using chiral prep-HPLC on a Lux iA1 column (30 mL/min, 60:20:20 hexane/IPA/MeOH) to afford two isomers: isomer 1 (t_{R}=2.97 min) and isomer 2 (t_{R}=3.88 min). Isomer 2: LC-MS calc. for C₂₅H₂₂D3N404 [M+H]⁺ m/z=448.2; Found 447.9.

### Step 4. 2-((1-(2-(3-Azabicyclo[3.1.0]hexan-3-yl)-6-methyl-3-(methyl-d3)-4-oxo-3,4-dihydroquinazolin-8-yl)ethyl)amino)benzoic acid (isomer 2)

The title compound was synthesized by procedures analogous to those outlined in Example 45, Step 2. ¹H NMR (400 MHz, DMSO) δ 7.78 (dd, J=7.9, 1.7 Hz, 1H), 7.66 (dd, J=2.1, 0.9 Hz, 1H), 7.42 (d, J=2.1 Hz, 1H), 7.17 (ddd, J=8.6, 7.1, 1.7 Hz, 1H), 6.54-6.43 (m, 2H), 5.36 (q, J=6.6 Hz, 1H), 3.99 (d, J=10.5 Hz, 1H), 3.82 (d, J=10.5 Hz, 1H), 3.59-3.29 (m, 2H), 2.29 (s, 3H), 1.61 (dt, J=7.3, 3.4 Hz, 2H), 1.55 (d, J=6.7 Hz, 3H), 0.59 (td, J=7.6, 4.6 Hz, 1H), 0.37 (q, J=4.1 Hz, 1H). LC-MS calc. for C₂₄H₂₄D₃N₄O₃ [M+H]⁺ m/z=422.2; Found 422.2.

### Example 105: 2-((1-(2-(3-Azabicyclo[3.1.0]hexan-3-yl)-6-chloro-3-methyl-4-oxo-3,4-dihydroquinazolin-8-yl)ethyl)amino)benzoic acid (isomer 2)

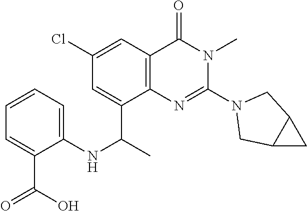 [SMILES: CC(Nc1ccccc1C(=O)O)c1cc(Cl)cc2c(=O)n(C)c(N3CC4CC4C3)nc12]

### Step 1. 1-(1-(2-(3-Azabicyclo[3.1.0]hexan-3-yl)-6-chloro-3-methyl-4-oxo-3,4-dihydroquinazolin-8-yl)ethyl)-2H-benzo[d][1,3]oxazine-2,4(1H)-dione

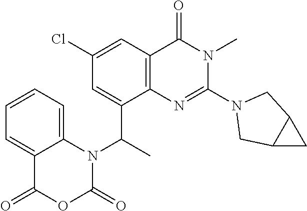 [SMILES: CC(c1cc(Cl)cc2c(=O)n(C)c(N3CC4CC4C3)nc12)n1c(=O)oc(=O)c2ccccc21]

The title compound was synthesized by procedures analogous to those outlined in Example 104, Step 1-2, substituting methyl-d3-amine hydrochloride for methylamine in Step 1 and substituting 3-azabicyclo[3.1.0]hexane hydrochloride for isoindoline in Step 4. LCMS calc. for C₂₄H₂₂ClN₄O₄ [M+H]⁺ m/z=465.1; Found 465.3.

### Step 2. 1-(1-(2-(3-Azabicyclo[3.1.0]hexan-3-yl)-6-chloro-3-methyl-4-oxo-3,4-dihydroquinazolin-8-yl)ethyl)-2H-benzo[d][1,3]oxazine-2,4(1H)-dione (isomer 2)

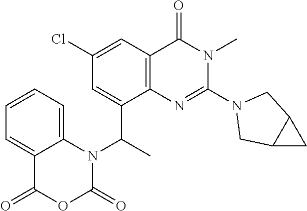 [SMILES: CC(c1cc(Cl)cc2c(=O)n(C)c(N3CC4CC4C3)nc12)n1c(=O)oc(=O)c2ccccc21]

The isomers were separated using chiral prep-HPLC on a Lux iA1 column (25 mL/min, 65:17.5:17.5 hexane/IPA/MeOH) to afford two isomers: isomer 1 (t_{R}=3.42 min) and isomer 2 (t_{R}=4.40 min). Isomer 2: LC-MS calc. for C₂₄H₂₂C₁N404 [M+H]⁺ m/z=465.1; Found 465.3.

### Step 3. 2-((1-(2-(3-Azabicyclo[3.1.0]hexan-3-yl)-6-chloro-3-methyl-4-oxo-3,4-dihydroquinazolin-8-yl)ethyl)amino)benzoic acid (isomer 2)

The title compound was synthesized by procedures analogous to those outlined in Example 45, Step 2. ¹H NMR (400 MHz, DMSO) δ 8.39 (s, 1H), 7.84-7.76 (m, 2H), 7.51 (d, J=2.6 Hz, 1H), 7.19 (ddd, J=8.7, 7.1, 1.8 Hz, 1H), 6.52 (t, J=7.3 Hz, 1H), 6.40 (d, J=8.5 Hz, 1H), 5.33 (q, J=6.8 Hz, 1H), 4.04 (d, J=10.6 Hz, 1H), 3.89 (d, J=10.6 Hz, 1H), 3.55 (dd, J=23.6, 10.5 Hz, 2H), 3.43 (s, 3H), 1.62 (dd, J=7.1, 3.4 Hz, 2H), 1.57 (d, J=6.7 Hz, 3H), 0.67-0.55 (m, 1H), 0.32 (q, J=4.2 Hz, 1H). LC-MS calc. for C₂₃H₂₄ClN₄O₃ [M+H]⁺ m/z=439.2; Found 439.1.

### Examples 106-117

Examples 106-117 listed in Tables 15 and 16 were synthesized according to procedures analogous to Example 41, [Step 4] (Method A), Example 69, (Method B), or Example 67 (Method C). The appropriate starting materials for Methods A, B, and C can be prepared according to procedures analogous to those described herein. All examples in this table were prepared as the TFA salt unless otherwise noted.

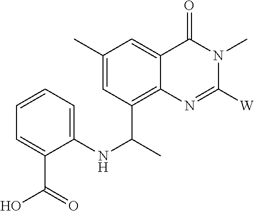

TABLE 15
Examples 106-117

| Example | W | Method | LCMS [M + H]⁺ |
|---|---|---|---|
| 106 | 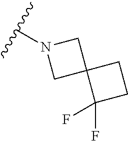 [SMILES: *N1CC2(CCC2(F)F)C1] | A | 469.4 |
| 107 | 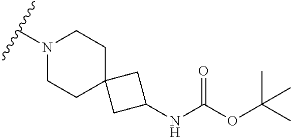 [SMILES: *N1CCC2(CC1)CC(NC(=O)OC(C)(C)C)C2] | A | 576.5 |
| 108 | 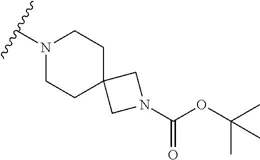 [SMILES: *N1CCC2(CC1)CN(C(=O)OC(C)(C)C)C2] | A | 562.6 |
| 109 | 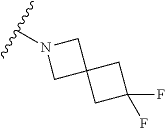 [SMILES: *N1CC2(C1)CC(F)(F)C2] | A | 469.4 |
| 110 | 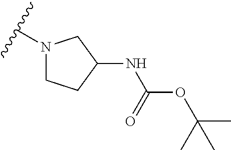 [SMILES: *N1CCC(NC(=O)OC(C)(C)C)C1] | A | 522.5 |
| 111 | 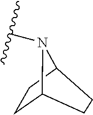 [SMILES: *N1C2CCC1CC2] | B | 433.4 |
| 112 | 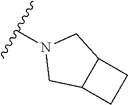 [SMILES: *N1CC2CCC2C1] | B | 433.2 |
| 113 | 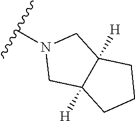 [SMILES: *N1C[C@H]2CCC[C@H]2C1] | B | 447.2 |
| 114 | 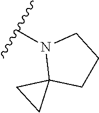 [SMILES: *N1CCCC12CC2] | C | 431.9 |
| 115 | 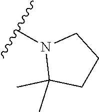 [SMILES: *N1CCCC1(C)C] | C | 433.8 |
| 116 | 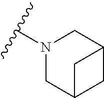 [SMILES: *N1CC2CC(C2)C1] | A | 433.2 |
| 117 | 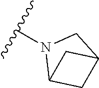 [SMILES: *N1CC2CC1C2] | A | 419.2 |

TABLE 16
Examples 106-117

| Example | Compound name | NMR |
|---|---|---|
| 106 | 2-[1-[2-(7,7-difluoro-2- | ¹H NMR (300 MHz, DMSO-d₆) δ 8.43 (s, |
| | azaspiro[3.3]heptan-2-yl)-3,6- | 1H), 7.77 (dd, J = 8.2, 1.7 Hz, 1H), 7.67 |
| | dimethyl-4-oxoquinazolin-8- | (d, J = 2.1 Hz, 1H), 7.42 (d, J = 2.1 Hz, |
| | yl]ethylamino]benzoic acid | 1H), 7.28-7.10 (m, 1H), 6.48 (t, J = 7.5 |
| | | Hz, 2H), 5.36 (d, J = 7.2 Hz, 1H), 4.46 |
| | | (dd, J = 9.3, 5.4 Hz, 2H), 4.25 (d, J = 9.3 |
| | | Hz, 2H), 3.38 (s, 3H), 2.50 (p, J = 1.9 Hz, |
| | | 2H), 2.29 (s, 3H), 2.09 (t, J = 6.0 Hz, 2H), |
| | | 1.56 (d, J = 6.6 Hz, 3H). |
| 107 | 2-[1-[3,6-dimethyl-2-[2-[(2- | ¹H NMR (300 MHz, DMSO-d₆) δ 8.41 (s, |
| | methylpropan-2-yl)oxycarbonyl- | 1H), 7.79 (dd, J = 8.2, 1.6 Hz, 1H), 7.71 |
| | amino]-7-azaspiro[3.5]nonan-7- | (s, 1H), 7.47 (d, J = 1.8 Hz, 1H), 7.25- |
| | yl]-4-oxoquinazolin-8-yl]ethyl- | 7.11 (m, 2H), 6.55-6.45 (m, 2H), 5.41 |
| | amino]benzoic acid | (d, J = 6.6 Hz, 1H), 4.05-3.88 (m, 1H), |
| | | 3.48 (s, 3H), 3.15 (d, J = 22.1 Hz, 4H), |
| | | 2.33 (s, 3H), 2.18 (t, J = 9.9 Hz, 2H), 1.70 |
| | | (d, J = 12.5 Hz, 6H), 1.57 (d, J = 6.6 Hz, |
| | | 3H), 1.39 (s, 9H). |
| 108 | 2-[1-[3,6-dimethyl-2-[2-[(2- | ¹H NMR (300 MHz, DMSO-d₆) δ 8.44 (s, |
| | methylpropan-2-yl)oxy- | 1H), 7.83-7.76 (m, 1H), 7.73 (s, 1H), |
| | carbonyl]-2,7-diazaspiro[3.5] | 7.48 (s, 1H), 7.20 (t, J = 7.8 Hz, 1H), 6.50 |
| | nonan-7-yl]-4-oxoquinazolin-8- | (dd, J = 12.7, 5.7 Hz, 2H), 5.40 (s, 1H), |
| | yl]ethylamino]benzoic acid | 3.65 (s, 4H), 3.49 (s, 3H), 3.12 (dd, J = |
| | | 7.3, 4.8 Hz, 2H), 2.33 (s, 3H), 1.91 (d, J = |
| | | 12.0 Hz, 4H), 1.57 (d, J = 6.7 Hz, 3H), |
| | | 1.40 (s, 9H), 1.19 (t, J = 7.3 Hz, 2H). |
| 109 | 2-[1-[2-(6,6-difluoro-2- | ¹H NMR (300 MHz, DMSO-d₆) δ 12.63 |
| | azaspiro[3.3]heptan-2-yl)-3,6- | (s, 1H), 8.45 (s, 1H), 7.79 (dd, J = 8.0, 1.5 |
| | dimethyl-4-oxoquinazolin-8- | Hz, 1H), 7.67 (d, J = 1.2 Hz, 1H), 7.42 (d, |
| | yl]ethylamino]benzoic acid | J = 1.9 Hz, 1H), 7.27-7.15 (m, 1H), 6.50 |
| | | (t, J = 7.7 Hz, 2H), 5.35 (d, J = 6.8 Hz, |
| | | 1H), 4.44-4.33 (m, 4H), 3.40 (s, 3H), |
| | | 2.91 (t, J = 12.5 Hz, 4H), 2.30 (s, 3H), |
| | | 1.57 (d, J = 6.6 Hz, 3H). |
| 110 | 2-[1-[3,6-dimethyl-2-[3-[(2- | ¹H NMR (300 MHz, DMSO-d6) δ 12.69 |
| | methylpropan-2-yl)oxycarbonyl- | (s, 1H), 8.43 (s, 1H), 7.79 (dd, J = 8.0, 1.6 |
| | amino]pyrrolidin-1-yl]-4- | Hz, 1H), 7.68 (s, 1H), 7.41 (s, 1H), 7.31- |
| | oxoquinazolin-8-yl]ethylamino] | 7.16 (m, 2H), 6.49 (dd, J = 13.6, 6.5 Hz, |
| | benzoic acid | 2H), 5.38 (s, 1H), 4.13 (s, 1H), 3.83- |
| | | 3.67 (m, 2H), 3.62 (d, J = 6.7 Hz, 1H), |
| | | 3.47 (s, 4H), 2.30 (s, 3H), 2.00 (ddd, J = |
| | | 41.8, 21.0, 5.9 Hz, 2H), 1.62-1.52 (m, |
| | | 3H), 1.41 (s, 9H). |
| 111 | 2-[1-[2-(7-azabicyclo[2.2.1] | ¹H NMR (300 MHz, DMSO-d₆) δ 7.76 |
| | heptan-7-yl)-3,6-dimethyl-4- | (d, J = 7.9 Hz, 1H), 7.66 (s, 1H), 7.40 |
| | oxoquinazolin-8-yl]ethylamino] | (d, J = 2.1 Hz, 1H), 7.12 (s, 1H), 6.48 (s, |
| | benzoic acid | 1H), 6.37 (d, J = 8.5 Hz, 1H), 5.40 (d, J = |
| | | 6.8 Hz, 1H), 4.34 (d, J = 4.2 Hz, 2H), |
| | | 3.51 (s, 3H), 2.27 (s, 3H), 1.90 (d, J = |
| | | 13.1 Hz, 4H), 1.49 (t, J = 6.6 Hz, 7H). |
| 112 | 2-[1-[2-(3-azabicyclo[3.2.0] | ¹H NMR (300 MHz, DMSO-d₆) δ 8.42 (s, |
| | heptan-3-yl)-3,6-dimethyl-4- | 1H), 7.76 (dd, J = 8.2, 1.7 Hz, 1H), 7.71 |
| | oxoquinazolin-8-yl]ethylamino] | (d, J = 2.1 Hz, 1H), 7.45 (d, J = 2.1 Hz, |
| | benzoic acid | 1H), 7.16 (d, J = 1.9 Hz, 1H), 6.47 (t, J = |
| | | 7.5 Hz, 2H), 5.38 (d, J = 6.9 Hz, 1H), |
| | | 3.70-3.62 (m, 2H), 3.61 (s, 3H), 3.21 |
| | | (dd, J = 10.1, 5.2 Hz, 2H), 2.99 (s, 2H), |
| | | 2.31 (s, 3H), 2.20 (d, J = 4.8 Hz, 2H), |
| | | 1.82 (d, J = 8.3 Hz, 2H), 1.55 (d, J = 6.6 |
| | | Hz, 3H). |
| 113 | 2-[1-[3,6-dimethyl-4-oxo-2- | ¹H NMR (300 MHz, DMSO-d₆) δ 8.40 (s, |
| | [(3aR,6aS)-3,3a,4,5,6,6a- | 1H), 7.77 (dd, J = 7.9, 1.7 Hz, 1H), 7.68 |
| | hexahydro-1H-cyclopenta[c] | (d, J = 2.0 Hz, 1H), 7.43 (d, J = 2.1 Hz, |
| | pyrrol-2-yl]quinazolin-8- | 1H), 7.17 (ddd, J = 8.6, 7.1, 1.7 Hz, 1H), |
| | yl]ethylamino]benzoic acid | 6.55-6.30 (m, 2H), 5.38 (d, J = 6.7 Hz, |
| | | 1H), 3.59 (td, J = 10.7, 7.3 Hz, 2H), 3.48 |
| | | (s, 3H), 3.24 (ddd, J = 15.9, 10.7, 3.4 Hz, |
| | | 2H), 2.71 (s, 2H), 2.30 (s, 3H), 1.75 |
| | | (q, J = 8.4, 5.9 Hz, 3H), 1.53 (dd, J = |
| | | 12.2, 7.2 Hz, 6H). |
| 114 | 2-((1-(3,6-dimethyl-4-oxo-2-(4- | ¹H NMR (400 MHz, DMSO-d₆) δ 12.64 |
| | azaspiro[2.4]heptan-4-yl)-3,4- | (s, 1H), 8.37 (s, 1H), 7.77 (dd, J = 8.0, 1.7 |
| | dihydroquinazolin-8-yl)ethyl) | Hz, 1H), 7.68 (d, J = 2.0 Hz, 1H), 7.41 (d, |
| | amino)benzoic acid | J = 2.1 Hz, 1H), 7.14 (ddd, J = 8.7, 7.0, |
| | | 1.7 Hz, 1H), 6.48 (t, J = 7.5 Hz, 1H), 6.38 |
| | | (d, J = 8.5 Hz, 1H), 5.53 (q, J = 6.7 Hz, |
| | | 1H), 3.59-3.54 (m, 2H), 3.43 (s, 3H), |
| | | 2.29 (s, 3H), 2.08-1.83 (m, 4H), 1.51 (d, |
| | | J = 6.7 Hz, 3H), 0.96-0.68 (m, 4H). |
| 115 | 2-((1-(2-(2,2-dimethyl- | ¹H NMR (600 MHz, DMSO-d₆) δ 12.69 |
| | pyrrolidin-1-yl)-3,6-dimethyl-4- | (s, 1H), 8.35 (s, 1H), 7.76 (dd, J = 7.9, 1.6 |
| | oxo-3,4-dihydroquinazolin-8- | Hz, 1H), 7.63 (dd, J = 2.2, 1.0 Hz, 1H), |
| | yl)ethyl)amino)benzoic acid | 7.36 (d, J = 2.1 Hz, 1H), 7.12 (ddd, J = |
| | | 8.7, 7.0, 1.7 Hz, 1H), 6.46 (ddd, J = 8.0, |
| | | 7.0, 1.0 Hz, 1H), 6.28 (dd, J = 8.7, 1.1 Hz, |
| | | 1H), 5.40 (q, J = 6.7 Hz, 1H), 3.52 (t, J = |
| | | 7.1 Hz, 2H), 3.40 (s, 3H), 2.25 (s, 3H), |
| | | 1.93 (pd, J = 6.9, 1.8 Hz, 2H), 1.77 (t, J = |
| | | 7.1 Hz, 2H), 1.57 (d, J = 11.7 Hz, 6H), |
| | | 1.47 (d, J = 6.7 Hz, 3H). |
| 116 | 2-((1-(2-(3-Azabicyclo[3.1.1] | — |
| | heptan-3-yl)-3,6-dimethyl-4- | |
| | oxo-3,4-dihydroquinazolin-8- | |
| | yl)ethyl)amino)benzoic acid | |
| 117 | 2-((1-(2-(2-Azabicyclo[2.1.1] | — |
| | hexan-2-yl)-3,6-dimethyl-4-oxo- | |
| | 3,4-dihydroquinazolin-8- | |
| | yl)ethyl)amino)benzoic acid | |

### Examples 118-126

Examples 118-126 listed in Tables 17 and 18 were synthesized according to procedures analogous to Example 41, [Step 4] (Method A) or Example 28 (Method B). All examples in this table were prepared as the TFA salt unless otherwise noted.

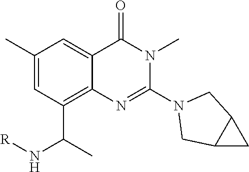

TABLE 17
Examples 118-126

| Example | R | Method | LCMS [M + H]⁺ |
|---|---|---|---|
| 118 | 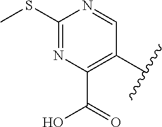 [SMILES: *c1cnc(SC)nc1C(=O)O] | B | 467.1 |
| 119 | 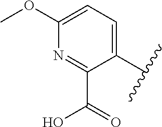 [SMILES: *c1ccc(OC)nc1C(=O)O] | A | 450.4 |
| 120 | 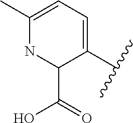 [SMILES: *C1=CC=C(C)NC1C(=O)O] | A | 434.0 |
| 121 | 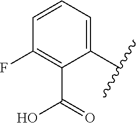 [SMILES: *c1cccc(F)c1C(=O)O] | A | 437.0 |
| 122 | 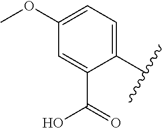 [SMILES: *c1ccc(OC)cc1C(=O)O] | A | 449.4 |
| 123 | 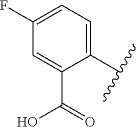 [SMILES: *c1ccc(F)cc1C(=O)O] | A | 436.9 |
| 124 | 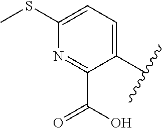 [SMILES: *c1ccc(SC)nc1C(=O)O] | A | 466.3 |
| 125 | 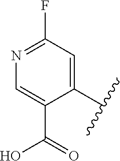 [SMILES: *c1cc(F)ncc1C(=O)O] | B | 438.1 |
| 126 | 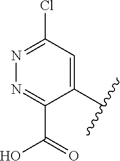 [SMILES: *c1cc(Cl)nnc1C(=O)O] | B | 455.1 |

TABLE 18
Examples 118-126

| Example | Compound name | NMR |
|---|---|---|
| 118 | 5-((1-(2-(3-Azabicyclo[3.1.0] | ¹H NMR (400 MHz, DMSO-d₆) δ 8.25 (s, 1H), |
| | hexan-3-yl)-3,6-dimethyl-4-oxo- | 7.69 (d, J = 2.1 Hz, 1H), 7.51 (d, J = 2.1 Hz, |
| | 3,4-dihydroquinazolin-8- | 1H), 5.45-5.34 (m, 1H), 3.97 (d, J = 10.5 Hz, |
| | yl)ethyl)amino)-2-(methylthio) | 1H), 3.80 (d, J = 10.4 Hz, 1H), 3.50 (t, J = 12.1 |
| | pyrimidine-4-carboxylic acid | Hz, 2H), 3.41 (s, 3H), 2.42 (s, 3H), 2.31 (s, 3H), |
| | | 1.67-1.54 (m, 5H), 0.62-0.50 (m, 1H), 0.43- |
| | | 0.26 (m, 1H). |
| 119 | 3-[1-[2-(3-Azabicyclo[3.1.0] | ¹H NMR (300 MHz, DMSO-d₆) δ 8.08 (s, 1H), |
| | hexan-3-yl)-3,6-dimethyl-4- | 7.66 (dd, J = 2.1, 0.9 Hz, 1H), 7.41 (d, J = 2.1 |
| | oxoquinazolin-8-yl]ethylamino]- | Hz, 1H), 7.05 (d, J = 9.2 Hz, 1H), 6.83 (d, J = |
| | 6-methoxypyridine-2-carboxylic | 9.1 Hz, 1H), 5.40-5.24 (m, 1H), 3.96 (d, J = |
| | acid | 10.5 Hz, 1H), 3.81 (d, J = 10.5 Hz, 1H), 3.77 (s, |
| | | 3H), 3.49 (t, J = 11.2 Hz, 2H), 3.41 (s, 3H), 2.29 |
| | | (s, 3H), 1.64-1.57 (m, 2H), 1.56 (d, J = 6.6 |
| | | Hz, 3H), 0.58 (td, J = 7.7, 4.6 Hz, 1H), 0.35 |
| | | (d, J = 4.2 Hz, 1H). |
| 120 | 3-[1-[2-(3-Azabicyclo[3.1.0] | — |
| | hexan-3-yl)-3,6-dimethyl-4- | |
| | oxoquinazolin-8-yl]ethylamino]- | |
| | 6-methylpyridine-2-carboxylic | |
| | acid | |
| 121 | 2-[1-[2-(3-Azabicyclo[3.1.0] | ¹H NMR (300 MHz, DMSO-d₆) δ 8.17 (s, 1H), |
| | hexan-3-yl)-3,6-dimethyl-4- | 7.66 (d, J = 2.1 Hz, 1H), 7.41 (d, J = 2.1 Hz, |
| | oxoquinazolin-8-yl]ethylamino]- | 1H), 7.19-7.06 (m, 1H), 6.29 (dd, J = 11.2, 8.2 |
| | 6-fluorobenzoic acid | Hz, 2H), 5.36-5.26 (m, 1H), 3.98 (d, J = 10.5 |
| | | Hz, 1H), 3.80 (d, J = 10.5 Hz, 1H), 3.58-3.45 |
| | | (m, 2H), 3.42 (s, 3H), 2.30 (s, 3H), 1.65-1.55 |
| | | (m, 2H), 1.53 (d, J = 6.6 Hz, 3H), 0.63-0.53 |
| | | (m, 1H), 0.40-0.30 (m, 1H). |
| 122 | 2-[1-[2-(3-Azabicyclo[3.1.0] | ¹H NMR (300 MHz, DMSO-d₆) δ 7.65 (s, 1H), |
| | hexan-3-yl)-3,6-dimethyl-4- | 7.39 (s, 1H), 7.29 (d, J = 2.9 Hz, 1H), 6.88 |
| | oxoquinazolin-8-yl]ethylamino]- | (dd, J = 9.1, 3.0 Hz, 1H), 6.39 (d, J = 9.2 Hz, |
| | 5-methoxybenzoic acid | 1H), 5.36-5.26 (m, 1H), 3.97 (d, J = 10.5 Hz, |
| | | 1H), 3.82 (d, J = 10.5 Hz, 1H), 3.61 (s, 3H), |
| | | 3.56-3.46 (m, 2H), 3.43 (s, 3H), 2.28 (s, 3H), |
| | | 1.65-1.55 (m, 2H), 1.52 (d, J = 6.5 Hz, 3H), |
| | | 0.64-0.54 (m, 1H), 0.42-0.32 (m, 1H). |
| 123 | 2-[1-[2-(3-Azabicyclo[3.1.0] | ¹H NMR (300 MHz, DMSO-d₆) δ 8.17 (s, 1H), |
| | hexan-3-yl)-3,6-dimethyl-4- | 7.66 (s, 1H), 7.48 (dd, J = 9.8, 3.1 Hz, 1H), 7.40 |
| | oxoquinazolin-8-yl]ethylamino]- | (d, J = 2.1 Hz, 1H), 7.10 (td, J = 8.6, 3.2 Hz, |
| | 5-fluorobenzoic acid | 1H), 6.42 (dd, J = 9.4, 4.5 Hz, 1H), 5.36-5.26 |
| | | (m, 1H), 3.97 (d, J = 10.5 Hz, 1H), 3.82 (d, J = |
| | | 10.5 Hz, 1H), 3.59-3.45 (m, 2H), 3.42 (s, 3H), |
| | | 2.29 (s, 3H), 1.65-1.55 (m, 2H), 1.54 (d, J = |
| | | 6.5 Hz, 3H), 0.65-0.55 (m, 1H), 0.41-0.31 |
| | | (m, 1H). |
| 124 | 3-[1-[2-(3-Azabicyclo[3.1.0] | ¹H NMR (300 MHz, DMSO-d₆) δ 8.23 (s, 1H), |
| | hexan-3-yl)-3,6-dimethyl-4- | 7.68 (dd, J = 2.1, 1.0 Hz, 1H), 7.44 (d, J = 2.1 |
| | oxoquinazolin-8-yl]ethylamino]- | Hz, 1H), 7.22 (d, J = 8.9 Hz, 1H), 6.96 (d, J = |
| | 6-methylsulfanylpyridine-2- | 9.1 Hz, 1H), 5.42-5.23 (m, 1H), 3.97 (d, J = |
| | carboxylic acid | 10.5 Hz, 1H), 3.83 (d, J = 10.4 Hz, 1H), 3.56- |
| | | 3.46 (m, 2H), 3.43 (s, 3H), 2.47 (s, 3H), 2.31 (s, |
| | | 3H), 1.68-1.61 (m, 2H), 1.59 (d, J = 6.7 Hz, |
| | | 3H), 0.60 (td, J = 7.6, 4.6 Hz, 1H), 0.37 (q, J = |
| | | 4.2 Hz, 1H). |
| 125 | 4-((1-(2-(3-Azabicyclo[3.1.0] | ¹H NMR (400 MHz, DMSO-d₆) δ 7.66 (s, 1H), |
| | hexan-3-yl)-3,6-dimethyl-4-oxo- | 7.61 (s, 1H), 7.42 (s, 1H), 6.52 (s, 1H), 5.92- |
| | 3,4-dihydroquinazolin-8-yl)ethyl) | 5.82 (m, 1H), 3.84 (t, J = 10.6 Hz, 2H), 3.54- |
| | amino)-6-fluoronicotinic acid | 3.43 (m, 1H), 3.41 (s, 3H), 2.31 (s, 3H), 1.65- |
| | | 1.53 (m, 5H), 0.61-0.50 (m, 1H), 0.40-0.32 |
| | | (m, 1H). |
| 126 | 4-((1-(2-(3-Azabicyclo[3.1.0] | — |
| | hexan-3-yl)-3,6-dimethyl-4-oxo- | |
| | 3,4-dihydroquinazolin-8-yl)ethyl) | |
| | amino)-6-chloropyridazine-3- | |
| | carboxylic acid | |

### Example A: PI3K Pathway Activation Assay

The inhibitory activity of compounds was evaluated by measuring phosphorylation of AKT on Ser473 as a readout of the PI3K pathway using HTRF (CisBio catalog number: 64AKSPE). These studies were conducted in the T-47D (heterozygous PIK3CA H1047R) and SKBR3 (PIK3CA WT) cell lines. Cells were maintained in a 37° C. incubator at 5% CO₂ in the following media: T-47D: RPMI 1640, ATCC© Modification (Gibco, A10491-01) supplemented with 10% v/v FBS (Gibco, 26140-079), 1% penicillin streptomycin (Gibco, 15140-122), and 7.4 ug/mL insulin (MilliporeSigma, I9278); SKBR3: McCoy's 5a (Modified) Medium (Gibco, 16600-082) supplemented with 10% v/v FBS (Gibco, 26140-079). Cells were seeded in 384-well plates at a density of 4,000 cells/well. Compounds dissolved in DMSO were added using a digital dispense (D300E, Tecan) 10-point serial dilution. After two hours of treatment, cells were lysed for thirty minutes and then incubated with detection reagents per HTRF kit material and manufacturer's instructions. Fluorescence signal was measured with a multimode plate reader (Envision 2105, Perkin Elmer). Fluorescent signal was normalized to background and DMSO controls to obtain percent inhibition/activity for each compound. The results are summarized in Table 19.

TABLE 19
IC₅₀ Values

| Example | T47D pAKT IC₅₀ | SKBR3 PAKT IC₅₀ |
|---|---|---|
| 1 | +++ | + |
| 2 | +++ | + |
| 3 | ++ | −− |
| 4 | ++ | + |
| 5 | ++ | −− |
| 6 | +++ | + |
| 7 | ++ | + |
| 8 | ++ | + |
| 9 | ++ | + |
| 10 | ++ | + |
| 11 | ++ | + |
| 12 | ++ | −− |
| 13 | ++ | −− |
| 14 | ++ | −− |
| 15 | +++ | −− |
| 16 | ++ | −− |
| 17 | +++ | −− |
| 18 | ++ | −− |
| 19 | ++ | + |
| 20 | +++ | + |
| 21 | +++ | + |
| 22 | +++ | + |
| 23 | ++ | + |
| 24 | +++ | + |
| 25 | ++ | −− |
| 26 | ++ | −− |
| 27 | +++ | + |
| 28 | ++ | + |
| 29 | ++ | −− |
| 30 | ++ | −− |
| 31 | +++ | + |
| 32 | +++ | −− |
| 33 | ++ | −− |
| 34 | ++ | + |
| 35 | +++ | + |
| 36 | ++ | −− |
| 37 | +++ | −− |
| 38 | ++ | −− |
| 39 | +++ | + |
| 41 | ++ | + |
| 42 | ++ | −− |
| 43 | ++ | + |
| 44 | ++ | −− |
| 45 | ++ | −− |
| 46 | ++ | + |
| 47 | ++ | −− |
| 48 | +++ | + |
| 49 | +++ | + |
| 50 | ++ | −− |
| 51 | ++ | −− |
| 52 | ++ | −− |
| 53 | ++ | −− |
| 54 | ++ | + |
| 55 | +++ | + |
| 56 | ++ | −− |
| 57 | ++ | −− |
| 58 | +++ | + |
| 59 | ++ | −− |
| 60 | ++ | −− |
| 61 | ++ | −− |
| 62 | ++ | −− |
| 63 | ++ | −− |
| 64 | ++ | −− |
| 65 | ++ | −− |
| 66 | ++ | −− |
| 67 | +++ | + |
| 68 | +++ | + |
| 69 | ++ | + |
| 70 | +++ | + |
| 71 | ++ | −− |
| 72 | ++ | −− |
| 73 | ++ | −− |
| 74 | +++ | −− |
| 75 | ++ | −− |
| 76 | ++ | −− |
| 77 | ++ | −− |
| 78 | ++ | −− |
| 79 | ++ | −− |
| 80 | ++ | −− |
| 81 | +++ | −− |
| 82 | +++ | + |
| 83 | +++ | −− |
| 84 | +++ | −− |
| 85 | +++ | −− |
| 86 | +++ | −− |
| 87 | ++ | −− |
| 88 | ++ | −− |
| 89 | ++ | −− |
| 90 | ++ | + |
| 91 | +++ | −− |
| 92 | ++ | −− |
| 93 | ++ | −− |
| 94 | +++ | −− |
| 95 | +++ | −− |
| 96 | ++ | −− |
| 97 | ++ | −− |
| 98 | +++ | + |
| 99 | +++ | + |
| 100 | +++ | + |
| 101 | ++ | −− |
| 102 | ++ | −− |
| 103 | ++ | −− |
| 104 | +++ | + |
| 105 | +++ | + |
| 106 | +++ | −− |
| 107 | ++ | −− |
| 108 | ++ | −− |
| 109 | +++ | −− |
| 110 | ++ | −− |
| 111 | +++ | −− |
| 112 | +++ | −− |
| 113 | +++ | −− |
| 114 | ++ | −− |
| 115 | +++ | −− |
| 116 | +++ | −− |
| 117 | ++ | −− |
| 118 | ++ | −− |
| 119 | ++ | + |
| 120 | ++ | + |
| 121 | +++ | −− |
| 122 | ++ | −− |
| 123 | +++ | + |
| 124 | +++ | −− |
| 125 | ++ | −− |
| 126 | ++ | −− |

In Table 9, a “+” denotes an IC₅₀ value of >10000 nM; a “++” denotes an IC₅₀ value of 500 nM < IC₅₀ ≤ 10000 nM; a “+++” denotes an IC₅₀ value of <500 nM.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I) 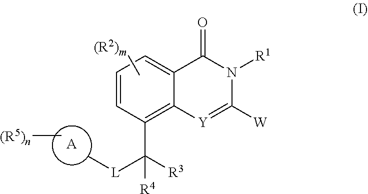
or a pharmaceutically acceptable salt thereof, wherein
ring A is aryl, a 3-8 membered cycloalkyl ring, a 5-7 membered heteroaryl ring comprising 1-4 heteroatoms selected from N, O, and S, or a 5-7 membered heterocyclic group comprising 1-4 heteroatoms selected from N, O, and S;
W is a 5-10 membered heteroaryl ring comprising 1-4 heteroatoms selected from N, O, and S, or a 5-12 membered heterocyclic group comprising 1-4 heteroatoms selected from N, O, and S, C₁-C₈ alkyl, haloalkyl, —C₂-C₆ alkenyl, —C₂-C₆ alkynyl, aryl, heteroaryl, cycloalkyl, cycloalkenyl, heterocycloalkenyl, NR^{c}R^{d}, OR^{b}, or SR^{b}; wherein each group is optionally substituted by 1-6 R^{f} groups;
Y is CR² or N;
n is 1, 2, 3, 4 or 5;
m is 1, 2 or 3;
L is a bond, O, S, NR^{a} or C₁-C₆ alkylene;
R¹ is H, D, OR^{a}, C₁-C₈ alkoxide, C₁-C₈ alkyl, haloalkyl, —C₃-C₈ cycloalkyl, —C₃-C₁₀ cycloalkenyl, aryl, or heteroaryl; wherein said C₁-C₈ alkoxide, C₁-C₈ alkyl, haloalkyl, C₃-C₈ cycloalkyl, —C₃-C₁₀ cycloalkenyl, aryl, and heteroaryl are optionally substituted by 1-6 R^{f} groups;
each R² and R⁵ is independently H, D, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
each R³ and R⁴ is independently H, D, halogen, C₁-C₈ alkoxide, C₁-C₈ alkyl, haloalkyl, or CN; wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d};
or R³ and R⁴ together with the atom to which they are attached are combined to form a C₃-C₇ cycloalkyl or C₄-C₈ heterocycloalkyl;
each R^{a} is independently H, D, —C(O)R^{b}, —C(O)OR^{c}, —C(O)NR^{c}R^{d}, —C(═NR^{b})NR^{b}R^{c}, —C(═NOR^{b})R^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group.
R^{e} is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
each R^{f} is independently H, D, oxo, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{b}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

2. The compound of claim 1, wherein ring A is aryl or a 5-7 membered heteroaryl ring comprising 1-4 heteroatoms selected from N, O, and S.

3-4. (canceled)

5. The compound of claim 1, wherein W is a 5-12 membered heterocyclic group comprising 1-4 heteroatoms selected from N, O, and S optionally substituted by 1-6 R^{f} groups or W is NR^{c}R^{d}.

6-7. (canceled)

8. The compound of claim 1, wherein Y is N.

9-16. (canceled)

17. The compound of claim 1, wherein L is NR^{a}.

18. (canceled)

19. The compound of claim 1, wherein R¹ is H or C₁-C₈ alkyl optionally substituted by 1-6 R^{f} groups.

20-23. (canceled)

24. The compound of claim 1, wherein at least one R² is H, C₁-C₈ alkyl, CF₃, Br, F, CN or CHF₂.

25-26. (canceled)

27. The compound of claim 1, wherein R³ is H or C₁-C₈ alkyl.

28-29. (canceled)

30. The compound of claim 1, wherein R⁴ is H or C₁-C₈ alkyl.

31. (canceled)

32. The compound of claim 1, wherein at least one R⁵ is H or —CO₂H.

33. (canceled)

34. The compound of claim 1, in the form of a pharmaceutically acceptable salt.

35. The compound of claim 1, that is a compound of formula (II) 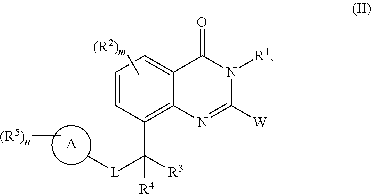
or a pharmaceutically acceptable salt or stereoisomer thereof.

36. The compound of claim 1, that is a compound of formula (III) 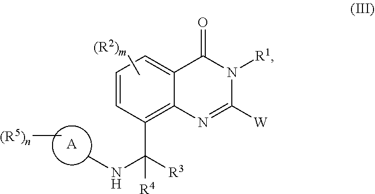
or a pharmaceutically acceptable salt or stereoisomer thereof.

37. The compound of claim 1, that is a compound of formula (IV) 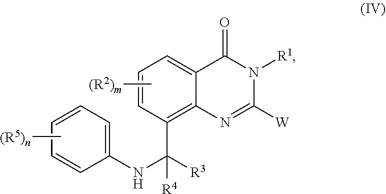
or a pharmaceutically acceptable salt or stereoisomer thereof.

38. The compound of claim 1, that is a compound of formula (V) 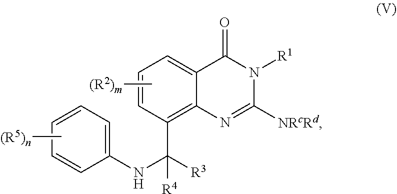
or a pharmaceutically acceptable salt or stereoisomer thereof.

39. The compound of claim 1, that is a compound of formula (VI) 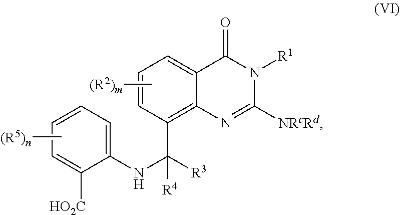
or a pharmaceutically acceptable salt or stereoisomer thereof.

40. The compound of claim 1 that is:
2-((1-(2-(isoindolin-2-yl)-3,6-dimethyl-4-oxo-3,4-dihydroquinazolin-8-yl)ethyl)amino) benzoic acid;
2-((1-(2-(5-fluoroisoindolin-2-yl)-3,6-dimethyl-4-oxo-3,4-dihydroquinazolin-8-yl)ethyl) amino)benzoic acid;
2-((1-(2-(4,4-dimethylpiperidin-1-yl)-3,6-dimethyl-4-oxo-3,4-dihydroquinazolin-8-yl)ethyl) amino)benzoic acid;
or a pharmaceutically acceptable salt thereof.

41. The compound of claim 1 that is:
2-((1-(2-(isoindolin-2-yl)-3,6-dimethyl-4-oxo-3,4-dihydroquinazolin-8-yl)ethyl)amino) benzamide;
2-((1-(2-(isoindolin-2-yl)-3,6-dimethyl-4-oxo-3,4-dihydroquinazolin-8-yl)ethyl)amino)-N-methoxybenzamide;
2-((1-(2-(isoindolin-2-yl)-3,6-dimethyl-4-oxo-3,4-dihydroquinazolin-8-yl)ethyl)amino) benzenesulfonamide;
2-((1-(2-(isoindolin-2-yl)-3,6-dimethyl-4-oxo-3,4-dihydroquinazolin-8-yl)ethyl)amino)-N-methylbenzenesulfonamide;
2-(isoindolin-2-yl)-3,6-dimethyl-8-(1-((2-(methylsulfonyl)phenyl)amino)ethyl) quinazolin-4(3H)-one;
8-(1-((2-(1H-tetrazol-5-yl)phenyl)amino)ethyl)-2-(isoindolin-2-yl)-3,6-dimethylquinazolin-4(3H)-one;
N-hydroxy-2-((1-(2-(isoindolin-2-yl)-3,6-dimethyl-4-oxo-3,4-dihydroquinazolin-8-yl)ethyl) amino)benzamide;
8-(1-((2,4-difluoro-3-hydroxyphenyl)amino)ethyl)-2-(isoindolin-2-yl)-3,6-dimethyl-quinazolin-4(3H)-one;
2-(isoindolin-2-yl)-3,6-dimethyl-8-(1-((2-(2,2,2-trifluoro-1-hydroxyethyl)phenyl)amino) ethyl)quinazolin-4(3H)-one;
2-((1-(2-(isoindolin-2-yl)-3,6-dimethyl-4-oxo-3,4-dihydroquinazolin-8-yl)ethyl)thio) benzoic acid;
2-((1-(2-(3-azabicyclo[3.1.0]hexan-3-yl)-5-cyano-3,6-dimethyl-4-oxo-3,4-dihydro-quinazolin-8-yl)ethyl)amino)benzoic acid;
2-((1-(2-(isoindolin-2-yl)-3-methyl-4-oxo-6-(trifluoromethyl)-3,4-dihydroquinazolin-8-yl)ethyl)amino)benzoic acid;
2-((1-(2-(3-azabicyclo[3.1.0]hexan-3-yl)-3-methyl-4-oxo-6-(trifluoromethyl)-3,4-dihydroquinazolin-8-yl)ethyl)amino)benzoic acid;
2-((1-(2-(4,4-dimethylpiperidin-1-yl)-3,6-dimethyl-4-oxo-3,4-dihydroquinazolin-8-yl) ethyl)amino)benzoic acid;
2-((1-(2-(isoindolin-2-yl)-6-methyl-4-oxo-3-(pyridin-3-yl)-3,4-dihydroquinazolin-8-yl) ethyl)amino)benzoic acid;
2-((1-(2-(isoindolin-2-yl)-3-(2-methoxyethyl)-6-methyl-4-oxo-3,4-dihydroquinazolin-8-yl) ethyl)amino)benzoic acid;
2,3-difluoro-6-((1-(3-(3-fluoropropyl)-2-(isoindolin-2-yl)-6-methyl-4-oxo-3,4-dihydro-quinazolin-8-yl)ethyl)amino)benzoic acid;
2-((1-(2-(isoindolin-2-yl)-3-isopropyl-6-methyl-4-oxo-3,4-dihydroquinazolin-8-yl)ethyl) amino)benzoic acid;
2-((1-(2-(isoindolin-2-yl)-6-methyl-4-oxo-3,4-dihydroquinazolin-8-yl)ethyl)amino) benzoic acid;
2-((1-(2-(3-azabicyclo[3.1.0]hexan-3-yl)-3,6-dimethyl-4-oxo-3,4-dihydroquinazolin-8-yl) ethyl)amino)-4-cyanobenzoic acid;
2-((1-(2-(3-azabicyclo[3.1.0]hexan-3-yl)-3,6-dimethyl-4-oxo-3,4-dihydroquinazolin-8-yl) ethyl)amino)benzoic acid;
2-((1-(3,6-dimethyl-4-oxo-2-(1H-pyrazol-5-yl)-3,4-dihydroquinazolin-8-yl)ethyl)amino) benzoic acid;
2-((1-(2-amino-3,6-dimethyl-4-oxo-3,4-dihydroquinazolin-8-yl)ethyl)amino)benzoic acid;
2-((1-(2-(isoindolin-2-yl)-3-isopropyl-6-methyl-4-oxo-3,4-dihydroquinazolin-8-yl)ethyl) amino)benzoic acid;
3-((1-(2-(isoindolin-2-yl)-3,6-dimethyl-4-oxo-3,4-dihydroquinazolin-8-yl)ethyl)amino) picolinic acid;
2-((1-(2-(isoindolin-2-yl)-3,6-dimethyl-4-oxo-3,4-dihydroquinazolin-8-yl)ethyl)amino)-6-methoxybenzoic acid;
2-((1-(2-(isoindolin-2-yl)-3,6-dimethyl-4-oxo-3,4-dihydroquinazolin-8-yl)ethyl)amino)-5-(trifluoromethyl)benzoic acid;
5-cyano-2-((1-(2-(isoindolin-2-yl)-3,6-dimethyl-4-oxo-3,4-dihydroquinazolin-8-yl)ethyl) amino)benzoic acid;
4-fluoro-2-((1-(2-(isoindolin-2-yl)-3,6-dimethyl-4-oxo-3,4-dihydroquinazolin-8-yl)ethyl) amino)benzoic acid;
5-chloro-2-((1-(2-(isoindolin-2-yl)-3,6-dimethyl-4-oxo-3,4-dihydroquinazolin-8-yl)ethyl) amino)benzoic acid;
4-((1-(2-(isoindolin-2-yl)-3,6-dimethyl-4-oxo-3,4-dihydroquinazolin-8-yl)ethyl)amino)-1-methyl-1H-pyrazole-3-carboxylic acid;
2,3-difluoro-6-((1-(2-(isoindolin-2-yl)-3,6-dimethyl-4-oxo-3,4-dihydroquinazolin-8-yl) ethyl)amino)benzoic acid;
2-((1-(2-(isoindolin-2-yl)-3,6-dimethyl-4-oxo-3,4-dihydroquinazolin-8-yl)ethyl)amino) nicotinic acid;
2-((1-(2-(4,4-difluoropiperidin-1-yl)-3,6-dimethyl-4-oxo-3,4-dihydroquinazolin-8-yl)ethyl)amino)benzoic acid;
2-((1-(5-acrylamido-2-(3-azabicyclo[3.1.0]hexan-3-yl)-3,6-dimethyl-4-oxo-3,4-dihydroquinazolin-8-yl)ethyl)amino)benzoic acid;
2-[1-[2-(5-azaspiro[2.4]heptan-5-yl)-3,6-dimethyl-4-oxoquinazolin-8-yl]ethylamino]benzoic acid;
2-[1-(2-methoxy-3,6-dimethyl-4-oxoquinazolin-8-yl)ethylamino]benzoic acid;
2-((1-(2-hydroxy-3,6-dimethyl-4-oxo-3,4-dihydroquinazolin-8-yl)ethyl)amino)benzoic acid;
2-[1-(3,6-dimethyl-2-methylsulfanyl-4-oxoquinazolin-8-yl)ethylamino]benzoic acid;
2-((1-(2-(4-cyanopiperidin-1-yl)-3,6-dimethyl-4-oxo-3,4-dihydroquinazolin-8-yl)ethyl) amino)benzoic acid;
3-fluoro-2-((1-(2-(isoindolin-2-yl)-3,6-dimethyl-4-oxo-3,4-dihydroquinazolin-8-yl)ethyl) amino)benzoic acid;
2-((1-(2-(4-acetylpiperazin-1-yl)-3,6-dimethyl-4-oxo-3,4-dihydroquinazolin-8-yl)ethyl) amino)benzoic acid;
2-[[1-[2-(3-azabicyclo[3.1.0]hexan-3-yl)-3,6-dimethyl-4-oxoquinazolin-8-yl]-2,2,2-trifluoroethyl]amino]benzoic acid;
((1-(3-((1r,3r)-3-Hydroxycyclobutyl)-2-(isoindolin-2-yl)-6-methyl-4-oxo-3,4-dihydro-quinazolin-8-yl)ethyl)amino)benzoic acid;
2-((1-(2-(3-azabicyclo[3.1.0]hexan-3-yl)-6-fluoro-3-methyl-4-oxo-3,4-dihydro-quinazolin-8-yl)ethyl)amino)benzoic acid;
2-((1-(6-cyano-2-(isoindolin-2-yl)-3-methyl-4-oxo-3,4-dihydroquinazolin-8-yl)ethyl) amino)benzoic acid;
2-[1-[2-(3,4-dihydro-1H-isoquinolin-2-yl)-3,6-dimethyl-4-oxoquinazolin-8-yl]ethyl-amino]benzoic acid 2-[1-[3,6-dimethyl-4-oxo-2-(3-phenylpyrrolidin-1-yl)quinazolin-8-yl]ethylamino]benzoic acid;
2-[1-[3,6-dimethyl-4-oxo-2-(3-phenylpyrrolidin-1-yl)quinazolin-8-yl]ethylamino]benzoic acid;
2-[1-[2-(dimethylamino)-3,6-dimethyl-4-oxoquinazolin-8-yl]ethylamino]benzoic acid;
2-((1-(2-(azepan-1-yl)-3,6-dimethyl-4-oxo-3,4-dihydroquinazolin-8-yl)ethyl)amino) benzoic acid;
2-((1-(2-(1,1-dioxidothiomorpholino)-3,6-dimethyl-4-oxo-3,4-dihydroquinazolin-8-yl) ethyl)amino)benzoic acid;
2-((1-(2-(4-hydroxypiperidin-1-yl)-3,6-dimethyl-4-oxo-3,4-dihydroquinazolin-8-yl) ethyl)amino)benzoic acid;
or a pharmaceutically acceptable salt thereof.

42. The compound of claim 1 that is:
2-((1-(2-((1R,5S,6s)-6-((methoxycarbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6s)-6-(((benzyloxy)carbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-4-oxo-2-((1R,5S,6s)-6-(((2,2,2-trifluoroethoxy)carbonyl)amino)-3-azabicyclo[3.1.0]hexan-3-yl)-3,4-dihydroquinazolin-8-yl)ethyl)amino)benzoic acid;
2-((1-(3,6-dimethyl-2-((1R,5S,6s)-6-(((2-morpholinoethoxy)carbonyl)amino)-3-azabicyclo[3.1.0]hexan-3-yl)-4-oxo-3,4-dihydroquinazolin-8-yl)ethyl)amino)benzoic acid;
2-((1-(2-((1R,5S,6s)-6-(((4-methoxyphenoxy)carbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6s)-6-((cyclopropoxycarbonyl)amino)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-4-oxo-2-((1R,5S,6s)-6-((((tetrahydrofuran-2-yl)methoxy)carbonyl) amino)-3-azabicyclo[3.1.0]hexan-3-yl)-3,4-dihydroquinazolin-8-yl)ethyl)amino)benzoic acid;
2-((1-(2-(5-(tert-butoxycarbonyl)-2,5-diazabicyclo[2.2.1]heptan-2-yl)-3,6-dimethyl-4-oxo-3,4-dihydroquinazolin-8-yl)ethyl)amino)benzoic acid;
2-((1-(2-((3aR,6aR)-5-acetylhexahydropyrrolo[3,4-c]pyrrol-2(1H)-yl)-3,6-dimethyl-4-oxo-3,4-dihydroquinazolin-8-yl)ethyl)amino)benzoic acid;
2-((1-(2-(5,7-dihydro-6H-pyrrolo[3,4-b]pyridin-6-yl)-3,6-dimethyl-4-oxo-3,4-dihydroquinazolin-8-yl)ethyl)amino)benzoic acid;
methyl 2-[1-[2-(3-azabicyclo[3.1.0]hexan-3-yl)-3-methyl-4-oxo-6-(trideuteriomethyl) quinazolin-8-yl]ethylamino]benzoate;
2-((1-(2-(isoindolin-2-yl)-3,6-dimethyl-4-oxo-3,4-dihydroquinazolin-8-yl)ethyl)amino)-4-methylbenzoic;
2-((1-(6-bromo-2-(isoindolin-2-yl)-3-methyl-4-oxo-3,4-dihydroquinazolin-8-yl)ethyl) amino)benzoic acid;
2-[1-[3,6-dimethyl-4-oxo-2-(1-prop-2-enoylpyrrolidin-2-yl)quinazolin-8-yl]ethylamino]benzoic acid;
2-((1-(2-(isoindolin-2-yl)-3-methyl-4-oxo-3,4-dihydroquinazolin-8-yl)ethyl)amino) benzoic acid;
2-[1-[2-(1-fluorosulfonylpyrrolidin-2-yl)-3,6-dimethyl-4-oxoquinazolin-8-yl]ethyl-amino]benzoic acid;
2-((1-(6-bromo-2-(4,4-difluoropiperidin-1-yl)-3-methyl-4-oxo-3,4-dihydroquinazolin-8-yl)ethyl)amino)benzoic acid;
2-((1-(3-((1r,3r)-3-cyanocyclobutyl)-2-(isoindolin-2-yl)-6-methyl-4-oxo-3,4-dihydro-quinazolin-8-yl)ethyl)amino)benzoic acid;
2-((1-(2-(3-azabicyclo[3.1.0]hexan-3-yl)-6-cyclopropyl-3-methyl-4-oxo-3,4-dihydro-quinazolin-8-yl)ethyl)amino)benzoic acid;
2-((1-(2-(3-azabicyclo[3.1.0]hexan-3-yl)-6-methoxy-3-methyl-4-oxo-3,4-dihydroquinazolin-8-yl)ethyl)amino)benzoic acid;
2-((1-(2-(3-azabicyclo[3.1.0]hexan-3-yl)-6-ethyl-3-methyl-4-oxo-3,4-dihydroquinazolin-8-yl)ethyl)amino)benzoic acid;
2-((1-(2-(3-azabicyclo[3.1.0]hexan-3-yl)-6-(difluoromethyl)-3-methyl-4-oxo-3,4-dihydro-quinazolin-8-yl)ethyl)amino)benzoic acid;
3-((1-(2-(3-azabicyclo[3.1.0]hexan-3-yl)-3,6-dimethyl-4-oxo-3,4-dihydroquinazolin-8-yl)ethyl)amino)-6-(trifluoromethyl)picolinic acid;
2-((1-(2-((1R,5S,6R)-6-(isopropyl(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-(cyclobutyl(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-(methoxy(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-((1R,5S,6R)-6-((2,2-difluoroethyl)(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-2-((1R,5S,6R)-6-((S)-2-methylpyrrolidine-1-carbonyl)-3-azabicyclo [3.1.0]hexan-3-yl)-4-oxo-3,4-dihydroquinazolin-8-yl)ethyl)amino)benzoic acid;
2-((1-(3,6-dimethyl-2-((1R,5S,6R)-6-((S)-3-methylmorpholine-4-carbonyl)-3-azabicyclo [3.1.0]hexan-3-yl)-4-oxo-3,4-dihydroquinazolin-8-yl)ethyl)amino)benzoic acid;
2-((1-(2-((1R,5S,6R)-6-((2-methoxyethyl)(methyl)carbamoyl)-3-azabicyclo[3.1.0]hexan-3-yl)-3,6-dimethyl-4-oxo-3,4-dihydroquinazolin-8-yl)ethyl)amino)benzoic acid;
2-((1-(3,6-dimethyl-2-((1R,5S,6R)-6-(methyl(pyridin-3-yl)carbamoyl)-3-azabicyclo[3.1.0] hexan-3-yl)-4-oxo-3,4-dihydroquinazolin-8-yl)ethyl)amino)benzoic acid;
2-((1-(3,6-dimethyl-2-((1R,5S,6R)-6-(methylcarbamoyl)-3-azabicyclo[3.1.0]hexan-3-yl)-4-oxo-3,4-dihydroquinazolin-8-yl)ethyl)amino)benzoic acid;
2-((1-(2-((1R,5S,6R)-6-(dimethylcarbamoyl)-3-azabicyclo[3.1.0]hexan-3-yl)-3,6-dimethyl-4-oxo-3,4-dihydroquinazolin-8-yl)ethyl)amino)benzoic acid;
2-((1-(2-(4-(cyclopentanecarbonyl)piperazin-1-yl)-3,6-dimethyl-4-oxo-3,4-dihydro-quinazolin-8-yl)ethyl)amino)benzoic acid;
2-((1-(2-(4-(3-fluorobicyclo[1.1.1]pentane-1-carbonyl)piperazin-1-yl)-3,6-dimethyl-4-oxo-3,4-dihydroquinazolin-8-yl)ethyl)amino)benzoic acid;
2-((1-(2-(4-(2-methoxyacetyl)piperazin-1-yl)-3,6-dimethyl-4-oxo-3,4-dihydroquinazolin-8-yl)ethyl)amino)benzoic acid;
2-((1-(3,6-dimethyl-2-(4-(6-methylnicotinoyl)piperazin-1-yl)-4-oxo-3,4-dihydroquinazolin-8-yl)ethyl)amino)benzoic acid;
2-((1-(2-(3-azabicyclo[3.1.0]hexan-3-yl)-5-fluoro-3,6-dimethyl-4-oxo-3,4-dihydro-quinazolin-8-yl)ethyl)amino)benzoic acid;
2-((1-(2-(3-azabicyclo[3.1.0]hexan-3-yl)-3,6-dimethyl-4-oxo-3,4-dihydroquinazolin-8-yl)-2,2-difluoroethyl)amino)benzoic acid;
2-((1-(2-(3-azabicyclo[3.1.0]hexan-3-yl)-3,6-dimethyl-4-oxo-3,4-dihydroquinazolin-8-yl)propyl)amino)benzoic acid;
2-((1-(3-(3,3-bis(hydroxymethyl)cyclobutyl)-2-(isoindolin-2-yl)-6-methyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(2-azabicyclo[2.2.1]heptan-2-yl)-3,6-dimethyl-4-oxo-3,4-dihydroquinazolin-8-yl)ethyl)amino)benzoic acid;
2-((1-(2-(3-azabicyclo[3.1.0]hexan-3-yl)-3-(difluoromethyl)-6-methyl-4-oxo-3,4-dihydroquinazolin-8-yl)ethyl)amino)benzoic acid;
2-((1-(2-(3-azabicyclo[3.1.0]hexan-3-yl)-6-methyl-4-oxo-3-(2,2,2-trifluoroethyl)-3,4-dihydroquinazolin-8-yl)ethyl)amino)benzoic acid;
2-((1-(2-(3-azabicyclo[3.1.0]hexan-3-yl)-6-methyl-3-(methyl-d3)-4-oxo-3,4-dihydro-quinazolin-8-yl)ethyl)amino)benzoic acid;
2-((1-(2-(3-azabicyclo[3.1.0]hexan-3-yl)-6-chloro-3-methyl-4-oxo-3,4-dihydroquinazolin-8-yl)ethyl)amino)benzoic acid;
2-[1-[2-(7,7-difluoro-2-azaspiro[3.3]heptan-2-yl)-3,6-dimethyl-4-oxoquinazolin-8-yl]ethyl-amino]benzoic acid;
2-[1-[3,6-dimethyl-2-[2-[(2-methylpropan-2-yl)oxycarbonylamino]-7-azaspiro[3.5]nonan-7-yl]-4-oxoquinazolin-8-yl]ethylamino]benzoic acid;
2-[1-[3,6-dimethyl-2-[2-[(2-methylpropan-2-yl)oxycarbonyl]-2,7-diazaspiro[3.5]nonan-7-yl]-4-oxoquinazolin-8-yl]ethylamino]benzoic acid;
2-[1-[2-(6,6-difluoro-2-azaspiro[3.3]heptan-2-yl)-3,6-dimethyl-4-oxoquinazolin-8-yl]ethyl-amino]benzoic acid;
2-[1-[3,6-dimethyl-2-[3-[(2-methylpropan-2-yl)oxycarbonylamino]pyrrolidin-1-yl]-4-oxoquinazolin-8-yl]ethylamino]benzoic acid;
2-[1-[2-(7-azabicyclo[2.2.1]heptan-7-yl)-3,6-dimethyl-4-oxoquinazolin-8-yl]ethyl-amino] benzoic acid;
2-[1-[2-(3-azabicyclo[3.2.0]heptan-3-yl)-3,6-dimethyl-4-oxoquinazolin-8-yl]ethyl-amino] benzoic acid;
2-[1-[3,6-dimethyl-4-oxo-2-[rac-(3aR,6aS)-3,3a,4,5,6,6a-hexahydro-1H-cyclopenta[c]pyrrol-2-yl]quinazolin-8-yl]ethylamino]benzoic acid;
2-((1-(3,6-dimethyl-4-oxo-2-(4-azaspiro[2.4]heptan-4-yl)-3,4-dihydroquinazolin-8-yl)ethyl) amino)benzoic acid;
2-((1-(2-(2,2-dimethylpyrrolidin-1-yl)-3,6-dimethyl-4-oxo-3,4-dihydroquinazolin-8-yl)ethyl) amino)benzoic acid;
2-((1-(2-(3-azabicyclo[3.1.1]heptan-3-yl)-3,6-dimethyl-4-oxo-3,4-dihydroquinazolin-8-yl)ethyl)amino)benzoic acid;
2-((1-(2-(2-azabicyclo[2.1.1]hexan-2-yl)-3,6-dimethyl-4-oxo-3,4-dihydroquinazolin-8-yl)ethyl)amino)benzoic acid;
5-((1-(2-(3-azabicyclo[3.1.0]hexan-3-yl)-3,6-dimethyl-4-oxo-3,4-dihydroquinazolin-8-yl)ethyl)amino)-2-(methylthio)pyrimidine-4-carboxylic acid;
3-[1-[2-(3-azabicyclo[3.1.0]hexan-3-yl)-3,6-dimethyl-4-oxoquinazolin-8-yl]ethyl-amino]-6-methoxypyridine-2-carboxylic acid;
3-[1-[2-(3-azabicyclo[3.1.0]hexan-3-yl)-3,6-dimethyl-4-oxoquinazolin-8-yl]ethyl-amino]-6-methylpyridine-2-carboxylic acid;
2-[1-[2-(3-azabicyclo[3.1.0]hexan-3-yl)-3,6-dimethyl-4-oxoquinazolin-8-yl]ethyl-amino]-6-fluorobenzoic acid;
2-[1-[2-(3-azabicyclo[3.1.0]hexan-3-yl)-3,6-dimethyl-4-oxoquinazolin-8-yl]ethyl-amino]-5-methoxybenzoic acid;
2-[1-[2-(3-azabicyclo[3.1.0]hexan-3-yl)-3,6-dimethyl-4-oxoquinazolin-8-yl]ethyl-amino]-5-fluorobenzoic acid;
3-[1-[2-(3-azabicyclo[3.1.0]hexan-3-yl)-3,6-dimethyl-4-oxoquinazolin-8-yl]ethyl-amino]-6-methylsulfanylpyridine-2-carboxylic acid;
4-((1-(2-(3-azabicyclo[3.1.0]hexan-3-yl)-3,6-dimethyl-4-oxo-3,4-dihydroquinazolin-8-yl)ethyl)amino)-6-fluoronicotinic acid;
4-((1-(2-(3-azabicyclo[3.1.0]hexan-3-yl)-3,6-dimethyl-4-oxo-3,4-dihydroquinazolin-8-yl)ethyl)amino)-6-chloropyridazine-3-carboxylic acid;
or a pharmaceutically acceptable salt thereof.

43. A pharmaceutical composition comprising a compound according to claim 1, or a pharmaceutically acceptable salt thereof, and a pharmaceutically acceptable excipient.

44. A method of treating a disease or disorder associated with modulation of phosphoinositide 3-kinase (PI3K), comprising administering to a patient in need thereof a therapeutically effective amount of a compound of claim 1 or a pharmaceutical composition comprising the compound.

45. The method of claim 44, wherein the PI3K is PI3Kα.

46. The method of claim 44, wherein the PI3K associated with the disease or disorder has a H1047R mutation.

47. The method of claim 44, wherein the disease or disorder is a cancer; optionally wherein the cancer is endometrial cancer, gastric cancer, leukemia, lymphoma, sarcoma, colorectal cancer, lung cancer, ovarian cancer, skin cancer, head and neck cancer, breast cancer, brain cancer, cervical cancer, bladder cancer, esophageal cancer, pancreatic cancer, bone cancer, hepatobiliary cancer, medulloblastoma, kidney cancer or prostate cancer.

48. (canceled)

49. The method of claim 44, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).

50. A method of inhibiting phosphoinositide 3-kinase (PI3K), comprising administering to a patient in need thereof a therapeutically effective amount of a compound of claim 1 or a pharmaceutical composition comprising the compound.

51. A method of treating cancer or a disorder, the method comprising administering to a patient in need thereof a therapeutically effective amount of a compound of claim 1 or a pharmaceutical composition comprising the compound.

52. The method of claim 51, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

53. (canceled)

54. The method of claim 51, wherein the disorder is CLOVES syndrome (congenital lipomatous overgrowth, vascular malformations, epidermal naevi, scoliosis/skeletal and spinal syndrome) or PIK3CA-related overgrowth syndrome (PROS).

55-56. (canceled)

57. A method of degrading a phosphoinositide 3-kinase (PI3K) protein comprising contacting the PI3K protein with a compound of claim 1 or a pharmaceutical composition comprising the compound.

58. The method of claim 57, wherein the PI3K is PI3Kα.
